SEQUENCE LISTING (127 sequences)

SEQ ID NO: 1 - protein, 2351 aa, Homo sapiens
  SIGNAL 1..19: signal
  wt human FVIII
MQIELSTCFFLCLLRFCFSATRRYYLGAVELSWDYMQSDLGELPVDARFPPRVPKSFPFNTSVVYKKTLFVEFTDHLFNIAKPRPPWMGLLGPTIQAEVYDTVVITLKNMASHPVSLHAVGVSYWKASEGAEYDDQTSQREKEDDKVFPGGSHTYVWQVLKENGPMASDPLCLTYSYLSHVDLVKDLNSGLIGALLVCREGSLAKEKTQTLHKFILLFAVFDEGKSWHSETKNSLMQDRDAASARAWPKMHTVNGYVNRSLPGLIGCHRKSVYWHVIGMGTTPEVHSIFLEGHTFLVRNHRQASLEISPITFLTAQTLLMDLGQFLLFCHISSHQHDGMEAYVKVDSCPEEPQLRMKNNEEAEDYDDDLTDSEMDVVRFDDDNSPSFIQIRSVAKKHPKTWVHYIAAEEEDWDYAPLVLAPDDRSYKSQYLNNGPQRIGRKYKKVRFMAYTDETFKTREAIQHESGILGPLLYGEVGDTLLIIFKNQASRPYNIYPHGITDVRPLYSRRLPKGVKHLKDFPILPGEIFKYKWTVTVEDGPTKSDPRCLTRYYSSFVNMERDLASGLIGPLLICYKESVDQRGNQIMSDKRNVILFSVFDENRSWYLTENIQRFLPNPAGVQLEDPEFQASNIMHSINGYVFDSLQLSVCLHEVAYWYILSIGAQTDFLSVFFSGYTFKHKMVYEDTLTLFPFSGETVFMSMENPGLWILGCHNSDFRNRGMTALLKVSSCDKNTGDYYEDSYEDISAYLLSKNNAIEPRSFSQNSRHPSTRQKQFNATTIPENDIEKTDPWFAHRTPMPKIQNVSSSDLLMLLRQSPTPHGLSLSDLQEAKYETFSDDPSPGAIDSNNSLSEMTHFRPQLHHSGDMVFTPESGLQLRLNEKLGTTAATELKKLDFKVSSTSNNLISTIPSDNLAAGTDNTSSLGPPSMPVHYDSQLDTTLFGKKSSPLTESGGPLSLSEENNDSKLLESGLMNSQESSWGKNVSSTESGRLFKGKRAHGPALLTKDNALFKVSISLLKTNKTSNNSATNRKTHIDGPSLLIENSPSVWQNILESDTEFKKVTPLIHDRMLMDKNATALRLNHMSNKTTSSKNMEMVQQKKEGPIPPDAQNPDMSFFKMLFLPESARWIQRTHGKNSLNSGQGPSPKQLVSLGPEKSVEGQNFLSEKNKVVVGKGEFTKDVGLKEMVFPSSRNLFLTNLDNLHENNTHNQEKKIQEEIEKKETLIQENVVLPQIHTVTGTKNFMKNLFLLSTRQNVEGSYDGAYAPVLQDFRSLNDSTNRTKKHTAHFSKKGEEENLEGLGNQTKQIVEKYACTTRISPNTSQQNFVTQRSKRALKQFRLPLEETELEKRIIVDDTSTQWSKNMKHLTPSTLTQIDYNEKEKGAITQSPLSDCLTRSHSIPQANRSPLPIAKVSSFPSIRPIYLTRVLFQDNSSHLPAASYRKKDSGVQESSHFLQGAKKNNLSLAILTLEMTGDQREVGSLGTSATNSVTYKKVENTVLPKPDLPKTSGKVELLPKVHIYQKDLFPTETSNGSPGHLDLVEGSLLQGTEGAIKWNEANRPGKVPFLRVATESSAKTPSKLLDPLAWDNHYGTQIPKEEWKSQEKSPEKTAFKKKDTILSLNACESNHAIAAINEGQNKPEIEVTWAKQGRTERLCSQNPPVLKRHQREITRTTLQSDQEEIDYDDTISVEMKKEDFDIYDEDENQSPRSFQKKTRHYFIAAVERLWDYGMSSSPHVLRNRAQSGSVPQFKKVVFQEFTDGSFTQPLYRGELNEHLGLLGPYIRAEVEDNIMVTFRNQASRPYSFYSSLISYEEDQRQGAEPRKNFVKPNETKTYFWKVQHHMAPTKDEFDCKAWAYFSDVDLEKDVHSGLIGPLLVCHTNTLNPAHGRQVTVQEFALFFTIFDETKSWYFTENMERNCRAPCNIQMEDPTFKENYRFHAINGYIMDTLPGLVMAQDQRIRWYLLSMGSNENIHSIHFSGHVFTVRKKEEYKMALYNLYPGVFETVEMLPSKAGIWRVECLIGEHLHAGMSTLFLVYSNKCQTPLGMASGHIRDFQITASGQYGQWAPKLARLHYSGSINAWSTKEPFSWIKVDLLAPMIIHGIKTQGARQKFSSLYISQFIIMYSLDGKKWQTYRGNSTGTLMVFFGNVDSSGIKHNIFNPPIIARYIRLHPTHYSIRSTLRMELMGCDLNSCSMPLGMESKAISDAQITASSYFTNMFATWSPSKARLHLQGRSNAWRPQVNNPKEWLQVDFQKTMKVTGVTTQGVKSLLTSMYVKEFLISSSQDGHQWTLFFQNGKVKVFQGNQDSFTPVVNSLDPPLLTRYLRIHPQSWVHQIALRMEVLGCEAQDLY

SEQ ID NO: 2 - protein, 9 aa, Artificial Sequence
  minimal processing sequence
PRSFSQNPP

SEQ ID NO: 3 - skipped

SEQ ID NO: 4 - protein, 11 aa, Artificial Sequence
  Processing sequence
PRSFSQNPPVL

SEQ ID NO: 5 - protein, 11 aa, Artificial Sequence
  Processing sequence
  VARIANT 4: can be varied for de-immunization Xaa can be any amino acid selected from the group consisting of Ala, Cys, Asp, Glu, Phe, Gly, His, Ile, Lys, Leu, Met, Asn, Pro, Gln, Arg, Ser, Thr, Val, Trp, or Tyr.
PRSXSQNPPVL

SEQ ID NO: 6 - protein, 11 aa, Artificial Sequence
  Processing sequence
  VARIANT 5: can be varied for de-immunization Xaa can be any amino acid selected from the group consisting of Ala, Cys, Asp, Glu, Phe, Gly, His, Ile, Lys, Leu, Met, Asn, Pro, Gln, Arg, Ser, Thr, Val, Trp, or Tyr.
PRSFXQNPPVL

SEQ ID NO: 7 - protein, 11 aa, Artificial Sequence
  Processing sequence
  VARIANT 6: can be varied for de-immunization Xaa can be any amino acid selected from the group consisting of Ala, Cys, Asp, Glu, Phe, Gly, His, Ile, Lys, Leu, Met, Asn, Pro, Gln, Arg, Ser, Thr, Val, Trp, or Tyr.
PRSFSXNPPVL

SEQ ID NO: 8 - protein, 11 aa, Artificial Sequence
  Processing sequence
  VARIANT 7: can be varied for de-immunization Xaa can be any amino acid selected from the group consisting of Ala, Cys, Asp, Glu, Phe, Gly, His, Ile, Lys, Leu, Met, Asn, Pro, Gln, Arg, Ser, Thr, Val, Trp, or Tyr.
PRSFSQXPPVL

SEQ ID NO: 9 - protein, 10 aa, Artificial Sequence
  Merging sequence, e.g., in V0
QSDQEEIDYD

SEQ ID NO: 10-15 - skipped (6 entries)

SEQ ID NO: 16 - protein, 1445 aa, Artificial Sequence
  SIGNAL 1..19
  V0 aa incl. signal sequence
MQIELSTCFFLCLLRFCFSATRRYYLGAVELSWDYMQSDLGELPVDARFPPRVPKSFPFNTSVVYKKTLFVEFTDHLFNIAKPRPPWMGLLGPTIQAEVYDTVVITLKNMASHPVSLHAVGVSYWKASEGAEYDDQTSQREKEDDKVFPGGSHTYVWQVLKENGPMASDPLCLTYSYLSHVDLVKDLNSGLIGALLVCREGSLAKEKTQTLHKFILLFAVFDEGKSWHSETKNSLMQDRDAASARAWPKMHTVNGYVNRSLPGLIGCHRKSVYWHVIGMGTTPEVHSIFLEGHTFLVRNHRQASLEISPITFLTAQTLLMDLGQFLLFCHISSHQHDGMEAYVKVDSCPEEPQLRMKNNEEAEDYDDDLTDSEMDVVRFDDDNSPSFIQIRSVAKKHPKTWVHYIAAEEEDWDYAPLVLAPDDRSYKSQYLNNGPQRIGRKYKKVRFMAYTDETFKTREAIQHESGILGPLLYGEVGDTLLIIFKNQASRPYNIYPHGITDVRPLYSRRLPKGVKHLKDFPILPGEIFKYKWTVTVEDGPTKSDPRCLTRYYSSFVNMERDLASGLIGPLLICYKESVDQRGNQIMSDKRNVILFSVFDENRSWYLTENIQRFLPNPAGVQLEDPEFQASNIMHSINGYVFDSLQLSVCLHEVAYWYILSIGAQTDFLSVFFSGYTFKHKMVYEDTLTLFPFSGETVFMSMENPGLWILGCHNSDFRNRGMTALLKVSSCDKNTGDYYEDSYEDISAYLLSKNNAIEPRSFSQNPPVLQSDQEEIDYDDTISVEMKKEDFDIYDEDENQSPRSFQKKTRHYFIAAVERLWDYGMSSSPHVLRNRAQSGSVPQFKKVVFQEFTDGSFTQPLYRGELNEHLGLLGPYIRAEVEDNIMVTFRNQASRPYSFYSSLISYEEDQRQGAEPRKNFVKPNETKTYFWKVQHHMAPTKDEFDCKAWAYFSDVDLEKDVHSGLIGPLLVCHTNTLNPAHGRQVTVQEFALFFTIFDETKSWYFTENMERNCRAPCNIQMEDPTFKENYRFHAINGYIMDTLPGLVMAQDQRIRWYLLSMGSNENIHSIHFSGHVFTVRKKEEYKMALYNLYPGVFETVEMLPSKAGIWRVECLIGEHLHAGMSTLFLVYSNKCQTPLGMASGHIRDFQITASGQYGQWAPKLARLHYSGSINAWSTKEPFSWIKVDLLAPMIIHGIKTQGARQKFSSLYISQFIIMYSLDGKKWQTYRGNSTGTLMVFFGNVDSSGIKHNIFNPPIIARYIRLHPTHYSIRSTLRMELMGCDLNSCSMPLGMESKAISDAQITASSYFTNMFATWSPSKARLHLQGRSNAWRPQVNNPKEWLQVDFQKTMKVTGVTTQGVKSLLTSMYVKEFLISSSQDGHQWTLFFQNGKVKVFQGNQDSFTPVVNSLDPPLLTRYLRIHPQSWVHQIALRMEVLGCEAQDLY

SEQ ID NO: 17-27 - skipped (11 entries)

SEQ ID NO: 28 - protein, 1457 aa, Artificial Sequence
  SIGNAL 1..19
  SIGNAL 1..19
  AC-6rs-REF aa incl. signal
MQIELSTCFFLCLLRFCFSATRRYYLGAVELSWDYMQSDLGELPVDARFPPRVPKSFPFNTSVVYKKTLFVEFTDHLFNIAKPRPPWMGLLGPTIQAEVYDTVVITLKNMASHPVSLHAVGVSYWKASEGAEYDDQTSQREKEDDKVFPGGSHTYVWQVLKENGPMASDPLCLTYSYLSHVDLVKDLNSGLIGALLVCREGSLAKEKTQTLHKFILLFAVFDEGKSWHSETKNSLMQDRDAASARAWPKMHTVNGYVNRSLPGLIGCHRKSVYWHVIGMGTTPEVHSIFLEGHTFLVRNHRQASLEISPITFLTAQTLLMDLGQFLLFCHISSHQHDGMEAYVKVDSCPEEPQLRMKNNEEAEDYDDDLTDSEMDVVRFDDDNSPSFIQIRSVAKKHPKTWVHYIAAEEEDWDYAPLVLAPDDRSYKSQYLNNGPQRIGRKYKKVRFMAYTDETFKTREAIQHESGILGPLLYGEVGDTLLIIFKNQASRPYNIYPHGITDVRPLYSRRLPKGVKHLKDFPILPGEIFKYKWTVTVEDGPTKSDPRCLTRYYSSFVNMERDLASGLIGPLLICYKESVDQRGNQIMSDKRNVILFSVFDENRSWYLTENIQRFLPNPAGVQLEDPEFQASNIMHSINGYVFDSLQLSVCLHEVAYWYILSIGAQTDFLSVFFSGYTFKHKMVYEDTLTLFPFSGETVFMSMENPGLWILGCHNSDFRNRGMTALLKVSSCDKNTGDYYEDSYEDISAYLLSKNNAIEPRSFSQNPPVLKRHQREITRTTLQSDQEEIDYDDTISVEMKKEDFDIYDEDENQSPRSFQKKTRHYFIAAVERLWDYGMSSSPHVLRNRAQSGSVPQFKKVVFQEFTDGSFTQPLYRGELNEHLGLLGPYIRAEVEDNIMVTFRNQASRPYSFYSSLISYEEDQRQGAEPRKNFVKPNETKTYFWKVQHHMAPTKDEFDCKAWAYFSDVDLEKDVHSGLIGPLLVCHTNTLNPAHGRQVTVQEFALFFTIFDETKSWYFTENMERNCRAPCNIQMEDPTFKENYRFHAINGYIMDTLPGLVMAQDQRIRWYLLSMGSNENIHSIHFSGHVFTVRKKEEYKMALYNLYPGVFETVEMLPSKAGIWRVECLIGEHLHAGMSTLFLVYSNKCQTPLGMASGHIRDFQITASGQYGQWAPKLARLHYSGSINAWSTKEPFSWIKVDLLAPMIIHGIKTQGARQKFSSLYISQFIIMYSLDGKKWQTYRGNSTGTLMVFFGNVDSSGIKHNIFNPPIIARYIRLHPTHYSIRSTLRMELMGCDLNSCSMPLGMESKAISDAQITASSYFTNMFATWSPSKARLHLQGRSNAWRPQVNNPKEWLQVDFQKTMKVTGVTTQGVKSLLTSMYVKEFLISSSQDGHQWTLFFQNGKVKVFQGNQDSFTPVVNSLDPPLLTRYLRIHPQSWVHQIALRMEVLGCEAQDLY

SEQ ID NO: 29-38 - skipped (10 entries)

SEQ ID NO: 39 - protein, 19 aa, Artificial Sequence
  Thrombin-cleavable linker
SKNNAIEPRSFSQNSRHPS

SEQ ID NO: 40 - protein, 14 aa, Artificial Sequence
  glycine-serine linker G1
  VARIANT 14: G in position 14 is present or absent
GSGGSGGSGGSGSG

SEQ ID NO: 41 - protein, 12 aa, Artificial Sequence
  glycine-serine linker G2
GGSGGGSGGGSG

SEQ ID NO: 42 - protein, 46 aa, Artificial Sequence
  GLG-1 linker
GSGGSGGSGGSGSGSKNNAIEPRSFSQNSRHPSGSGGSGGSGGSGS

SEQ ID NO: 43 - protein, 43 aa, Artificial Sequence
  GLG-2 linker
GGSGGGSGGGSGSKNNAIEPRSFSQNSRHPSGGSGGGSGGGSG

SEQ ID NO: 44 - protein, 46 aa, Artificial Sequence
  ABD consensus sequence
  VARIANT 3: X3 is selected from E, S, Q and C
  VARIANT 6: X6 is selected from E, S, C and V
  VARIANT 7: X7 is selected from A, S and L
  VARIANT 10: X10 is selected from A, S and R
  VARIANT 14: X14 is selected from A, S, C and K
  VARIANT 26: X26 is selected from D, E and N
  VARIANT 39: X39 is selected from D, E and L
  VARIANT 40: X40 is selected from A, E and H
  VARIANT 43: X43 is selected from A and K
  VARIANT 44: X44 is selected from A, S and E
  VARIANT 45: L in position 45 is present or absent
  VARIANT 46: P in position 46 is present or absent
LAXAKXXANXELDXYGVSDFYKRLIXKAKTVEGVEALKXXILXXLP

SEQ ID NO: 45 - protein, 46 aa, Artificial Sequence
  ABD1
LAEAKVLANRELDKYGVSDFYKRLINKAKTVEGVEALKLHILAALP

SEQ ID NO: 46 - protein, 46 aa, Artificial Sequence
  ABD2
LAEAKEAANAELDSYGVSDFYKRLIDKAKTVEGVEALKDAILAALP

SEQ ID NO: 47 - protein, 1575 aa, Artificial Sequence
  ADLCLD_SC aa
MQIELSTCFFLCLLRFCFSATRRYYLGAVELSWDYMQSDLGELPVDARFPPRVPKSFPFNTSVVYKKTLFVEFTDHLFNIAKPRPPWMGLLGPTIQAEVYDTVVITLKNMASHPVSLHAVGVSYWKASEGAEYDDQTSQREKEDDKVFPGGSHTYVWQVLKENGPMASDPLCLTYSYLSHVDLVKDLNSGLIGALLVCREGSLAKEKTQTLHKFILLFAVFDEGKSWHSETKNSLMQDRDAASARAWPKMHTVNGYVNRSLPGLIGCHRKSVYWHVIGMGTTPEVHSIFLEGHTFLVRNHRQASLEISPITFLTAQTLLMDLGQFLLFCHISSHQHDGMEAYVKVDSCPEEPQLRMKNNEEAEDYDDDLTDSEMDVVRFDDDNSPSFIQIRSVAKKHPKTWVHYIAAEEEDWDYAPLVLAPDDRSYKSQYLNNGPQRIGRKYKKVRFMAYTDETFKTREAIQHESGILGPLLYGEVGDTLLIIFKNQASRPYNIYPHGITDVRPLYSRRLPKGVKHLKDFPILPGEIFKYKWTVTVEDGPTKSDPRCLTRYYSSFVNMERDLASGLIGPLLICYKESVDQRGNQIMSDKRNVILFSVFDENRSWYLTENIQRFLPNPAGVQLEDPEFQASNIMHSINGYVFDSLQLSVCLHEVAYWYILSIGAQTDFLSVFFSGYTFKHKMVYEDTLTLFPFSGETVFMSMENPGLWILGCHNSDFRNRGMTALLKVSSCDKNTGDYYEDSYEDISAYLLSKNNAIEPRSFSQNPPVLAEAKEAANAELDSYGVSDFYKRLIDKAKTVEGVEALKDAILAALPSKNNAIEPRSFSQNSRHPSLQSDQEEIDYDDTISVEMKKEDFDIYDEDENQSPRSFQKKTRHYFIAAVERLWDYGMSSSPHVLRNRAQSGSVPQFKKVVFQEFTDGSFTQPLYRGELNEHLGLLGPYIRAEVEDNIMVTFRNQASRPYSFYSSLISYEEDQRQGAEPRKNFVKPNETKTYFWKVQHHMAPTKDEFDCKAWAYFSDVDLEKDVHSGLIGPLLVCHTNTLNPAHGRQVTVQEFALFFTIFDETKSWYFTENMERNCRAPCNIQMEDPTFKENYRFHAINGYIMDTLPGLVMAQDQRIRWYLLSMGSNENIHSIHFSGHVFTVRKKEEYKMALYNLYPGVFETVEMLPSKAGIWRVECLIGEHLHAGMSTLFLVYSNKCQTPLGMASGHIRDFQITASGQYGQWAPKLARLHYSGSINAWSTKEPFSWIKVDLLAPMIIHGIKTQGARQKFSSLYISQFIIMYSLDGKKWQTYRGNSTGTLMVFFGNVDSSGIKHNIFNPPIIARYIRLHPTHYSIRSTLRMELMGCDLNSCSMPLGMESKAISDAQITASSYFTNMFATWSPSKARLHLQGRSNAWRPQVNNPKEWLQVDFQKTMKVTGVTTQGVKSLLTSMYVKEFLISSSQDGHQWTLFFQNGKVKVFQGNQDSFTPVVNSLDPPLLTRYLRIHPQSWVHQIALRMEVLGCEAQDLYSKNNAIEPRSFSQNSRHPSLAEAKEAANAELDSYGVSDFYKRLIDKAKTVEGVEALKDAILAALP

SEQ ID NO: 48 - protein, 1778 aa, Artificial Sequence
  AD2CD2_SC aa
MQIELSTCFFLCLLRFCFSATRRYYLGAVELSWDYMQSDLGELPVDARFPPRVPKSFPFNTSVVYKKTLFVEFTDHLFNIAKPRPPWMGLLGPTIQAEVYDTVVITLKNMASHPVSLHAVGVSYWKASEGAEYDDQTSQREKEDDKVFPGGSHTYVWQVLKENGPMASDPLCLTYSYLSHVDLVKDLNSGLIGALLVCREGSLAKEKTQTLHKFILLFAVFDEGKSWHSETKNSLMQDRDAASARAWPKMHTVNGYVNRSLPGLIGCHRKSVYWHVIGMGTTPEVHSIFLEGHTFLVRNHRQASLEISPITFLTAQTLLMDLGQFLLFCHISSHQHDGMEAYVKVDSCPEEPQLRMKNNEEAEDYDDDLTDSEMDVVRFDDDNSPSFIQIRSVAKKHPKTWVHYIAAEEEDWDYAPLVLAPDDRSYKSQYLNNGPQRIGRKYKKVRFMAYTDETFKTREAIQHESGILGPLLYGEVGDTLLIIFKNQASRPYNIYPHGITDVRPLYSRRLPKGVKHLKDFPILPGEIFKYKWTVTVEDGPTKSDPRCLTRYYSSFVNMERDLASGLIGPLLICYKESVDQRGNQIMSDKRNVILFSVFDENRSWYLTENIQRFLPNPAGVQLEDPEFQASNIMHSINGYVFDSLQLSVCLHEVAYWYILSIGAQTDFLSVFFSGYTFKHKMVYEDTLTLFPFSGETVFMSMENPGLWILGCHNSDFRNRGMTALLKVSSCDKNTGDYYEDSYEDISAYLLSKNNAIEPRSFSQNPPVLSKNNAIEPRSFSQNSRHPSLAEAKEAANAELDSYGVSDFYKRLIDKAKTVEGVEALKDAILAALPGSGGSGGSGGSGSGSKNNAIEPRSFSQNSRHPSGSGGSGGSGGSGSLAEAKEAANAELDSYGVSDFYKRLIDKAKTVEGVEALKDAILAALPSKNNAIEPRSFSQNSRHPSQSDQEEIDYDDTISVEMKKEDFDIYDEDENQSPRSFQKKTRHYFIAAVERLWDYGMSSSPHVLRNRAQSGSVPQFKKVVFQEFTDGSFTQPLYRGELNEHLGLLGPYIRAEVEDNIMVTFRNQASRPYSFYSSLISYEEDQRQGAEPRKNFVKPNETKTYFWKVQHHMAPTKDEFDCKAWAYFSDVDLEKDVHSGLIGPLLVCHTNTLNPAHGRQVTVQEFALFFTIFDETKSWYFTENMERNCRAPCNIQMEDPTFKENYRFHAINGYIMDTLPGLVMAQDQRIRWYLLSMGSNENIHSIHFSGHVFTVRKKEEYKMALYNLYPGVFETVEMLPSKAGIWRVECLIGEHLHAGMSTLFLVYSNKCQTPLGMASGHIRDFQITASGQYGQWAPKLARLHYSGSINAWSTKEPFSWIKVDLLAPMIIHGIKTQGARQKFSSLYISQFIIMYSLDGKKWQTYRGNSTGTLMVFFGNVDSSGIKHNIFNPPIIARYIRLHPTHYSIRSTLRMELMGCDLNSCSMPLGMESKAISDAQITASSYFTNMFATWSPSKARLHLQGRSNAWRPQVNNPKEWLQVDFQKTMKVTGVTTQGVKSLLTSMYVKEFLISSSQDGHQWTLFFQNGKVKVFQGNQDSFTPVVNSLDPPLLTRYLRIHPQSWVHQIALRMEVLGCEAQDLYSKNNAIEPRSFSQNSRHPSLAEAKEAANAELDSYGVSDFYKRLIDKAKTVEGVEALKDAILAALPGSGGSGGSGGSGSGSKNNAIEPRSFSQNSRHPSGSGGSGGSGGSGSLAEAKEAANAELDSYGVSDFYKRLIDKAKTVEGVEALKDAILAALP

SEQ ID NO: 49 - protein, 1694 aa, Artificial Sequence
  AD2CD2woL_SC aa
MQIELSTCFFLCLLRFCFSATRRYYLGAVELSWDYMQSDLGELPVDARFPPRVPKSFPFNTSVVYKKTLFVEFTDHLFNIAKPRPPWMGLLGPTIQAEVYDTVVITLKNMASHPVSLHAVGVSYWKASEGAEYDDQTSQREKEDDKVFPGGSHTYVWQVLKENGPMASDPLCLTYSYLSHVDLVKDLNSGLIGALLVCREGSLAKEKTQTLHKFILLFAVFDEGKSWHSETKNSLMQDRDAASARAWPKMHTVNGYVNRSLPGLIGCHRKSVYWHVIGMGTTPEVHSIFLEGHTFLVRNHRQASLEISPITFLTAQTLLMDLGQFLLFCHISSHQHDGMEAYVKVDSCPEEPQLRMKNNEEAEDYDDDLTDSEMDVVRFDDDNSPSFIQIRSVAKKHPKTWVHYIAAEEEDWDYAPLVLAPDDRSYKSQYLNNGPQRIGRKYKKVRFMAYTDETFKTREAIQHESGILGPLLYGEVGDTLLIIFKNQASRPYNIYPHGITDVRPLYSRRLPKGVKHLKDFPILPGEIFKYKWTVTVEDGPTKSDPRCLTRYYSSFVNMERDLASGLIGPLLICYKESVDQRGNQIMSDKRNVILFSVFDENRSWYLTENIQRFLPNPAGVQLEDPEFQASNIMHSINGYVFDSLQLSVCLHEVAYWYILSIGAQTDFLSVFFSGYTFKHKMVYEDTLTLFPFSGETVFMSMENPGLWILGCHNSDFRNRGMTALLKVSSCDKNTGDYYEDSYEDISAYLLSKNNAIEPRSFSQNPPVLGSGGSGGSGGSGSLAEAKEAANAELDSYGVSDFYKRLIDKAKTVEGVEALKDAILAALPGSGGSGGSGGSGSLAEAKEAANAELDSYGVSDFYKRLIDKAKTVEGVEALKDAILAALPGSGGSGGSGGSGSQSDQEEIDYDDTISVEMKKEDFDIYDEDENQSPRSFQKKTRHYFIAAVERLWDYGMSSSPHVLRNRAQSGSVPQFKKVVFQEFTDGSFTQPLYRGELNEHLGLLGPYIRAEVEDNIMVTFRNQASRPYSFYSSLISYEEDQRQGAEPRKNFVKPNETKTYFWKVQHHMAPTKDEFDCKAWAYFSDVDLEKDVHSGLIGPLLVCHTNTLNPAHGRQVTVQEFALFFTIFDETKSWYFTENMERNCRAPCNIQMEDPTFKENYRFHAINGYIMDTLPGLVMAQDQRIRWYLLSMGSNENIHSIHFSGHVFTVRKKEEYKMALYNLYPGVFETVEMLPSKAGIWRVECLIGEHLHAGMSTLFLVYSNKCQTPLGMASGHIRDFQITASGQYGQWAPKLARLHYSGSINAWSTKEPFSWIKVDLLAPMIIHGIKTQGARQKFSSLYISQFIIMYSLDGKKWQTYRGNSTGTLMVFFGNVDSSGIKHNIFNPPIIARYIRLHPTHYSIRSTLRMELMGCDLNSCSMPLGMESKAISDAQITASSYFTNMFATWSPSKARLHLQGRSNAWRPQVNNPKEWLQVDFQKTMKVTGVTTQGVKSLLTSMYVKEFLISSSQDGHQWTLFFQNGKVKVFQGNQDSFTPVVNSLDPPLLTRYLRIHPQSWVHQIALRMEVLGCEAQDLYGSGGSGGSGGSGSLAEAKEAANAELDSYGVSDFYKRLIDKAKTVEGVEALKDAILAALPGSGGSGGSGGSGSLAEAKEAANAELDSYGVSDFYKRLIDKAKTVEGVEALKDAILAALP

SEQ ID NO: 50 - protein, 1629 aa, Artificial Sequence
  AD2CD2woLG_SC aa
MQIELSTCFFLCLLRFCFSATRRYYLGAVELSWDYMQSDLGELPVDARFPPRVPKSFPFNTSVVYKKTLFVEFTDHLFNIAKPRPPWMGLLGPTIQAEVYDTVVITLKNMASHPVSLHAVGVSYWKASEGAEYDDQTSQREKEDDKVFPGGSHTYVWQVLKENGPMASDPLCLTYSYLSHVDLVKDLNSGLIGALLVCREGSLAKEKTQTLHKFILLFAVFDEGKSWHSETKNSLMQDRDAASARAWPKMHTVNGYVNRSLPGLIGCHRKSVYWHVIGMGTTPEVHSIFLEGHTFLVRNHRQASLEISPITFLTAQTLLMDLGQFLLFCHISSHQHDGMEAYVKVDSCPEEPQLRMKNNEEAEDYDDDLTDSEMDVVRFDDDNSPSFIQIRSVAKKHPKTWVHYIAAEEEDWDYAPLVLAPDDRSYKSQYLNNGPQRIGRKYKKVRFMAYTDETFKTREAIQHESGILGPLLYGEVGDTLLIIFKNQASRPYNIYPHGITDVRPLYSRRLPKGVKHLKDFPILPGEIFKYKWTVTVEDGPTKSDPRCLTRYYSSFVNMERDLASGLIGPLLICYKESVDQRGNQIMSDKRNVILFSVFDENRSWYLTENIQRFLPNPAGVQLEDPEFQASNIMHSINGYVFDSLQLSVCLHEVAYWYILSIGAQTDFLSVFFSGYTFKHKMVYEDTLTLFPFSGETVFMSMENPGLWILGCHNSDFRNRGMTALLKVSSCDKNTGDYYEDSYEDISAYLLSKNNAIEPRSFSQNPPVLLAEAKEAANAELDSYGVSDFYKRLIDKAKTVEGVEALKDAILAALPLAEAKEAANAELDSYGVSDFYKRLIDKAKTVEGVEALKDAILAALPQSDQEEIDYDDTISVEMKKEDFDIYDEDENQSPRSFQKKTRHYFIAAVERLWDYGMSSSPHVLRNRAQSGSVPQFKKVVFQEFTDGSFTQPLYRGELNEHLGLLGPYIRAEVEDNIMVTFRNQASRPYSFYSSLISYEEDQRQGAEPRKNFVKPNETKTYFWKVQHHMAPTKDEFDCKAWAYFSDVDLEKDVHSGLIGPLLVCHTNTLNPAHGRQVTVQEFALFFTIFDETKSWYFTENMERNCRAPCNIQMEDPTFKENYRFHAINGYIMDTLPGLVMAQDQRIRWYLLSMGSNENIHSIHFSGHVFTVRKKEEYKMALYNLYPGVFETVEMLPSKAGIWRVECLIGEHLHAGMSTLFLVYSNKCQTPLGMASGHIRDFQITASGQYGQWAPKLARLHYSGSINAWSTKEPFSWIKVDLLAPMIIHGIKTQGARQKFSSLYISQFIIMYSLDGKKWQTYRGNSTGTLMVFFGNVDSSGIKHNIFNPPIIARYIRLHPTHYSIRSTLRMELMGCDLNSCSMPLGMESKAISDAQITASSYFTNMFATWSPSKARLHLQGRSNAWRPQVNNPKEWLQVDFQKTMKVTGVTTQGVKSLLTSMYVKEFLISSSQDGHQWTLFFQNGKVKVFQGNQDSFTPVVNSLDPPLLTRYLRIHPQSWVHQIALRMEVLGCEAQDLYLAEAKEAANAELDSYGVSDFYKRLIDKAKTVEGVEALKDAILAALPLAEAKEAANAELDSYGVSDFYKRLIDKAKTVEGVEALKDAILAALP

SEQ ID NO: 51 - protein, 1778 aa, Artificial Sequence
  AbD2CD2_SC aa
MQIELSTCFFLCLLRFCFSATRRYYLGAVELSWDYMQSDLGELPVDARFPPRVPKSFPFNTSVVYKKTLFVEFTDHLFNIAKPRPPWMGLLGPTIQAEVYDTVVITLKNMASHPVSLHAVGVSYWKASEGAEYDDQTSQREKEDDKVFPGGSHTYVWQVLKENGPMASDPLCLTYSYLSHVDLVKDLNSGLIGALLVCREGSLAKEKTQTLHKFILLFAVFDEGKSWHSETKNSLMQDRDAASARAWPKMHTVNGYVNRSLPGLIGCHRKSVYWHVIGMGTTPEVHSIFLEGHTFLVRNHRQASLEISPITFLTAQTLLMDLGQFLLFCHISSHQHDGMEAYVKVDSCPEEPQLRMKNNEEAEDYDDDLTDSEMDVVRFDDDNSPSFIQIRSVAKKHPKTWVHYIAAEEEDWDYAPLVLAPDDRSYKSQYLNNGPQRIGRKYKKVRFMAYTDETFKTREAIQHESGILGPLLYGEVGDTLLIIFKNQASRPYNIYPHGITDVRPLYSRRLPKGVKHLKDFPILPGEIFKYKWTVTVEDGPTKSDPRCLTRYYSSFVNMERDLASGLIGPLLICYKESVDQRGNQIMSDKRNVILFSVFDENRSWYLTENIQRFLPNPAGVQLEDPEFQASNIMHSINGYVFDSLQLSVCLHEVAYWYILSIGAQTDFLSVFFSGYTFKHKMVYEDTLTLFPFSGETVFMSMENPGLWILGCHNSDFRNRGMTALLKVSSCDKNTGDYYEDSYEDISAYLLSKNNAIEPRSFSQNPPVLSKNNAIEPRSFSQNSRHPSLAEAKEAANAELDSYGVSDFYKRLIDKAKTVEGVEALKDAILAALPGSGGSGGSGGSGSGSKNNAIEPRSFSQNSRHPSGSGGSGGSGGSGSLAEAKEAANAELDSYGVSDFYKRLIDKAKTVEGVEALKDAILAALPSKNNAIEPRSFSQNSRHPSQSDQEEIDFDDTISVEMKKEDFDIFDEDENQSPRSFQKKTRHYFIAAVERLWDYGMSSSPHVLRNRAQSGSVPQFKKVVFQEFTDGSFTQPLYRGELNEHLGLLGPYIRAEVEDNIMVTFRNQASRPYSFYSSLISYEEDQRQGAEPRKNFVKPNETKTYFWKVQHHMAPTKDEFDCKAWAYFSDVDLEKDVHSGLIGPLLVCHTNTLNPAHGRQVTVQEFALFFTIFDETKSWYFTENMERNCRAPCNIQMEDPTFKENYRFHAINGYIMDTLPGLVMAQDQRIRWYLLSMGSNENIHSIHFSGHVFTVRKKEEYKMALYNLYPGVFETVEMLPSKAGIWRVECLIGEHLHAGMSTLFLVYSNKCQTPLGMASGHIRDFQITASGQYGQWAPKLARLHYSGSINAWSTKEPFSWIKVDLLAPMIIHGIKTQGARQKFSSLYISQFIIMYSLDGKKWQTYRGNSTGTLMVFFGNVDSSGIKHNIFNPPIIARYIRLHPTHYSIRSTLRMELMGCDLNSCSMPLGMESKAISDAQITASSYFTNMFATWSPSKARLHLQGRSNAWRPQVNNPKEWLQVDFQKTMKVTGVTTQGVKSLLTSMYVKEFLISSSQDGHQWTLFFQNGKVKVFQGNQDSFTPVVNSLDPPLLTRYLRIHPQSWVHQIALRMEVLGCEAQDLYSKNNAIEPRSFSQNSRHPSLAEAKEAANAELDSYGVSDFYKRLIDKAKTVEGVEALKDAILAALPGSGGSGGSGGSGSGSKNNAIEPRSFSQNSRHPSGSGGSGGSGGSGSLAEAKEAANAELDSYGVSDFYKRLIDKAKTVEGVEALKDAILAALP

SEQ ID NO: 52 - DNA, 4728 bp, Artificial Sequence
  ADLCLD_SC na
atgcagatcgagctgtctacctgcttcttcctgtgcctgctgcggttctgcttcagcgccacccggcggtactacctgggcgccgtggaactgagctgggactacatgcagagcgacctgggcgagctgcccgtggacgccagattccccccaagagtgcccaagagcttccccttcaacacctccgtggtgtacaagaaaaccctgttcgtcgagttcaccgaccacctgttcaatatcgccaagcccagacccccctggatgggcctgctgggccctacaatccaggccgaggtgtacgacaccgtggtcatcaccctgaagaacatggccagccaccccgtgtccctgcacgccgtgggcgtgtcctactggaaggccagcgagggcgccgagtacgacgaccagaccagccagcgcgagaaagaggacgacaaagtctttcctggcggcagccatacctacgtgtggcaggtcctgaaagaaaacggccccatggcctccgaccccctgtgcctgacctacagctacctgagccacgtggacctggtcaaggacctgaacagcggcctgatcggcgccctgctcgtgtgcagagagggcagcctggccaaagagaaaacccagaccctgcacaagttcatcctgctgttcgccgtgttcgacgagggcaagagctggcacagcgagacaaagaacagcctgatgcaggaccgggacgccgcctctgccagagcctggcctaagatgcacaccgtgaacggctacgtgaacagaagcctgcccggactgatcggctgccaccggaagtccgtgtactggcacgtgatcggcatgggtaccacccccgaggtgcacagcatctttctggaaggacacaccttcctcgtgcggaaccaccggcaggccagcctggaaatcagccctatcaccttcctgaccgcccagacactgctgatggacctgggccagttcctgctgttttgccacatcagcagccaccagcacgacggcatggaagcctacgtgaaggtggacagctgccccgaggaaccccagctgcggatgaagaacaacgaggaagccgaggactacgacgacgacctgaccgacagcgagatggacgtcgtcagattcgatgacgacaacagccccagcttcatccagatcagaagcgtggccaagaagcaccccaagacctgggtgcactatatcgccgccgaggaagaggactgggactacgcccctctggtgctggcccccgacgacagaagctacaagagccagtacctgaacaatggcccccagcggatcggccggaagtacaagaaagtgcggttcatggcctacaccgacgagacattcaagaccagagaggccatccagcacgagagcggcatcctgggccccctgctgtacggcgaagtgggcgacaccctgctgattatcttcaagaaccaggccagccggccctacaacatctacccccacggcatcaccgacgtgcggcccctgtacagcagacggctgcccaagggcgtgaagcacctgaaggacttccccatcctgcccggggagatcttcaagtacaagtggaccgtgaccgtggaagatggccccaccaagagcgaccccagatgcctgacccggtactacagcagcttcgtgaacatggaacgggacctggcctccgggctgatcggccctctgctgatctgctacaaagaaagcgtggaccagcggggcaaccagatcatgagcgacaagcggaacgtgatcctgttcagcgtgttcgatgagaacagaagctggtatctgaccgagaatatccagcggttcctgcccaaccctgccggcgtgcagctggaagatcccgagttccaggccagcaacatcatgcactccatcaatggctacgtgttcgacagcctgcagctgagcgtgtgcctgcacgaggtggcctactggtacatcctgagcatcggcgcccagaccgacttcctgagcgtgttcttcagcggctacaccttcaagcacaagatggtgtacgaggataccctgaccctgttccccttctccggcgaaaccgtgttcatgagcatggaaaaccccggcctgtggattctgggctgccacaacagcgacttccggaaccggggcatgaccgccctgctgaaggtgtccagctgcgacaagaacaccggcgactactacgaggacagctacgaggatatcagcgcctacctgctgagcaagaacaacgccatcgagccccggtccttcagccagaacccccccgtgctggccgaggccaaggaggccgccaacgccgagctggacagctacggcgtgagcgacttctacaagcgcctgatcgacaaggccaagaccgtggagggcgtggaggccctgaaggacgccatcctggccgccctgcccagcaaaaataacgctatagagcccaggagctttagtcagaatagcaggcacccgagtctgcagagcgaccaggaagagatcgactacgacgacaccatcagcgtcgagatgaagaaagaggacttcgacatctacgacgaggacgagaaccagagcccccggtccttccagaagaaaacccggcactactttatcgccgccgtggaacggctgtgggactacggcatgagcagcagcccccacgtgctgcggaatagagcccagagcggcagcgtgccccagttcaagaaagtggtgttccaggagttcaccgacggcagcttcacccagcctctgtaccggggcgagctgaacgagcacctgggcctgctgggcccctacatccgggccgaggtggaagataacatcatggtcaccttccggaaccaggccagccggccctacagcttctacagcagcctgatctcctacgaagaggaccagcggcagggcgccgagccccggaagaacttcgtgaagcccaacgagacaaagacctacttctggaaggtgcagcaccacatggcccccaccaaggacgaattcgactgcaaggcctgggcctacttcagcgacgtggacctggaaaaggacgtgcacagcggcctgatcggccccctgctcgtgtgccacaccaacaccctgaaccccgcccacggccggcaggtcacagtgcaggaatttgccctgttcttcaccatcttcgacgagacaaagagctggtacttcaccgagaacatggaacggaactgcagagccccctgcaacatccagatggaagatcccaccttcaaagagaactaccggttccacgccatcaacggctacatcatggacaccctgcccggcctggtcatggcccaggaccagcggatccggtggtatctgctgagcatgggcagcaacgagaacatccacagcatccacttcagcggccacgtgttcaccgtgcggaagaaagaagagtacaagatggccctgtacaacctgtaccccggcgtgttcgagacagtggaaatgctgcccagcaaggccggcatctggcgggtggaatgtctgatcggcgagcatctgcacgccggcatgagcaccctgtttctggtgtacagcaacaagtgccagacccccctgggcatggccagcggccacatccgggatttccagatcaccgcctccggccagtacggccagtgggcccctaaactggcccggctgcactacagcggcagcatcaacgcctggtccaccaaagagcccttcagctggatcaaggtggacctgctggcccccatgatcatccacggcatcaagacccagggcgccagacagaagttcagcagcctgtacatcagccagttcatcatcatgtacagcctggacggcaagaagtggcagacctaccggggcaacagcaccggcaccctgatggtgttcttcggcaacgtggacagcagcggcatcaagcacaacatcttcaacccccccatcattgcccggtacatccggctgcaccccacccactacagcatccggtccaccctgcggatggaactgatgggctgcgacctgaacagctgcagcatgcctctgggcatggaaagcaaggccatcagcgacgcccagatcacagccagcagctacttcaccaacatgttcgccacctggtccccatccaaggccagactgcatctgcagggcagaagcaatgcctggcggcctcaggtcaacaaccccaaagagtggctgcaggtcgacttccagaaaaccatgaaggtcacaggcgtgaccacccagggcgtgaagtccctgctgacctctatgtacgtgaaagagttcctgatcagcagcagccaggacggccaccagtggaccctgttctttcagaacggcaaagtgaaggtgttccagggcaaccaggacagcttcacccccgtggtcaactccctggaccccccactgctgaccagatacctgcggatccacccccagagctgggtgcaccagatcgccctgagaatggaagtgctgggatgcgaggcccaggatctgtacagcaagaacaacgccatcgagcccagaagcttcagccagaacagccggcaccctagcctggccgaggccaaagaagccgccaacgccgagctggacagctacggcgtgtccgacttctacaagcggctgatcgacaaggccaagaccgtggaaggcgtggaagccctgaaggacgccatcctggccgccctgccctga

SEQ ID NO: 53 - DNA, 5337 bp, Artificial Sequence
  AD2CD2_SC na
atgcagatcgagctgtctacctgcttcttcctgtgcctgctgcggttctgcttcagcgccacccggcggtactacctgggcgccgtggaactgagctgggactacatgcagagcgacctgggcgagctgcccgtggacgccagattccccccaagagtgcccaagagcttccccttcaacacctccgtggtgtacaagaaaaccctgttcgtcgagttcaccgaccacctgttcaatatcgccaagcccagacccccctggatgggcctgctgggccctacaatccaggccgaggtgtacgacaccgtggtcatcaccctgaagaacatggccagccaccccgtgtccctgcacgccgtgggcgtgtcctactggaaggccagcgagggcgccgagtacgacgaccagaccagccagcgcgagaaagaggacgacaaagtctttcctggcggcagccatacctacgtgtggcaggtcctgaaagaaaacggccccatggcctccgaccccctgtgcctgacctacagctacctgagccacgtggacctggtcaaggacctgaacagcggcctgatcggcgccctgctcgtgtgcagagagggcagcctggccaaagagaaaacccagaccctgcacaagttcatcctgctgttcgccgtgttcgacgagggcaagagctggcacagcgagacaaagaacagcctgatgcaggaccgggacgccgcctctgccagagcctggcctaagatgcacaccgtgaacggctacgtgaacagaagcctgcccggactgatcggctgccaccggaagtccgtgtactggcacgtgatcggcatgggtaccacccccgaggtgcacagcatctttctggaaggacacaccttcctcgtgcggaaccaccggcaggccagcctggaaatcagccctatcaccttcctgaccgcccagacactgctgatggacctgggccagttcctgctgttttgccacatcagcagccaccagcacgacggcatggaagcctacgtgaaggtggacagctgccccgaggaaccccagctgcggatgaagaacaacgaggaagccgaggactacgacgacgacctgaccgacagcgagatggacgtcgtcagattcgatgacgacaacagccccagcttcatccagatcagaagcgtggccaagaagcaccccaagacctgggtgcactatatcgccgccgaggaagaggactgggactacgcccctctggtgctggcccccgacgacagaagctacaagagccagtacctgaacaatggcccccagcggatcggccggaagtacaagaaagtgcggttcatggcctacaccgacgagacattcaagaccagagaggccatccagcacgagagcggcatcctgggccccctgctgtacggcgaagtgggcgacaccctgctgattatcttcaagaaccaggccagccggccctacaacatctacccccacggcatcaccgacgtgcggcccctgtacagcagacggctgcccaagggcgtgaagcacctgaaggacttccccatcctgcccggggagatcttcaagtacaagtggaccgtgaccgtggaagatggccccaccaagagcgaccccagatgcctgacccggtactacagcagcttcgtgaacatggaacgggacctggcctccgggctgatcggccctctgctgatctgctacaaagaaagcgtggaccagcggggcaaccagatcatgagcgacaagcggaacgtgatcctgttcagcgtgttcgatgagaacagaagctggtatctgaccgagaatatccagcggttcctgcccaaccctgccggcgtgcagctggaagatcccgagttccaggccagcaacatcatgcactccatcaatggctacgtgttcgacagcctgcagctgagcgtgtgcctgcacgaggtggcctactggtacatcctgagcatcggcgcccagaccgacttcctgagcgtgttcttcagcggctacaccttcaagcacaagatggtgtacgaggataccctgaccctgttccccttctccggcgaaaccgtgttcatgagcatggaaaaccccggcctgtggattctgggctgccacaacagcgacttccggaaccggggcatgaccgccctgctgaaggtgtccagctgcgacaagaacaccggcgactactacgaggacagctacgaggatatcagcgcctacctgctgagcaagaacaacgccatcgagcctcggagcttcagccagaatcctcctgtgctgtccaaaaacaatgccattgagccccggtcattctcccagaactctagacacccaagcctggccgaagccaaagaagccgctaacgccgagctggatagctacggcgtgtccgacttctacaagcggctgatcgataaggccaagaccgtggaaggcgtggaagccctgaaagatgctattctggccgctctgcctggaagcggaggtagcggaggatctggcggttctggcagcggatctaagaacaatgctatcgaaccccgctcctttagccagaacagcagacacccttctggaagtggtggttctggtggatccggcggatctggatctctggcagaggctaaagaggccgccaacgctgaactcgactcctatggggtgtccgatttttacaaacgcctcattgacaaggccaaaacagtcgagggcgtcgaggctctgaaggatgccattcttgctgccctgccatctaaaaacaacgcaatagagcccagatccttcagtcagaacagtcggcaccccagccagagcgaccaagaggaaatcgactacgacgacaccatcagcgtcgagatgaagaaagaggacttcgacatctacgacgaggacgagaatcagagccccagaagcttccagaaaaagacccggcactactttatcgccgccgtggaaagactgtgggactacggaatgagcagcagccctcacgtgctgagaaatagagcacagagcggcagcgtgccccagttcaagaaagtggtgttccaagagttcaccgacggcagcttcacccagcctctgtatagaggcgagctgaatgagcacctgggcctgctgggaccttatatcagagccgaggtggaagataacatcatggtcaccttccggaaccaggccagcagaccctacagcttctacagcagcctgatcagctacgaagaggaccagagacagggcgccgagcctagaaagaacttcgtgaagcccaacgagacaaagacctacttttggaaggtgcagcaccacatggcccctaccaaggacgaattcgattgcaaggcctgggcctacttcagcgacgtggacctggaaaaggatgtgcactctggcctgatcggccctctgctcgtgtgtcacaccaacacactgaaccccgctcacggcagacaagtgaccgtgcaagagttcgccctgttcttcaccatcttcgacgagacaaagagctggtacttcaccgagaacatggaacggaactgcagagccccttgcaacatccagatggaagatcccaccttcaaagagaactaccggttccacgccatcaacggctacatcatggataccctgcctggcctggttatggcccaggaccagagaatcagatggtatctgctgagcatgggcagcaacgagaacatccacagcatccacttcagcggccacgtgttcaccgtgcggaagaaagaagagtacaagatggccctgtacaatctgtaccccggcgtgttcgagacagtggaaatgctgccttctaaggccggcatttggagagtggaatgtctgatcggagagcatctgcacgccggcatgagcaccctgtttctggtgtacagcaacaagtgtcagacccctctcggcatggcctccggccacatcagagattttcagatcacagccagcggccagtatggccagtgggctcctaaactggccagactgcactacagcggcagcatcaatgcctggtccaccaaagagcccttcagctggattaaggtggacctgctggcccctatgatcatccacggcatcaaaacacagggcgccagacagaagttcagcagcctgtacatcagccagttcatcatcatgtacagcctggacggcaagaagtggcagacctacagaggcaatagcaccggcacactgatggtgttcttcggcaatgtggacagcagcggcatcaagcacaacatcttcaaccctccaatcattgcccggtacatccggctgcaccccacacactacagcatccggtctaccctgagaatggaactgatgggctgcgacctgaacagctgtagcatgcccctcggaatggaaagcaaggccatcagcgacgcccagatcaccgcctccagctacttcaccaacatgttcgccacttggagcccctccaaagccagactccatctgcaaggcagatctaacgcttggaggccccaagtgaacaaccccaaagagtggctgcaggtcgacttccagaaaaccatgaaggtcacaggcgtgacaacccagggcgtgaagtctctgctgacctctatgtacgtgaaagagttcctgatcagcagcagccaggacggccaccagtggaccctgtttttccagaacggcaaagtgaaggtgttccagggcaaccaggacagcttcacccctgtggtcaatagcctggatcctccactgctgaccagatacctgcggattcaccctcagagctgggtgcaccagatcgctctcagaatggaagtgctgggatgtgaagcccaggatctgtactccaagaacaacgccatcgagcccagaagcttcagccagaactctagacacccaagcctggccgaagccaaagaagccgctaacgccgagctggatagctacggcgtgtccgacttctacaagcggctgatcgataaggccaagaccgtggaaggcgtggaagccctgaaagatgccattctggctgctctgcctggaagcggaggaagtggtggatctggcggttctggctccggcagcaaaaacaatgccattgagcctcggagcttctcccagaacagcagacatccttctggatcaggcggtagcggaggatccggcggatctggatctctggctgaggctaaagaggccgccaacgctgaactcgactcctatggggtgtccgatttttacaaacgcctcattgacaaggccaaaacagtcgagggcgtcgaggctctgaaggatgctattcttgccgcactgccctga

SEQ ID NO: 54 - DNA, 5085 bp, Artificial Sequence
  AD2CD2woL_SC na
atgcagatcgagctgtctacctgcttcttcctgtgcctgctgcggttctgcttcagcgccacccggcggtactacctgggcgccgtggaactgagctgggactacatgcagagcgacctgggcgagctgcccgtggacgccagattccccccaagagtgcccaagagcttccccttcaacacctccgtggtgtacaagaaaaccctgttcgtcgagttcaccgaccacctgttcaatatcgccaagcccagacccccctggatgggcctgctgggccctacaatccaggccgaggtgtacgacaccgtggtcatcaccctgaagaacatggccagccaccccgtgtccctgcacgccgtgggcgtgtcctactggaaggccagcgagggcgccgagtacgacgaccagaccagccagcgcgagaaagaggacgacaaagtctttcctggcggcagccatacctacgtgtggcaggtcctgaaagaaaacggccccatggcctccgaccccctgtgcctgacctacagctacctgagccacgtggacctggtcaaggacctgaacagcggcctgatcggcgccctgctcgtgtgcagagagggcagcctggccaaagagaaaacccagaccctgcacaagttcatcctgctgttcgccgtgttcgacgagggcaagagctggcacagcgagacaaagaacagcctgatgcaggaccgggacgccgcctctgccagagcctggcctaagatgcacaccgtgaacggctacgtgaacagaagcctgcccggactgatcggctgccaccggaagtccgtgtactggcacgtgatcggcatgggtaccacccccgaggtgcacagcatctttctggaaggacacaccttcctcgtgcggaaccaccggcaggccagcctggaaatcagccctatcaccttcctgaccgcccagacactgctgatggacctgggccagttcctgctgttttgccacatcagcagccaccagcacgacggcatggaagcctacgtgaaggtggacagctgccccgaggaaccccagctgcggatgaagaacaacgaggaagccgaggactacgacgacgacctgaccgacagcgagatggacgtcgtcagattcgatgacgacaacagccccagcttcatccagatcagaagcgtggccaagaagcaccccaagacctgggtgcactatatcgccgccgaggaagaggactgggactacgcccctctggtgctggcccccgacgacagaagctacaagagccagtacctgaacaatggcccccagcggatcggccggaagtacaagaaagtgcggttcatggcctacaccgacgagacattcaagaccagagaggccatccagcacgagagcggcatcctgggccccctgctgtacggcgaagtgggcgacaccctgctgattatcttcaagaaccaggccagccggccctacaacatctacccccacggcatcaccgacgtgcggcccctgtacagcagacggctgcccaagggcgtgaagcacctgaaggacttccccatcctgcccggggagatcttcaagtacaagtggaccgtgaccgtggaagatggccccaccaagagcgaccccagatgcctgacccggtactacagcagcttcgtgaacatggaacgggacctggcctccgggctgatcggccctctgctgatctgctacaaagaaagcgtggaccagcggggcaaccagatcatgagcgacaagcggaacgtgatcctgttcagcgtgttcgatgagaacagaagctggtatctgaccgagaatatccagcggttcctgcccaaccctgccggcgtgcagctggaagatcccgagttccaggccagcaacatcatgcactccatcaatggctacgtgttcgacagcctgcagctgagcgtgtgcctgcacgaggtggcctactggtacatcctgagcatcggcgcccagaccgacttcctgagcgtgttcttcagcggctacaccttcaagcacaagatggtgtacgaggataccctgaccctgttccccttctccggcgaaaccgtgttcatgagcatggaaaaccccggcctgtggattctgggctgccacaacagcgacttccggaaccggggcatgaccgccctgctgaaggtgtccagctgcgacaagaacaccggcgactactacgaggacagctacgaggatatcagcgcctacctgctgagcaagaacaacgccatcgagccccggtccttcagccagaacccccccgtgctgggaagcggaggatctggcggctcaggcggatctggatctctggccgaagccaaagaagccgccaacgccgagctggacagctatggcgtgtccgacttctacaagcggctgatcgacaaggccaagacagtggaaggcgtggaagctctgaaggatgccatcctggccgctctgcctggaagtgggggatcaggcggcagcggaggaagtggaagtctggctgaggctaaagaggctgctaacgctgaactggactcttacggggtgtccgatttttacaaaagactgattgataaggctaaaactgtggaaggggtggaagccctgaaagacgctattctggctgccctgcctggctccgggggctctggcggaagtggcggcagtggatctcagagcgaccaggaagagatcgactacgacgacaccatcagcgtcgagatgaagaaagaggacttcgacatctacgacgaggacgagaaccagagcccccggtccttccagaagaaaacccggcactactttatcgccgccgtggaacggctgtgggactacggcatgagcagcagcccccacgtgctgcggaatagagcccagagcggcagcgtgccccagttcaagaaagtggtgttccaggagttcaccgacggcagcttcacccagcctctgtaccggggcgagctgaacgagcacctgggcctgctgggcccctacatccgggccgaggtggaagataacatcatggtcaccttccggaaccaggccagccggccctacagcttctacagcagcctgatctcctacgaagaggaccagcggcagggcgccgagccccggaagaacttcgtgaagcccaacgagacaaagacctacttctggaaggtgcagcaccacatggcccccaccaaggacgaattcgactgcaaggcctgggcctacttcagcgacgtggacctggaaaaggacgtgcacagcggcctgatcggccccctgctcgtgtgccacaccaacaccctgaaccccgcccacggccggcaggtcacagtgcaggaatttgccctgttcttcaccatcttcgacgagactaagagctggtacttcaccgagaacatggaacggaactgcagagccccctgcaacatccagatggaagatcccaccttcaaagagaactaccggttccacgccatcaatggctacatcatggacaccctgcccggcctggtcatggcccaggaccagagaatccggtggtatctgctgagcatgggcagcaacgagaacatccacagcatccacttcagcggccacgtgttcaccgtgcggaagaaagaagagtacaagatggccctgtacaacctgtaccccggcgtgttcgaaacagtggaaatgctgcccagcaaggccggcatctggcgggtggaatgtctgatcggcgagcatctgcacgccggcatgtccaccctgtttctggtgtacagcaacaagtgccagacccccctgggcatggccagcggccacatccgggatttccagatcaccgcctccggccagtacggccagtgggcccctaaactggcccggctgcactacagcggcagcatcaacgcctggtccaccaaagagcccttcagctggatcaaggtggacctgctggcccccatgattatccacggcatcaagacacagggcgccagacagaagttcagcagcctgtacatcagccagttcatcatcatgtacagcctggatggcaagaagtggcagacctaccggggcaacagcaccggcaccctgatggtgttcttcggcaacgtggacagcagcggcatcaagcacaacatcttcaacccccccatcattgcccggtacatccggctgcaccccacccactacagcatccggtccaccctgcggatggaactgatgggctgcgacctgaacagctgctccatgcctctgggcatggaaagcaaggccatcagcgacgcccagatcacagccagcagctacttcaccaacatgttcgccacctggtccccatccaaggccagactgcatctgcagggcagaagcaatgcctggcggcctcaggtcaacaaccccaaagaatggctccaggtggacttccagaaaaccatgaaggtcacaggcgtgaccacccagggcgtgaagtccctgctgacctctatgtacgtgaaagagttcctgatctccagcagccaggacggccaccagtggaccctgttctttcagaacggcaaagtgaaagtgttccagggcaaccaggactccttcacccccgtggtcaactccctggaccccccactgctgaccagatacctgagaatccacccccagagctgggtgcaccagatcgccctgagaatggaagtgctgggatgcgaggcccaggatctgtacggatctgggggcagtggcggatccgggggaagcggctcactggccgaggcaaaagaggccgcaaacgccgaactggattcatatggcgtgtcagatttctataagagactgattgacaaagcaaagaccgtggaaggggtggaagcactgaaggacgcaatcctggcagccctgcccggatctggcggaagcggggggagtggcggctctggaagcctggcagaagcaaaagaggcagctaacgcagagctggatagttacggcgtgtcagacttttacaaacgcctgattgacaaggccaaaaccgtggaaggcgtggaagcactgaaagatgcaattctggccgcactgccctga

SEQ ID NO: 55 - DNA, 4890 bp, Artificial Sequence
  AD2CD2woLG_SC na
atgcagatcgagctgtctacctgcttcttcctgtgcctgctgcggttctgcttcagcgccacccggcggtactacctgggcgccgtggaactgagctgggactacatgcagagcgacctgggcgagctgcccgtggacgccagattccccccaagagtgcccaagagcttccccttcaacacctccgtggtgtacaagaaaaccctgttcgtcgagttcaccgaccacctgttcaatatcgccaagcccagacccccctggatgggcctgctgggccctacaatccaggccgaggtgtacgacaccgtggtcatcaccctgaagaacatggccagccaccccgtgtccctgcacgccgtgggcgtgtcctactggaaggccagcgagggcgccgagtacgacgaccagaccagccagcgcgagaaagaggacgacaaagtctttcctggcggcagccatacctacgtgtggcaggtcctgaaagaaaacggccccatggcctccgaccccctgtgcctgacctacagctacctgagccacgtggacctggtcaaggacctgaacagcggcctgatcggcgccctgctcgtgtgcagagagggcagcctggccaaagagaaaacccagaccctgcacaagttcatcctgctgttcgccgtgttcgacgagggcaagagctggcacagcgagacaaagaacagcctgatgcaggaccgggacgccgcctctgccagagcctggcctaagatgcacaccgtgaacggctacgtgaacagaagcctgcccggactgatcggctgccaccggaagtccgtgtactggcacgtgatcggcatgggtaccacccccgaggtgcacagcatctttctggaaggacacaccttcctcgtgcggaaccaccggcaggccagcctggaaatcagccctatcaccttcctgaccgcccagacactgctgatggacctgggccagttcctgctgttttgccacatcagcagccaccagcacgacggcatggaagcctacgtgaaggtggacagctgccccgaggaaccccagctgcggatgaagaacaacgaggaagccgaggactacgacgacgacctgaccgacagcgagatggacgtcgtcagattcgatgacgacaacagccccagcttcatccagatcagaagcgtggccaagaagcaccccaagacctgggtgcactatatcgccgccgaggaagaggactgggactacgcccctctggtgctggcccccgacgacagaagctacaagagccagtacctgaacaatggcccccagcggatcggccggaagtacaagaaagtgcggttcatggcctacaccgacgagacattcaagaccagagaggccatccagcacgagagcggcatcctgggccccctgctgtacggcgaagtgggcgacaccctgctgattatcttcaagaaccaggccagccggccctacaacatctacccccacggcatcaccgacgtgcggcccctgtacagcagacggctgcccaagggcgtgaagcacctgaaggacttccccatcctgcccggggagatcttcaagtacaagtggaccgtgaccgtggaagatggccccaccaagagcgaccccagatgcctgacccggtactacagcagcttcgtgaacatggaacgggacctggcctccgggctgatcggccctctgctgatctgctacaaagaaagcgtggaccagcggggcaaccagatcatgagcgacaagcggaacgtgatcctgttcagcgtgttcgatgagaacagaagctggtatctgaccgagaatatccagcggttcctgcccaaccctgccggcgtgcagctggaagatcccgagttccaggccagcaacatcatgcactccatcaatggctacgtgttcgacagcctgcagctgagcgtgtgcctgcacgaggtggcctactggtacatcctgagcatcggcgcccagaccgacttcctgagcgtgttcttcagcggctacaccttcaagcacaagatggtgtacgaggataccctgaccctgttccccttctccggcgaaaccgtgttcatgagcatggaaaaccccggcctgtggattctgggctgccacaacagcgacttccggaaccggggcatgaccgccctgctgaaggtgtccagctgcgacaagaacaccggcgactactacgaggacagctacgaggatatcagcgcctacctgctgagcaagaacaacgccatcgagccccggtccttcagccagaacccccccgtgctgctggccgaagccaaagaagccgccaacgccgagctggacagctatggcgtgtccgacttctacaagcggctgatcgacaaggccaagacagtggaaggcgtggaagctctgaaggatgccatcctggccgctctgcctctggccgaggctaaagaggctgctaacgcagaactggactcttacggggtgtccgatttttacaaaagactgattgataaggctaaaactgtggaaggggtggaagccctgaaagacgctattctggctgccctgccccagagcgaccaggaagagatcgactacgacgacaccatcagcgtcgagatgaagaaagaggacttcgacatctacgacgaggacgagaaccagagcccccggtccttccagaagaaaacccggcactactttatcgccgccgtggaacggctgtgggactacggcatgagcagcagcccccacgtgctgcggaatagagcccagagcggcagcgtgccccagttcaagaaagtggtgttccaggagttcaccgacggcagcttcacccagcctctgtaccggggcgagctgaacgagcacctgggcctgctgggcccctacatccgggccgaggtggaagataacatcatggtcaccttccggaaccaggccagccggccctacagcttctacagcagcctgatctcctacgaagaggaccagcggcagggcgccgagccccggaagaacttcgtgaagcccaacgagacaaagacctacttctggaaggtgcagcaccacatggcccccaccaaggacgaattcgactgcaaggcctgggcctacttcagcgacgtggacctggaaaaggacgtgcacagcggcctgatcggccccctgctcgtgtgccacaccaacaccctgaaccccgcccacggccggcaggtcacagtgcaggaatttgccctgttcttcaccatcttcgacgagactaagagctggtacttcaccgagaacatggaacggaactgcagagccccctgcaacatccagatggaagatcccaccttcaaagagaactaccggttccacgccatcaatggctacatcatggacaccctgcccggcctggtcatggcccaggaccagagaatccggtggtatctgctgagcatgggcagcaacgagaacatccacagcatccacttcagcggccacgtgttcaccgtgcggaagaaagaagagtacaagatggccctgtacaacctgtaccccggcgtgttcgaaacagtggaaatgctgcccagcaaggccggcatctggcgggtggaatgtctgatcggcgagcatctgcacgccggcatgtccaccctgtttctggtgtacagcaacaagtgccagacccccctgggcatggccagcggccacatccgggatttccagatcaccgcctccggccagtacggccagtgggcccctaaactggcccggctgcactacagcggcagcatcaacgcctggtccaccaaagagcccttcagctggatcaaggtggacctgctggcccccatgattatccacggcatcaagacacagggcgccagacagaagttcagcagcctgtacatcagccagttcatcatcatgtacagcctggatggcaagaagtggcagacctaccggggcaacagcaccggcaccctgatggtgttcttcggcaacgtggacagcagcggcatcaagcacaacatcttcaacccccccatcattgcccggtacatccggctgcaccccacccactacagcatccggtccaccctgcggatggaactgatgggctgcgacctgaacagctgctccatgcctctgggcatggaaagcaaggccatcagcgacgcccagatcacagccagcagctacttcaccaacatgttcgccacctggtccccatccaaggccagactgcatctgcagggcagaagcaatgcctggcggcctcaggtcaacaaccccaaagaatggctccaggtggacttccagaaaaccatgaaggtcacaggcgtgaccacccagggcgtgaagtccctgctgacctctatgtacgtgaaagagttcctgatctccagcagccaggacggccaccagtggaccctgttctttcagaacggcaaagtgaaagtgttccagggcaaccaggactccttcacccccgtggtcaactccctggaccccccactgctgaccagatacctgagaatccacccccagagctgggtgcaccagatcgccctgagaatggaagtgctgggatgcgaggcccaggatctgtacctggctgaggcaaaagaagcagcaaacgccgaactggattcctacggcgtgtcagacttctataagagactgattgacaaagccaaaaccgtggaaggcgtggaagcactgaaggacgcaattctggccgcactgcccctggcagaagcaaaagaggccgctaacgctgagctggattcatatggggtgtcagatttctacaaacgcctgatcgacaaagcaaaaacagtggaaggggtggaagccctgaaggacgccatcctggcagccctgccatga

SEQ ID NO: 56 - DNA, 5337 bp, Artificial Sequence
  AbD2CD2_SC na
atgcagatcgagctgtctacctgcttcttcctgtgcctgctgcggttctgcttcagcgccacccggcggtactacctgggcgccgtggaactgagctgggactacatgcagagcgacctgggcgagctgcccgtggacgccagattccccccaagagtgcccaagagcttccccttcaacacctccgtggtgtacaagaaaaccctgttcgtcgagttcaccgaccacctgttcaatatcgccaagcccagacccccctggatgggcctgctgggccctacaatccaggccgaggtgtacgacaccgtggtcatcaccctgaagaacatggccagccaccccgtgtccctgcacgccgtgggcgtgtcctactggaaggccagcgagggcgccgagtacgacgaccagaccagccagcgcgagaaagaggacgacaaagtctttcctggcggcagccatacctacgtgtggcaggtcctgaaagaaaacggccccatggcctccgaccccctgtgcctgacctacagctacctgagccacgtggacctggtcaaggacctgaacagcggcctgatcggcgccctgctcgtgtgcagagagggcagcctggccaaagagaaaacccagaccctgcacaagttcatcctgctgttcgccgtgttcgacgagggcaagagctggcacagcgagacaaagaacagcctgatgcaggaccgggacgccgcctctgccagagcctggcctaagatgcacaccgtgaacggctacgtgaacagaagcctgcccggactgatcggctgccaccggaagtccgtgtactggcacgtgatcggcatgggtaccacccccgaggtgcacagcatctttctggaaggacacaccttcctcgtgcggaaccaccggcaggccagcctggaaatcagccctatcaccttcctgaccgcccagacactgctgatggacctgggccagttcctgctgttttgccacatcagcagccaccagcacgacggcatggaagcctacgtgaaggtggacagctgccccgaggaaccccagctgcggatgaagaacaacgaggaagccgaggactacgacgacgacctgaccgacagcgagatggacgtcgtcagattcgatgacgacaacagccccagcttcatccagatcagaagcgtggccaagaagcaccccaagacctgggtgcactatatcgccgccgaggaagaggactgggactacgcccctctggtgctggcccccgacgacagaagctacaagagccagtacctgaacaatggcccccagcggatcggccggaagtacaagaaagtgcggttcatggcctacaccgacgagacattcaagaccagagaggccatccagcacgagagcggcatcctgggccccctgctgtacggcgaagtgggcgacaccctgctgattatcttcaagaaccaggccagccggccctacaacatctacccccacggcatcaccgacgtgcggcccctgtacagcagacggctgcccaagggcgtgaagcacctgaaggacttccccatcctgcccggggagatcttcaagtacaagtggaccgtgaccgtggaagatggccccaccaagagcgaccccagatgcctgacccggtactacagcagcttcgtgaacatggaacgggacctggcctccgggctgatcggccctctgctgatctgctacaaagaaagcgtggaccagcggggcaaccagatcatgagcgacaagcggaacgtgatcctgttcagcgtgttcgatgagaacagaagctggtatctgaccgagaatatccagcggttcctgcccaaccctgccggcgtgcagctggaagatcccgagttccaggccagcaacatcatgcactccatcaatggctacgtgttcgacagcctgcagctgagcgtgtgcctgcacgaggtggcctactggtacatcctgagcatcggcgcccagaccgacttcctgagcgtgttcttcagcggctacaccttcaagcacaagatggtgtacgaggataccctgaccctgttccccttctccggcgaaaccgtgttcatgagcatggaaaaccccggcctgtggattctgggctgccacaacagcgacttccggaaccggggcatgaccgccctgctgaaggtgtccagctgcgacaagaacaccggcgactactacgaggacagctacgaggatatcagcgcctacctgctgagcaagaacaacgccatcgagcctcggagcttcagccagaatcctcctgtgctgtccaaaaacaatgccattgagccccggtcattctcccagaactctagacacccaagcctggccgaagccaaagaagccgctaacgccgagctggatagctacggcgtgtccgacttctacaagcggctgatcgataaggccaagaccgtggaaggcgtggaagccctgaaagatgctattctggccgctctgcctggaagcggaggtagcggaggatctggcggttctggcagcggatctaagaacaatgctatcgaaccccgctcctttagccagaacagcagacacccttctggaagtggtggttctggtggatccggcggatctggatctctggcagaggctaaagaggccgccaacgctgaactcgactcctatggggtgtccgatttttacaaacgcctcattgacaaggccaaaacagtcgagggcgtcgaggctctgaaggatgccattcttgctgccctgccatctaaaaacaacgcaatagagcccagatccttcagtcagaacagtcggcaccccagccagagcgaccaagaggaaatcgacttcgacgacaccatcagcgtcgagatgaagaaagaggacttcgacatcttcgacgaggacgagaatcagagccccagaagcttccagaaaaagacccggcactactttatcgccgccgtggaaagactgtgggactacggaatgagcagcagccctcacgtgctgagaaatagagcacagagcggcagcgtgccccagttcaagaaagtggtgttccaagagttcaccgacggcagcttcacccagcctctgtatagaggcgagctgaatgagcacctgggcctgctgggaccttatatcagagccgaggtggaagataacatcatggtcaccttccggaaccaggccagcagaccctacagcttctacagcagcctgatcagctacgaagaggaccagagacagggcgccgagcctagaaagaacttcgtgaagcccaacgagacaaagacctacttttggaaggtgcagcaccacatggcccctaccaaggacgaattcgattgcaaggcctgggcctacttcagcgacgtggacctggaaaaggatgtgcactctggcctgatcggccctctgctcgtgtgtcacaccaacacactgaaccccgctcacggcagacaagtgaccgtgcaagagttcgccctgttcttcaccatcttcgacgagacaaagagctggtacttcaccgagaacatggaacggaactgcagagccccttgcaacatccagatggaagatcccaccttcaaagagaactaccggttccacgccatcaacggctacatcatggataccctgcctggcctggttatggcccaggaccagagaatcagatggtatctgctgagcatgggcagcaacgagaacatccacagcatccacttcagcggccacgtgttcaccgtgcggaagaaagaagagtacaagatggccctgtacaatctgtaccccggcgtgttcgagacagtggaaatgctgccttctaaggccggcatttggagagtggaatgtctgatcggagagcatctgcacgccggcatgagcaccctgtttctggtgtacagcaacaagtgtcagacccctctcggcatggcctccggccacatcagagattttcagatcacagccagcggccagtatggccagtgggctcctaaactggccagactgcactacagcggcagcatcaatgcctggtccaccaaagagcccttcagctggattaaggtggacctgctggcccctatgatcatccacggcatcaaaacacagggcgccagacagaagttcagcagcctgtacatcagccagttcatcatcatgtacagcctggacggcaagaagtggcagacctacagaggcaatagcaccggcacactgatggtgttcttcggcaatgtggacagcagcggcatcaagcacaacatcttcaaccctccaatcattgcccggtacatccggctgcaccccacacactacagcatccggtctaccctgagaatggaactgatgggctgcgacctgaacagctgtagcatgcccctcggaatggaaagcaaggccatcagcgacgcccagatcaccgcctccagctacttcaccaacatgttcgccacttggagcccctccaaagccagactccatctgcaaggcagatctaacgcttggaggccccaagtgaacaaccccaaagagtggctgcaggtcgacttccagaaaaccatgaaggtcacaggcgtgacaacccagggcgtgaagtctctgctgacctctatgtacgtgaaagagttcctgatcagcagcagccaggacggccaccagtggaccctgtttttccagaacggcaaagtgaaggtgttccagggcaaccaggacagcttcacccctgtggtcaatagcctggatcctccactgctgaccagatacctgcggattcaccctcagagctgggtgcaccagatcgctctcagaatggaagtgctgggatgtgaagcccaggatctgtactccaagaacaacgccatcgagcccagaagcttcagccagaactctagacacccaagcctggccgaagccaaagaagccgctaacgccgagctggatagctacggcgtgtccgacttctacaagcggctgatcgataaggccaagaccgtggaaggcgtggaagccctgaaagatgccattctggctgctctgcctggaagcggaggaagtggtggatctggcggttctggctccggcagcaaaaacaatgccattgagcctcggagcttctcccagaacagcagacatccttctggatcaggcggtagcggaggatccggcggatctggatctctggctgaggctaaagaggccgccaacgctgaactcgactcctatggggtgtccgatttttacaaacgcctcattgacaaggccaaaacagtcgagggcgtcgaggctctgaaggatgctattcttgccgcactgccctga

SEQ ID NO: 57 - DNA, 138 bp, Artificial Sequence
  optimized DNA sequence encoding SEQ ID NO: 46
ctggctgaagcaaaagaagcagccaatgcggaactggattcttatggcgtgagcgatttttataaacgcctgatcgataaagcaaaaaccgttgaaggtgttgaagcactgaaagatgcgattctggcagcactgccg

SEQ ID NO: 58 - DNA, 42 bp, Artificial Sequence
  exemplary DNA encoding Glycine-serine Linker G1 of SEQ ID NO:40
ggatcaggaggttcaggaggaagtggtggatctggaagtgga

SEQ ID NO: 59 - DNA, 36 bp, Artificial Sequence
  exemplary DNA encoding Glycine-serine Linker G2 of SEQ ID NO:41
ggcggcagcggcggcggcagcggcggcggcagcggc

SEQ ID NO: 60 - protein, 1533 aa, Artificial Sequence
  SIGNAL 1..19: Signal sequence
  FVIII-6rs - human FVIII B-domain deleted
  DOMAIN 20..348: A1
  DOMAIN 349..398: a1
  DOMAIN 399..730: A2
  DOMAIN 731..759: a2
  DOMAIN 760..849: truncated B-domain
  DOMAIN 850..894: a3
  DOMAIN 895..1221: A3
  DOMAIN 1222..1374: C1
  DOMAIN 1375..1533: C2
MQIELSTCFFLCLLRFCFSATRRYYLGAVELSWDYMQSDLGELPVDARFPPRVPKSFPFNTSVVYKKTLFVEFTDHLFNIAKPRPPWMGLLGPTIQAEVYDTVVITLKNMASHPVSLHAVGVSYWKASEGAEYDDQTSQREKEDDKVFPGGSHTYVWQVLKENGPMASDPLCLTYSYLSHVDLVKDLNSGLIGALLVCREGSLAKEKTQTLHKFILLFAVFDEGKSWHSETKNSLMQDRDAASARAWPKMHTVNGYVNRSLPGLIGCHRKSVYWHVIGMGTTPEVHSIFLEGHTFLVRNHRQASLEISPITFLTAQTLLMDLGQFLLFCHISSHQHDGMEAYVKVDSCPEEPQLRMKNNEEAEDYDDDLTDSEMDVVRFDDDNSPSFIQIRSVAKKHPKTWVHYIAAEEEDWDYAPLVLAPDDRSYKSQYLNNGPQRIGRKYKKVRFMAYTDETFKTREAIQHESGILGPLLYGEVGDTLLIIFKNQASRPYNIYPHGITDVRPLYSRRLPKGVKHLKDFPILPGEIFKYKWTVTVEDGPTKSDPRCLTRYYSSFVNMERDLASGLIGPLLICYKESVDQRGNQIMSDKRNVILFSVFDENRSWYLTENIQRFLPNPAGVQLEDPEFQASNIMHSINGYVFDSLQLSVCLHEVAYWYILSIGAQTDFLSVFFSGYTFKHKMVYEDTLTLFPFSGETVFMSMENPGLWILGCHNSDFRNRGMTALLKVSSCDKNTGDYYEDSYEDISAYLLSKNNAIEPRSFSQDPLAWDNHYGTQIPKEEWKSQEKSPEKTAFKKKDTILSLNACESNHAIAAINEGQNKPEIEVTWAKQGRTERLCSQNPPVLKRHQREITRTTLQSDQEEIDYDDTISVEMKKEDFDIYDEDENQSPRSFQKKTRHYFIAAVERLWDYGMSSSPHVLRNRAQSGSVPQFKKVVFQEFTDGSFTQPLYRGELNEHLGLLGPYIRAEVEDNIMVTFRNQASRPYSFYSSLISYEEDQRQGAEPRKNFVKPNETKTYFWKVQHHMAPTKDEFDCKAWAYFSDVDLEKDVHSGLIGPLLVCHTNTLNPAHGRQVTVQEFALFFTIFDETKSWYFTENMERNCRAPCNIQMEDPTFKENYRFHAINGYIMDTLPGLVMAQDQRIRWYLLSMGSNENIHSIHFSGHVFTVRKKEEYKMALYNLYPGVFETVEMLPSKAGIWRVECLIGEHLHAGMSTLFLVYSNKCQTPLGMASGHIRDFQITASGQYGQWAPKLARLHYSGSINAWSTKEPFSWIKVDLLAPMIIHGIKTQGARQKFSSLYISQFIIMYSLDGKKWQTYRGNSTGTLMVFFGNVDSSGIKHNIFNPPIIARYIRLHPTHYSIRSTLRMELMGCDLNSCSMPLGMESKAISDAQITASSYFTNMFATWSPSKARLHLQGRSNAWRPQVNNPKEWLQVDFQKTMKVTGVTTQGVKSLLTSMYVKEFLISSSQDGHQWTLFFQNGKVKVFQGNQDSFTPVVNSLDPPLLTRYLRIHPQSWVHQIALRMEVLGCEAQDLY

SEQ ID NO: 61 - protein, 1457 aa, Artificial Sequence; identical to SEQ ID NO: 28
  Refacto - human FVIII B-domain deleted

SEQ ID NO: 62 - protein, 1463 aa, Artificial Sequence
  SIGNAL 1..19: signal sequence
  Afstyla - human FVIII B-domain deleted single chain
MQIELSTCFFLCLLRFCFSATRRYYLGAVELSWDYMQSDLGELPVDARFPPRVPKSFPFNTSVVYKKTLFVEFTDHLFNIAKPRPPWMGLLGPTIQAEVYDTVVITLKNMASHPVSLHAVGVSYWKASEGAEYDDQTSQREKEDDKVFPGGSHTYVWQVLKENGPMASDPLCLTYSYLSHVDLVKDLNSGLIGALLVCREGSLAKEKTQTLHKFILLFAVFDEGKSWHSETKNSLMQDRDAASARAWPKMHTVNGYVNRSLPGLIGCHRKSVYWHVIGMGTTPEVHSIFLEGHTFLVRNHRQASLEISPITFLTAQTLLMDLGQFLLFCHISSHQHDGMEAYVKVDSCPEEPQLRMKNNEEAEDYDDDLTDSEMDVVRFDDDNSPSFIQIRSVAKKHPKTWVHYIAAEEEDWDYAPLVLAPDDRSYKSQYLNNGPQRIGRKYKKVRFMAYTDETFKTREAIQHESGILGPLLYGEVGDTLLIIFKNQASRPYNIYPHGITDVRPLYSRRLPKGVKHLKDFPILPGEIFKYKWTVTVEDGPTKSDPRCLTRYYSSFVNMERDLASGLIGPLLICYKESVDQRGNQIMSDKRNVILFSVFDENRSWYLTENIQRFLPNPAGVQLEDPEFQASNIMHSINGYVFDSLQLSVCLHEVAYWYILSIGAQTDFLSVFFSGYTFKHKMVYEDTLTLFPFSGETVFMSMENPGLWILGCHNSDFRNRGMTALLKVSSCDKNTGDYYEDSYEDISAYLLSKNNAIEPRSFSQNSRHPSTRQKQFNATTIPENTTLQSDQEEIDYDDTISVEMKKEDFDIYDEDENQSPRSFQKKTRHYFIAAVERLWDYGMSSSPHVLRNRAQSGSVPQFKKVVFQEFTDGSFTQPLYRGELNEHLGLLGPYIRAEVEDNIMVTFRNQASRPYSFYSSLISYEEDQRQGAEPRKNFVKPNETKTYFWKVQHHMAPTKDEFDCKAWAYFSDVDLEKDVHSGLIGPLLVCHTNTLNPAHGRQVTVQEFALFFTIFDETKSWYFTENMERNCRAPCNIQMEDPTFKENYRFHAINGYIMDTLPGLVMAQDQRIRWYLLSMGSNENIHSIHFSGHVFTVRKKEEYKMALYNLYPGVFETVEMLPSKAGIWRVECLIGEHLHAGMSTLFLVYSNKCQTPLGMASGHIRDFQITASGQYGQWAPKLARLHYSGSINAWSTKEPFSWIKVDLLAPMIIHGIKTQGARQKFSSLYISQFIIMYSLDGKKWQTYRGNSTGTLMVFFGNVDSSGIKHNIFNPPIIARYIRLHPTHYSIRSTLRMELMGCDLNSCSMPLGMESKAISDAQITASSYFTNMFATWSPSKARLHLQGRSNAWRPQVNNPKEWLQVDFQKTMKVTGVTTQGVKSLLTSMYVKEFLISSSQDGHQWTLFFQNGKVKVFQGNQDSFTPVVNSLDPPLLTRYLRIHPQSWVHQIALRMEVLGCEAQDLY

SEQ ID NO: 63 - protein, 1533 aa, Artificial Sequence
  SIGNAL 1..19
  FVIII-19M
  DOMAIN 20..348: A1
  MUTAGEN 79: N79S
  MUTAGEN 112: S112T
  MUTAGEN 160: L160S
  MUTAGEN 171: L171Q
  MUTAGEN 184: V184A
  MUTAGEN 223: N233D
  MUTAGEN 265: I265T
  MUTAGEN 299: N299D
  DOMAIN 349..398: a1
  DOMAIN 399..730: A2
  MUTAGEN 426: Y426H
  MUTAGEN 507: S507E
  MUTAGEN 555: F555H
  MUTAGEN 616: N616E
  MUTAGEN 706: L706N
  DOMAIN 731..759: a2
  MUTAGEN 748: Y748S
  DOMAIN 760..849: truncated B-domain
  DOMAIN 850..894: a3
  DOMAIN 895..1221: A3
  MUTAGEN 1019: K1837E
  MUTAGEN 1220: N2038D
  DOMAIN 1222..1374: C1
  MUTAGEN 1259: S2077G
  DOMAIN 1375..1533: C2
  MUTAGEN 1497: S2315T
  MUTAGEN 1515: V2333A
MQIELSTCFFLCLLRFCFSATRRYYLGAVELSWDYMQSDLGELPVDARFPPRVPKSFPFNTSVVYKKTLFVEFTDHLFSIAKPRPPWMGLLGPTIQAEVYDTVVITLKNMATHPVSLHAVGVSYWKASEGAEYDDQTSQREKEDDKVFPGGSHTYVWQVSKENGPMASDPQCLTYSYLSHVDLAKDLNSGLIGALLVCREGSLAKEKTQTLHKFILLFAVFDEGKSWHSETKDSLMQDRDAASARAWPKMHTVNGYVNRSLPGLTGCHRKSVYWHVIGMGTTPEVHSIFLEGHTFLVRDHRQASLEISPITFLTAQTLLMDLGQFLLFCHISSHQHDGMEAYVKVDSCPEEPQLRMKNNEEAEDYDDDLTDSEMDVVRFDDDNSPSFIQIRSVAKKHPKTWVHYIAAEEEDWDYAPLVLAPDDRSHKSQYLNNGPQRIGRKYKKVRFMAYTDETFKTREAIQHESGILGPLLYGEVGDTLLIIFKNQASRPYNIYPHGITDVRPLYERRLPKGVKHLKDFPILPGEIFKYKWTVTVEDGPTKSDPRCLTRYYSSHVNMERDLASGLIGPLLICYKESVDQRGNQIMSDKRNVILFSVFDENRSWYLTENIQRFLPEPAGVQLEDPEFQASNIMHSINGYVFDSLQLSVCLHEVAYWYILSIGAQTDFLSVFFSGYTFKHKMVYEDTLTLFPFSGETVFMSMENPGNWILGCHNSDFRNRGMTALLKVSSCDKNTGDYYEDSYEDISASLLSKNNAIEPRSFSQDPLAWDNHYGTQIPKEEWKSQEKSPEKTAFKKKDTILSLNACESNHAIAAINEGQNKPEIEVTWAKQGRTERLCSQNPPVLKRHQREITRTTLQSDQEEIDYDDTISVEMKKEDFDIYDEDENQSPRSFQKKTRHYFIAAVERLWDYGMSSSPHVLRNRAQSGSVPQFKKVVFQEFTDGSFTQPLYRGELNEHLGLLGPYIRAEVEDNIMVTFRNQASRPYSFYSSLISYEEDQRQGAEPRKNFVKPNETKTYFWEVQHHMAPTKDEFDCKAWAYFSDVDLEKDVHSGLIGPLLVCHTNTLNPAHGRQVTVQEFALFFTIFDETKSWYFTENMERNCRAPCNIQMEDPTFKENYRFHAINGYIMDTLPGLVMAQDQRIRWYLLSMGSNENIHSIHFSGHVFTVRKKEEYKMALYNLYPGVFETVEMLPSKAGIWRVECLIGEHLHAGMSTLFLVYSDKCQTPLGMASGHIRDFQITASGQYGQWAPKLARLHYSGGINAWSTKEPFSWIKVDLLAPMIIHGIKTQGARQKFSSLYISQFIIMYSLDGKKWQTYRGNSTGTLMVFFGNVDSSGIKHNIFNPPIIARYIRLHPTHYSIRSTLRMELMGCDLNSCSMPLGMESKAISDAQITASSYFTNMFATWSPSKARLHLQGRSNAWRPQVNNPKEWLQVDFQKTMKVTGVTTQGVKSLLTSMYVKEFLISSSQDGHQWTLFFQNGKVKVFQGNQDSFTPVVNTLDPPLLTRYLRIHPQSWAHQIALRMEVLGCEAQDLY

SEQ ID NO: 64 - protein, 1533 aa, Artificial Sequence
  SIGNAL 1..19
  FVIII-18M
MQIELSTCFFLCLLRFCFSATRRYYLGAVELSWDYMQSDLGELPVDARFPPRVPKSFPFNTSVVYKKTLFVEFTDHLFSIAKPRPPWMGLLGPTIQAEVYDTVVITLKNMATHPVSLHAVGVSYWKASEGAEYDDQTSQREKEDDKVFPGGSHTYVWQVSKENGPMASDPQCLTYSYLSHVDLAKDLNSGLIGALLVCREGSLAKEKTQTLHKFILLFAVFDEGKSWHSETKDSLMQDRDAASARAWPKMHTVNGYVNRSLPGLTGCHRKSVYWHVIGMGTTPEVHSIFLEGHTFLVRDHRQASLEISPITFLTAQTLLMDLGQFLLFCHISSHQHDGMEAYVKVDSCPEEPQLRMKNNEEAEDYDDDLTDSEMDVVRFDDDNSPSFIQIRSVAKKHPKTWVHYIAAEEEDWDYAPLVLAPDDRSHKSQYLNNGPQRIGRKYKKVRFMAYTDETFKTREAIQHESGILGPLLYGEVGDTLLIIFKNQASRPYNIYPHGITDVRPLYERRLPKGVKHLKDFPILPGEIFKYKWTVTVEDGPTKSDPRCLTRYYSSHVNMERDLASGLIGPLLICYKESVDQRGNQIMSDKRNVILFSVFDENRSWYLTENIQRFLPEPAGVQLEDPEFQASNIMHSINGYVFDSLQLSVCLHEVAYWYILSIGAQTDFLSVFFSGYTFKHKMVYEDTLTLFPFSGETVFMSMENPGNWILGCHNSDFRNRGMTALLKVSSCDKNTGDYYEDSYEDISASLLSKNNAIEPRSFSQDPLAWDNHYGTQIPKEEWKSQEKSPEKTAFKKKDTILSLNACESNHAIAAINEGQNKPEIEVTWAKQGRTERLCSQNPPVLKRHQREITRTTLQSDQEEIDYDDTISVEMKKEDFDIYDEDENQSPRSFQKKTRHYFIAAVERLWDYGMSSSPHVLRNRAQSGSVPQFKKVVFQEFTDGSFTQPLYRGELNEHLGLLGPYIRAEVEDNIMVTFRNQASRPYSFYSSLISYEEDQRQGAEPRKNFVKPNETKTYFWKVQHHMAPTKDEFDCKAWAYFSDVDLEKDVHSGLIGPLLVCHTNTLNPAHGRQVTVQEFALFFTIFDETKSWYFTENMERNCRAPCNIQMEDPTFKENYRFHAINGYIMDTLPGLVMAQDQRIRWYLLSMGSNENIHSIHFSGHVFTVRKKEEYKMALYNLYPGVFETVEMLPSKAGIWRVECLIGEHLHAGMSTLFLVYSDKCQTPLGMASGHIRDFQITASGQYGQWAPKLARLHYSGGINAWSTKEPFSWIKVDLLAPMIIHGIKTQGARQKFSSLYISQFIIMYSLDGKKWQTYRGNSTGTLMVFFGNVDSSGIKHNIFNPPIIARYIRLHPTHYSIRSTLRMELMGCDLNSCSMPLGMESKAISDAQITASSYFTNMFATWSPSKARLHLQGRSNAWRPQVNNPKEWLQVDFQKTMKVTGVTTQGVKSLLTSMYVKEFLISSSQDGHQWTLFFQNGKVKVFQGNQDSFTPVVNTLDPPLLTRYLRIHPQSWAHQIALRMEVLGCEAQDLY

SEQ ID NO: 65 - protein, 1533 aa, Artificial Sequence
  SIGNAL 1..19
  FVIII-15M
MQIELSTCFFLCLLRFCFSATRRYYLGAVELSWDYMQSDLGELPVDARFPPRVPKSFPFNTSVVYKKTLFVEFTDHLFSIAKPRPPWMGLLGPTIQAEVYDTVVITLKNMATHPVSLHAVGVSYWKASEGAEYDDQTSQREKEDDKVFPGGSHTYVWQVSKENGPMASDPQCLTYSYLSHVDLAKDLNSGLIGALLVCREGSLAKEKTQTLHKFILLFAVFDEGKSWHSETKDSLMQDRDAASARAWPKMHTVNGYVNRSLPGLTGCHRKSVYWHVIGMGTTPEVHSIFLEGHTFLVRDHRQASLEISPITFLTAQTLLMDLGQFLLFCHISSHQHDGMEAYVKVDSCPEEPQLRMKNNEEAEDYDDDLTDSEMDVVRFDDDNSPSFIQIRSVAKKHPKTWVHYIAAEEEDWDYAPLVLAPDDRSHKSQYLNNGPQRIGRKYKKVRFMAYTDETFKTREAIQHESGILGPLLYGEVGDTLLIIFKNQASRPYNIYPHGITDVRPLYERRLPKGVKHLKDFPILPGEIFKYKWTVTVEDGPTKSDPRCLTRYYSSHVNMERDLASGLIGPLLICYKESVDQRGNQIMSDKRNVILFSVFDENRSWYLTENIQRFLPEPAGVQLEDPEFQASNIMHSINGYVFDSLQLSVCLHEVAYWYILSIGAQTDFLSVFFSGYTFKHKMVYEDTLTLFPFSGETVFMSMENPGNWILGCHNSDFRNRGMTALLKVSSCDKNTGDYYEDSYEDISASLLSKNNAIEPRSFSQDPLAWDNHYGTQIPKEEWKSQEKSPEKTAFKKKDTILSLNACESNHAIAAINEGQNKPEIEVTWAKQGRTERLCSQNPPVLKRHQREITRTTLQSDQEEIDYDDTISVEMKKEDFDIYDEDENQSPRSFQKKTRHYFIAAVERLWDYGMSSSPHVLRNRAQSGSVPQFKKVVFQEFTDGSFTQPLYRGELNEHLGLLGPYIRAEVEDNIMVTFRNQASRPYSFYSSLISYEEDQRQGAEPRKNFVKPNETKTYFWEVQHHMAPTKDEFDCKAWAYFSDVDLEKDVHSGLIGPLLVCHTNTLNPAHGRQVTVQEFALFFTIFDETKSWYFTENMERNCRAPCNIQMEDPTFKENYRFHAINGYIMDTLPGLVMAQDQRIRWYLLSMGSNENIHSIHFSGHVFTVRKKEEYKMALYNLYPGVFETVEMLPSKAGIWRVECLIGEHLHAGMSTLFLVYSNKCQTPLGMASGHIRDFQITASGQYGQWAPKLARLHYSGSINAWSTKEPFSWIKVDLLAPMIIHGIKTQGARQKFSSLYISQFIIMYSLDGKKWQTYRGNSTGTLMVFFGNVDSSGIKHNIFNPPIIARYIRLHPTHYSIRSTLRMELMGCDLNSCSMPLGMESKAISDAQITASSYFTNMFATWSPSKARLHLQGRSNAWRPQVNNPKEWLQVDFQKTMKVTGVTTQGVKSLLTSMYVKEFLISSSQDGHQWTLFFQNGKVKVFQGNQDSFTPVVNSLDPPLLTRYLRIHPQSWVHQIALRMEVLGCEAQDLY

SEQ ID NO: 66 - protein, 1533 aa, Artificial Sequence
  SIGNAL 1..19
  FVIII-A1-7M
MQIELSTCFFLCLLRFCFSATRRYYLGAVELSWDYMQSDLGELPVDARFPPRVPKSFPFNTSVVYKKTLFVEFTDHLFSIAKPRPPWMGLLGPTIQAEVYDTVVITLKNMATHPVSLHAVGVSYWKASEGAEYDDQTSQREKEDDKVFPGGSHTYVWQVSKENGPMASDPQCLTYSYLSHVDLAKDLNSGLIGALLVCREGSLAKEKTQTLHKFILLFAVFDEGKSWHSETKDSLMQDRDAASARAWPKMHTVNGYVNRSLPGLTGCHRKSVYWHVIGMGTTPEVHSIFLEGHTFLVRNHRQASLEISPITFLTAQTLLMDLGQFLLFCHISSHQHDGMEAYVKVDSCPEEPQLRMKNNEEAEDYDDDLTDSEMDVVRFDDDNSPSFIQIRSVAKKHPKTWVHYIAAEEEDWDYAPLVLAPDDRSYKSQYLNNGPQRIGRKYKKVRFMAYTDETFKTREAIQHESGILGPLLYGEVGDTLLIIFKNQASRPYNIYPHGITDVRPLYSRRLPKGVKHLKDFPILPGEIFKYKWTVTVEDGPTKSDPRCLTRYYSSFVNMERDLASGLIGPLLICYKESVDQRGNQIMSDKRNVILFSVFDENRSWYLTENIQRFLPNPAGVQLEDPEFQASNIMHSINGYVFDSLQLSVCLHEVAYWYILSIGAQTDFLSVFFSGYTFKHKMVYEDTLTLFPFSGETVFMSMENPGLWILGCHNSDFRNRGMTALLKVSSCDKNTGDYYEDSYEDISAYLLSKNNAIEPRSFSQDPLAWDNHYGTQIPKEEWKSQEKSPEKTAFKKKDTILSLNACESNHAIAAINEGQNKPEIEVTWAKQGRTERLCSQNPPVLKRHQREITRTTLQSDQEEIDYDDTISVEMKKEDFDIYDEDENQSPRSFQKKTRHYFIAAVERLWDYGMSSSPHVLRNRAQSGSVPQFKKVVFQEFTDGSFTQPLYRGELNEHLGLLGPYIRAEVEDNIMVTFRNQASRPYSFYSSLISYEEDQRQGAEPRKNFVKPNETKTYFWKVQHHMAPTKDEFDCKAWAYFSDVDLEKDVHSGLIGPLLVCHTNTLNPAHGRQVTVQEFALFFTIFDETKSWYFTENMERNCRAPCNIQMEDPTFKENYRFHAINGYIMDTLPGLVMAQDQRIRWYLLSMGSNENIHSIHFSGHVFTVRKKEEYKMALYNLYPGVFETVEMLPSKAGIWRVECLIGEHLHAGMSTLFLVYSNKCQTPLGMASGHIRDFQITASGQYGQWAPKLARLHYSGSINAWSTKEPFSWIKVDLLAPMIIHGIKTQGARQKFSSLYISQFIIMYSLDGKKWQTYRGNSTGTLMVFFGNVDSSGIKHNIFNPPIIARYIRLHPTHYSIRSTLRMELMGCDLNSCSMPLGMESKAISDAQITASSYFTNMFATWSPSKARLHLQGRSNAWRPQVNNPKEWLQVDFQKTMKVTGVTTQGVKSLLTSMYVKEFLISSSQDGHQWTLFFQNGKVKVFQGNQDSFTPVVNSLDPPLLTRYLRIHPQSWVHQIALRMEVLGCEAQDLY

SEQ ID NO: 67 - protein, 1533 aa, Artificial Sequence
  SIGNAL 1..19
  FVIII-A2-4M
MQIELSTCFFLCLLRFCFSATRRYYLGAVELSWDYMQSDLGELPVDARFPPRVPKSFPFNTSVVYKKTLFVEFTDHLFNIAKPRPPWMGLLGPTIQAEVYDTVVITLKNMASHPVSLHAVGVSYWKASEGAEYDDQTSQREKEDDKVFPGGSHTYVWQVLKENGPMASDPLCLTYSYLSHVDLVKDLNSGLIGALLVCREGSLAKEKTQTLHKFILLFAVFDEGKSWHSETKNSLMQDRDAASARAWPKMHTVNGYVNRSLPGLIGCHRKSVYWHVIGMGTTPEVHSIFLEGHTFLVRNHRQASLEISPITFLTAQTLLMDLGQFLLFCHISSHQHDGMEAYVKVDSCPEEPQLRMKNNEEAEDYDDDLTDSEMDVVRFDDDNSPSFIQIRSVAKKHPKTWVHYIAAEEEDWDYAPLVLAPDDRSYKSQYLNNGPQRIGRKYKKVRFMAYTDETFKTREAIQHESGILGPLLYGEVGDTLLIIFKNQASRPYNIYPHGITDVRPLYSRRLPKGVKHLKDFPILPGEIFKYKWTVTVEDGPTKSDPRCLTRYYSSHVNMERDLASGLIGPLLICYKESVDQRGNQIMSDKRNVILFSVFDENRSWYLTENIQRFLPEPAGVQLEDPEFQASNIMHSINGYVFDSLQLSVCLHEVAYWYILSIGAQTDFLSVFFSGYTFKHKMVYEDTLTLFPFSGETVFMSMENPGNWILGCHNSDFRNRGMTALLKVSSCDKNTGDYYEDSYEDISASLLSKNNAIEPRSFSQDPLAWDNHYGTQIPKEEWKSQEKSPEKTAFKKKDTILSLNACESNHAIAAINEGQNKPEIEVTWAKQGRTERLCSQNPPVLKRHQREITRTTLQSDQEEIDYDDTISVEMKKEDFDIYDEDENQSPRSFQKKTRHYFIAAVERLWDYGMSSSPHVLRNRAQSGSVPQFKKVVFQEFTDGSFTQPLYRGELNEHLGLLGPYIRAEVEDNIMVTFRNQASRPYSFYSSLISYEEDQRQGAEPRKNFVKPNETKTYFWKVQHHMAPTKDEFDCKAWAYFSDVDLEKDVHSGLIGPLLVCHTNTLNPAHGRQVTVQEFALFFTIFDETKSWYFTENMERNCRAPCNIQMEDPTFKENYRFHAINGYIMDTLPGLVMAQDQRIRWYLLSMGSNENIHSIHFSGHVFTVRKKEEYKMALYNLYPGVFETVEMLPSKAGIWRVECLIGEHLHAGMSTLFLVYSNKCQTPLGMASGHIRDFQITASGQYGQWAPKLARLHYSGSINAWSTKEPFSWIKVDLLAPMIIHGIKTQGARQKFSSLYISQFIIMYSLDGKKWQTYRGNSTGTLMVFFGNVDSSGIKHNIFNPPIIARYIRLHPTHYSIRSTLRMELMGCDLNSCSMPLGMESKAISDAQITASSYFTNMFATWSPSKARLHLQGRSNAWRPQVNNPKEWLQVDFQKTMKVTGVTTQGVKSLLTSMYVKEFLISSSQDGHQWTLFFQNGKVKVFQGNQDSFTPVVNSLDPPLLTRYLRIHPQSWVHQIALRMEVLGCEAQDLY

SEQ ID NO: 68 - protein, 1533 aa, Artificial Sequence
  SIGNAL 1..19
  FVIII-BA3-1M
MQIELSTCFFLCLLRFCFSATRRYYLGAVELSWDYMQSDLGELPVDARFPPRVPKSFPFNTSVVYKKTLFVEFTDHLFNIAKPRPPWMGLLGPTIQAEVYDTVVITLKNMASHPVSLHAVGVSYWKASEGAEYDDQTSQREKEDDKVFPGGSHTYVWQVLKENGPMASDPLCLTYSYLSHVDLVKDLNSGLIGALLVCREGSLAKEKTQTLHKFILLFAVFDEGKSWHSETKNSLMQDRDAASARAWPKMHTVNGYVNRSLPGLIGCHRKSVYWHVIGMGTTPEVHSIFLEGHTFLVRNHRQASLEISPITFLTAQTLLMDLGQFLLFCHISSHQHDGMEAYVKVDSCPEEPQLRMKNNEEAEDYDDDLTDSEMDVVRFDDDNSPSFIQIRSVAKKHPKTWVHYIAAEEEDWDYAPLVLAPDDRSYKSQYLNNGPQRIGRKYKKVRFMAYTDETFKTREAIQHESGILGPLLYGEVGDTLLIIFKNQASRPYNIYPHGITDVRPLYSRRLPKGVKHLKDFPILPGEIFKYKWTVTVEDGPTKSDPRCLTRYYSSFVNMERDLASGLIGPLLICYKESVDQRGNQIMSDKRNVILFSVFDENRSWYLTENIQRFLPNPAGVQLEDPEFQASNIMHSINGYVFDSLQLSVCLHEVAYWYILSIGAQTDFLSVFFSGYTFKHKMVYEDTLTLFPFSGETVFMSMENPGLWILGCHNSDFRNRGMTALLKVSSCDKNTGDYYEDSYEDISAYLLSKNNAIEPRSFSQDPLAWDNHYGTQIPKEEWKSQEKSPEKTAFKKKDTILSLNACESNHAIAAINEGQNKPEIEVTWAKQGRTERLCSQNPPVLKRHQREITRTTLQSDQEEIDYDDTISVEMKKEDFDIYDEDENQSPRSFQKKTRHYFIAAVERLWDYGMSSSPHVLRNRAQSGSVPQFKKVVFQEFTDGSFTQPLYRGELNEHLGLLGPYIRAEVEDNIMVTFRNQASRPYSFYSSLISYEEDQRQGAEPRKNFVKPNETKTYFWEVQHHMAPTKDEFDCKAWAYFSDVDLEKDVHSGLIGPLLVCHTNTLNPAHGRQVTVQEFALFFTIFDETKSWYFTENMERNCRAPCNIQMEDPTFKENYRFHAINGYIMDTLPGLVMAQDQRIRWYLLSMGSNENIHSIHFSGHVFTVRKKEEYKMALYNLYPGVFETVEMLPSKAGIWRVECLIGEHLHAGMSTLFLVYSNKCQTPLGMASGHIRDFQITASGQYGQWAPKLARLHYSGSINAWSTKEPFSWIKVDLLAPMIIHGIKTQGARQKFSSLYISQFIIMYSLDGKKWQTYRGNSTGTLMVFFGNVDSSGIKHNIFNPPIIARYIRLHPTHYSIRSTLRMELMGCDLNSCSMPLGMESKAISDAQITASSYFTNMFATWSPSKARLHLQGRSNAWRPQVNNPKEWLQVDFQKTMKVTGVTTQGVKSLLTSMYVKEFLISSSQDGHQWTLFFQNGKVKVFQGNQDSFTPVVNSLDPPLLTRYLRIHPQSWVHQIALRMEVLGCEAQDLY

SEQ ID NO: 69 - protein, 1533 aa, Artificial Sequence
  SIGNAL 1..19
  FVIII-A3C2-4M
MQIELSTCFFLCLLRFCFSATRRYYLGAVELSWDYMQSDLGELPVDARFPPRVPKSFPFNTSVVYKKTLFVEFTDHLFNIAKPRPPWMGLLGPTIQAEVYDTVVITLKNMASHPVSLHAVGVSYWKASEGAEYDDQTSQREKEDDKVFPGGSHTYVWQVLKENGPMASDPLCLTYSYLSHVDLVKDLNSGLIGALLVCREGSLAKEKTQTLHKFILLFAVFDEGKSWHSETKNSLMQDRDAASARAWPKMHTVNGYVNRSLPGLIGCHRKSVYWHVIGMGTTPEVHSIFLEGHTFLVRNHRQASLEISPITFLTAQTLLMDLGQFLLFCHISSHQHDGMEAYVKVDSCPEEPQLRMKNNEEAEDYDDDLTDSEMDVVRFDDDNSPSFIQIRSVAKKHPKTWVHYIAAEEEDWDYAPLVLAPDDRSYKSQYLNNGPQRIGRKYKKVRFMAYTDETFKTREAIQHESGILGPLLYGEVGDTLLIIFKNQASRPYNIYPHGITDVRPLYSRRLPKGVKHLKDFPILPGEIFKYKWTVTVEDGPTKSDPRCLTRYYSSFVNMERDLASGLIGPLLICYKESVDQRGNQIMSDKRNVILFSVFDENRSWYLTENIQRFLPNPAGVQLEDPEFQASNIMHSINGYVFDSLQLSVCLHEVAYWYILSIGAQTDFLSVFFSGYTFKHKMVYEDTLTLFPFSGETVFMSMENPGLWILGCHNSDFRNRGMTALLKVSSCDKNTGDYYEDSYEDISAYLLSKNNAIEPRSFSQDPLAWDNHYGTQIPKEEWKSQEKSPEKTAFKKKDTILSLNACESNHAIAAINEGQNKPEIEVTWAKQGRTERLCSQNPPVLKRHQREITRTTLQSDQEEIDYDDTISVEMKKEDFDIYDEDENQSPRSFQKKTRHYFIAAVERLWDYGMSSSPHVLRNRAQSGSVPQFKKVVFQEFTDGSFTQPLYRGELNEHLGLLGPYIRAEVEDNIMVTFRNQASRPYSFYSSLISYEEDQRQGAEPRKNFVKPNETKTYFWKVQHHMAPTKDEFDCKAWAYFSDVDLEKDVHSGLIGPLLVCHTNTLNPAHGRQVTVQEFALFFTIFDETKSWYFTENMERNCRAPCNIQMEDPTFKENYRFHAINGYIMDTLPGLVMAQDQRIRWYLLSMGSNENIHSIHFSGHVFTVRKKEEYKMALYNLYPGVFETVEMLPSKAGIWRVECLIGEHLHAGMSTLFLVYSDKCQTPLGMASGHIRDFQITASGQYGQWAPKLARLHYSGGINAWSTKEPFSWIKVDLLAPMIIHGIKTQGARQKFSSLYISQFIIMYSLDGKKWQTYRGNSTGTLMVFFGNVDSSGIKHNIFNPPIIARYIRLHPTHYSIRSTLRMELMGCDLNSCSMPLGMESKAISDAQITASSYFTNMFATWSPSKARLHLQGRSNAWRPQVNNPKEWLQVDFQKTMKVTGVTTQGVKSLLTSMYVKEFLISSSQDGHQWTLFFQNGKVKVFQGNQDSFTPVVNTLDPPLLTRYLRIHPQSWAHQIALRMEVLGCEAQDLY

SEQ ID NO: 70 - protein, 1533 aa, Artificial Sequence
  SIGNAL 1..19
  FVIII-GOF1
MQIELSTCFFLCLLRFCFSATRRYYLGAVELSWDYMQSDLGELPVDARFPPRVPKSFPFNTSVVYKKTLFVEFTDHLFNIAKPRPPWMGLLGPTIQAEVYDTVVITLKNMASHPVSLHAVGVSYWKASEGAEYDDQTSQREKEDDKVFPGGSHTYVWQVLKENGPMASDPQCLTYSYLSHVDLVKDLNSGLIGALLVCREGSLAKEKTQTLHKFILLFAVFDEGKSWHSETKNSLMQDRDAASARAWPKMHTVNGYVNRSLPGLIGCHRKSVYWHVIGMGTTPEVHSIFLEGHTFLVRNHRQASLEISPITFLTAQTLLMDLGQFLLFCHISSHQHDGMEAYVKVDSCPEEPQLRMKNNEEAEDYDDDLTDSEMDVVRFDDDNSPSFIQIRSVAKKHPKTWVHYIAAEEEDWDYAPLVLAPDDRSYKSQYLNNGPQRIGRKYKKVRFMAYTDETFKTREAIQHESGILGPLLYGEVGDTLLIIFKNQASRPYNIYPHGITDVRPLYERRLPKGVKHLKDFPILPGEIFKYKWTVTVEDGPTKSDPRCLTRYYSSFVNMERDLASGLIGPLLICYKESVDQRGNQIMSDKRNVILFSVFDENRSWYLTENIQRFLPNPAGVQLEDPEFQASNIMHSINGYVFDSLQLSVCLHEVAYWYILSIGAQTDFLSVFFSGYTFKHKMVYEDTLTLFPFSGETVFMSMENPGLWILGCHNSDFRNRGMTALLKVSSCDKNTGDYYEDSYEDISASLLSKNNAIEPRSFSQDPLAWDNHYGTQIPKEEWKSQEKSPEKTAFKKKDTILSLNACESNHAIAAINEGQNKPEIEVTWAKQGRTERLCSQNPPVLKRHQREITRTTLQSDQEEIDYDDTISVEMKKEDFDIYDEDENQSPRSFQKKTRHYFIAAVERLWDYGMSSSPHVLRNRAQSGSVPQFKKVVFQEFTDGSFTQPLYRGELNEHLGLLGPYIRAEVEDNIMVTFRNQASRPYSFYSSLISYEEDQRQGAEPRKNFVKPNETKTYFWKVQHHMAPTKDEFDCKAWAYFSDVDLEKDVHSGLIGPLLVCHTNTLNPAHGRQVTVQEFALFFTIFDETKSWYFTENMERNCRAPCNIQMEDPTFKENYRFHAINGYIMDTLPGLVMAQDQRIRWYLLSMGSNENIHSIHFSGHVFTVRKKEEYKMALYNLYPGVFETVEMLPSKAGIWRVECLIGEHLHAGMSTLFLVYSNKCQTPLGMASGHIRDFQITASGQYGQWAPKLARLHYSGSINAWSTKEPFSWIKVDLLAPMIIHGIKTQGARQKFSSLYISQFIIMYSLDGKKWQTYRGNSTGTLMVFFGNVDSSGIKHNIFNPPIIARYIRLHPTHYSIRSTLRMELMGCDLNSCSMPLGMESKAISDAQITASSYFTNMFATWSPSKARLHLQGRSNAWRPQVNNPKEWLQVDFQKTMKVTGVTTQGVKSLLTSMYVKEFLISSSQDGHQWTLFFQNGKVKVFQGNQDSFTPVVNSLDPPLLTRYLRIHPQSWAHQIALRMEVLGCEAQDLY

SEQ ID NO: 71 - protein, 1533 aa, Artificial Sequence
  SIGNAL 1..19
  FVIII-GOF2
MQIELSTCFFLCLLRFCFSATRRYYLGAVELSWDYMQSDLGELPVDARFPPRVPKSFPFNTSVVYKKTLFVEFTDHLFNIAKPRPPWMGLLGPTIQAEVYDTVVITLKNMASHPVSLHAVGVSYWKASEGAEYDDQTSQREKEDDKVFPGGSHTYVWQVLKENGPMASDPQCLTYSYLSHVDLVKDLNSGLIGALLVCREGSLAKEKTQTLHKFILLFAVFDEGKSWHSETKNSLMQDRDAASARAWPKMHTVNGYVNRSLPGLIGCHRKSVYWHVIGMGTTPEVHSIFLEGHTFLVRDHRQASLEISPITFLTAQTLLMDLGQFLLFCHISSHQHDGMEAYVKVDSCPEEPQLRMKNNEEAEDYDDDLTDSEMDVVRFDDDNSPSFIQIRSVAKKHPKTWVHYIAAEEEDWDYAPLVLAPDDRSYKSQYLNNGPQRIGRKYKKVRFMAYTDETFKTREAIQHESGILGPLLYGEVGDTLLIIFKNQASRPYNIYPHGITDVRPLYSRRLPKGVKHLKDFPILPGEIFKYKWTVTVEDGPTKSDPRCLTRYYSSFVNMERDLASGLIGPLLICYKESVDQRGNQIMSDKRNVILFSVFDENRSWYLTENIQRFLPEPAGVQLEDPEFQASNIMHSINGYVFDSLQLSVCLHEVAYWYILSIGAQTDFLSVFFSGYTFKHKMVYEDTLTLFPFSGETVFMSMENPGLWILGCHNSDFRNRGMTALLKVSSCDKNTGDYYEDSYEDISAYLLSKNNAIEPRSFSQDPLAWDNHYGTQIPKEEWKSQEKSPEKTAFKKKDTILSLNACESNHAIAAINEGQNKPEIEVTWAKQGRTERLCSQNPPVLKRHQREITRTTLQSDQEEIDYDDTISVEMKKEDFDIYDEDENQSPRSFQKKTRHYFIAAVERLWDYGMSSSPHVLRNRAQSGSVPQFKKVVFQEFTDGSFTQPLYRGELNEHLGLLGPYIRAEVEDNIMVTFRNQASRPYSFYSSLISYEEDQRQGAEPRKNFVKPNETKTYFWKVQHHMAPTKDEFDCKAWAYFSDVDLEKDVHSGLIGPLLVCHTNTLNPAHGRQVTVQEFALFFTIFDETKSWYFTENMERNCRAPCNIQMEDPTFKENYRFHAINGYIMDTLPGLVMAQDQRIRWYLLSMGSNENIHSIHFSGHVFTVRKKEEYKMALYNLYPGVFETVEMLPSKAGIWRVECLIGEHLHAGMSTLFLVYSNKCQTPLGMASGHIRDFQITASGQYGQWAPKLARLHYSGSINAWSTKEPFSWIKVDLLAPMIIHGIKTQGARQKFSSLYISQFIIMYSLDGKKWQTYRGNSTGTLMVFFGNVDSSGIKHNIFNPPIIARYIRLHPTHYSIRSTLRMELMGCDLNSCSMPLGMESKAISDAQITASSYFTNMFATWSPSKARLHLQGRSNAWRPQVNNPKEWLQVDFQKTMKVTGVTTQGVKSLLTSMYVKEFLISSSQDGHQWTLFFQNGKVKVFQGNQDSFTPVVNSLDPPLLTRYLRIHPQSWAHQIALRMEVLGCEAQDLY

SEQ ID NO: 72 - protein, 1533 aa, Artificial Sequence
  SIGNAL 1..19
  FVIII-LS1
MQIELSTCFFLCLLRFCFSATRRYYLGAVELSWDYMQSDLGELPVDARFPPRVPKSFPFNTSVVYKKTLFVEFTDHLFNIAKPRPPWMGLLGPTIQAEVYDTVVITLKNMATHPVSLHAVGVSYWKASEGAEYDDQTSQREKEDDKVFPGGSHTYVWQVLKENGPMASDPLCLTYSYLSHVDLVKDLNSGLIGALLVCREGSLAKEKTQTLHKFILLFAVFDEGKSWHSETKNSLMQDRDAASARAWPKMHTVNGYVNRSLPGLIGCHRKSVYWHVIGMGTTPEVHSIFLEGHTFLVRNHRQASLEISPITFLTAQTLLMDLGQFLLFCHISSHQHDGMEAYVKVDSCPEEPQLRMKNNEEAEDYDDDLTDSEMDVVRFDDDNSPSFIQIRSVAKKHPKTWVHYIAAEEEDWDYAPLVLAPDDRSYKSQYLNNGPQRIGRKYKKVRFMAYTDETFKTREAIQHESGILGPLLYGEVGDTLLIIFKNQASRPYNIYPHGITDVRPLYERRLPKGVKHLKDFPILPGEIFKYKWTVTVEDGPTKSDPRCLTRYYSSFVNMERDLASGLIGPLLICYKESVDQRGNQIMSDKRNVILFSVFDENRSWYLTENIQRFLPNPAGVQLEDPEFQASNIMHSINGYVFDSLQLSVCLHEVAYWYILSIGAQTDFLSVFFSGYTFKHKMVYEDTLTLFPFSGETVFMSMENPGLWILGCHNSDFRNRGMTALLKVSSCDKNTGDYYEDSYEDISASLLSKNNAIEPRSFSQDPLAWDNHYGTQIPKEEWKSQEKSPEKTAFKKKDTILSLNACESNHAIAAINEGQNKPEIEVTWAKQGRTERLCSQNPPVLKRHQREITRTTLQSDQEEIDYDDTISVEMKKEDFDIYDEDENQSPRSFQKKTRHYFIAAVERLWDYGMSSSPHVLRNRAQSGSVPQFKKVVFQEFTDGSFTQPLYRGELNEHLGLLGPYIRAEVEDNIMVTFRNQASRPYSFYSSLISYEEDQRQGAEPRKNFVKPNETKTYFWEVQHHMAPTKDEFDCKAWAYFSDVDLEKDVHSGLIGPLLVCHTNTLNPAHGRQVTVQEFALFFTIFDETKSWYFTENMERNCRAPCNIQMEDPTFKENYRFHAINGYIMDTLPGLVMAQDQRIRWYLLSMGSNENIHSIHFSGHVFTVRKKEEYKMALYNLYPGVFETVEMLPSKAGIWRVECLIGEHLHAGMSTLFLVYSDKCQTPLGMASGHIRDFQITASGQYGQWAPKLARLHYSGSINAWSTKEPFSWIKVDLLAPMIIHGIKTQGARQKFSSLYISQFIIMYSLDGKKWQTYRGNSTGTLMVFFGNVDSSGIKHNIFNPPIIARYIRLHPTHYSIRSTLRMELMGCDLNSCSMPLGMESKAISDAQITASSYFTNMFATWSPSKARLHLQGRSNAWRPQVNNPKEWLQVDFQKTMKVTGVTTQGVKSLLTSMYVKEFLISSSQDGHQWTLFFQNGKVKVFQGNQDSFTPVVNSLDPPLLTRYLRIHPQSWVHQIALRMEVLGCEAQDLY

SEQ ID NO: 73 - protein, 1533 aa, Artificial Sequence
  SIGNAL 1..19
  FVIII-LS2
  MUTAGEN 112: S112T
  MUTAGEN 426: Y426H
  MUTAGEN 754: N754D
  MUTAGEN 1019: K1837E
  MUTAGEN 1220: N2038D
MQIELSTCFFLCLLRFCFSATRRYYLGAVELSWDYMQSDLGELPVDARFPPRVPKSFPFNTSVVYKKTLFVEFTDHLFNIAKPRPPWMGLLGPTIQAEVYDTVVITLKNMATHPVSLHAVGVSYWKASEGAEYDDQTSQREKEDDKVFPGGSHTYVWQVLKENGPMASDPLCLTYSYLSHVDLVKDLNSGLIGALLVCREGSLAKEKTQTLHKFILLFAVFDEGKSWHSETKNSLMQDRDAASARAWPKMHTVNGYVNRSLPGLIGCHRKSVYWHVIGMGTTPEVHSIFLEGHTFLVRNHRQASLEISPITFLTAQTLLMDLGQFLLFCHISSHQHDGMEAYVKVDSCPEEPQLRMKNNEEAEDYDDDLTDSEMDVVRFDDDNSPSFIQIRSVAKKHPKTWVHYIAAEEEDWDYAPLVLAPDDRSHKSQYLNNGPQRIGRKYKKVRFMAYTDETFKTREAIQHESGILGPLLYGEVGDTLLIIFKNQASRPYNIYPHGITDVRPLYSRRLPKGVKHLKDFPILPGEIFKYKWTVTVEDGPTKSDPRCLTRYYSSFVNMERDLASGLIGPLLICYKESVDQRGNQIMSDKRNVILFSVFDENRSWYLTENIQRFLPNPAGVQLEDPEFQASNIMHSINGYVFDSLQLSVCLHEVAYWYILSIGAQTDFLSVFFSGYTFKHKMVYEDTLTLFPFSGETVFMSMENPGLWILGCHNSDFRNRGMTALLKVSSCDKNTGDYYEDSYEDISAYLLSKNDAIEPRSFSQDPLAWDNHYGTQIPKEEWKSQEKSPEKTAFKKKDTILSLNACESNHAIAAINEGQNKPEIEVTWAKQGRTERLCSQNPPVLKRHQREITRTTLQSDQEEIDYDDTISVEMKKEDFDIYDEDENQSPRSFQKKTRHYFIAAVERLWDYGMSSSPHVLRNRAQSGSVPQFKKVVFQEFTDGSFTQPLYRGELNEHLGLLGPYIRAEVEDNIMVTFRNQASRPYSFYSSLISYEEDQRQGAEPRKNFVKPNETKTYFWEVQHHMAPTKDEFDCKAWAYFSDVDLEKDVHSGLIGPLLVCHTNTLNPAHGRQVTVQEFALFFTIFDETKSWYFTENMERNCRAPCNIQMEDPTFKENYRFHAINGYIMDTLPGLVMAQDQRIRWYLLSMGSNENIHSIHFSGHVFTVRKKEEYKMALYNLYPGVFETVEMLPSKAGIWRVECLIGEHLHAGMSTLFLVYSDKCQTPLGMASGHIRDFQITASGQYGQWAPKLARLHYSGSINAWSTKEPFSWIKVDLLAPMIIHGIKTQGARQKFSSLYISQFIIMYSLDGKKWQTYRGNSTGTLMVFFGNVDSSGIKHNIFNPPIIARYIRLHPTHYSIRSTLRMELMGCDLNSCSMPLGMESKAISDAQITASSYFTNMFATWSPSKARLHLQGRSNAWRPQVNNPKEWLQVDFQKTMKVTGVTTQGVKSLLTSMYVKEFLISSSQDGHQWTLFFQNGKVKVFQGNQDSFTPVVNSLDPPLLTRYLRIHPQSWVHQIALRMEVLGCEAQDLY

SEQ ID NO: 74 - protein, 15 aa, Homo sapiens
  Immunogenic cluster identified in FVIII
DHLFNIAKPRPPWMG

SEQ ID NO: 75 - protein, 18 aa, Homo sapiens
  Immunogenic cluster identified in FVIII
YDTVVITLKNMASHPVSL

SEQ ID NO: 76 - protein, 16 aa, Homo sapiens
  Immunogenic cluster identified in FVIII
VWQVLKENGPMASDPL

SEQ ID NO: 77 - protein, 18 aa, Homo sapiens
  Immunogenic cluster identified in FVIII
SDPLCLTYSYLSHVDLVK

SEQ ID NO: 78 - protein, 16 aa, Homo sapiens
  Immunogenic cluster identified in FVIII
HVDLVKDLNSGLIGAL

SEQ ID NO: 79 - protein, 18 aa, Homo sapiens
  Immunogenic cluster identified in FVIII
TQTLHKFILLFAVFDEGK

SEQ ID NO: 80 - protein, 15 aa, Homo sapiens
  Immunogenic cluster identified in FVIII
GKSWHSETKNSLMQD

SEQ ID NO: 81 - protein, 17 aa, Homo sapiens
  Immunogenic cluster identified in FVIII
MHTVNGYVNRSLPGLIG

SEQ ID NO: 82 - protein, 17 aa, Homo sapiens
  Immunogenic cluster identified in FVIII
GHTFLVRNHRQASLEIS

SEQ ID NO: 83 - protein, 17 aa, Homo sapiens
  Immunogenic cluster identified in FVIII
ISPITFLTAQTLLMDLG

SEQ ID NO: 84 - protein, 19 aa, Homo sapiens
  Immunogenic cluster identified in FVIII
DRSYKSQYLNNGPQRIGRK

SEQ ID NO: 85 - protein, 19 aa, Homo sapiens
  Immunogenic cluster identified in FVIII
GDTLLIIFKNQASRPYNIY

SEQ ID NO: 86 - protein, 19 aa, Homo sapiens
  Immunogenic cluster identified in FVIII
ITDVRPLYSRRLPKGVKHL

SEQ ID NO: 87 - protein, 22 aa, Homo sapiens
  Immunogenic cluster identified in FVIII
PRCLTRYYSSFVNMERDLASGL

SEQ ID NO: 88 - protein, 18 aa, Homo sapiens
  Immunogenic cluster identified in FVIII
TENIQRFLPNPAGVQLED

SEQ ID NO: 89 - protein, 19 aa, Homo sapiens
  Immunogenic cluster identified in FVIII
PEFQASNIMHSINGYVFDS

SEQ ID NO: 90 - protein, 17 aa, Homo sapiens
  Immunogenic cluster identified in FVIII
EVAYWYILSIGAQTDFL

SEQ ID NO: 91 - protein, 15 aa, Homo sapiens
  Immunogenic cluster identified in FVIII
ETVFMSMENPGLWIL

SEQ ID NO: 92 - protein, 16 aa, Homo sapiens
  Immunogenic cluster identified in FVIII
ISAYLLSKNNAIEPRS

SEQ ID NO: 93 - protein, 15 aa, Homo sapiens
  Immunogenic cluster identified in FVIII
PRSFQKKTRHYFIAA

SEQ ID NO: 94 - protein, 16 aa, Homo sapiens
  Immunogenic cluster identified in FVIII
MVTFRNQASRPYSFYS

SEQ ID NO: 95 - protein, 16 aa, Homo sapiens
  Immunogenic cluster identified in FVIII
KTYFWKVQHHMAPTKD

SEQ ID NO: 96 - protein, 21 aa, Homo sapiens
  Immunogenic cluster identified in FVIII
DPTFKENYRFHAINGYIMDTL

SEQ ID NO: 97 - protein, 19 aa, Homo sapiens
  Immunogenic cluster identified in FVIII
DQRIRWYLLSMGSNENIHS

SEQ ID NO: 98 - protein, 16 aa, Homo sapiens
  Immunogenic cluster identified in FVIII
MSTLFLVYSNKCQTPL

SEQ ID NO: 99 - protein, 19 aa, Homo sapiens
  Immunogenic cluster identified in FVIII
PKLARLHYSGSINAWSTKE

SEQ ID NO: 100 - protein, 19 aa, Homo sapiens
  Immunogenic cluster identified in FVIII
SSLYISQFIIMYSLDGKKW

SEQ ID NO: 101 - protein, 18 aa, Homo sapiens
  Immunogenic cluster identified in FVIII
GKKWQTYRGNSTGTLMVF

SEQ ID NO: 102 - protein, 17 aa, Homo sapiens
  Immunogenic cluster identified in FVIII
IARYIRLHPTHYSIRST

SEQ ID NO: 103 - protein, 15 aa, Homo sapiens
  Immunogenic cluster identified in FVIII
SSYFTNMFATWSPSK

SEQ ID NO: 104 - protein, 17 aa, Homo sapiens
  Immunogenic cluster identified in FVIII
KARLHLQGRSNAWRPQV

SEQ ID NO: 105 - protein, 17 aa, Homo sapiens
  Immunogenic cluster identified in FVIII
WLQVDFQKTMKVTGVTT

SEQ ID NO: 106 - protein, 19 aa, Homo sapiens
  Immunogenic cluster identified in FVIII
TSMYVKEFLISSSQDGHQW

SEQ ID NO: 107 - protein, 19 aa, Homo sapiens
  Immunogenic cluster identified in FVIII
QDSFTPVVNSLDPPLLTRY

SEQ ID NO: 108 - protein, 15 aa, Homo sapiens
  Immunogenic cluster identified in FVIII
PQSWVHQIALRMEVL

SEQ ID NO: 109 - protein, 1533 aa, Artificial Sequence
  SIGNAL 1..19
  FVIII-A1A2-3M
MQIELSTCFFLCLLRFCFSATRRYYLGAVELSWDYMQSDLGELPVDARFPPRVPKSFPFNTSVVYKKTLFVEFTDHLFNIAKPRPPWMGLLGPTIQAEVYDTVVITLKNMASHPVSLHAVGVSYWKASEGAEYDDQTSQREKEDDKVFPGGSHTYVWQVLKENGPMASDPLCLTYSYLSHVDLVKDLNSGLIGALLVCREGSLAKEKTQTLHKFILLFAVFDEGKSWHSETKNSLMQDRDAASARAWPKMHTVNGYVNRSLPGLIGCHRKSVYWHVIGMGTTPEVHSIFLEGHTFLVRDHRQASLEISPITFLTAQTLLMDLGQFLLFCHISSHQHDGMEAYVKVDSCPEEPQLRMKNNEEAEDYDDDLTDSEMDVVRFDDDNSPSFIQIRSVAKKHPKTWVHYIAAEEEDWDYAPLVLAPDDRSHKSQYLNNGPQRIGRKYKKVRFMAYTDETFKTREAIQHESGILGPLLYGEVGDTLLIIFKNQASRPYNIYPHGITDVRPLYERRLPKGVKHLKDFPILPGEIFKYKWTVTVEDGPTKSDPRCLTRYYSSFVNMERDLASGLIGPLLICYKESVDQRGNQIMSDKRNVILFSVFDENRSWYLTENIQRFLPNPAGVQLEDPEFQASNIMHSINGYVFDSLQLSVCLHEVAYWYILSIGAQTDFLSVFFSGYTFKHKMVYEDTLTLFPFSGETVFMSMENPGLWILGCHNSDFRNRGMTALLKVSSCDKNTGDYYEDSYEDISAYLLSKNNAIEPRSFSQDPLAWDNHYGTQIPKEEWKSQEKSPEKTAFKKKDTILSLNACESNHAIAAINEGQNKPEIEVTWAKQGRTERLCSQNPPVLKRHQREITRTTLQSDQEEIDYDDTISVEMKKEDFDIYDEDENQSPRSFQKKTRHYFIAAVERLWDYGMSSSPHVLRNRAQSGSVPQFKKVVFQEFTDGSFTQPLYRGELNEHLGLLGPYIRAEVEDNIMVTFRNQASRPYSFYSSLISYEEDQRQGAEPRKNFVKPNETKTYFWKVQHHMAPTKDEFDCKAWAYFSDVDLEKDVHSGLIGPLLVCHTNTLNPAHGRQVTVQEFALFFTIFDETKSWYFTENMERNCRAPCNIQMEDPTFKENYRFHAINGYIMDTLPGLVMAQDQRIRWYLLSMGSNENIHSIHFSGHVFTVRKKEEYKMALYNLYPGVFETVEMLPSKAGIWRVECLIGEHLHAGMSTLFLVYSNKCQTPLGMASGHIRDFQITASGQYGQWAPKLARLHYSGSINAWSTKEPFSWIKVDLLAPMIIHGIKTQGARQKFSSLYISQFIIMYSLDGKKWQTYRGNSTGTLMVFFGNVDSSGIKHNIFNPPIIARYIRLHPTHYSIRSTLRMELMGCDLNSCSMPLGMESKAISDAQITASSYFTNMFATWSPSKARLHLQGRSNAWRPQVNNPKEWLQVDFQKTMKVTGVTTQGVKSLLTSMYVKEFLISSSQDGHQWTLFFQNGKVKVFQGNQDSFTPVVNSLDPPLLTRYLRIHPQSWVHQIALRMEVLGCEAQDLY

SEQ ID NO: 110 - DNA, 4605 bp, Artificial Sequence
  FVIII-19M na
atgcagatcgagctgtctacctgcttcttcctgtgcctgctgcggttctgcttcagcgccacccggcggtactacctgggcgccgtggaactgagctgggactacatgcagagcgacctgggcgagctgcccgtggacgccagattccccccaagagtgcccaagagcttccccttcaacacctccgtggtgtacaagaaaaccctgttcgtcgagttcaccgaccacctgttctccatcgccaagcccagacccccctggatgggcctgctgggccctacaatccaggccgaggtgtacgacaccgtggtcatcaccctgaagaacatggccacccaccccgtgtccctgcacgccgtgggcgtgtcctactggaaggccagcgagggcgccgagtacgacgaccagaccagccagcgcgagaaagaggacgacaaagtctttcctggcggcagccatacctacgtgtggcaggtctccaaagaaaacggccccatggcctccgacccccagtgcctgacctacagctacctgagccacgtggacctggccaaggacctgaacagcggcctgatcggcgccctgctcgtgtgcagagagggcagcctggccaaagagaaaacccagaccctgcacaagttcatcctgctgttcgccgtgttcgacgagggcaagagctggcacagcgagacaaaggacagcctgatgcaggaccgggacgccgcctctgccagagcctggcctaagatgcacaccgtgaacggctacgtgaacagaagcctgcccggactgaccggctgccaccggaagtccgtgtactggcacgtgatcggcatgggtaccacccccgaggtgcacagcatctttctggaaggacacaccttcctcgtgcgggaccaccggcaggccagcctggaaatcagccctatcaccttcctgaccgcccagacactgctgatggacctgggccagttcctgctgttttgccacatcagcagccaccagcacgacggcatggaagcctacgtgaaggtggacagctgccccgaggaaccccagctgcggatgaagaacaacgaggaagccgaggactacgacgacgacctgaccgacagcgagatggacgtcgtcagattcgatgacgacaacagccccagcttcatccagatcagaagcgtggccaagaagcaccccaagacctgggtgcactatatcgccgccgaggaagaggactgggactacgcccctctggtgctggcccccgacgacagaagccacaagagccagtacctgaacaatggcccccagcggatcggccggaagtacaagaaagtgcggttcatggcctacaccgacgagacattcaagaccagagaggccatccagcacgagagcggcatcctgggccccctgctgtacggcgaagtgggcgacaccctgctgattatcttcaagaaccaggccagccggccctacaacatctacccccacggcatcaccgacgtgcggcccctgtacgagagacggctgcccaagggcgtgaagcacctgaaggacttccccatcctgcccggggagatcttcaagtacaagtggaccgtgaccgtggaagatggccccaccaagagcgaccccagatgcctgacccggtactacagcagccacgtgaacatggaacgggacctggcctccgggctgatcggccctctgctgatctgctacaaagaaagcgtggaccagcggggcaaccagatcatgagcgacaagcggaacgtgatcctgttcagcgtgttcgatgagaacagaagctggtatctgaccgagaatatccagcggttcctgcccgagcctgccggcgtgcagctggaagatcccgagttccaggccagcaacatcatgcactccatcaatggctacgtgttcgacagcctgcagctgagcgtgtgcctgcacgaggtggcctactggtacatcctgagcatcggcgcccagaccgacttcctgagcgtgttcttcagcggctacaccttcaagcacaagatggtgtacgaggataccctgaccctgttccccttctccggcgaaaccgtgttcatgagcatggaaaaccccggcaactggattctgggctgccacaacagcgacttccggaaccggggcatgaccgccctgctgaaggtgtccagctgcgacaagaacaccggcgactactacgaggacagctacgaggatatcagcgccagcctgctgagcaagaacaacgccatcgagcctcggagcttctcccaggatcctctggcctgggacaaccactacggcacccagatccccaaagaggaatggaagtcccaggaaaagagccccgagaaaaccgccttcaagaagaaggacaccatcctgagcctgaacgcctgcgagagcaaccacgccattgccgccatcaacgagggccagaacaagcccgagatcgaagtgacctgggctaagcagggccggaccgagagactgtgcagccagaacccccccgtgctgaagcggcaccagagagagatcacccggaccaccctgcagagcgaccaggaagagatcgactacgacgacaccatcagcgtcgagatgaagaaagaggacttcgacatctacgacgaggacgagaaccagagcccccggtccttccagaagaaaacccggcactactttatcgccgccgtggaacggctgtgggactacggcatgagcagcagcccccacgtgctgcggaatagagcccagagcggcagcgtgccccagttcaagaaagtggtgttccaggagttcaccgacggcagcttcacccagcctctgtaccggggcgagctgaacgagcacctgggcctgctgggcccctacatccgggccgaggtggaagataacatcatggtcaccttccggaaccaggccagccggccctacagcttctacagcagcctgatctcctacgaagaggaccagcggcagggcgccgagccccggaagaacttcgtgaagcccaacgagacaaagacctacttctgggaggtgcagcaccacatggcccccaccaaggacgaattcgactgcaaggcctgggcctacttcagcgacgtggacctggaaaaggacgtgcacagcggcctgatcggccccctgctcgtgtgccacaccaacaccctgaaccccgcccacggccggcaggtcacagtgcaggaatttgccctgttcttcaccatcttcgacgagactaagagctggtacttcaccgagaacatggaacggaactgcagagccccctgcaacatccagatggaagatcccaccttcaaagagaactaccggttccacgccatcaatggctacatcatggacaccctgcccggcctggtcatggcccaggaccagagaatccggtggtatctgctgagcatgggcagcaacgagaacatccacagcatccacttcagcggccacgtgttcaccgtgcggaagaaagaagagtacaagatggccctgtacaacctgtaccccggcgtgttcgagacagtggaaatgctgcccagcaaggccggcatctggcgggtggaatgtctgatcggcgagcatctgcacgccggcatgtccaccctgtttctggtgtacagcgacaagtgccagacccccctgggcatggccagcggccacatccgggatttccagatcaccgcctccggccagtacggccagtgggcccctaaactggcccggctgcactacagcggcggcatcaacgcctggtccaccaaagagcccttcagctggatcaaggtggacctgctggcccccatgattatccacggcatcaagacacagggcgccagacagaagttcagcagcctgtacatcagccagttcatcatcatgtacagcctggatggcaagaagtggcagacctaccggggcaacagcaccggcaccctgatggtgttcttcggcaacgtggacagcagcggcatcaagcacaacatcttcaacccccccatcattgcccggtacatccggctgcaccccacccactacagcatccggtccaccctgcggatggaactgatgggctgcgacctgaacagctgctccatgcctctgggcatggaaagcaaggccatcagcgacgcccagatcacagccagcagctacttcaccaacatgttcgccacctggtccccatccaaggccagactgcatctgcagggcagaagcaatgcctggcggcctcaggtcaacaaccccaaagaatggctccaggtggacttccagaaaaccatgaaggtcacaggcgtgaccacccagggcgtgaagtccctgctgacctctatgtacgtgaaagagttcctgatctccagcagccaggacggccaccagtggaccctgttctttcagaacggcaaagtgaaagtgttccagggcaaccaggactccttcacccccgtggtcaacaccctggaccccccactgctgaccagatacctgagaatccacccccagagctgggcccaccagatcgccctgagaatggaagtgctgggatgcgaggcccaggatctgtactgatag

SEQ ID NO: 111 - DNA, 4605 bp, Artificial Sequence
  FVIII-6rs na
atgcagatcgagctgtctacctgcttcttcctgtgcctgctgcggttctgcttcagcgccacccggcggtactacctgggcgccgtggaactgagctgggactacatgcagagcgacctgggcgagctgcccgtggacgccagattccccccaagagtgcccaagagcttccccttcaacacctccgtggtgtacaagaaaaccctgttcgtcgagttcaccgaccacctgttcaatatcgccaagcccagacccccctggatgggcctgctgggccctacaatccaggccgaggtgtacgacaccgtggtcatcaccctgaagaacatggccagccaccccgtgtccctgcacgccgtgggcgtgtcctactggaaggccagcgagggcgccgagtacgacgaccagaccagccagcgcgagaaagaggacgacaaagtctttcctggcggcagccatacctacgtgtggcaggtcctgaaagaaaacggccccatggcctccgaccccctgtgcctgacctacagctacctgagccacgtggacctggtcaaggacctgaacagcggcctgatcggcgccctgctcgtgtgcagagagggcagcctggccaaagagaaaacccagaccctgcacaagttcatcctgctgttcgccgtgttcgacgagggcaagagctggcacagcgagacaaagaacagcctgatgcaggaccgggacgccgcctctgccagagcctggcctaagatgcacaccgtgaacggctacgtgaacagaagcctgcccggactgatcggctgccaccggaagtccgtgtactggcacgtgatcggcatgggtaccacccccgaggtgcacagcatctttctggaaggacacaccttcctcgtgcggaaccaccggcaggccagcctggaaatcagccctatcaccttcctgaccgcccagacactgctgatggacctgggccagttcctgctgttttgccacatcagcagccaccagcacgacggcatggaagcctacgtgaaggtggacagctgccccgaggaaccccagctgcggatgaagaacaacgaggaagccgaggactacgacgacgacctgaccgacagcgagatggacgtcgtcagattcgatgacgacaacagccccagcttcatccagatcagaagcgtggccaagaagcaccccaagacctgggtgcactatatcgccgccgaggaagaggactgggactacgcccctctggtgctggcccccgacgacagaagctacaagagccagtacctgaacaatggcccccagcggatcggccggaagtacaagaaagtgcggttcatggcctacaccgacgagacattcaagaccagagaggccatccagcacgagagcggcatcctgggccccctgctgtacggcgaagtgggcgacaccctgctgattatcttcaagaaccaggccagccggccctacaacatctacccccacggcatcaccgacgtgcggcccctgtacagcagacggctgcccaagggcgtgaagcacctgaaggacttccccatcctgcccggggagatcttcaagtacaagtggaccgtgaccgtggaagatggccccaccaagagcgaccccagatgcctgacccggtactacagcagcttcgtgaacatggaacgggacctggcctccgggctgatcggccctctgctgatctgctacaaagaaagcgtggaccagcggggcaaccagatcatgagcgacaagcggaacgtgatcctgttcagcgtgttcgatgagaacagaagctggtatctgaccgagaatatccagcggttcctgcccaaccctgccggcgtgcagctggaagatcccgagttccaggccagcaacatcatgcactccatcaatggctacgtgttcgacagcctgcagctgagcgtgtgcctgcacgaggtggcctactggtacatcctgagcatcggcgcccagaccgacttcctgagcgtgttcttcagcggctacaccttcaagcacaagatggtgtacgaggataccctgaccctgttccccttctccggcgaaaccgtgttcatgagcatggaaaaccccggcctgtggattctgggctgccacaacagcgacttccggaaccggggcatgaccgccctgctgaaggtgtccagctgcgacaagaacaccggcgactactacgaggacagctacgaggatatcagcgcctacctgctgagcaagaacaacgccatcgagcctcggagcttctcccaggatcctctggcctgggacaaccactacggcacccagatccccaaagaggaatggaagtcccaggaaaagagccccgagaaaaccgccttcaagaagaaggacaccatcctgagcctgaacgcctgcgagagcaaccacgccattgccgccatcaacgagggccagaacaagcccgagatcgaagtgacctgggctaagcagggccggaccgagagactgtgcagccagaacccccccgtgctgaagcggcaccagagagagatcacccggaccaccctgcagagcgaccaggaagagatcgactacgacgacaccatcagcgtcgagatgaagaaagaggacttcgacatctacgacgaggacgagaaccagagcccccggtccttccagaagaaaacccggcactactttatcgccgccgtggaacggctgtgggactacggcatgagcagcagcccccacgtgctgcggaatagagcccagagcggcagcgtgccccagttcaagaaagtggtgttccaggagttcaccgacggcagcttcacccagcctctgtaccggggcgagctgaacgagcacctgggcctgctgggcccctacatccgggccgaggtggaagataacatcatggtcaccttccggaaccaggccagccggccctacagcttctacagcagcctgatctcctacgaagaggaccagcggcagggcgccgagccccggaagaacttcgtgaagcccaacgagacaaagacctacttctggaaggtgcagcaccacatggcccccaccaaggacgaattcgactgcaaggcctgggcctacttcagcgacgtggacctggaaaaggacgtgcacagcggcctgatcggccccctgctcgtgtgccacaccaacaccctgaaccccgcccacggccggcaggtcacagtgcaggaatttgccctgttcttcaccatcttcgacgagactaagagctggtacttcaccgagaacatggaacggaactgcagagccccctgcaacatccagatggaagatcccaccttcaaagagaactaccggttccacgccatcaatggctacatcatggacaccctgcccggcctggtcatggcccaggaccagagaatccggtggtatctgctgagcatgggcagcaacgagaacatccacagcatccacttcagcggccacgtgttcaccgtgcggaagaaagaagagtacaagatggccctgtacaacctgtaccccggcgtgttcgagacagtggaaatgctgcccagcaaggccggcatctggcgggtggaatgtctgatcggcgagcatctgcacgccggcatgtccaccctgtttctggtgtacagcaacaagtgccagacccccctgggcatggccagcggccacatccgggatttccagatcaccgcctccggccagtacggccagtgggcccctaaactggcccggctgcactacagcggcagcatcaacgcctggtccaccaaagagcccttcagctggatcaaggtggacctgctggcccccatgattatccacggcatcaagacacagggcgccagacagaagttcagcagcctgtacatcagccagttcatcatcatgtacagcctggatggcaagaagtggcagacctaccggggcaacagcaccggcaccctgatggtgttcttcggcaacgtggacagcagcggcatcaagcacaacatcttcaacccccccatcattgcccggtacatccggctgcaccccacccactacagcatccggtccaccctgcggatggaactgatgggctgcgacctgaacagctgctccatgcctctgggcatggaaagcaaggccatcagcgacgcccagatcacagccagcagctacttcaccaacatgttcgccacctggtccccatccaaggccagactgcatctgcagggcagaagcaatgcctggcggcctcaggtcaacaaccccaaagaatggctccaggtggacttccagaaaaccatgaaggtcacaggcgtgaccacccagggcgtgaagtccctgctgacctctatgtacgtgaaagagttcctgatctccagcagccaggacggccaccagtggaccctgttctttcagaacggcaaagtgaaagtgttccagggcaaccaggactccttcacccccgtggtcaactccctggaccccccactgctgaccagatacctgagaatccacccccagagctgggtgcaccagatcgccctgagaatggaagtgctgggatgcgaggcccaggatctgtactgatag

SEQ ID NO: 112 - protein, 16 aa, Artificial Sequence
  Immunogenic cluster identified in FVIII
GEHLHAGMSTLFLVYS

SEQ ID NO: 113 - protein, 1445 aa, Artificial Sequence
  Single chain V0-19M (AC-19M_SC)
MQIELSTCFFLCLLRFCFSATRRYYLGAVELSWDYMQSDLGELPVDARFPPRVPKSFPFNTSVVYKKTLFVEFTDHLFSIAKPRPPWMGLLGPTIQAEVYDTVVITLKNMATHPVSLHAVGVSYWKASEGAEYDDQTSQREKEDDKVFPGGSHTYVWQVSKENGPMASDPQCLTYSYLSHVDLAKDLNSGLIGALLVCREGSLAKEKTQTLHKFILLFAVFDEGKSWHSETKDSLMQDRDAASARAWPKMHTVNGYVNRSLPGLTGCHRKSVYWHVIGMGTTPEVHSIFLEGHTFLVRDHRQASLEISPITFLTAQTLLMDLGQFLLFCHISSHQHDGMEAYVKVDSCPEEPQLRMKNNEEAEDYDDDLTDSEMDVVRFDDDNSPSFIQIRSVAKKHPKTWVHYIAAEEEDWDYAPLVLAPDDRSHKSQYLNNGPQRIGRKYKKVRFMAYTDETFKTREAIQHESGILGPLLYGEVGDTLLIIFKNQASRPYNIYPHGITDVRPLYERRLPKGVKHLKDFPILPGEIFKYKWTVTVEDGPTKSDPRCLTRYYSSHVNMERDLASGLIGPLLICYKESVDQRGNQIMSDKRNVILFSVFDENRSWYLTENIQRFLPEPAGVQLEDPEFQASNIMHSINGYVFDSLQLSVCLHEVAYWYILSIGAQTDFLSVFFSGYTFKHKMVYEDTLTLFPFSGETVFMSMENPGNWILGCHNSDFRNRGMTALLKVSSCDKNTGDYYEDSYEDISASLLSKNNAIEPRSFSQNPPVLQSDQEEIDYDDTISVEMKKEDFDIYDEDENQSPRSFQKKTRHYFIAAVERLWDYGMSSSPHVLRNRAQSGSVPQFKKVVFQEFTDGSFTQPLYRGELNEHLGLLGPYIRAEVEDNIMVTFRNQASRPYSFYSSLISYEEDQRQGAEPRKNFVKPNETKTYFWEVQHHMAPTKDEFDCKAWAYFSDVDLEKDVHSGLIGPLLVCHTNTLNPAHGRQVTVQEFALFFTIFDETKSWYFTENMERNCRAPCNIQMEDPTFKENYRFHAINGYIMDTLPGLVMAQDQRIRWYLLSMGSNENIHSIHFSGHVFTVRKKEEYKMALYNLYPGVFETVEMLPSKAGIWRVECLIGEHLHAGMSTLFLVYSDKCQTPLGMASGHIRDFQITASGQYGQWAPKLARLHYSGGINAWSTKEPFSWIKVDLLAPMIIHGIKTQGARQKFSSLYISQFIIMYSLDGKKWQTYRGNSTGTLMVFFGNVDSSGIKHNIFNPPIIARYIRLHPTHYSIRSTLRMELMGCDLNSCSMPLGMESKAISDAQITASSYFTNMFATWSPSKARLHLQGRSNAWRPQVNNPKEWLQVDFQKTMKVTGVTTQGVKSLLTSMYVKEFLISSSQDGHQWTLFFQNGKVKVFQGNQDSFTPVVNTLDPPLLTRYLRIHPQSWAHQIALRMEVLGCEAQDLY

SEQ ID NO: 114 - protein, 1778 aa, Artificial Sequence
  AD2CD2-19M_SC
MQIELSTCFFLCLLRFCFSATRRYYLGAVELSWDYMQSDLGELPVDARFPPRVPKSFPFNTSVVYKKTLFVEFTDHLFSIAKPRPPWMGLLGPTIQAEVYDTVVITLKNMATHPVSLHAVGVSYWKASEGAEYDDQTSQREKEDDKVFPGGSHTYVWQVSKENGPMASDPQCLTYSYLSHVDLAKDLNSGLIGALLVCREGSLAKEKTQTLHKFILLFAVFDEGKSWHSETKDSLMQDRDAASARAWPKMHTVNGYVNRSLPGLTGCHRKSVYWHVIGMGTTPEVHSIFLEGHTFLVRDHRQASLEISPITFLTAQTLLMDLGQFLLFCHISSHQHDGMEAYVKVDSCPEEPQLRMKNNEEAEDYDDDLTDSEMDVVRFDDDNSPSFIQIRSVAKKHPKTWVHYIAAEEEDWDYAPLVLAPDDRSHKSQYLNNGPQRIGRKYKKVRFMAYTDETFKTREAIQHESGILGPLLYGEVGDTLLIIFKNQASRPYNIYPHGITDVRPLYERRLPKGVKHLKDFPILPGEIFKYKWTVTVEDGPTKSDPRCLTRYYSSHVNMERDLASGLIGPLLICYKESVDQRGNQIMSDKRNVILFSVFDENRSWYLTENIQRFLPEPAGVQLEDPEFQASNIMHSINGYVFDSLQLSVCLHEVAYWYILSIGAQTDFLSVFFSGYTFKHKMVYEDTLTLFPFSGETVFMSMENPGNWILGCHNSDFRNRGMTALLKVSSCDKNTGDYYEDSYEDISASLLSKNNAIEPRSFSQNPPVLSKNNAIEPRSFSQNSRHPSLAEAKEAANAELDSYGVSDFYKRLIDKAKTVEGVEALKDAILAALPGSGGSGGSGGSGSGSKNNAIEPRSFSQNSRHPSGSGGSGGSGGSGSLAEAKEAANAELDSYGVSDFYKRLIDKAKTVEGVEALKDAILAALPSKNNAIEPRSFSQNSRHPSQSDQEEIDYDDTISVEMKKEDFDIYDEDENQSPRSFQKKTRHYFIAAVERLWDYGMSSSPHVLRNRAQSGSVPQFKKVVFQEFTDGSFTQPLYRGELNEHLGLLGPYIRAEVEDNIMVTFRNQASRPYSFYSSLISYEEDQRQGAEPRKNFVKPNETKTYFWEVQHHMAPTKDEFDCKAWAYFSDVDLEKDVHSGLIGPLLVCHTNTLNPAHGRQVTVQEFALFFTIFDETKSWYFTENMERNCRAPCNIQMEDPTFKENYRFHAINGYIMDTLPGLVMAQDQRIRWYLLSMGSNENIHSIHFSGHVFTVRKKEEYKMALYNLYPGVFETVEMLPSKAGIWRVECLIGEHLHAGMSTLFLVYSDKCQTPLGMASGHIRDFQITASGQYGQWAPKLARLHYSGGINAWSTKEPFSWIKVDLLAPMIIHGIKTQGARQKFSSLYISQFIIMYSLDGKKWQTYRGNSTGTLMVFFGNVDSSGIKHNIFNPPIIARYIRLHPTHYSIRSTLRMELMGCDLNSCSMPLGMESKAISDAQITASSYFTNMFATWSPSKARLHLQGRSNAWRPQVNNPKEWLQVDFQKTMKVTGVTTQGVKSLLTSMYVKEFLISSSQDGHQWTLFFQNGKVKVFQGNQDSFTPVVNTLDPPLLTRYLRIHPQSWAHQIALRMEVLGCEAQDLYSKNNAIEPRSFSQNSRHPSLAEAKEAANAELDSYGVSDFYKRLIDKAKTVEGVEALKDAILAALPGSGGSGGSGGSGSGSKNNAIEPRSFSQNSRHPSGSGGSGGSGGSGSLAEAKEAANAELDSYGVSDFYKRLIDKAKTVEGVEALKDAILAALP

SEQ ID NO: 115 - protein, 1594 aa, Artificial Sequence
  ALDLCLD-19M_SC
MQIELSTCFFLCLLRFCFSATRRYYLGAVELSWDYMQSDLGELPVDARFPPRVPKSFPFNTSVVYKKTLFVEFTDHLFSIAKPRPPWMGLLGPTIQAEVYDTVVITLKNMATHPVSLHAVGVSYWKASEGAEYDDQTSQREKEDDKVFPGGSHTYVWQVSKENGPMASDPQCLTYSYLSHVDLAKDLNSGLIGALLVCREGSLAKEKTQTLHKFILLFAVFDEGKSWHSETKDSLMQDRDAASARAWPKMHTVNGYVNRSLPGLTGCHRKSVYWHVIGMGTTPEVHSIFLEGHTFLVRDHRQASLEISPITFLTAQTLLMDLGQFLLFCHISSHQHDGMEAYVKVDSCPEEPQLRMKNNEEAEDYDDDLTDSEMDVVRFDDDNSPSFIQIRSVAKKHPKTWVHYIAAEEEDWDYAPLVLAPDDRSHKSQYLNNGPQRIGRKYKKVRFMAYTDETFKTREAIQHESGILGPLLYGEVGDTLLIIFKNQASRPYNIYPHGITDVRPLYERRLPKGVKHLKDFPILPGEIFKYKWTVTVEDGPTKSDPRCLTRYYSSHVNMERDLASGLIGPLLICYKESVDQRGNQIMSDKRNVILFSVFDENRSWYLTENIQRFLPEPAGVQLEDPEFQASNIMHSINGYVFDSLQLSVCLHEVAYWYILSIGAQTDFLSVFFSGYTFKHKMVYEDTLTLFPFSGETVFMSMENPGNWILGCHNSDFRNRGMTALLKVSSCDKNTGDYYEDSYEDISASLLSKNNAIEPRSFSQNPPVLSKNNAIEPRSFSQNSRHPSLAEAKEAANAELDSYGVSDFYKRLIDKAKTVEGVEALKDAILAALPSKNNAIEPRSFSQNSRHPSQSDQEEIDYDDTISVEMKKEDFDIYDEDENQSPRSFQKKTRHYFIAAVERLWDYGMSSSPHVLRNRAQSGSVPQFKKVVFQEFTDGSFTQPLYRGELNEHLGLLGPYIRAEVEDNIMVTFRNQASRPYSFYSSLISYEEDQRQGAEPRKNFVKPNETKTYFWEVQHHMAPTKDEFDCKAWAYFSDVDLEKDVHSGLIGPLLVCHTNTLNPAHGRQVTVQEFALFFTIFDETKSWYFTENMERNCRAPCNIQMEDPTFKENYRFHAINGYIMDTLPGLVMAQDQRIRWYLLSMGSNENIHSIHFSGHVFTVRKKEEYKMALYNLYPGVFETVEMLPSKAGIWRVECLIGEHLHAGMSTLFLVYSDKCQTPLGMASGHIRDFQITASGQYGQWAPKLARLHYSGGINAWSTKEPFSWIKVDLLAPMIIHGIKTQGARQKFSSLYISQFIIMYSLDGKKWQTYRGNSTGTLMVFFGNVDSSGIKHNIFNPPIIARYIRLHPTHYSIRSTLRMELMGCDLNSCSMPLGMESKAISDAQITASSYFTNMFATWSPSKARLHLQGRSNAWRPQVNNPKEWLQVDFQKTMKVTGVTTQGVKSLLTSMYVKEFLISSSQDGHQWTLFFQNGKVKVFQGNQDSFTPVVNTLDPPLLTRYLRIHPQSWAHQIALRMEVLGCEAQDLYSKNNAIEPRSFSQNSRHPSLAEAKEAANAELDSYGVSDFYKRLIDKAKTVEGVEALKDAILAALP

SEQ ID NO: 116 - protein, 1575 aa, Artificial Sequence
  ADLCLD-19M_SC-V1
MQIELSTCFFLCLLRFCFSATRRYYLGAVELSWDYMQSDLGELPVDARFPPRVPKSFPFNTSVVYKKTLFVEFTDHLFSIAKPRPPWMGLLGPTIQAEVYDTVVITLKNMATHPVSLHAVGVSYWKASEGAEYDDQTSQREKEDDKVFPGGSHTYVWQVSKENGPMASDPQCLTYSYLSHVDLAKDLNSGLIGALLVCREGSLAKEKTQTLHKFILLFAVFDEGKSWHSETKDSLMQDRDAASARAWPKMHTVNGYVNRSLPGLTGCHRKSVYWHVIGMGTTPEVHSIFLEGHTFLVRDHRQASLEISPITFLTAQTLLMDLGQFLLFCHISSHQHDGMEAYVKVDSCPEEPQLRMKNNEEAEDYDDDLTDSEMDVVRFDDDNSPSFIQIRSVAKKHPKTWVHYIAAEEEDWDYAPLVLAPDDRSHKSQYLNNGPQRIGRKYKKVRFMAYTDETFKTREAIQHESGILGPLLYGEVGDTLLIIFKNQASRPYNIYPHGITDVRPLYERRLPKGVKHLKDFPILPGEIFKYKWTVTVEDGPTKSDPRCLTRYYSSHVNMERDLASGLIGPLLICYKESVDQRGNQIMSDKRNVILFSVFDENRSWYLTENIQRFLPEPAGVQLEDPEFQASNIMHSINGYVFDSLQLSVCLHEVAYWYILSIGAQTDFLSVFFSGYTFKHKMVYEDTLTLFPFSGETVFMSMENPGNWILGCHNSDFRNRGMTALLKVSSCDKNTGDYYEDSYEDISASLLSKNNAIEPRSFSQNPPVLLAEAKEAANAELDSYGVSDFYKRLIDKAKTVEGVEALKDAILAALPSKNNAIEPRSFSQNSRHPSQSDQEEIDYDDTISVEMKKEDFDIYDEDENQSPRSFQKKTRHYFIAAVERLWDYGMSSSPHVLRNRAQSGSVPQFKKVVFQEFTDGSFTQPLYRGELNEHLGLLGPYIRAEVEDNIMVTFRNQASRPYSFYSSLISYEEDQRQGAEPRKNFVKPNETKTYFWEVQHHMAPTKDEFDCKAWAYFSDVDLEKDVHSGLIGPLLVCHTNTLNPAHGRQVTVQEFALFFTIFDETKSWYFTENMERNCRAPCNIQMEDPTFKENYRFHAINGYIMDTLPGLVMAQDQRIRWYLLSMGSNENIHSIHFSGHVFTVRKKEEYKMALYNLYPGVFETVEMLPSKAGIWRVECLIGEHLHAGMSTLFLVYSDKCQTPLGMASGHIRDFQITASGQYGQWAPKLARLHYSGGINAWSTKEPFSWIKVDLLAPMIIHGIKTQGARQKFSSLYISQFIIMYSLDGKKWQTYRGNSTGTLMVFFGNVDSSGIKHNIFNPPIIARYIRLHPTHYSIRSTLRMELMGCDLNSCSMPLGMESKAISDAQITASSYFTNMFATWSPSKARLHLQGRSNAWRPQVNNPKEWLQVDFQKTMKVTGVTTQGVKSLLTSMYVKEFLISSSQDGHQWTLFFQNGKVKVFQGNQDSFTPVVNTLDPPLLTRYLRIHPQSWAHQIALRMEVLGCEAQDLYSKNNAIEPRSFSQNSRHPSLAEAKEAANAELDSYGVSDFYKRLIDKAKTVEGVEALKDAILAALP

SEQ ID NO: 117 - protein, 1575 aa, Artificial Sequence
  ADLCLD-19M_SC-V2
MQIELSTCFFLCLLRFCFSATRRYYLGAVELSWDYMQSDLGELPVDARFPPRVPKSFPFNTSVVYKKTLFVEFTDHLFSIAKPRPPWMGLLGPTIQAEVYDTVVITLKNMATHPVSLHAVGVSYWKASEGAEYDDQTSQREKEDDKVFPGGSHTYVWQVSKENGPMASDPQCLTYSYLSHVDLAKDLNSGLIGALLVCREGSLAKEKTQTLHKFILLFAVFDEGKSWHSETKDSLMQDRDAASARAWPKMHTVNGYVNRSLPGLTGCHRKSVYWHVIGMGTTPEVHSIFLEGHTFLVRDHRQASLEISPITFLTAQTLLMDLGQFLLFCHISSHQHDGMEAYVKVDSCPEEPQLRMKNNEEAEDYDDDLTDSEMDVVRFDDDNSPSFIQIRSVAKKHPKTWVHYIAAEEEDWDYAPLVLAPDDRSHKSQYLNNGPQRIGRKYKKVRFMAYTDETFKTREAIQHESGILGPLLYGEVGDTLLIIFKNQASRPYNIYPHGITDVRPLYERRLPKGVKHLKDFPILPGEIFKYKWTVTVEDGPTKSDPRCLTRYYSSHVNMERDLASGLIGPLLICYKESVDQRGNQIMSDKRNVILFSVFDENRSWYLTENIQRFLPEPAGVQLEDPEFQASNIMHSINGYVFDSLQLSVCLHEVAYWYILSIGAQTDFLSVFFSGYTFKHKMVYEDTLTLFPFSGETVFMSMENPGNWILGCHNSDFRNRGMTALLKVSSCDKNTGDYYEDSYEDISASLLSKNNAIEPRSFSQNPPVLAEAKEAANAELDSYGVSDFYKRLIDKAKTVEGVEALKDAILAALPSKNNAIEPRSFSQNSRHPSLQSDQEEIDYDDTISVEMKKEDFDIYDEDENQSPRSFQKKTRHYFIAAVERLWDYGMSSSPHVLRNRAQSGSVPQFKKVVFQEFTDGSFTQPLYRGELNEHLGLLGPYIRAEVEDNIMVTFRNQASRPYSFYSSLISYEEDQRQGAEPRKNFVKPNETKTYFWEVQHHMAPTKDEFDCKAWAYFSDVDLEKDVHSGLIGPLLVCHTNTLNPAHGRQVTVQEFALFFTIFDETKSWYFTENMERNCRAPCNIQMEDPTFKENYRFHAINGYIMDTLPGLVMAQDQRIRWYLLSMGSNENIHSIHFSGHVFTVRKKEEYKMALYNLYPGVFETVEMLPSKAGIWRVECLIGEHLHAGMSTLFLVYSDKCQTPLGMASGHIRDFQITASGQYGQWAPKLARLHYSGGINAWSTKEPFSWIKVDLLAPMIIHGIKTQGARQKFSSLYISQFIIMYSLDGKKWQTYRGNSTGTLMVFFGNVDSSGIKHNIFNPPIIARYIRLHPTHYSIRSTLRMELMGCDLNSCSMPLGMESKAISDAQITASSYFTNMFATWSPSKARLHLQGRSNAWRPQVNNPKEWLQVDFQKTMKVTGVTTQGVKSLLTSMYVKEFLISSSQDGHQWTLFFQNGKVKVFQGNQDSFTPVVNTLDPPLLTRYLRIHPQSWAHQIALRMEVLGCEAQDLYSKNNAIEPRSFSQNSRHPSLAEAKEAANAELDSYGVSDFYKRLIDKAKTVEGVEALKDAILAALP

SEQ ID NO: 118 - protein, 1686 aa, Artificial Sequence
  AD2CD-19M_SC
MQIELSTCFFLCLLRFCFSATRRYYLGAVELSWDYMQSDLGELPVDARFPPRVPKSFPFNTSVVYKKTLFVEFTDHLFSIAKPRPPWMGLLGPTIQAEVYDTVVITLKNMATHPVSLHAVGVSYWKASEGAEYDDQTSQREKEDDKVFPGGSHTYVWQVSKENGPMASDPQCLTYSYLSHVDLAKDLNSGLIGALLVCREGSLAKEKTQTLHKFILLFAVFDEGKSWHSETKDSLMQDRDAASARAWPKMHTVNGYVNRSLPGLTGCHRKSVYWHVIGMGTTPEVHSIFLEGHTFLVRDHRQASLEISPITFLTAQTLLMDLGQFLLFCHISSHQHDGMEAYVKVDSCPEEPQLRMKNNEEAEDYDDDLTDSEMDVVRFDDDNSPSFIQIRSVAKKHPKTWVHYIAAEEEDWDYAPLVLAPDDRSHKSQYLNNGPQRIGRKYKKVRFMAYTDETFKTREAIQHESGILGPLLYGEVGDTLLIIFKNQASRPYNIYPHGITDVRPLYERRLPKGVKHLKDFPILPGEIFKYKWTVTVEDGPTKSDPRCLTRYYSSHVNMERDLASGLIGPLLICYKESVDQRGNQIMSDKRNVILFSVFDENRSWYLTENIQRFLPEPAGVQLEDPEFQASNIMHSINGYVFDSLQLSVCLHEVAYWYILSIGAQTDFLSVFFSGYTFKHKMVYEDTLTLFPFSGETVFMSMENPGNWILGCHNSDFRNRGMTALLKVSSCDKNTGDYYEDSYEDISASLLSKNNAIEPRSFSQNPPVLSKNNAIEPRSFSQNSRHPSLAEAKEAANAELDSYGVSDFYKRLIDKAKTVEGVEALKDAILAALPGSGGSGGSGGSGSGSKNNAIEPRSFSQNSRHPSGSGGSGGSGGSGSLAEAKEAANAELDSYGVSDFYKRLIDKAKTVEGVEALKDAILAALPSKNNAIEPRSFSQNSRHPSQSDQEEIDYDDTISVEMKKEDFDIYDEDENQSPRSFQKKTRHYFIAAVERLWDYGMSSSPHVLRNRAQSGSVPQFKKVVFQEFTDGSFTQPLYRGELNEHLGLLGPYIRAEVEDNIMVTFRNQASRPYSFYSSLISYEEDQRQGAEPRKNFVKPNETKTYFWEVQHHMAPTKDEFDCKAWAYFSDVDLEKDVHSGLIGPLLVCHTNTLNPAHGRQVTVQEFALFFTIFDETKSWYFTENMERNCRAPCNIQMEDPTFKENYRFHAINGYIMDTLPGLVMAQDQRIRWYLLSMGSNENIHSIHFSGHVFTVRKKEEYKMALYNLYPGVFETVEMLPSKAGIWRVECLIGEHLHAGMSTLFLVYSDKCQTPLGMASGHIRDFQITASGQYGQWAPKLARLHYSGGINAWSTKEPFSWIKVDLLAPMIIHGIKTQGARQKFSSLYISQFIIMYSLDGKKWQTYRGNSTGTLMVFFGNVDSSGIKHNIFNPPIIARYIRLHPTHYSIRSTLRMELMGCDLNSCSMPLGMESKAISDAQITASSYFTNMFATWSPSKARLHLQGRSNAWRPQVNNPKEWLQVDFQKTMKVTGVTTQGVKSLLTSMYVKEFLISSSQDGHQWTLFFQNGKVKVFQGNQDSFTPVVNTLDPPLLTRYLRIHPQSWAHQIALRMEVLGCEAQDLYSKNNAIEPRSFSQNSRHPSLAEAKEAANAELDSYGVSDFYKRLIDKAKTVEGVEALKDAILAALP

SEQ ID NO: 119 - protein, 1778 aa, Artificial Sequence
  AD2CD2-15M_SC
MQIELSTCFFLCLLRFCFSATRRYYLGAVELSWDYMQSDLGELPVDARFPPRVPKSFPFNTSVVYKKTLFVEFTDHLFSIAKPRPPWMGLLGPTIQAEVYDTVVITLKNMATHPVSLHAVGVSYWKASEGAEYDDQTSQREKEDDKVFPGGSHTYVWQVSKENGPMASDPQCLTYSYLSHVDLAKDLNSGLIGALLVCREGSLAKEKTQTLHKFILLFAVFDEGKSWHSETKDSLMQDRDAASARAWPKMHTVNGYVNRSLPGLTGCHRKSVYWHVIGMGTTPEVHSIFLEGHTFLVRDHRQASLEISPITFLTAQTLLMDLGQFLLFCHISSHQHDGMEAYVKVDSCPEEPQLRMKNNEEAEDYDDDLTDSEMDVVRFDDDNSPSFIQIRSVAKKHPKTWVHYIAAEEEDWDYAPLVLAPDDRSHKSQYLNNGPQRIGRKYKKVRFMAYTDETFKTREAIQHESGILGPLLYGEVGDTLLIIFKNQASRPYNIYPHGITDVRPLYERRLPKGVKHLKDFPILPGEIFKYKWTVTVEDGPTKSDPRCLTRYYSSHVNMERDLASGLIGPLLICYKESVDQRGNQIMSDKRNVILFSVFDENRSWYLTENIQRFLPEPAGVQLEDPEFQASNIMHSINGYVFDSLQLSVCLHEVAYWYILSIGAQTDFLSVFFSGYTFKHKMVYEDTLTLFPFSGETVFMSMENPGNWILGCHNSDFRNRGMTALLKVSSCDKNTGDYYEDSYEDISASLLSKNNAIEPRSFSQNPPVLSKNNAIEPRSFSQNSRHPSLAEAKEAANAELDSYGVSDFYKRLIDKAKTVEGVEALKDAILAALPGSGGSGGSGGSGSGSKNNAIEPRSFSQNSRHPSGSGGSGGSGGSGSLAEAKEAANAELDSYGVSDFYKRLIDKAKTVEGVEALKDAILAALPSKNNAIEPRSFSQNSRHPSQSDQEEIDYDDTISVEMKKEDFDIYDEDENQSPRSFQKKTRHYFIAAVERLWDYGMSSSPHVLRNRAQSGSVPQFKKVVFQEFTDGSFTQPLYRGELNEHLGLLGPYIRAEVEDNIMVTFRNQASRPYSFYSSLISYEEDQRQGAEPRKNFVKPNETKTYFWEVQHHMAPTKDEFDCKAWAYFSDVDLEKDVHSGLIGPLLVCHTNTLNPAHGRQVTVQEFALFFTIFDETKSWYFTENMERNCRAPCNIQMEDPTFKENYRFHAINGYIMDTLPGLVMAQDQRIRWYLLSMGSNENIHSIHFSGHVFTVRKKEEYKMALYNLYPGVFETVEMLPSKAGIWRVECLIGEHLHAGMSTLFLVYSNKCQTPLGMASGHIRDFQITASGQYGQWAPKLARLHYSGSINAWSTKEPFSWIKVDLLAPMIIHGIKTQGARQKFSSLYISQFIIMYSLDGKKWQTYRGNSTGTLMVFFGNVDSSGIKHNIFNPPIIARYIRLHPTHYSIRSTLRMELMGCDLNSCSMPLGMESKAISDAQITASSYFTNMFATWSPSKARLHLQGRSNAWRPQVNNPKEWLQVDFQKTMKVTGVTTQGVKSLLTSMYVKEFLISSSQDGHQWTLFFQNGKVKVFQGNQDSFTPVVNSLDPPLLTRYLRIHPQSWVHQIALRMEVLGCEAQDLYSKNNAIEPRSFSQNSRHPSLAEAKEAANAELDSYGVSDFYKRLIDKAKTVEGVEALKDAILAALPGSGGSGGSGGSGSGSKNNAIEPRSFSQNSRHPSGSGGSGGSGGSGSLAEAKEAANAELDSYGVSDFYKRLIDKAKTVEGVEALKDAILAALP

SEQ ID NO: 120 - DNA, 4338 bp, Artificial Sequence
  AC-19M_SC
atgcagatcgagctgtctacctgcttcttcctgtgcctgctgcggttctgcttcagcgccaccagacggtactatctgggcgccgtggaactgagctgggactacatgcagagcgacctgggcgagctgcccgtggatgccagattccctccaagagtgcccaagagcttccccttcaacacctccgtggtgtacaagaaaaccctgttcgtggagttcaccgaccacctgttctctatcgccaagcccagacccccctggatgggactgctgggccctacaattcaggccgaggtgtacgacaccgtcgtgatcaccctgaagaacatggccacccaccccgtgtctctgcatgccgtgggagtgtcctactggaaggcctctgagggcgccgagtacgacgatcagaccagccagcgcgagaaagaggacgacaaggtgttccctggcggcagccacacctacgtgtggcaggtgtccaaagaaaacggccccatggccagcgacccccagtgtctgacatacagctacctgagccacgtggacctggccaaggacctgaactctggcctgatcggagccctgctcgtgtgcagagaaggcagcctggccaaagagaaaacccagaccctgcacaagttcatcctgctgttcgccgtgttcgacgagggcaagagctggcacagcgagacaaaggacagcctgatgcaggacagggacgccgcctctgctagagcctggcccaaaatgcacaccgtgaacggctacgtgaacagaagcctgcctggcctgaccggctgccacagaaagtccgtgtactggcacgtgatcggcatgggtaccacccccgaggtgcacagcatctttctggaaggacacacctttctcgtgcgggaccacagacaggccagcctggaaatcagccctatcaccttcctgaccgcccagacactgctgatggacctgggccagtttctgctgttctgccacatcagctcccaccagcacgacggcatggaagcctacgtgaaggtggacagctgccccgaggaaccccagctgcggatgaagaacaacgaggaagccgaggactacgacgacgacctgaccgacagcgagatggacgtggtgcgcttcgacgacgataacagccccagcttcatccagatcagaagcgtggccaagaagcaccccaagacctgggtgcactatatcgccgccgaggaagaggactgggattacgcccctctggtgctggcccccgacgacagatctcacaagagccagtacctgaacaacggcccccagcggatcggccggaagtataagaaagtgcggttcatggcctacaccgacgagacattcaagaccagagaggccatccagcacgagagcggcatcctgggccctctgctgtatggcgaagtgggcgacaccctgctgatcatcttcaagaaccaggccagcagaccctacaacatctaccctcacggcatcaccgacgtgcggcccctgtatgagagaaggctgcccaagggcgtgaagcacctgaaggacttccctatcctgcccggcgagatcttcaagtacaagtggaccgtgaccgtggaagatggccccaccaagagcgaccccagatgcctgacccggtactacagcagccacgtgaacatggaacgggatctggccagcggactgatcggcccactgctgatctgctacaaagaaagcgtggaccagcggggcaaccagatcatgagcgacaagcggaacgtgatcctgtttagcgtgttcgatgagaaccggtcctggtatctgaccgagaatatccagcggttcctgcccgaacctgccggcgtgcagctggaagatcctgagttccaggcctccaacatcatgcactccatcaatggctatgtgttcgacagcctgcagctgagcgtgtgcctgcacgaggtggcctactggtacatcctgagcatcggggcccagaccgacttcctgtccgtgttcttctccggctacaccttcaagcacaagatggtgtacgaggataccctgaccctgttcccctttagcggcgaaaccgtgttcatgagcatggaaaaccccggcaactggatcctgggctgtcacaacagcgacttccggaacagaggcatgaccgccctgctgaaggtgtccagctgcgacaagaacaccggcgactactacgaggacagctatgaggatatcagcgccagcctgctgagcaagaacaatgccatcgagccccggtccttcagccagaaccctcctgtgctgcagtccgaccaggaagagatcgattacgacgacaccatcagcgtggaaatgaagaaagaagatttcgacatctacgacgaggacgagaaccagagcccccggtctttccagaaaaagacccggcactacttcattgccgctgtggaacggctgtgggactacggcatgagcagcagccctcacgtgctgagaaacagggcccagagcggcagcgtgccccagttcaagaaagtggtgttccaggagtttaccgacggcagcttcacccagcccctgtacagaggcgagctgaacgagcatctgggcctgctgggaccttacatccgggccgaagtggaagataacatcatggtcaccttccggaatcaggcctcccggccctacagcttctacagctccctgatcagctacgaagaggaccagaggcagggggccgagcccagaaagaacttcgtgaagcccaacgagactaagacctacttctgggaggtgcagcaccacatggcccctacaaaggacgaattcgactgcaaggcctgggcctacttctccgatgtggatctggaaaaggacgtgcacagcggcctgattggacctctgctcgtgtgtcacaccaacaccctgaaccccgcccacggcagacaagtgacagtgcaggaatttgccctgttcttcaccatcttcgacgaaacaaagagctggtacttcaccgaaaacatggaaagaaactgccgggctccctgcaacatccagatggaagatcccaccttcaaagagaactaccggttccacgccatcaacggctacatcatggacacactgcccggcctcgtgatggctcaggatcagcggatccggtggtatctgctgtccatgggctccaacgagaacatccacagcatccacttcagcggccacgtgttcaccgtgcggaaaaaagaagagtacaaaatggccctgtacaacctgtaccctggggtgttcgagacagtggaaatgctgcccagcaaggccggcatctggcgggtggaatgtctgatcggggagcatctgcacgctgggatgagcacactgtttctggtgtacagcgacaagtgccagacacctctgggcatggcctctggccacatccgggactttcagatcacagccagcggccagtatggccagtgggcccctaaactggccagactgcactacagcggcggcatcaatgcctggtccaccaaagagcccttcagctggatcaaggtggacctgctggctcccatgatcatccacggaatcaagacccagggcgccagacagaagttcagcagcctgtacatcagccagttcatcatcatgtacagcctggacggcaagaagtggcagacctaccggggcaatagcaccggcaccctgatggtgttcttcggcaacgtggactcctccggcattaagcacaacatcttcaacccccccatcattgcccggtacatccggctgcaccccacccactacagcatccggtccaccctgcggatggaactgatgggctgcgacctgaacagctgcagcatgcccctgggaatggaaagcaaggccatctccgacgcccagatcaccgcctccagctacttcaccaacatgttcgccacctggtccccatccaaggcccggctgcatctgcagggcagaagcaatgcttggaggccccaagtgaacaaccccaaagaatggctgcaggtggactttcagaaaaccatgaaagtgaccggcgtgaccacccagggcgtgaaaagcctgctgacctctatgtacgtgaaagagttcctgatctccagcagccaggacggccaccagtggaccctgtttttccagaacggcaaagtgaaagtgtttcaggggaaccaggactccttcacccccgtcgtgaacacactggatcccccactgctgaccagatacctgcggatccaccctcagagctgggcccaccagatcgctctgagaatggaagtgctgggatgcgaggcccaggacctgtactga

SEQ ID NO: 121 - DNA, 5337 bp, Artificial Sequence
  AD2CD2-19M
atgcagatcgagctgtctacctgcttcttcctgtgcctgctgcggttctgcttcagcgccaccagacggtactatctgggcgccgtggaactgagctgggactacatgcagagcgacctgggcgagctgcccgtggatgccagattccctccaagagtgcccaagagcttccccttcaacacctccgtggtgtacaagaaaaccctgttcgtggagttcaccgaccacctgttctctatcgccaagcccagacccccctggatgggactgctgggccctacaattcaggccgaggtgtacgacaccgtcgtgatcaccctgaagaacatggccacccaccccgtgtctctgcatgccgtgggagtgtcctactggaaggcctctgagggcgccgagtacgacgatcagaccagccagcgcgagaaagaggacgacaaggtgttccctggcggcagccacacctacgtgtggcaggtgtccaaagaaaacggccccatggccagcgacccccagtgtctgacatacagctacctgagccacgtggacctggccaaggacctgaactctggcctgatcggagccctgctcgtgtgcagagaaggcagcctggccaaagagaaaacccagaccctgcacaagttcatcctgctgttcgccgtgttcgacgagggcaagagctggcacagcgagacaaaggacagcctgatgcaggacagggacgccgcctctgctagagcctggcccaaaatgcacaccgtgaacggctacgtgaacagaagcctgcctggcctgaccggctgccacagaaagtccgtgtactggcacgtgatcggcatgggtaccacccccgaggtgcacagcatctttctggaaggacacacctttctcgtgcgggaccacagacaggccagcctggaaatcagccctatcaccttcctgaccgcccagacactgctgatggacctgggccagtttctgctgttctgccacatcagctcccaccagcacgacggcatggaagcctacgtgaaggtggacagctgccccgaggaaccccagctgcggatgaagaacaacgaggaagccgaggactacgacgacgacctgaccgacagcgagatggacgtggtgcgcttcgacgacgataacagccccagcttcatccagatcagaagcgtggccaagaagcaccccaagacctgggtgcactatatcgccgccgaggaagaggactgggattacgcccctctggtgctggcccccgacgacagatctcacaagagccagtacctgaacaacggcccccagcggatcggccggaagtataagaaagtgcggttcatggcctacaccgacgagacattcaagaccagagaggccatccagcacgagagcggcatcctgggccctctgctgtatggcgaagtgggcgacaccctgctgatcatcttcaagaaccaggccagcagaccctacaacatctaccctcacggcatcaccgacgtgcggcccctgtatgagagaaggctgcccaagggcgtgaagcacctgaaggacttccctatcctgcccggcgagatcttcaagtacaagtggaccgtgaccgtggaagatggccccaccaagagcgaccccagatgcctgacccggtactacagcagccacgtgaacatggaacgggatctggccagcggactgatcggcccactgctgatctgctacaaagaaagcgtggaccagcggggcaaccagatcatgagcgacaagcggaacgtgatcctgtttagcgtgttcgatgagaaccggtcctggtatctgaccgagaatatccagcggttcctgcccgaacctgccggcgtgcagctggaagatcctgagttccaggcctccaacatcatgcactccatcaatggctatgtgttcgacagcctgcagctgagcgtgtgcctgcacgaggtggcctactggtacatcctgagcatcggggcccagaccgacttcctgtccgtgttcttctccggctacaccttcaagcacaagatggtgtacgaggataccctgaccctgttcccctttagcggcgaaaccgtgttcatgagcatggaaaaccccggcaactggatcctgggctgtcacaacagcgacttccggaacagaggcatgaccgccctgctgaaggtgtccagctgcgacaagaacaccggcgactactacgaggacagctatgaggatatcagcgccagcctgctgagcaagaacaatgccatcgagccccggtccttcagccagaaccctcctgtgctgtccaaaaacaacgccattgagcctcggagcttctcccagaactcccggcatccatctctggccgaggccaaagaagccgccaacgccgagctggatagctacggcgtgtccgacttctacaagcggctgatcgacaaggccaagacagtggaaggcgtggaagctctgaaggatgccatcctggctgccctgcctggaagcggaggatctggcggctcaggcggatctggctctggctctaaaaacaatgctattgaacctagatcttttagccagaacagccggcacccctccggctctggcggaagtgggggaagtggcggctctggatctctggcagaagctaaagaggctgctaacgctgaactggactcctatggggtgtccgatttttacaaaagactgattgataaggctaaaactgtggaaggggtggaagccctgaaagacgctattctggccgctctgccttctaagaacaacgcaatcgaaccacgcagcttttctcagaatagcagacaccccagccagtccgaccaggaagagatcgattacgacgacaccatcagcgtggaaatgaagaaagaagatttcgacatctacgacgaggacgagaaccagagcccccggtctttccagaaaaagacccggcactacttcattgccgctgtggaacggctgtgggactacggcatgagcagcagccctcacgtgctgagaaacagggcccagagcggcagcgtgccccagttcaagaaagtggtgttccaggagtttaccgacggcagcttcacccagcccctgtacagaggcgagctgaacgagcatctgggcctgctgggaccttacatccgggccgaagtggaagataacatcatggtcaccttccggaatcaggcctcccggccctacagcttctacagctccctgatcagctacgaagaggaccagaggcagggggccgagcccagaaagaacttcgtgaagcccaacgagactaagacctacttctgggaggtgcagcaccacatggcccctacaaaggacgaattcgactgcaaggcctgggcctacttctccgatgtggatctggaaaaggacgtgcacagcggcctgattggacctctgctcgtgtgtcacaccaacaccctgaaccccgcccacggcagacaagtgacagtgcaggaatttgccctgttcttcaccatcttcgacgaaacaaagagctggtacttcaccgaaaacatggaaagaaactgccgggctccctgcaacatccagatggaagatcccaccttcaaagagaactaccggttccacgccatcaacggctacatcatggacacactgcccggcctcgtgatggctcaggatcagcggatccggtggtatctgctgtccatgggctccaacgagaacatccacagcatccacttcagcggccacgtgttcaccgtgcggaaaaaagaagagtacaaaatggccctgtacaacctgtaccctggggtgttcgagacagtggaaatgctgcccagcaaggccggcatctggcgggtggaatgtctgatcggggagcatctgcacgctgggatgagcacactgtttctggtgtacagcgacaagtgccagacacctctgggcatggcctctggccacatccgggactttcagatcacagccagcggccagtatggccagtgggcccctaaactggccagactgcactacagcggcggcatcaatgcctggtccaccaaagagcccttcagctggatcaaggtggacctgctggctcccatgatcatccacggaatcaagacccagggcgccagacagaagttcagcagcctgtacatcagccagttcatcatcatgtacagcctggacggcaagaagtggcagacctaccggggcaatagcaccggcaccctgatggtgttcttcggcaacgtggactcctccggcattaagcacaacatcttcaacccccccatcattgcccggtacatccggctgcaccccacccactacagcatccggtccaccctgcggatggaactgatgggctgcgacctgaacagctgcagcatgcccctgggaatggaaagcaaggccatctccgacgcccagatcaccgcctccagctacttcaccaacatgttcgccacctggtccccatccaaggcccggctgcatctgcagggcagaagcaatgcttggaggccccaagtgaacaaccccaaagaatggctgcaggtggactttcagaaaaccatgaaagtgaccggcgtgaccacccagggcgtgaaaagcctgctgacctctatgtacgtgaaagagttcctgatctccagcagccaggacggccaccagtggaccctgtttttccagaacggcaaagtgaaagtgtttcaggggaaccaggactccttcacccccgtcgtgaacacactggatcccccactgctgaccagatacctgcggatccaccctcagagctgggcccaccagatcgctctgagaatggaagtgctgggatgcgaggcccaggacctgtacagcaagaacaacgctattgagccacgctcattcagccagaactccaggcaccctagcctggctgaggcaaaagaggcagcaaacgcagagctggattcttatggcgtgtcagatttctataagcgcctgattgacaaagcaaaaaccgtggaaggcgtggaagcactgaaggacgcaattctggcagccctgccaggcagtggcggatcagggggcagcggaggaagcggaagcggctcaaagaacaatgcaattgagccaagaagtttctctcagaacagtaggcacccaagcggctccgggggaagcgggggctcagggggctccggaagcctggcagaagccaaagaagcagcaaacgccgaactggacagttatggggtgtcagacttctacaagagactgattgataaggcaaagaccgtggaaggcgtggaagccctgaaagacgccatcctggcagcactgccatga

SEQ ID NO: 122 - DNA, 4785 bp, Artificial Sequence
  ALDLCLD-19M_SC
atgcagatcgagctgtctacctgcttcttcctgtgcctgctgcggttctgcttcagcgccaccagacggtactatctgggcgccgtggaactgagctgggactacatgcagagcgacctgggcgagctgcccgtggatgccagattccctccaagagtgcccaagagcttccccttcaacacctccgtggtgtacaagaaaaccctgttcgtggagttcaccgaccacctgttctctatcgccaagcccagacccccctggatgggactgctgggccctacaattcaggccgaggtgtacgacaccgtcgtgatcaccctgaagaacatggccacccaccccgtgtctctgcatgccgtgggagtgtcctactggaaggcctctgagggcgccgagtacgacgatcagaccagccagcgcgagaaagaggacgacaaggtgttccctggcggcagccacacctacgtgtggcaggtgtccaaagaaaacggccccatggccagcgacccccagtgtctgacatacagctacctgagccacgtggacctggccaaggacctgaactctggcctgatcggagccctgctcgtgtgcagagaaggcagcctggccaaagagaaaacccagaccctgcacaagttcatcctgctgttcgccgtgttcgacgagggcaagagctggcacagcgagacaaaggacagcctgatgcaggacagggacgccgcctctgctagagcctggcccaaaatgcacaccgtgaacggctacgtgaacagaagcctgcctggcctgaccggctgccacagaaagtccgtgtactggcacgtgatcggcatgggtaccacccccgaggtgcacagcatctttctggaaggacacacctttctcgtgcgggaccacagacaggccagcctggaaatcagccctatcaccttcctgaccgcccagacactgctgatggacctgggccagtttctgctgttctgccacatcagctcccaccagcacgacggcatggaagcctacgtgaaggtggacagctgccccgaggaaccccagctgcggatgaagaacaacgaggaagccgaggactacgacgacgacctgaccgacagcgagatggacgtggtgcgcttcgacgacgataacagccccagcttcatccagatcagaagcgtggccaagaagcaccccaagacctgggtgcactatatcgccgccgaggaagaggactgggattacgcccctctggtgctggcccccgacgacagatctcacaagagccagtacctgaacaacggcccccagcggatcggccggaagtataagaaagtgcggttcatggcctacaccgacgagacattcaagaccagagaggccatccagcacgagagcggcatcctgggccctctgctgtatggcgaagtgggcgacaccctgctgatcatcttcaagaaccaggccagcagaccctacaacatctaccctcacggcatcaccgacgtgcggcccctgtatgagagaaggctgcccaagggcgtgaagcacctgaaggacttccctatcctgcccggcgagatcttcaagtacaagtggaccgtgaccgtggaagatggccccaccaagagcgaccccagatgcctgacccggtactacagcagccacgtgaacatggaacgggatctggccagcggactgatcggcccactgctgatctgctacaaagaaagcgtggaccagcggggcaaccagatcatgagcgacaagcggaacgtgatcctgtttagcgtgttcgatgagaaccggtcctggtatctgaccgagaatatccagcggttcctgcccgaacctgccggcgtgcagctggaagatcctgagttccaggcctccaacatcatgcactccatcaatggctatgtgttcgacagcctgcagctgagcgtgtgcctgcacgaggtggcctactggtacatcctgagcatcggggcccagaccgacttcctgtccgtgttcttctccggctacaccttcaagcacaagatggtgtacgaggataccctgaccctgttcccctttagcggcgaaaccgtgttcatgagcatggaaaaccccggcaactggatcctgggctgtcacaacagcgacttccggaacagaggcatgaccgccctgctgaaggtgtccagctgcgacaagaacaccggcgactactacgaggacagctatgaggatatcagcgccagcctgctgagcaagaacaatgccatcgagccccggtccttcagccagaaccctcctgtgctgtccaaaaacaacgccattgagcctcggagcttctcccagaactcccggcatccatctctggccgaggccaaagaagccgccaacgccgagctggatagctacggcgtgtccgacttctacaagcggctgatcgacaaggccaagacagtggaaggcgtggaagctctgaaggatgccatcctggctgccctgccttctaagaacaacgcaatcgaaccacgcagcttttctcagaatagcagacaccccagccagtccgaccaggaagagatcgattacgacgacaccatcagcgtggaaatgaagaaagaagatttcgacatctacgacgaggacgagaaccagagcccccggtctttccagaaaaagacccggcactacttcattgccgctgtggaacggctgtgggactacggcatgagcagcagccctcacgtgctgagaaacagggcccagagcggcagcgtgccccagttcaagaaagtggtgttccaggagtttaccgacggcagcttcacccagcccctgtacagaggcgagctgaacgagcatctgggcctgctgggaccttacatccgggccgaagtggaagataacatcatggtcaccttccggaatcaggcctcccggccctacagcttctacagctccctgatcagctacgaagaggaccagaggcagggggccgagcccagaaagaacttcgtgaagcccaacgagactaagacctacttctgggaggtgcagcaccacatggcccctacaaaggacgaattcgactgcaaggcctgggcctacttctccgatgtggatctggaaaaggacgtgcacagcggcctgattggacctctgctcgtgtgtcacaccaacaccctgaaccccgcccacggcagacaagtgacagtgcaggaatttgccctgttcttcaccatcttcgacgaaacaaagagctggtacttcaccgaaaacatggaaagaaactgccgggctccctgcaacatccagatggaagatcccaccttcaaagagaactaccggttccacgccatcaacggctacatcatggacacactgcccggcctcgtgatggctcaggatcagcggatccggtggtatctgctgtccatgggctccaacgagaacatccacagcatccacttcagcggccacgtgttcaccgtgcggaaaaaagaagagtacaaaatggccctgtacaacctgtaccctggggtgttcgagacagtggaaatgctgcccagcaaggccggcatctggcgggtggaatgtctgatcggggagcatctgcacgctgggatgagcacactgtttctggtgtacagcgacaagtgccagacacctctgggcatggcctctggccacatccgggactttcagatcacagccagcggccagtatggccagtgggcccctaaactggccagactgcactacagcggcggcatcaatgcctggtccaccaaagagcccttcagctggatcaaggtggacctgctggctcccatgatcatccacggaatcaagacccagggcgccagacagaagttcagcagcctgtacatcagccagttcatcatcatgtacagcctggacggcaagaagtggcagacctaccggggcaatagcaccggcaccctgatggtgttcttcggcaacgtggactcctccggcattaagcacaacatcttcaacccccccatcattgcccggtacatccggctgcaccccacccactacagcatccggtccaccctgcggatggaactgatgggctgcgacctgaacagctgcagcatgcccctgggaatggaaagcaaggccatctccgacgcccagatcaccgcctccagctacttcaccaacatgttcgccacctggtccccatccaaggcccggctgcatctgcagggcagaagcaatgcttggaggccccaagtgaacaaccccaaagaatggctgcaggtggactttcagaaaaccatgaaagtgaccggcgtgaccacccagggcgtgaaaagcctgctgacctctatgtacgtgaaagagttcctgatctccagcagccaggacggccaccagtggaccctgtttttccagaacggcaaagtgaaagtgtttcaggggaaccaggactccttcacccccgtcgtgaacacactggatcccccactgctgaccagatacctgcggatccaccctcagagctgggcccaccagatcgctctgagaatggaagtgctgggatgcgaggcccaggacctgtacagcaagaacaacgctattgagccacgctcattcagccagaactccaggcaccctagcctggctgaggcaaaagaggcagcaaacgcagagctggattcttatggcgtgtcagatttctataagcgcctgattgacaaagcaaaaaccgtggaaggcgtggaagcactgaaggacgcaattctggcagccctgccatga

SEQ ID NO: 123 - DNA, 4728 bp, Artificial Sequence
  ADLCLD-19M_SC-V1
atgcagatcgagctgtctacctgcttcttcctgtgcctgctgcggttctgcttcagcgccaccagacggtactatctgggcgccgtggaactgagctgggactacatgcagagcgacctgggcgagctgcccgtggatgccagattccctccaagagtgcccaagagcttccccttcaacacctccgtggtgtacaagaaaaccctgttcgtggagttcaccgaccacctgttctctatcgccaagcccagacccccctggatgggactgctgggccctacaattcaggccgaggtgtacgacaccgtcgtgatcaccctgaagaacatggccacccaccccgtgtctctgcatgccgtgggagtgtcctactggaaggcctctgagggcgccgagtacgacgatcagaccagccagcgcgagaaagaggacgacaaggtgttccctggcggcagccacacctacgtgtggcaggtgtccaaagaaaacggccccatggccagcgacccccagtgtctgacatacagctacctgagccacgtggacctggccaaggacctgaactctggcctgatcggagccctgctcgtgtgcagagaaggcagcctggccaaagagaaaacccagaccctgcacaagttcatcctgctgttcgccgtgttcgacgagggcaagagctggcacagcgagacaaaggacagcctgatgcaggacagggacgccgcctctgctagagcctggcccaaaatgcacaccgtgaacggctacgtgaacagaagcctgcctggcctgaccggctgccacagaaagtccgtgtactggcacgtgatcggcatgggtaccacccccgaggtgcacagcatctttctggaaggacacacctttctcgtgcgggaccacagacaggccagcctggaaatcagccctatcaccttcctgaccgcccagacactgctgatggacctgggccagtttctgctgttctgccacatcagctcccaccagcacgacggcatggaagcctacgtgaaggtggacagctgccccgaggaaccccagctgcggatgaagaacaacgaggaagccgaggactacgacgacgacctgaccgacagcgagatggacgtggtgcgcttcgacgacgataacagccccagcttcatccagatcagaagcgtggccaagaagcaccccaagacctgggtgcactatatcgccgccgaggaagaggactgggattacgcccctctggtgctggcccccgacgacagatctcacaagagccagtacctgaacaacggcccccagcggatcggccggaagtataagaaagtgcggttcatggcctacaccgacgagacattcaagaccagagaggccatccagcacgagagcggcatcctgggccctctgctgtatggcgaagtgggcgacaccctgctgatcatcttcaagaaccaggccagcagaccctacaacatctaccctcacggcatcaccgacgtgcggcccctgtatgagagaaggctgcccaagggcgtgaagcacctgaaggacttccctatcctgcccggcgagatcttcaagtacaagtggaccgtgaccgtggaagatggccccaccaagagcgaccccagatgcctgacccggtactacagcagccacgtgaacatggaacgggatctggccagcggactgatcggcccactgctgatctgctacaaagaaagcgtggaccagcggggcaaccagatcatgagcgacaagcggaacgtgatcctgtttagcgtgttcgatgagaaccggtcctggtatctgaccgagaatatccagcggttcctgcccgaacctgccggcgtgcagctggaagatcctgagttccaggcctccaacatcatgcactccatcaatggctatgtgttcgacagcctgcagctgagcgtgtgcctgcacgaggtggcctactggtacatcctgagcatcggggcccagaccgacttcctgtccgtgttcttctccggctacaccttcaagcacaagatggtgtacgaggataccctgaccctgttcccctttagcggcgaaaccgtgttcatgagcatggaaaaccccggcaactggatcctgggctgtcacaacagcgacttccggaacagaggcatgaccgccctgctgaaggtgtccagctgcgacaagaacaccggcgactactacgaggacagctatgaggatatcagcgccagcctgctgagcaagaacaatgccatcgagccccggtccttcagccagaaccctcctgtgctgctggccgaggccaaagaagccgccaacgccgagctggatagctacggcgtgtccgacttctacaagcggctgatcgacaaggccaagacagtggaaggcgtggaagctctgaaggatgccatcctggctgccctgccttctaagaacaacgcaatcgaaccacgcagcttttctcagaatagcagacaccccagccagtccgaccaggaagagatcgattacgacgacaccatcagcgtggaaatgaagaaagaagatttcgacatctacgacgaggacgagaaccagagcccccggtctttccagaaaaagacccggcactacttcattgccgctgtggaacggctgtgggactacggcatgagcagcagccctcacgtgctgagaaacagggcccagagcggcagcgtgccccagttcaagaaagtggtgttccaggagtttaccgacggcagcttcacccagcccctgtacagaggcgagctgaacgagcatctgggcctgctgggaccttacatccgggccgaagtggaagataacatcatggtcaccttccggaatcaggcctcccggccctacagcttctacagctccctgatcagctacgaagaggaccagaggcagggggccgagcccagaaagaacttcgtgaagcccaacgagactaagacctacttctgggaggtgcagcaccacatggcccctacaaaggacgaattcgactgcaaggcctgggcctacttctccgatgtggatctggaaaaggacgtgcacagcggcctgattggacctctgctcgtgtgtcacaccaacaccctgaaccccgcccacggcagacaagtgacagtgcaggaatttgccctgttcttcaccatcttcgacgaaacaaagagctggtacttcaccgaaaacatggaaagaaactgccgggctccctgcaacatccagatggaagatcccaccttcaaagagaactaccggttccacgccatcaacggctacatcatggacacactgcccggcctcgtgatggctcaggatcagcggatccggtggtatctgctgtccatgggctccaacgagaacatccacagcatccacttcagcggccacgtgttcaccgtgcggaaaaaagaagagtacaaaatggccctgtacaacctgtaccctggggtgttcgagacagtggaaatgctgcccagcaaggccggcatctggcgggtggaatgtctgatcggggagcatctgcacgctgggatgagcacactgtttctggtgtacagcgacaagtgccagacacctctgggcatggcctctggccacatccgggactttcagatcacagccagcggccagtatggccagtgggcccctaaactggccagactgcactacagcggcggcatcaatgcctggtccaccaaagagcccttcagctggatcaaggtggacctgctggctcccatgatcatccacggaatcaagacccagggcgccagacagaagttcagcagcctgtacatcagccagttcatcatcatgtacagcctggacggcaagaagtggcagacctaccggggcaatagcaccggcaccctgatggtgttcttcggcaacgtggactcctccggcattaagcacaacatcttcaacccccccatcattgcccggtacatccggctgcaccccacccactacagcatccggtccaccctgcggatggaactgatgggctgcgacctgaacagctgcagcatgcccctgggaatggaaagcaaggccatctccgacgcccagatcaccgcctccagctacttcaccaacatgttcgccacctggtccccatccaaggcccggctgcatctgcagggcagaagcaatgcttggaggccccaagtgaacaaccccaaagaatggctgcaggtggactttcagaaaaccatgaaagtgaccggcgtgaccacccagggcgtgaaaagcctgctgacctctatgtacgtgaaagagttcctgatctccagcagccaggacggccaccagtggaccctgtttttccagaacggcaaagtgaaagtgtttcaggggaaccaggactccttcacccccgtcgtgaacacactggatcccccactgctgaccagatacctgcggatccaccctcagagctgggcccaccagatcgctctgagaatggaagtgctgggatgcgaggcccaggacctgtacagcaagaacaacgctattgagccacgctcattcagccagaactccaggcaccctagcctggctgaggcaaaagaggcagcaaacgcagagctggattcttatggcgtgtcagatttctataagcgcctgattgacaaagcaaaaaccgtggaaggcgtggaagcactgaaggacgcaattctggcagccctgccatga

SEQ ID NO: 124 - DNA, 4728 bp, Artificial Sequence
  ADLCLD-19M_SC-V2
atgcagatcgagctgtctacctgcttcttcctgtgcctgctgcggttctgcttcagcgccaccagacggtactatctgggcgccgtggaactgagctgggactacatgcagagcgacctgggcgagctgcccgtggatgccagattccctccaagagtgcccaagagcttccccttcaacacctccgtggtgtacaagaaaaccctgttcgtggagttcaccgaccacctgttctctatcgccaagcccagacccccctggatgggactgctgggccctacaattcaggccgaggtgtacgacaccgtcgtgatcaccctgaagaacatggccacccaccccgtgtctctgcatgccgtgggagtgtcctactggaaggcctctgagggcgccgagtacgacgatcagaccagccagcgcgagaaagaggacgacaaggtgttccctggcggcagccacacctacgtgtggcaggtgtccaaagaaaacggccccatggccagcgacccccagtgtctgacatacagctacctgagccacgtggacctggccaaggacctgaactctggcctgatcggagccctgctcgtgtgcagagaaggcagcctggccaaagagaaaacccagaccctgcacaagttcatcctgctgttcgccgtgttcgacgagggcaagagctggcacagcgagacaaaggacagcctgatgcaggacagggacgccgcctctgctagagcctggcccaaaatgcacaccgtgaacggctacgtgaacagaagcctgcctggcctgaccggctgccacagaaagtccgtgtactggcacgtgatcggcatgggtaccacccccgaggtgcacagcatctttctggaaggacacacctttctcgtgcgggaccacagacaggccagcctggaaatcagccctatcaccttcctgaccgcccagacactgctgatggacctgggccagtttctgctgttctgccacatcagctcccaccagcacgacggcatggaagcctacgtgaaggtggacagctgccccgaggaaccccagctgcggatgaagaacaacgaggaagccgaggactacgacgacgacctgaccgacagcgagatggacgtggtgcgcttcgacgacgataacagccccagcttcatccagatcagaagcgtggccaagaagcaccccaagacctgggtgcactatatcgccgccgaggaagaggactgggattacgcccctctggtgctggcccccgacgacagatctcacaagagccagtacctgaacaacggcccccagcggatcggccggaagtataagaaagtgcggttcatggcctacaccgacgagacattcaagaccagagaggccatccagcacgagagcggcatcctgggccctctgctgtatggcgaagtgggcgacaccctgctgatcatcttcaagaaccaggccagcagaccctacaacatctaccctcacggcatcaccgacgtgcggcccctgtatgagagaaggctgcccaagggcgtgaagcacctgaaggacttccctatcctgcccggcgagatcttcaagtacaagtggaccgtgaccgtggaagatggccccaccaagagcgaccccagatgcctgacccggtactacagcagccacgtgaacatggaacgggatctggccagcggactgatcggcccactgctgatctgctacaaagaaagcgtggaccagcggggcaaccagatcatgagcgacaagcggaacgtgatcctgtttagcgtgttcgatgagaaccggtcctggtatctgaccgagaatatccagcggttcctgcccgaacctgccggcgtgcagctggaagatcctgagttccaggcctccaacatcatgcactccatcaatggctatgtgttcgacagcctgcagctgagcgtgtgcctgcacgaggtggcctactggtacatcctgagcatcggggcccagaccgacttcctgtccgtgttcttctccggctacaccttcaagcacaagatggtgtacgaggataccctgaccctgttcccctttagcggcgaaaccgtgttcatgagcatggaaaaccccggcaactggatcctgggctgtcacaacagcgacttccggaacagaggcatgaccgccctgctgaaggtgtccagctgcgacaagaacaccggcgactactacgaggacagctatgaggatatcagcgccagcctgctgagcaagaacaatgccatcgagccccggtccttcagccagaaccctcctgtgctggccgaggccaaagaagccgccaacgccgagctggatagctacggcgtgtccgacttctacaagcggctgatcgacaaggccaagacagtggaaggcgtggaagctctgaaggatgccatcctggctgccctgccttctaagaacaacgcaatcgaaccacgcagcttttctcagaatagcagacaccccagcctgcagtccgaccaggaagagatcgattacgacgacaccatcagcgtggaaatgaagaaagaagatttcgacatctacgacgaggacgagaaccagagcccccggtctttccagaaaaagacccggcactacttcattgccgctgtggaacggctgtgggactacggcatgagcagcagccctcacgtgctgagaaacagggcccagagcggcagcgtgccccagttcaagaaagtggtgttccaggagtttaccgacggcagcttcacccagcccctgtacagaggcgagctgaacgagcatctgggcctgctgggaccttacatccgggccgaagtggaagataacatcatggtcaccttccggaatcaggcctcccggccctacagcttctacagctccctgatcagctacgaagaggaccagaggcagggggccgagcccagaaagaacttcgtgaagcccaacgagactaagacctacttctgggaggtgcagcaccacatggcccctacaaaggacgaattcgactgcaaggcctgggcctacttctccgatgtggatctggaaaaggacgtgcacagcggcctgattggacctctgctcgtgtgtcacaccaacaccctgaaccccgcccacggcagacaagtgacagtgcaggaatttgccctgttcttcaccatcttcgacgaaacaaagagctggtacttcaccgaaaacatggaaagaaactgccgggctccctgcaacatccagatggaagatcccaccttcaaagagaactaccggttccacgccatcaacggctacatcatggacacactgcccggcctcgtgatggctcaggatcagcggatccggtggtatctgctgtccatgggctccaacgagaacatccacagcatccacttcagcggccacgtgttcaccgtgcggaaaaaagaagagtacaaaatggccctgtacaacctgtaccctggggtgttcgagacagtggaaatgctgcccagcaaggccggcatctggcgggtggaatgtctgatcggggagcatctgcacgctgggatgagcacactgtttctggtgtacagcgacaagtgccagacacctctgggcatggcctctggccacatccgggactttcagatcacagccagcggccagtatggccagtgggcccctaaactggccagactgcactacagcggcggcatcaatgcctggtccaccaaagagcccttcagctggatcaaggtggacctgctggctcccatgatcatccacggaatcaagacccagggcgccagacagaagttcagcagcctgtacatcagccagttcatcatcatgtacagcctggacggcaagaagtggcagacctaccggggcaatagcaccggcaccctgatggtgttcttcggcaacgtggactcctccggcattaagcacaacatcttcaacccccccatcattgcccggtacatccggctgcaccccacccactacagcatccggtccaccctgcggatggaactgatgggctgcgacctgaacagctgcagcatgcccctgggaatggaaagcaaggccatctccgacgcccagatcaccgcctccagctacttcaccaacatgttcgccacctggtccccatccaaggcccggctgcatctgcagggcagaagcaatgcttggaggccccaagtgaacaaccccaaagaatggctgcaggtggactttcagaaaaccatgaaagtgaccggcgtgaccacccagggcgtgaaaagcctgctgacctctatgtacgtgaaagagttcctgatctccagcagccaggacggccaccagtggaccctgtttttccagaacggcaaagtgaaagtgtttcaggggaaccaggactccttcacccccgtcgtgaacacactggatcccccactgctgaccagatacctgcggatccaccctcagagctgggcccaccagatcgctctgagaatggaagtgctgggatgcgaggcccaggacctgtacagcaagaacaacgctattgagccacgctcattcagccagaactccaggcaccctagcctggctgaggcaaaagaggcagcaaacgcagagctggattcttatggcgtgtcagatttctataagcgcctgattgacaaagcaaaaaccgtggaaggcgtggaagcactgaaggacgcaattctggcagccctgccatga

SEQ ID NO: 125 - DNA, 5061 bp, Artificial Sequence
  AD2CD-19M_SC
atgcagatcgagctgtctacctgcttcttcctgtgcctgctgcggttctgcttcagcgccaccagacggtactatctgggcgccgtggaactgagctgggactacatgcagagcgacctgggcgagctgcccgtggatgccagattccctccaagagtgcccaagagcttccccttcaacacctccgtggtgtacaagaaaaccctgttcgtggagttcaccgaccacctgttctctatcgccaagcccagacccccctggatgggactgctgggccctacaattcaggccgaggtgtacgacaccgtcgtgatcaccctgaagaacatggccacccaccccgtgtctctgcatgccgtgggagtgtcctactggaaggcctctgagggcgccgagtacgacgatcagaccagccagcgcgagaaagaggacgacaaggtgttccctggcggcagccacacctacgtgtggcaggtgtccaaagaaaacggccccatggccagcgacccccagtgtctgacatacagctacctgagccacgtggacctggccaaggacctgaactctggcctgatcggagccctgctcgtgtgcagagaaggcagcctggccaaagagaaaacccagaccctgcacaagttcatcctgctgttcgccgtgttcgacgagggcaagagctggcacagcgagacaaaggacagcctgatgcaggacagggacgccgcctctgctagagcctggcccaaaatgcacaccgtgaacggctacgtgaacagaagcctgcctggcctgaccggctgccacagaaagtccgtgtactggcacgtgatcggcatgggtaccacccccgaggtgcacagcatctttctggaaggacacacctttctcgtgcgggaccacagacaggccagcctggaaatcagccctatcaccttcctgaccgcccagacactgctgatggacctgggccagtttctgctgttctgccacatcagctcccaccagcacgacggcatggaagcctacgtgaaggtggacagctgccccgaggaaccccagctgcggatgaagaacaacgaggaagccgaggactacgacgacgacctgaccgacagcgagatggacgtggtgcgcttcgacgacgataacagccccagcttcatccagatcagaagcgtggccaagaagcaccccaagacctgggtgcactatatcgccgccgaggaagaggactgggattacgcccctctggtgctggcccccgacgacagatctcacaagagccagtacctgaacaacggcccccagcggatcggccggaagtataagaaagtgcggttcatggcctacaccgacgagacattcaagaccagagaggccatccagcacgagagcggcatcctgggccctctgctgtatggcgaagtgggcgacaccctgctgatcatcttcaagaaccaggccagcagaccctacaacatctaccctcacggcatcaccgacgtgcggcccctgtatgagagaaggctgcccaagggcgtgaagcacctgaaggacttccctatcctgcccggcgagatcttcaagtacaagtggaccgtgaccgtggaagatggccccaccaagagcgaccccagatgcctgacccggtactacagcagccacgtgaacatggaacgggatctggccagcggactgatcggcccactgctgatctgctacaaagaaagcgtggaccagcggggcaaccagatcatgagcgacaagcggaacgtgatcctgtttagcgtgttcgatgagaaccggtcctggtatctgaccgagaatatccagcggttcctgcccgaacctgccggcgtgcagctggaagatcctgagttccaggcctccaacatcatgcactccatcaatggctatgtgttcgacagcctgcagctgagcgtgtgcctgcacgaggtggcctactggtacatcctgagcatcggggcccagaccgacttcctgtccgtgttcttctccggctacaccttcaagcacaagatggtgtacgaggataccctgaccctgttcccctttagcggcgaaaccgtgttcatgagcatggaaaaccccggcaactggatcctgggctgtcacaacagcgacttccggaacagaggcatgaccgccctgctgaaggtgtccagctgcgacaagaacaccggcgactactacgaggacagctatgaggatatcagcgccagcctgctgagcaagaacaatgccatcgagccccggtccttcagccagaaccctcctgtgctgtccaaaaacaacgccattgagcctcggagcttctcccagaactcccggcatccatctctggccgaggccaaagaagccgccaacgccgagctggatagctacggcgtgtccgacttctacaagcggctgatcgacaaggccaagacagtggaaggcgtggaagctctgaaggatgccatcctggctgccctgcctggaagcggaggatctggcggctcaggcggatctggctctggctctaaaaacaatgctattgaacctagatcttttagccagaacagccggcacccctccggctctggcggaagtgggggaagtggcggctctggatctctggcagaagctaaagaggctgctaacgctgaactggactcctatggggtgtccgatttttacaaaagactgattgataaggctaaaactgtggaaggggtggaagccctgaaagacgctattctggccgctctgccttctaagaacaacgcaatcgaaccacgcagcttttctcagaatagcagacaccccagccagtccgaccaggaagagatcgattacgacgacaccatcagcgtggaaatgaagaaagaagatttcgacatctacgacgaggacgagaaccagagcccccggtctttccagaaaaagacccggcactacttcattgccgctgtggaacggctgtgggactacggcatgagcagcagccctcacgtgctgagaaacagggcccagagcggcagcgtgccccagttcaagaaagtggtgttccaggagtttaccgacggcagcttcacccagcccctgtacagaggcgagctgaacgagcatctgggcctgctgggaccttacatccgggccgaagtggaagataacatcatggtcaccttccggaatcaggcctcccggccctacagcttctacagctccctgatcagctacgaagaggaccagaggcagggggccgagcccagaaagaacttcgtgaagcccaacgagactaagacctacttctgggaggtgcagcaccacatggcccctacaaaggacgaattcgactgcaaggcctgggcctacttctccgatgtggatctggaaaaggacgtgcacagcggcctgattggacctctgctcgtgtgtcacaccaacaccctgaaccccgcccacggcagacaagtgacagtgcaggaatttgccctgttcttcaccatcttcgacgaaacaaagagctggtacttcaccgaaaacatggaaagaaactgccgggctccctgcaacatccagatggaagatcccaccttcaaagagaactaccggttccacgccatcaacggctacatcatggacacactgcccggcctcgtgatggctcaggatcagcggatccggtggtatctgctgtccatgggctccaacgagaacatccacagcatccacttcagcggccacgtgttcaccgtgcggaaaaaagaagagtacaaaatggccctgtacaacctgtaccctggggtgttcgagacagtggaaatgctgcccagcaaggccggcatctggcgggtggaatgtctgatcggggagcatctgcacgctgggatgagcacactgtttctggtgtacagcgacaagtgccagacacctctgggcatggcctctggccacatccgggactttcagatcacagccagcggccagtatggccagtgggcccctaaactggccagactgcactacagcggcggcatcaatgcctggtccaccaaagagcccttcagctggatcaaggtggacctgctggctcccatgatcatccacggaatcaagacccagggcgccagacagaagttcagcagcctgtacatcagccagttcatcatcatgtacagcctggacggcaagaagtggcagacctaccggggcaatagcaccggcaccctgatggtgttcttcggcaacgtggactcctccggcattaagcacaacatcttcaacccccccatcattgcccggtacatccggctgcaccccacccactacagcatccggtccaccctgcggatggaactgatgggctgcgacctgaacagctgcagcatgcccctgggaatggaaagcaaggccatctccgacgcccagatcaccgcctccagctacttcaccaacatgttcgccacctggtccccatccaaggcccggctgcatctgcagggcagaagcaatgcttggaggccccaagtgaacaaccccaaagaatggctgcaggtggactttcagaaaaccatgaaagtgaccggcgtgaccacccagggcgtgaaaagcctgctgacctctatgtacgtgaaagagttcctgatctccagcagccaggacggccaccagtggaccctgtttttccagaacggcaaagtgaaagtgtttcaggggaaccaggactccttcacccccgtcgtgaacacactggatcccccactgctgaccagatacctgcggatccaccctcagagctgggcccaccagatcgctctgagaatggaagtgctgggatgcgaggcccaggacctgtacagcaagaacaacgctattgagccacgctcattcagccagaactccaggcaccctagcctggctgaggcaaaagaggcagcaaacgcagagctggattcttatggcgtgtcagatttctataagcgcctgattgacaaagcaaaaaccgtggaaggcgtggaagcactgaaggacgcaattctggcagccctgccatga

SEQ ID NO: 126 - DNA, 5337 bp, Artificial Sequence
  AD2CD2-15M_SC
atgcagatcgagctgtctacctgcttcttcctgtgcctgctgcggttctgcttcagcgccaccagacggtactatctgggcgccgtggaactgagctgggactacatgcagagcgacctgggcgagctgcccgtggatgccagattccctccaagagtgcccaagagcttccccttcaacacctccgtggtgtacaagaaaaccctgttcgtggagttcaccgaccacctgttctctatcgccaagcccagacccccctggatgggactgctgggccctacaattcaggccgaggtgtacgacaccgtcgtgatcaccctgaagaacatggccacccaccccgtgtctctgcatgccgtgggagtgtcctactggaaggcctctgagggcgccgagtacgacgatcagaccagccagcgcgagaaagaggacgacaaggtgttccctggcggcagccacacctacgtgtggcaggtgtccaaagaaaacggccccatggccagcgacccccagtgtctgacatacagctacctgagccacgtggacctggccaaggacctgaactctggcctgatcggagccctgctcgtgtgcagagaaggcagcctggccaaagagaaaacccagaccctgcacaagttcatcctgctgttcgccgtgttcgacgagggcaagagctggcacagcgagacaaaggacagcctgatgcaggacagggacgccgcctctgctagagcctggcccaaaatgcacaccgtgaacggctacgtgaacagaagcctgcctggcctgaccggctgccacagaaagtccgtgtactggcacgtgatcggcatgggtaccacccccgaggtgcacagcatctttctggaaggacacacctttctcgtgcgggaccacagacaggccagcctggaaatcagccctatcaccttcctgaccgcccagacactgctgatggacctgggccagtttctgctgttctgccacatcagctcccaccagcacgacggcatggaagcctacgtgaaggtggacagctgccccgaggaaccccagctgcggatgaagaacaacgaggaagccgaggactacgacgacgacctgaccgacagcgagatggacgtggtgcgcttcgacgacgataacagccccagcttcatccagatcagaagcgtggccaagaagcaccccaagacctgggtgcactatatcgccgccgaggaagaggactgggattacgcccctctggtgctggcccccgacgacagatctcacaagagccagtacctgaacaacggcccccagcggatcggccggaagtataagaaagtgcggttcatggcctacaccgacgagacattcaagaccagagaggccatccagcacgagagcggcatcctgggccctctgctgtatggcgaagtgggcgacaccctgctgatcatcttcaagaaccaggccagcagaccctacaacatctaccctcacggcatcaccgacgtgcggcccctgtatgagagaaggctgcccaagggcgtgaagcacctgaaggacttccctatcctgcccggcgagatcttcaagtacaagtggaccgtgaccgtggaagatggccccaccaagagcgaccccagatgcctgacccggtactacagcagccacgtgaacatggaacgggatctggccagcggactgatcggcccactgctgatctgctacaaagaaagcgtggaccagcggggcaaccagatcatgagcgacaagcggaacgtgatcctgtttagcgtgttcgatgagaaccggtcctggtatctgaccgagaatatccagcggttcctgcccgaacctgccggcgtgcagctggaagatcctgagttccaggcctccaacatcatgcactccatcaatggctatgtgttcgacagcctgcagctgagcgtgtgcctgcacgaggtggcctactggtacatcctgagcatcggggcccagaccgacttcctgtccgtgttcttctccggctacaccttcaagcacaagatggtgtacgaggataccctgaccctgttcccctttagcggcgaaaccgtgttcatgagcatggaaaaccccggcaactggatcctgggctgtcacaacagcgacttccggaacagaggcatgaccgccctgctgaaggtgtccagctgcgacaagaacaccggcgactactacgaggacagctatgaggatatcagcgccagcctgctgagcaagaacaatgccatcgagccccggtccttcagccagaaccctcctgtgctgtccaaaaacaacgccattgagcctcggagcttctcccagaactcccggcatccatctctggccgaggccaaagaagccgccaacgccgagctggatagctacggcgtgtccgacttctacaagcggctgatcgacaaggccaagacagtggaaggcgtggaagctctgaaggatgccatcctggctgccctgcctggaagcggaggatctggcggctcaggcggatctggctctggctctaaaaacaatgctattgaacctagatcttttagccagaacagccggcacccctccggctctggcggaagtgggggaagtggcggctctggatctctggcagaagctaaagaggctgctaacgctgaactggactcctatggggtgtccgatttttacaaaagactgattgataaggctaaaactgtggaaggggtggaagccctgaaagacgctattctggccgctctgccttctaagaacaacgcaatcgaaccacgcagcttttctcagaatagcagacaccccagccagtccgaccaggaagagatcgattacgacgacaccatcagcgtggaaatgaagaaagaagatttcgacatctacgacgaggacgagaaccagagcccccggtctttccagaaaaagacccggcactacttcattgccgctgtggaacggctgtgggactacggcatgagcagcagccctcacgtgctgagaaacagggcccagagcggcagcgtgccccagttcaagaaagtggtgttccaggagtttaccgacggcagcttcacccagcccctgtacagaggcgagctgaacgagcatctgggcctgctgggaccttacatccgggccgaagtggaagataacatcatggtcaccttccggaatcaggcctcccggccctacagcttctacagctccctgatcagctacgaagaggaccagaggcagggggccgagcccagaaagaacttcgtgaagcccaacgagactaagacctacttctgggaggtgcagcaccacatggcccctacaaaggacgaattcgactgcaaggcctgggcctacttctccgatgtggatctggaaaaggacgtgcacagcggcctgattggacctctgctcgtgtgtcacaccaacaccctgaaccccgcccacggcagacaagtgacagtgcaggaatttgccctgttcttcaccatcttcgacgaaacaaagagctggtacttcaccgaaaacatggaaagaaactgccgggctccctgcaacatccagatggaagatcccaccttcaaagagaactaccggttccacgccatcaacggctacatcatggacacactgcccggcctcgtgatggctcaggatcagcggatccggtggtatctgctgtccatgggctccaacgagaacatccacagcatccacttcagcggccacgtgttcaccgtgcggaaaaaagaagagtacaaaatggccctgtacaacctgtaccctggggtgttcgagacagtggaaatgctgcccagcaaggccggcatctggcgggtggaatgtctgatcggggagcatctgcacgctgggatgagcacactgtttctggtgtacagcaacaagtgccagacacctctgggcatggcctctggccacatccgggactttcagatcacagccagcggccagtatggccagtgggcccctaaactggccagactgcactacagcggcagcatcaatgcctggtccaccaaagagcccttcagctggatcaaggtggacctgctggctcccatgatcatccacggaatcaagacccagggcgccagacagaagttcagcagcctgtacatcagccagttcatcatcatgtacagcctggacggcaagaagtggcagacctaccggggcaatagcaccggcaccctgatggtgttcttcggcaacgtggactcctccggcattaagcacaacatcttcaacccccccatcattgcccggtacatccggctgcaccccacccactacagcatccggtccaccctgcggatggaactgatgggctgcgacctgaacagctgcagcatgcccctgggaatggaaagcaaggccatctccgacgcccagatcaccgcctccagctacttcaccaacatgttcgccacctggtccccatccaaggcccggctgcatctgcagggcagaagcaatgcttggaggccccaagtgaacaaccccaaagaatggctgcaggtggactttcagaaaaccatgaaagtgaccggcgtgaccacccagggcgtgaaaagcctgctgacctctatgtacgtgaaagagttcctgatctccagcagccaggacggccaccagtggaccctgtttttccagaacggcaaagtgaaagtgtttcaggggaaccaggactccttcacccccgtcgtgaacagcctggatcccccactgctgaccagatacctgcggatccaccctcagagctgggtgcaccagatcgctctgagaatggaagtgctgggatgcgaggcccaggacctgtacagcaagaacaacgctattgagccacgctcattcagccagaactccaggcaccctagcctggctgaggcaaaagaggcagcaaacgcagagctggattcttatggcgtgtcagatttctataagcgcctgattgacaaagcaaaaaccgtggaaggcgtggaagcactgaaggacgcaattctggcagccctgccaggcagtggcggatcagggggcagcggaggaagcggaagcggctcaaagaacaatgcaattgagccaagaagtttctctcagaacagtaggcacccaagcggctccgggggaagcgggggctcagggggctccggaagcctggcagaagccaaagaagcagcaaacgccgaactggacagttatggggtgtcagacttctacaagagactgattgataaggcaaagaccgtggaaggcgtggaagccctgaaagacgccatcctggcagcactgccatga

SEQ ID NO: 127 - protein, 447 aa, Homo sapiens [sequence omitted]

(19) United States
(12) Patent Application Publication
(10) Pub. No.: US 2023/0190881 A1
(43) Pub. Date: Jun. 22, 2023

(54) SUBCUTANEOUS ADMINISTRATION OF FACTOR VIII

(71) Applicant: Biotest AG, Dreieich (DE)

(72) Inventors: Peter HERBENER, Kirchhain (DE); Steffen KISTNER, Frankfurt/Main (DE)

(21) Appl. No.: 17/800,453

(22) PCT Filed: Feb. 16, 2021
(86) PCT No.: PCT/EP2021/053709

§ 371 Date: Aug. 17, 2022

(30) Foreign Application Priority Data
Feb. 17, 2020 (EP) 20157774.9

(51) Int. Cl. A61K 38/37; A61K 9/00; A61P 7/04; C07K 14/755; A61K 38/38

(52) U.S. Cl. CPC A61K 38/37; A61K 9/0019; A61P 7/04; C07K 14/755; A61K 38/38

(57) ABSTRACT

The present invention relates to the treatment of hemophilia A, in particular to means and methods for subcutaneous administration of Factor VIII (FVIII) proteins. More specifically, the invention relates to FVIII proteins comprising at least one albumin binding domain, which could be shown to have a high bioavailability after subcutaneous administration, in particular, for use in subcutaneous administration to a subject with hemophilia A. The invention also relates to the use of further agents enhancing the bioavailability of FVIII proteins comprising at least one albumin binding domain after subcutaneous administration of such FVIII proteins, in articular human albumin, hyaluronidase and derivatives thereof. The invention also relates to pharmaceutical compositions, combined administration, combined preparations, packages and kits.

The present invention relates to the treatment of hemophilia A, in particular to means and methods for subcutaneous administration of Factor VIII (FVIII) proteins. More specifically, the invention relates to FVIII proteins comprising at least one albumin binding domain, which could be shown to have a high bioavailability after subcutaneous administration, in particular, for use in subcutaneous administration to a subject with hemophilia A. The invention also relates to the use of further agents enhancing the bioavailability of FVIII proteins comprising at least one albumin binding domain after subcutaneous administration of such FVIII proteins, in particular human albumin, hyaluronidase and derivatives thereof. The invention also relates to pharmaceutical compositions, combined administration, combined preparations, packages and kits.

Hemophilia A mainly is a genetic bleeding disorder linked to the X-chromosome, occurring in 1 of 5000 newborn males. However, hemophilia A can also occur spontaneously due to an auto-immune response against FVIII. As a result, hemophilia patients do not have sufficient levels of endogenous FVIII. Patients with hemophilia A suffer from longer bleeding durations, spontaneous and internal bleedings, affecting their everyday life. Small internal bleedings can lead to damage in joints, resulting in painful and crippling deformations. Hemophilia A patients are generally treated by administration of FVIII. Depending on the severity of the disease (mild, moderate or severe), treatment is on demand or prophylactic. Therapeutic FVIII products are either purified from human plasma (pFVIII) or the products are produced recombinantly in cell culture (rFVIII).

FVIII is an important co-factor in the coagulation cascade. Wildtype (wt) human FVIII is synthesized as a single chain consisting of 2351 amino acids and comprises three A domains (A1-A3), one B domain and two C domains (C1 and C2), interrupted by short acidic sequences (a1-a3). The first 19 amino acids are the signal sequence, which is cleaved by intracellular proteases during secretion, leading to a FVIII molecule of 2332 amino acids. The resulting domain structure is A1-a1-A2-a2-B-a3-A3-C1-C2. During post-translational modification, FVIII becomes glycosylated, sulfated and proteolytically processed. Sulfation is important for the extracellular interaction with different proteins, especially thrombin and von Willebrand factor (vWF). It takes place on six tyrosines in the acidic regions a1, a2 and a3. Intracellular cleavage, by the serine protease furin, divides FVIII into a heavy chain (A1-a1-A2-a2-B) and a light chain (a3-A3-C1-C2). During this cleavage, parts of the B domain can be lost. Therefore, the light chain has a molecular weight of 80 kDa, whereas the heavy chain can be slightly heterogeneous, with a molecular weight around 210 kDa. The binding between heavy and light chain is not covalent, but mediated by the divalent metal ion Cu²⁺ between the A1 and A3 domain.

In the circulation, FVIII is bound to vWF via the a3, C1 and C2 domain, which protects FVIII from early activation as well as degradation.

Upon activation, FVIII is cleaved by thrombin at three positions, leading to a heterotrimer and loss of the B domain (heterotrimeric FVIIIa). The heterotrimer forms a complex with the activated coagulation Factor IXa and coagulation Factor X, and the light chain binds to a phospholipid bilayer, e.g., the cell membrane of (activated) platelets.

During the development of recombinant FVIII molecules for therapy, B-domain deleted FVIII molecules have been designed, because the B-domain is not important for the functionality of FVIII in clotting. This predominantly leads to a reduction in size, which has advantages in recombinant production. One of the most common B-domain deleted FVIII product is ReFacto® or ReFacto AF® (Moroctocog alfa) produced by Pfizer. This FVIII variant lacks 894 amino acids of the B domain.

Wildtype FVIIII and commercially available FVIII proteins suitable for human administration have a short half-life in the human circulation. Thus, in order to reach suitable substitution levels, FVIII needs to be administered daily or every second day.

Further complicating treatment is the fact that the wt FVIII protein is a very large molecule. As such, commercially available FVIII proteins typically need to be administered intravenously. In comparison to subcutaneous administration, intravenous administration is very cumbersome. First, a suitable vein must be found. This may be practicable in a hospital setting in treatment of otherwise healthy adult patients, but it is a significant burden when veins are small or difficult to be found, such as in case of infants or patients with certain co-morbidities. In the long term, multiple intravenous administrations can result in lesions of the respective veins, making administration even more difficult. Patients need to be trained in self-administration at a juvenile age. In addition, due to the direct access to the blood stream, intravenous administration bears a much higher risk of causing a severe systemic infection than the more convenient subcutaneous administration. In summary, despite its advantages, current FVIII treatment means a high burden for patients and their families.

Additionally, up to 30% of patients with severe Hemophilia A develop inhibitory anti-FVIII antibodies against therapeutic FVIII during intravenous substitution therapy, rendering the drug inactive. This is due to the fact that the immune system of these patients recognizes the applied therapeutic FVIII as foreign, because the patients produce an altered endogenous FVIII variant, which can be mutated or truncated, or no FVIII at all. In general, there is an increased risk of anti-drug antibody (ADA) development after subcutaneous administration of a drug compared to its intravenous administration.

Alternative routes of administration and especially a subcutaneous administration route, if possible, combined with an extended half-life, would be a considerable relief for the concerned patients and their families.

To increase the ease of administration of FVIII for the patient, and, thus, to increase the quality of life, there have been numerous tries to provide variants of FVIII protein that can be administered subcutaneously.

For example, WO 2011/020866 A2 of CSL Behring relates to albumin-fused coagulation factors for non-intravenous administration. WO 95/01804 A1 describes a pharmaceutical formulation described as suitable for subcutaneous, intramuscular or intradermal administration, comprising highly purified recombinant coagulation factor VIII in a concentration of at least 1000 IU/ml. WO 95/26750 A1 describes a pharmaceutical composition for subcutaneous, intramuscular or intradermal administration comprising at least 500 IU/ml of coagulation factor VIII, or an active derivative thereof and an organic additive providing a plasma level of factor VIII which is at least 1.5% of the normal plasma level in the blood for at least 6 hours after administration. The organic additive is selected from amino acids, peptides, proteins, polysaccharides, emulsions, dispersions of polar lipids, and combinations thereof.

WO 2013/156488 A2 describes a subcutaneous dosage form of a pharmaceutical composition comprising therapeutic agents such as factor VIII directly or indirectly conjugated to a polyethylene glycol molecule.

Rode et al. (2018. J Thromb Haemost—16(6):1141-1152) describes preclinical pharmacokinetics and biodistribution of subcutaneously administered glycoPEGylated recombinant factor VIII (N8-GP). A phase I clinical trial sponsored by Novo Nordisk is based on this factor VIII molecule. Results were published by Klamroth et al. (J Thromb Haemost. 2020; 18: 341— 351.), demonstrating that daily prophylaxis with s.c. N8-GP appeared well tolerated and efficacious, achieving a mean trough FVIII activity close to 10% at steady state. However, subcutaneous administration of N8-GP is associated with a high incidence of antibodies in previously treated patients (PTPs) with severe hemophilia A. Further clinical development of s.c. N8-GP has thus been suspended.

Octapharma provided a combination of human cell line-derived rFVIII and a dimerized recombinant human von Willebrand Factor fragment dimer (OCTA12), together designated OCTA101, that shows increased bioavailability in animal models and is undergoing clinical review for subcutaneous administration in a phase I/II study (ClinicalTrials.gov Identifier: NCT04046848). However, no clinical results have yet been disclosed.

In summary, despite decades of research, any attempts of subcutaneous administration of FVIII have failed to reach a sufficient level of bioavailability, resulting in intravenous administration of FVIII still being the standard of care.

In light of this lack of progress, other means of treatment have been explored. E.g. bifunctional antibodies have been developed, which can be subcutaneously administered, e.g., concizumab or emicizumab (Hemlibra®), or fitusiran, a siRNA for gene silencing of antithrombin, for prophylaxis and treatment in haemophilia patients, in particular, patients who have developed inhibitory antibodies (inhibitors) to FVIII (Shapiro et al., 2019. Blood 134(22):1973-1982; Spadarella et al., Blood Rev. 2019 Oct. 15:100618).

However, such “non-factor” therapies have significant disadvantages. In particular, they are usually not able to provide more than a baseline level of coagulation activity, which is insufficient for many patients and may not allow to lead a fully active life.

Also, gene therapy approaches focusing on replacement of FVIIII are making progress, but present attempts still suffer from hardly predictable levels and subsequently declining levels of FVIII activity. Furthermore, long-term effects are not yet explored.

Thus, there is a strong need in the art to provide improved means and methods for treatment of hemophilia A, In particular, it would be highly desirable to make efficient subcutaneous administration of FVIII molecules possible, resulting in high bioavailability of the compound. Preferably, such means and methods would also lead to an increased half-life to reduce the frequency of administration and/or to increase the trough levels of FVIII activity.

This problem was addressed by the inventors and it is solved by the present invention, e.g., by the claimed subject matter.

### DESCRIPTION OF THE INVENTION

The present invention provides Factor VIII proteins comprising at least one albumin binding domain, which show a high bioavailability after subcutaneous administration. When testing subcutaneous administration of diverse recombinant FVIII proteins in the presence of certain compounds which were hoped to enhance bioavailability, the inventors observed unexpectedly that FVIII proteins comprising one or more albumin-binding domains (FVIII-ABD) showed a particularly enhanced bioavailability. More surprisingly, an enhanced bioavailability could be observed even in absence of bioavailability enhancing compounds. Even more advantageously, certain FVIII proteins comprising one or more albumin binding domains have an increased plasma half-life. Thus, the advantage of an increased bioavailability can be combined with an increased half-life, thus making more convenient and less frequent administration possible. The bioavailability can be further enhanced by adding albumin or hyaluronidase or derivatives thereof, either by co-administration, or by combined preparations.

Such advantages may furthermore come along with a reduced immunogenicity, as albumin bound to FVIII might have recruit regulatory T-cells and exert a shielding effect with respect to the bound FVIII.

Without intending to be bound by any theory, the inventors assume that albumin binding to the FVIII-ABD is particularly effective in inhibiting breakdown of the FVIII in the subcutaneous tissue. Thus, introducing one or more albumin binding domains would not only increase the half-life of FVIII in the blood stream, but also in the subcutaneous tissue. Alternatively or in addition, the binding to albumin might enhance the transport of FVIII-ABD to the blood vessel and thus into the circulation.

The term subcutaneous administration is understood by the skilled person. Generally, subcutaneous administration refers to administration of a drug (in the present case FVIII-ABD). Typically, subcutaneous administration is achieved by subcutaneous injection. A subcutaneous injection is administered as a bolus into the subcutis, the layer of skin directly below the dermis and epidermis, collectively referred to as the cutis. The volume injected may be, e.g., in the range of 0.1-50 mL, such as, 0.2-10 mL.

As mentioned, the inventors have found a very high bioavailability for FVIII-ABD. Bioavailability designates the fraction of an administered dose of unchanged drug that reaches the systemic circulation, one of the principal pharmacokinetic properties of drugs. By definition, when a medication is administered intravenously, its bioavailability is 100%. However, when a medication is administered via other routes (such as subcutaneously), its bioavailability generally decreases. The term bioavailability relates to absolute bioavailability, which is preferably calculated as shown in the examples described herein. In brief, the absolute bioavailability is the dose-corrected area under curve (AUC divided by dose) non-intravenous divided by AUC intravenous resulting from bolus injections, wherein the period from predose (which is at maximum 2 h before injection) over the maximum observed blood concentration possible until the lower limit of quantification (LLOQ) is reached, but at least until a concentration of 0.01 U/mL, is used for the calculation.

In first studies, Factor VIII proteins comprising at least one albumin binding domain were tested in hemophilia A-mice and in minipigs in comparison to ReFacto® AF (Pfizer), which is one of the most common B-domain deleted FVIII products. A high bioavailability of at least about 15% in mice and about 30-60% in Göttingen minipigs (minipigs) was found for FVIII-ABD. Minipigs are the best available model for subcutaneous administration in human subjects, and thus allow for prediction of a similar bioavailability in humans.

The invention thus provides a recombinant Factor VIII protein comprising at least one albumin binding domain (FVIII-ABD), wherein the bioavailability of the Factor VIII-ABD protein after subcutaneous administration is at least 25% as measured in minipigs, in particular, for use in treatment of hemophilia A. The invention also provides a pharmaceutical composition comprising said FVIII-ABD. More particularly, the bioavailability of the Factor VIII-ABD protein after subcutaneous administration is at least 30% as measured in minipigs. More particularly, the bioavailability of the Factor VIII protein after subcutaneous administration is at least 35%, more particularly 40% as measured in minipigs. For example, the bioavailability of the Factor VIII-ABD protein after subcutaneous administration can be in the range of 25-80%, e.g., 30-60% as measured in minipigs.

Bioavailability can be measured in minipigs, e.g., following the protocol laid out below:

To measure bioavailability in a minipig after subcutaneous (s.c.) administration, naïve Göttingen minipigs (Ellegaard, Dalmos, DK) are used. The bioavailability is measured in a group of minipigs, e.g., at least 3 minipigs per group or, preferably, at least 10 minipigs per group. 1 h prior to FVIII administration, all animals are dosed via i.v. injection (saphenous vein) with 1.25 mL Albiomin® 20% (a 20% HSA solution, Biotest AG, Dreieich)/kg bodyweight. FVIII compositions (300 U FVIII:Ag/kg bodyweight) in the desired formulation of the invention are administered by a single s.c. bolus injection of a Göttingen minipig (e.g., at least 3 per group, preferably, at least 10 per group) behind the ear, or for comparison, via intravenous bolus injection (i.v.). Blood samples are collected from the vena cava into vacuum blood collection tubes containing sodium citrate at the time points: pre-dose, 0.5, 4, 12, 24, 36, 48, 72, 96, 120, 144, 192 and 240 h post dose.

As the minipigs are not hemophilia minipigs, they have endogenous FVIII activity. Thus, as explained in more detail in the Examples section below, FVIII concentration in the plasma is measured by an FVIII antigen ELISA specific for human FVIII adapted for use based on minipig plasma (essentially, performing all predilutions in minipig plasma, including the dilutions of the calibrator, controls and samples; a last 1:2 dilution step has to be done using the kit-supplied phosphate buffer e.g., as described in more detail in the method section). As albumin-binding to FVIII proteins as used in the invention has an influence on antibody-binding, this is taken into account by a correction factor (e.g., determined by spiking the application solution into minipig plasma and evaluating the decrease in FVIII:Ag detection). Bioavailability is calculated according to the formula:

$$F [ \% ] = \frac{{AUC}_{0 - inf , sc}}{{AUC}_{0 - inf , iv}} * 100$$

Where

AUC_{0-inf} is the AUC from dosing time extrapolated to infinity, based on the last observed concentration (Cₗₐₛₜ), i.e., the elimination rate constant λ_{z} is used to estimate the AUC_{t-inf} (Cₗₐₛₜ/λ_{z}) from the last observed concentration until the time point of concentration zero is reached, which is added to the AUC₀₋ₜ, calculated for the period from predose, which is at maximum 2 h before injection, over the maximum observed blood concentration possible until the lower limit of quantification (LLOQ), but at least until a concentration of 0.01 U/mL is reached:

$${AUC}_{0 - inf} = {AUC}_{0 - t} + \frac{{C}_{last}}{{λ}_{z}}$$

The invention also provides a Factor VIII protein comprising at least one albumin binding domain, e.g., as described above, for use in treatment of hemophilia A, wherein a dose of 1-1000 U/kg bodyweight is administered to a subject having hemophilia A subcutaneously, optionally, 5-1000 U/kg bodyweight. U are equivalent to international units, and all measurements were based on an international WHO standard. Bodyweight relates to the bodyweight of the subject to which the FVIII protein is to be administered. For example, the dose may be 10-900 U/kg bodyweight. Optionally, the dose is 10-700 U/kg bodyweight. It may also be 50-500 U/kg bodyweight.

The invention further provides a recombinant Factor VIII protein comprising at least one albumin binding domain, wherein the bioavailability of the Factor VIII protein after subcutaneous administration is at least 25% as measured in minipigs, for use in treatment of a subject having hemophilia A, wherein a dose of 1-1000 U/kg bodyweight is administered to the subject subcutaneously, optionally, 5-1000 U/kg bodyweight. For example, the dose may be 10-900 U/kg bodyweight. Optionally, the dose is 10-700 U/kg bodyweight. It may also be 50-500 U/kg bodyweight.

The subcutaneous dosing of the FVIIII-ABD depends on several factors and can be adapted by the skilled person according to the need of the patient. The first main factor of course is the bioavailability of the factor VIII protein after subcutaneous administration. While the subject may also be a mammal such as a dog, minipig, or mouse subject, preferably, throughout the invention, the subject is a human subject. Based on the very similar minipig model, the bioavailability of the Factor VIII protein used in the pharmaceutical composition of the invention, after subcutaneous administration in human subjects may be at least 15%, preferably, at least 20%, optionally, at least 25%. It is advantageous if the bioavailability of the Factor VIII protein after subcutaneous administration in human subjects is 30-80%, e.g., 30-60%. For example, the bioavailability of the Factor VIII protein after subcutaneous administration in a human subject may be at least 40%, or 40-50%. Bioavailability in a human may be determined in a controlled trial comparing intravenous and subcutaneous administration of a pharmaceutical composition of the invention in humans.

The bioavailability may be determined for individual human subjects receiving FVIII via intravenous injection and subcutaneous injection separately for a defined time frame, but it may also be determined over groups of human subjects receiving FVIII either via intravenous injection or subcutaneous injection. Such group comparison may include groups from one clinical trial, which is preferred, or various trials. Groups may comprise at least 5, preferably, at least 10 subjects each.

The second main factor is the need of the subject (or patient), which varies depending on the weight, FVIII status, severity of disease etc. For example, a patient may only have the need to substitute a small amount of FVIII to reach satisfactory levels, or the patient may be absolutely deficient in FVIII. The patient may also have an acute bleeding event, necessi-tating administration of a larger amount of FVIII than typically needed. Typically, the aim of therapy is to achieve a norm level of FVIII in the blood plasma of 0.3-1.5 U/mL. Maximum levels that are to be achieved may be about 1.5 U/mL, and minimum levels may be about 0.05 U/mL. Depending on the route of administration, FVIII peak levels can be achieved for example within 30 seconds or up to 12 hours after administration. Generally, the aim of the therapy is to maintain a FVIII activity level in blood plasma greater than 0.1 U/ml over time.

Dosages and treatment schemes may be chosen as appropriate, e.g., for prophylaxis of bleeding or with intermittent, on-demand therapy for bleeding events.

For example, the FVIII of the invention may be administered subcutaneously in dosages of 5 to 750 U/kg body weight every 0.5 to 14 or every 6-7 days depending on the severity of the disease, typically, 5 to 500 U/kg body weight.

The medical practitioner may test bioavailability after subcutaneous administration in a specific patient, and adapt the dose after testing to achieve the desired levels of FVIII in the blood. However, the knowledge of an expected bioavailability is essential to avoid gross misdosing, which can lead either to an increased risk of bleeding events due to non-sufficient levels or to thrombosis, if too much FVIII is administered.

The bioavailability of the Factor VIII protein employed in the pharmaceutical compositions after subcutaneous administration in a mouse may be at least 10%, preferably, at least 10-60%. For example, the bioavailability of the Factor VIII protein after subcutaneous administration in a mouse may be 10-30%, e.g., 15-20%

Factors relevant for bioavailability of a protein comprise the distribution of the protein to different compartments after administration and the in vivo half-life.

Preferably, FVIII-ABDs having a longer in vivo half-life than standard FVIII proteins such as ReFacto AF® and excellent specific activity as evidenced by different biological activity assays are employed in the invention. These proteins further have a high level of expression and a low profile of fragments and side products. Further advantages and preferred embodiments are explained elsewhere in this description.

Under physiological conditions wt FVIII proteins are associated with von Willebrand factor (vWF) in blood. FVIII not associated with vWF is typically degraded much more rapidly. wt Factor VIII in complex with vWF has an in vivo half-life of about 12 hours.

Albumin has an in vivo half-life of about 19 days. By introducing at least two albumin binding domains in the FVIII sequence, it was possible to obtain a significant half-life prolongation. Different positions and different numbers of the albumin binding moiety have been tested in order to identify the optimal positions and numbers of integrated albumin binding moieties.

The inventors have found that a particular arrangement of ABDs particularly contributes to an increase in in vivo plasma half-life: recombinant Factor VIII protein comprising a heavy chain portion and a light chain portion of Factor VIII and at least two albumin binding domains, wherein at least one albumin binding domain is C-terminal to the heavy chain portion and at least one albumin binding domain is C-terminal to the light chain portion. If the protein is a single chain protein, the albumin binding domain(s) C-terminal to the heavy chain portion (and not C-terminal to the light chain portion) is/are N-terminal to the light chain portion. In other words, if the protein is a single chain portion, in preferred embodiments at least one albumin binding domain may be positioned between the heavy chain portion and the light chain portion, and at least one albumin binding domain C-terminal to the light chain portion.

It is however noted that, while variants of FVIII comprising at least two albumin binding domains, as described, are preferred, incorporation of at least one albumin binding domain already enhances bioavailability after subcutaneous administration.

Without intending to be bound by the theory, it is believed that albumin binding to the FVIII protein of the invention through the albumin binding domains in the specific positions described herein is particularly effective in inhibiting breakdown of the FVIII protein of the invention. This appears to increase in vivo half-life more than association with vWF associated with native FVIII in blood. Advantageously, the FVIII proteins of the invention also have a high stability in human tissue, in particular, in human skin, which also contributes to the high bioavailability seen.

### FVIII

The skilled person understands the term FVIII (or Factor VIII) and is aware of the structure and biological functions of wild type FVIII and typical variants thereof. As noted, the FVIII protein used for subcutaneous administration should comprise at least one albumin binding domain, in order to achieve a high bioavailability. Preferred examples of suitable FVIII or FVIII-ABD proteins are further disclosed herein. Apart from this, the FVIII protein used in the context of the invention may be designed as deemed appropriate and advantageous by the skilled person.

Factor VIII proteins of the invention will typically be recombinant Factor VIII proteins, i.e., proteins produced by genetically engineered cells. They may also be synthesized by chemical synthesis.

The FVIII protein employed in the invention may be produced by a host cell that preferably is a mammalian, more preferably a human cell comprising an expression vector suitable for expression of said recombinant Factor VIII protein in said human cell. The cell may be transiently or stably transfected with the nucleic acid of the invention. Said stably transfected cells may have integrated the nucleic acid expressing the FVIII proteins of the invention, but may not further comprise an extragenomic expression vector. The cell may be a cell line, a primary cell or a stem cell. For production of the protein, the cell typically is a cell line such as a HEK cell, such as a HEK-293 cell, a CHO cell, a BHK cell, a human embryonic retinal cell such as Crucell's Per.C6 or a human amniocyte cell such as CAP. For treatment of human patients with the protein, the host cell preferably is a human cell, e.g., a HEK293 cell line or a CAP cell line (e.g. a CAP-T cell or a CAP-Go cell). The inventors have found that in a CAP cell line, a particularly high single chain content of FVIII-ABD protein can be produced. Among the CAP cells, CAP-T cells are preferred for transient expression, while CAP-Go cells may be used for creation of stable cell lines conveying an advantageous glycosylation profile to the FVIII-ABD molecule.

In particular, the Factor VIII-ABD protein of the invention should typically comprise all necessary portions and domains known to be important for biological function. For example, preferably, the FVIII protein should comprise domains corresponding to, substantially corresponding to, and/or functionally corresponding to the A and C domains of wild type FVIII, especially to A1, A2, A3, C1 and C2 domains. It may further comprise additional portions and domains. For example, preferably, the FVIII protein further comprises an a1 domain between the A1 and the A2 domains and an a2 domain C-terminal to the A2 domain. For a double chain protein, on a separate chain, or for a single chain protein, C-terminal to said domain, and, optionally, C-terminal to the B-domain or a truncated B-domain and to at least one albumin binding domain, the FVIII protein comprises at least a truncated a3 domain. Before secretion, the Factor VIII protein of the invention may also comprise a signal sequence.

Thus, the heavy chain portion preferably comprises at least the domains A1 and A2, and typically comprises the domains A1-a1-A2-a2 or A1-a1-A2-a2-B. Preferably, the B-domain of the Factor VIII protein is at least partly deleted. Deletion of the B-domain facilitates recombinant manufacture of the FVIII protein. The light chain portion preferably comprises the domains A3 and C1 and C2, and typically comprises the domains a3-A3-C1-C2, wherein the a3 domain may be truncated. Any or all of said domains may be wildtype (wt) FVIII domains, or they may differ from the wildtype domains, e.g., as further disclosed in the present application, as known in the state of the art or deemed appropriate by the skilled person.

The domains are preferably contained in the protein in the order outlined above, i.e., from N-terminus to C-terminus of the protein.

While parts of the FVIII-ABD protein of the invention can be designed as desired by the skilled person, the FVIII-ABD preferably maintains a high FVIII biological activity. As shown in the examples, the invention allows for generation of a FVIII-ABD protein with a high biological activity, as measured e.g. by the chromogenic activity. Therefore, preferably the FVIII-ABD protein according to the invention has a chromogenic activity which is at least comparable to the activity of the wt FVIII protein, i.e., it has at least 50% of the specific chromogenic activity of the wt protein (SEQ ID NO: 1). Preferably, the FVIII-ABD protein according to the invention has at least 80%, at least 100% or more than 100% of the specific chromogenic activity of the wt protein. Preferably, the chromogenic activity also is at least 50%, at least 80%, at least 90%, at least 100% or more than 100% of the chromogenic activity of ReFacto AF® (international non-proprietary name: Moroctocog Alfa), a commercially available B-domain deleted FVIII (Pfizer). More preferably, the FVIII-ABD protein has 80% to 120% of the chromogenic activity of ReFacto AF®.

A FVIII-ABD protein according to the present invention shall have at least one biological activity or function of a wt FVIII protein, in particular the function in coagulation. The FVIII protein should be cleavable by thrombin, leading to activation. Preferably, the FVIII protein according to the invention comprises at least one thrombin recognition and/or thrombin cleavage site, wherein said thrombin recognition and/or thrombin cleavage sites may correspond to or substantially correspond to those of wild type FVIII. It is then capable of forming a complex with the activated coagulation Factor IXa and coagulation Factor X, and the light chain is capable of binding to a phospholipid bilayer, e.g., the cell membrane of (activated) platelets.

The biological activity of FVIII can be determined by analyzing the activity of the protein in a one-stage clotting assay (clotting or coagulant activity) or a two-stage chromogenic assay (chromogenic activity), as described herein. Typically, the chromogenic activity is taken as a measure of biological activity.

It is known in the art that the B-domain is not required for proper coagulant function of FVIII, and therefore, various B-domain deleted FVIII proteins are well known. In the context of the present invention, a B-domain deleted FVIII protein may comprise full or partial deletion(s) of the B-domain. The B-domain deleted FVIII protein may still contain amino-terminal sequences of the B-domain which may e.g. be important for proteolytic processing of the translation product. Moreover, the B-domain deleted FVIII protein may contain one or more fragments of the B-domain in order to retain one or more N-linked glycosylation sites. Optionally, the FVIII protein does not contain any furin cleavage sites, resulting in a single chain protein in which light and heavy chains of the protein are covalently linked.

For example, the B-domain deleted FVIII-ABD protein may still comprise 0-200 residues, e.g., 1-100 residues, preferably 8 to 90 residues of the B-domain. The remaining residues of the B-domain may derive from the N-terminus and/or the C-terminus and/or from internal regions of the B-domain. For example, the remaining residues from the C-terminus of the B-domain may contain 1-100, preferably 20-90, more preferably 86 residues. In other embodiments the remaining residues from the C-terminus may contain 1-20 residues, e.g. 4 residues. For example, the remaining residues from the N-terminus of the B-domain may contain 1-100, preferably 2-20 residues, more preferably 2-10 residues, more preferably 4 residues. For example, the remaining residues from internal regions of the B-domain may contain 2-20, preferably 2-10, more preferably 4 to 8 residues. In a preferred embodiment, the FVIII protein comprises 86 C-terminal residues of the B-domain and 4 residues from the N-terminus of the B-domain, e.g., as in FVIII-19M.

Double chain proteins which may form a basis for FVIII proteins of the invention are known in the art, e.g., wt FVIII or B-domain deleted or truncated versions thereof, e.g., ReFacto AF®.

Moreover, the inventors have found that single chain proteins may be used advantageously. In particular, production of FVIII as a single chain may be beneficial for purification. To simplify purification, the FVIII protein of the invention may be a single chain protein or at least have a proportion of at least 40%, at least 50%, at least 60%, at least 70%, at least 80%, at least 90% or at least 95% single chain protein. Preferably, the FVIII protein used in the invention is a single chain B-domain deleted Factor VIII protein.

Single chain Factor VIII proteins are known in the art. In general, single chain FVIII proteins do not comprise a functional furin cleavage site and thus, before activation, remain in the circulation as a single chain. In exemplary single chain FVIII proteins known in the art, e.g., at least part of the B-domain and 4 amino acids of the adjacent acidic a3 domain (e.g., residues 784-1671 of full length FVIII) are removed, in particular, removing the furin cleavage-site (EMA/CHMP/699390/2016—Assessment report AFSTYLA). Such proteins are also disclosed in EP application No. 19173440 or taught herein.

For example, a single chain FVIII molecule may be used, in which several amino acids including the furin cleavage site (positions R1664-R1667, wherein the signal peptide is also counted) have been deleted. The B domain is deleted to a large extent, wherein an internal fragment (at least NPP) of the B-domain is maintained and an intact thrombin cleavage site is preceding the internal fragment.

The inventors could show that stability of such single chain FVIII proteins (not comprising albumin-binding domains) is at least comparable to stability of ReFacto AF® in a suitable pharmaceutical composition. Thus Factor VIII protein of the invention may comprise, in a single chain, a heavy chain portion comprising an A1 and an A2 domain and a light chain portion comprising an A3, C1 and C2 domain of Factor VIII, wherein

a) in said recombinant Factor VIII protein, 894 amino acids corresponding to consecutive amino acids between F761 and P1659 of wild type Factor VIII as defined in SEQ ID NO: 1 are deleted, leading to a first deletion;

b) said recombinant Factor VIII protein comprises, spanning the site of the first deletion, a processing sequence comprising SEQ ID NO: 2 or a sequence having at most one amino acid substitution in SEQ ID NO: 2, wherein said processing sequence comprises a first thrombin cleavage site;

c) in said recombinant Factor VIII protein, at least the amino acids corresponding to amino acids R1664 to R1667 of wild type Factor VIII are deleted, leading to a second deletion; and

d) said recombinant Factor VIII protein comprises, C-terminal to the second deletion and N-terminal of the A3 domain, a second thrombin cleavage site.

As defined in a), in the FVIII of the invention, 894 amino acids corresponding to consecutive amino acids between F761 and P1659 of wild type Factor VIII as defined in SEQ ID NO: 1 are deleted in the Factor VIII protein of the invention, leading to a first deletion. In certain embodiments, in particular, starting from a numbering of amino acids in FVIII without dele-tions or insertions, the term “corresponding to” should be understood to mean “identical to”.

For specific amino acids which may be mutated compared to the wt, an amino acid corresponding to the wild type aa is determined by an alignment e.g. using EMBOSS Needle (based on the Needleman-Wunsch algorithm; settings: MATRIX: “BLOSUM62”, GAP OPEN: “20”, GAP EXTEND:“0.5”, END GAP PENALTY: “false”, END GAP OPEN: “10”, END GAP EXTEND: “0.5”).

In order to assess sequence identities of two polypeptides, such an alignment may be performed in a two-step approach: I. A global protein alignment is performed using EMBOSS Needle (settings: MATRIX: “BLOSUM62”, GAP OPEN: “20”, GAP EXTEND:“0.5”, END GAP PENALTY: “false”, END GAP OPEN: “10”, END GAP EXTEND: “0.5”) to identify a particular region having the highest similarity. II. The exact sequence identity is defined by a second alignment using EMBOSS Needle (settings: MATRIX: “BLOSUM62”, GAP OPEN: “20”, GAP EXTEND:“0.5”, END GAP PENALTY: “false”, END GAP OPEN: “10”, END GAP EXTEND: “0.5”) comparing the fully overlapping polypeptide sequences identified in (I) while excluding non-paired amino acids.

“between” excludes the recited amino acids, e.g., it means that the recited amino acids are maintained. “deletion” or “deleted” does not necessitate that the protein was actually prepared by deleting amino acids previously present in a predecessor molecule, but it merely defines that the amino acids are absent, independent from the preparation of the molecule. For example, the protein can be produced based on nucleic acids prepared by de novo synthesis or by genetic engineering techniques.

As defined in b), the

recombinant Factor VIII protein may comprise, spanning the site of the first deletion, a processing sequence comprising SEQ ID NO: 2 (PRSFSQNPP) or a sequence having at most one amino acid substitution in SEQ ID NO: 2, wherein said processing sequence comprises a first thrombin cleavage site. Accordingly, at least one amino acid of the processing sequence corresponds to an amino acid C-terminal to the deletion and at least one amino acid of the processing sequence corresponds to an amino acid N-terminal to the deletion. The processing sequence comprises SEQ ID NO: 2 or a sequence having at most one amino acid substitution in SEQ ID NO: 2, i.e., the processing sequence can be longer. In particular, the processing sequence is selected from the group comprising SEQ ID NO: 2, 4, 5, 6, 7 or 8. The inventors have found that a processing sequence of the invention enables a particularly good cleavage by thrombin.

In certain embodiments, the processing sequence is no longer than SEQ ID NO: 4. The processing sequence may be directly C-terminal to sequences from the a2 domain, e.g., wt a2 domain sequences. The first N-terminal two amino acids of the processing sequence may already belong to the a2 domain. Preferably, the amino acid directly N-terminal to the processing sequence is E.

One amino acid in SEQ ID NO: 2 can be substituted, e.g., to reduce immunogenicity. Optionally, the F, the S C-terminal to the F, the Q or the N are substituted.

The processing sequence may be SEQ ID NO: 4 (PRSFSQNPPVL) or a sequence having at most one amino acid substitution in said sequence, wherein, optionally, the F, the S C-terminal to the F, the Q or the N are substituted. Moreover, the present inventors have shown that an L at the C-terminus of the processing sequence, as in SEQ ID NO: 4, 5, 6, 7 or 8, endows the FVIII with particularly good activity. One especially preferred example of a single chain FVIII protein, which may form the backbone for the protein of the invention, is shown in the examples in further detail under the name V0 (SEQ ID NO: 16). The processing sequence of the FVIII protein V0, which has been found to be particularly advantageous, consists of SEQ ID NO: 4, which is a specific embodiment of SEQ ID NO: 5-8.

The alternative processing sequences SEQ ID NO: 5 (PRSXSQNPPVL), SEQ ID NO: 6 (PRSFXQNPPVL), SEQ ID NO: 7 (PRSFSXNPPVL) and SEQ ID NO: 8 (PRSFSQXPPVL) are variants in which X can be any naturally occurring amino acid. Optionally, X is a conser-vative substitution compared to the corresponding amino acid in SEQ ID NO: 4, i.e. a hydrophobic amino acid is substituted by a hydrophobic amino acid, a hydrophilic amino acid is substituted by a hydrophilic amino acid, an aromatic amino acid by an aromatic amino acid, an acid amino acid by an acid amino acid and a basic amino acid by a basic amino acid.

As defined in c), in the FVIII protein of the invention, the amino acids corresponding to amino acids R1664 to R1667 of wild type Factor VIII are deleted, leading to a second deletion. These amino acid correspond to the furin cleavage recognition site of wt FVIII. Accordingly, the protein is essentially not cleaved by furin. In a composition, at least 80%, optionally, at least 90% or at least 95% of the FVIII protein of the invention are present in a single chain form.

As defined in d), the recombinant Factor VIII protein of the invention comprises, C-terminal to the second deletion and N-terminal of the A3 domain, a second thrombin cleavage site. Accordingly, upon activation, the part of the FVIII protein between the thrombin cleavage site in the processing sequence and the second thrombin cleavage site is excised from the activated FVIII protein.

Further, the invention provides a recombinant Factor VIII-ABD protein comprising, in a single chain, a heavy chain portion comprising an A1 and an A2 domain and a light chain portion comprising an A3, C1 and C2 domain of Factor VIII, wherein,

a) said recombinant Factor VIII protein comprises a processing sequence comprising SEQ ID NO: 2 or a sequence having at most one amino acid substitution in SEQ ID NO: 2, wherein said processing sequence comprises a first thrombin cleavage site;

b) directly C-terminal to said processing sequence, said Factor VIII protein comprises a heterologous sequence comprising at least one, preferably, two albumin binding domain(s);

c) directly C-terminal to said heterologous sequence, said Factor VIII protein comprises a merging sequence having at least 90% sequence identity to SEQ ID NO: 9 (e.g., SEQ ID NO: 9); and

d) said recombinant Factor VIII protein comprises, C-terminal to SEQ ID NO: 9, a second thrombin cleavage site; and

e) said recombinant Factor VIII protein comprises, C-terminal to the light chain portion, at least one, preferably, two albumin binding domain(s).

Said recombinant FVIII protein may be a FVIII protein as described above. The FVIII protein typically comprises at least one further thrombin cleavage site.

In one embodiment, a FVIII-ABD protein of the invention comprises a heavy chain portion having at least 90% sequence identity to aa20-aa1667 of SEQ ID NO: 1, and a light chain portion having at least 90% sequence identity to aa1668-aa2351 of SEQ ID NO: 1. Optionally, the respective sequence identity aa20-aa1667 of SEQ ID NO: 1 and sequence identity to aa1668-aa2351 of SEQ ID NO: 1 are at least 95%. The respective sequence identity to aa20-aa1667 of SEQ ID NO: 1 and sequence identity to aa1668-aa2351 of SEQ ID NO: 1 may be at least 98%. Optionally, the respective sequence identity to said sequences is at least 99%. The invention also provides a FVIII-ABD protein of the invention comprising a heavy chain portion having aa20-aa1667 of SEQ ID NO: 1 and a light chain portion having aa1668-aa2351 of SEQ ID NO: 1.

Several experiments performed by the inventors have been carried out with a single chain FVIII of the invention on the basis of the V0 single chain construct (SEQ ID NO: 16) with at least one albumin binding domain located C-terminal to the heavy chain portion and not C-terminal to the light chain portion (i.e., between the heavy chain portion and the light chain portion), and at least one albumin binding domain C-terminal to the light chain portion), as described herein. Such proteins have shown advantageous characteristics with regard to expression, stability, in vivo half-life and purification. Accordingly, a preferred FVIII protein of the invention comprises a heavy chain portion having at least 90% sequence identity to aa20-aa768 of SEQ ID NO: 16, and a light chain portion having at least 90% sequence identity to aa769-aa1445 of SEQ ID NO: 16. Optionally, the respective sequence identity to aa20-aa768 of SEQ ID NO: 16 and sequence identity to aa769-aa1445 of SEQ ID NO: 16 are at least 95%. The respective sequence identity to aa20-aa768 of SEQ ID NO: 16 and sequence identity to aa769-aa1445 of SEQ ID NO: 16 may be at least 98%. Optionally, the respective sequence identity to said sequences is at least 99%. The invention also provides a FVIII protein of the invention comprising a heavy chain portion having aa20-aa768 of SEQ ID NO: 16 and a light chain portion having aa769-aa1445 of SEQ ID NO: 16. Preferably, said FVIII-ABD is a single chain protein.

Another exemplary single chain FVIII protein is provided as SEQ ID NO: 62 (Afstyla®, CSL Behring, Marburg (Ionoctocog alfa)). An exemplary FVIII-ABD single chain protein of the invention based on SEQ ID NO: 62 which has been de-immunized by incorporation of 19 mutations as described elsewhere, lacks 4 amino acids of the a3 domains of FVIII-19M, i.e., it has 99.72% (at least 99% sequence identity) to SEQ ID NO: 63, wherein only the A1, a1, A2, a2, a3, A3, C1 and C2 domains are considered for calculation of sequence identity. Said protein may be B-domain deleted, and it comprises at least one albumin-binding domain, e.g., as described herein.

wt FVIII typically is bound by vWF. vWF shields FVIII from proteolytic degradation and receptor-mediated clearance, e.g. via low-density lipoprotein (LDL) receptor-related protein (LRP1), LDL-receptor (LDLR) and heparan-sulfate proteoglycans (HSPG), within the liver (Lenting et al., 2007. J Thromb Haematol 5:1353-60). However, it has been shown that the half-live of vWF is approx. 15 h, thereby limiting the FVIII:vWF complex half-life to the vWF-related clearance pathway. The inventors found that vWF binding potency of the FVIII protein of the invention may be diminished compared to wt FVIII or ReFacto AF®, which may be explained by sterical hindrance due to albumin binding. For example, FVIII proteins of the invention may have 0%-90%, 10%-80%, 20-70%, 30-60%, or 40-50% of the binding potency of ReFacto AF® to vWF, which can be determined by an assay as described below. Preferably, in the presence of human serum albumin in physiological concentrations, said binding potency is less than 50% of the binding potency of ReFacto AF® to vWF.

vWF binding is mediated in particular by amino acid positions Y1683 and Y1699. To avoid vWF binding, e.g. amino acids corresponding to Y1683 and/or Y1699 of wt FVIII of SEQ ID NO: 1 may be mutated. For example, the amino acid corresponding to Y1683 and/or Y1699 of wt FVIII of SEQ ID NO: 1 may be mutated to a C or F, e.g., Y1699C or Y1699F. In particular, a mutation of the amino acid corresponding to Y1699 to F and a mutation of the amino acid corresponding to Y1683 to F, both mutations together also designated “b mutation” have been confirmed to further decrease binding of vWF to FVIII proteins of the invention. Beside the “b mutation”, the inventors have additionally tested an “a mutation” comprising the amino acid substitutions Y737F, Y738F, and Y742F of wt FVIII of SEQ ID NO: 1 and a “c mutation” comprising the amino acid substitutions I2117S and R2169H of wt FVIII of SEQ ID NO: 1. Additionally, the inventors have tested combinations of “a mutation” and “b mutation” and further combinations of “a mutation” and “b mutation” and “c mutation”. It was observed, that the “c mutation” negatively influenced the protein expression and functionality. The “b mutation” either alone or in combination with the “a mutation” did not influence the protein expression and functionality, but strongly decreased the binding to vWF. In comparison, the “a mutation” did not decrease the binding to vWF.

Thus, to further decrease vWF binding, the Factor VIII protein of the invention may have a suitable mutation as described herein, e.g., a “b mutation”, i.e., a mutation of the amino acid corresponding to Y1699 to F at position 1699 and a mutation of the amino acid corresponding to Y1683 to F at position 1683 in wt Factor VIII protein of SEQ ID NO: 1. For example, the FVIII protein of the invention may comprise a heavy chain portion and a light chain portion of Factor VIII and at least two albumin binding domains, wherein at least two albumin binding domains (e.g., two) are C-terminal to the heavy chain portion and not C-terminal to the light chain portion, and at least two albumin binding domains (e.g., two) are C-terminal to the light chain portion, wherein the FVIII protein further comprises a b mutation. Such a FVIII protein may further comprise linkers, e.g., a thrombin cleavable linker optionally flanked by a glycine-serine linker, between the albumin binding domains and other parts of the protein and between the albumin binding domains. Alternatively, such a FVIII protein does not comprise linkers. In the context of the invention, “flanked” means that the relevant portions are in a close vicinity, preferably, with a distance of at most 10, 5 or 2 amino acids positions. Optionally, the relevant portions are immediately adjacent.

Albumin Binding Domains

The Factor VIII protein of the invention comprises at least one albumin binding domain.

Albumin-binding domains (ABDs) are generally known to the skilled person, and different ABDs may be employed in the context of the invention. As used herein, an albumin binding domain is capable of binding, preferably specifically binding, human serum albumin under physiological conditions. The ABD may have, e.g., an affinity of at most 10⁻⁷M, preferably at most 10⁻⁸ M, at most 10⁻⁹ M, 10⁻¹⁰ M, 10⁻¹¹ M or 10⁻¹² M to human serum albumin. Preferred ABDs are peptides or polypeptides, which can be easily incorporated into a FVIII protein, e.g. by recombinant methods. Preferably, the FVIII-ABD proteins of the invention are fusion proteins of FVIII and at least one ABD.

Suitable examples for such ABDs are known. Historically, first ABDs identified were small, three-helical protein domains derived from one of various surface proteins expressed by gram-positive bacteria. For example, domains derived from streptococcal protein G and protein PAB from Finegoldia magna, which share a common origin and therefore represent an interesting evolutionary system, have been thoroughly studied structurally and functionally. Their albumin-binding sites have been mapped and these domains form the basis for a wide range of protein engineering approaches. By substitution-mutagenesis they have been engineered to achieve a broader specificity, an increased stability or an improved binding affinity, respectively.

For example, albumin binding domains disclosed by Nilvebrant et al. (2013, Comput Struct Biotechnol J. 6: e201303009), Johansson et al. (2001, JBC 277: 8114-8120), Jacobs et al. (2015, Protein Engineering, Design and Selection 28 (10); 385-393), WO 91/01743 A1, WO 2009/016043 A2, WO 2010/054699 A1, WO 2012/004384 A2, WO 2014/048977 A1 or WO 2015/091957 A1 may be used. Alternatively, the albumin-binding domain comprises any of the sequences disclosed as SEQ ID NO: 4-40 of U.S. Pat. No. 10,364,419.

A preferred ABD suitable for use in the present invention comprises a sequence according to SEQ ID NO: 44:

LAX₃AKX₆X₇ANX₁₀ELDX₁₄YGVSDFYKRLIX₂₆KAKTVEGVEALKX₃₉X₄₀
ILX₄₃X₄₄LP

wherein independently of each other

X₃ is selected from E, S, Q and C, preferably, E;

X₆ is selected from E, S, C and V, preferably, E;

X₇ is selected from A, S and L, preferably, A;

X₁₀ is selected from A, S and R, preferably, A;

X₁₄ is selected from A, S, C and K, preferably, S;

X₂₆ is selected from D, E and N, preferably, D;

X₃₉ is selected from D, E and L, preferably, D;

X₄₀ is selected from A, E and H, preferably, A;

X₄₃ is selected from A and K, preferably, A;

X₄₄ is selected from A, S and E, preferably, A;

L in position 45 is present or absent, preferably, present; and

P in position 46 is present or absent, preferably, present.

Alternatively, the albumin binding domain may comprise an amino acid sequence which has at least 95% identity to the sequence of SEQ ID NO: 44.

The inventors have achieved good results using an albumin binding domain designated ABD1 (SEQ ID NO: 45). It is preferred to use the sequence of ABD2 (SEQ ID NO: 46) that has been de-immunized for the human immune system, i.e., adapted to avoid immune responses in humans. If not otherwise mentioned, said albumin binding domain is used in the experiments shown herein. ABD2 may be encoded by a nucleic acid of SEQ ID NO: 57, which is codon-optimized for expression in mammalian, e.g., human, cells.

According to the invention, the FVIII-ABD used for treatment may comprise one or more ABDs, e.g. 1, 2, 3, 4, or 5 ABDs. The ABDs may be located in tandem or multiples at the same location in FVIII (optionally separated by a suitable linker) or at different locations in the FVIII protein.

If more than one ABD is employed in the FVIII-ABD, it is possible to use different or the same ABDs. Typically, for simplicity and better control, all ABDs in the FVIII protein will have the same sequence, preferably ABD2 (SEQ ID NO: 46). Alternatively, it is possible to use different albumin binding domains, e.g. to achieve binding with different affinities or to bind albumin at different locations of the albumin surface. Thus, one may achieve multivalent albumin binding, resulting in increased affinity or a desired folding of the FVIII protein (e.g. shielding certain sites in FVIII).

Preferably, said albumin binding domain(s) may be located C-terminal to the heavy chain portion and/or C-terminal to the light chain portion of FVIII. If the protein is a single chain protein, the albumin binding domain(s) is/are between the heavy chain portion and the light chain portion and/or C-terminal to the light chain portion. In other words, in a single chain protein, if there are albumin binding domains C-terminal to the heavy chain portion and not C-terminal to the light chain portion, such domains are N-terminal to the light chain portion. In this application, in single chain FVIII-ABD proteins, reference to ABD(s) C-terminal to the heavy chain portion means that the ABD(s) is/are between the heavy chain portion and the light chain portion.

For example, the recombinant Factor VIII protein of the invention, e.g., a single chain protein, may comprise a heavy chain portion and a light chain portion of Factor VIII and one albumin binding domain C-terminal to the heavy chain portion. The recombinant Factor VIII protein of the invention, e.g., a single chain protein, may comprise a heavy chain portion and a light chain portion of Factor VIII and one albumin binding domain C-terminal to the light chain portion.

For example, the recombinant Factor VIII protein of the invention, e.g., a single chain protein, may comprise a heavy chain portion and a light chain portion of Factor VIII and two albumin binding domains C-terminal to the heavy chain portion (i.e., in a single chain protein, between the heavy chain portion and the light chain portion). The recombinant Factor VIII protein of the invention, e.g., a single chain protein, may comprise a heavy chain portion and a light chain portion of Factor VIII and two albumin binding domains C-terminal to the light chain portion.

Alternatively, the recombinant Factor VIII protein of the invention, e.g., a single chain protein, may comprise a heavy chain portion and a light chain portion of Factor VIII and three albumin binding domains C-terminal to the heavy chain portion. The recombinant Factor VIII protein of the invention, e.g., a single chain protein, may comprise a heavy chain portion and a light chain portion of Factor VIII and three albumin binding domains C-terminal to the light chain portion.

The recombinant Factor VIII protein of the invention, e.g., a single chain protein, may comprise a heavy chain portion and a light chain portion of Factor VIII and four albumin binding domains C-terminal to the heavy chain portion. The recombinant Factor VIII protein of the invention, e.g., a single chain protein, may comprise a heavy chain portion and a light chain portion of Factor VIII and four albumin binding domains C-terminal to the light chain portion.

Good bioavailability after subcutaneous injection in minipigs and particularly long half-lives have been found for recombinant Factor VIII protein of the invention comprising a heavy chain portion and a light chain portion of Factor VIII and at least two albumin binding domains, wherein at least one albumin binding domain is C-terminal to the heavy chain portion, and at least one albumin binding domain is C-terminal to the light chain portion. Such FVIII proteins are thus preferably used.

For example, in the FVIII protein of the invention, one albumin binding domain may be C-terminal to the heavy chain portion, and one albumin binding domain C-terminal to the light chain portion. Alternatively, in one of the two selected positions, there may be one albumin binding domain, and two, three, four or more albumin binding domains in the other. For example, one albumin binding domain may be C-terminal to the heavy chain portion, and two albumin binding domains C-terminal to the light chain portion, or one albumin binding domain may be C-terminal to the heavy chain portion, and three albumin binding domains C-terminal to the light chain portion, or one albumin binding domain may be C-terminal to the heavy chain portion, and four albumin binding domains C-terminal to the light chain portion.

In a FVIII protein of the invention, two albumin binding domains may be C-terminal to the heavy chain portion, and one albumin binding domain C-terminal to the light chain portion, or three albumin binding domains may be C-terminal to the heavy chain portion, and one albumin binding domain C-terminal to the light chain portion, or four albumin binding domains may be C-terminal to the heavy chain portion, and one albumin binding domain C-terminal to the light chain portion.

Preferably, the number of albumin binding domains in each of the two positions is the same. It has also been shown to be advantageous if the FVIII protein of the invention comprises at least four albumin binding domains. The inventors have found a still better increase in the half-life for Factor VIII protein of the invention comprising at least two albumin binding domains C-terminal to the heavy chain portion, and at least two albumin binding domains C-terminal to the light chain portion, preferably, two albumin binding domains C-terminal to the heavy chain portion, and two albumin binding domains C-terminal to the light chain portion.

The invention also provides Factor VIII proteins of the invention with two albumin binding domains C-terminal to the heavy chain portion, and three albumin binding domains C-terminal to the light chain portion, or with two albumin binding domains C-terminal to the heavy chain portion, and four albumin binding domains C-terminal to the light chain portion, or with three albumin binding domains C-terminal to the heavy chain portion, and two albumin binding domains C-terminal to the light chain portion, or with four albumin binding domains C-terminal to the heavy chain portion, and two albumin binding domains C-terminal to the light chain portion. Optionally, there is an even number of albumin-binding domains both C-terminal to the heavy chain, and C-terminal to the light chain portion.

Linker

While the inventors have shown that linkers are not principally required for activity and stability of the FVIII-ABD proteins of the invention, to increase accessibility of all domains of the FVIII of the invention, in particular, access to albumin, linkers were introduced into some FVIII-ABD proteins of the invention. The inventors have shown that the linkers, in particular, inclusion of at least glycine-serine linker sections, further improve expression and function. In particular, in addition to access to albumin, access for thrombin also appears to be improved. Accordingly, preferably, albumin binding domains may be separated from the heavy chain portion and/or the light chain portion and/or other albumin-binding domains by a linker, wherein, optionally, albumin-binding domains are separated from the heavy chain portion and the light chain portion and (if directly adjacent otherwise) other albumin-binding domains by a linker. It is also possible that albumin-binding domains are separated from the heavy chain portion and the light chain portion and (if directly adjacent otherwise) other albumin-binding domains by a linker, except that there is no linker N-terminal to the light chain.

In one preferred embodiment, the linker comprises a thrombin-cleavable linker section. For example, such thrombin cleavable linker section has the sequence of SEQ ID NO: 39 (abbr. L). Further thrombin cleavable sites are known in the art, e.g., disclosed in Gallwitz et al. (2012, PLoS ONE 7 (2): e31756). Thrombin cleavable linkers may thus also comprise any of these cleavable sites. Thrombin-cleavable linkers have the advantage that in generation of the active protein, i.e., after activation by thrombin, the linkers may be cleaved and, consequently, the albumin binding domains may be removed from the active protein.

Alternatively, uncleavable glycine-serine linker sections may be used to introduce flexible, sterical distance between motifs to avoid structural influences. Thus, optionally, the linker comprises a glycine-serine linker section that optionally has the sequence of SEQ ID NO: 40 (abbr. G1, preferred) or SEQ ID NO: 41 (abbr. G2). The linker G1 may, e.g., be encoded by SEQ ID NO: 58. The linker G2 may, e.g., be encoded by SEQ ID NO: 59.

In preferred embodiments, different linker sections are combined. E.g. uncleavable linker sections were used to flank a central thrombin cleavable linker section to maintain thrombin accessibility of the thrombin cleavable linker section. Thus, in some embodiments, the linker comprises a thrombin cleavable linker section flanked on each side by a glycine-serine linker section, wherein said combined linker optionally has the sequence of SEQ ID NO: 42 or SEQ ID NO: 43, preferably, SEQ ID NO: 42.

The polynucleotide sequence for all linkers preferably is codon-optimized for expression in mammalian or human cell culture. Exemplary codon-optimized sequences are provided herein and may be used for preparing FVIII-ABD proteins used in the invention.

Specific FVIII-ABD Proteins Used in the Invention

All FVIII-ABD proteins used in the invention demonstrated good in vitro functionality, wherein the FVIII-ABD proteins showed reduced vWF binding in correlation to increasing numbers of albumin binding domains. vWF has a major impact on the half-life of FVIII. It was found that shielding FVIII from vWF by albumin positively influences the half-life of the FVIII protein. A broad distribution of albumin binding domains with one position between heavy chain and light chain and one position at the C-terminus of the protein was shown to enhance the shielding of FVIII from vWF.

Preferably, the recombinant Factor VIII-ABD protein used in the invention comprises one albumin binding domain C-terminal to the light chain portion (i.e., in a single chain protein, between the heavy chain portion and the light chain portion) and one albumin binding domain C-terminal to the light chain portion, wherein the sequence has at least 70%, optionally, at least 80%, at least 90%, at least 95%, at least 99% or 100% sequence identity sequence identity to SEQ ID NO: 47. Preferably, said protein is a single chain protein. The single chain protein having SEQ ID NO: 47 is designated ADLCLD_SC. It was shown to have an in vivo half-life increased by a factor of about 1.5 compared to ReFacto AF®.

In an especially preferred embodiment, the recombinant Factor VIII protein of the invention comprises at least two albumin binding domains C-terminal to the light chain portion (i.e., in a single chain protein, between the heavy chain portion and the light chain portion) and at least two albumin binding domain C-terminal to the light chain portion, wherein, preferably, the protein has at least 80% sequence identity, optionally, at least 90%, at least 95% or at least 99% sequence identity to any of SEQ ID NO: 48, 49, 51. Preferably, the recombinant Factor VIII protein has at least 80% sequence identity, optionally, at least 90%, at least 95%, or at least 99% sequence identity to SEQ ID NO: 48. Preferably, said protein is a single chain protein.

The recombinant Factor VIII protein may also have at least 80% sequence identity, optionally, at least 90%, at least 95%, or at least 99% sequence identity to SEQ ID NO: 49. Preferably, said protein is a single chain protein.

The recombinant Factor VIII protein may also have at least 80% sequence identity, optionally, at least 90%, at least 95%, or at least 99% sequence identity to SEQ ID NO: 51. Preferably, said protein is a single chain protein.

For example, the invention provides a recombinant FVIII-ABD protein having SEQ ID NO: 48 (AD2CD2_SC), SEQ ID NO: 49 (AD2CD2woL_SC), or SEQ ID NO: 51 (AbD2CD2_SC). Such FVIII proteins have been shown to have a particularly good bioavailability after subcutaneous administration, as shown herein. They also have an extended in vivo half-life, e.g., for AD2CD2_SC, an in vivo half-life extended by a factor of 2.5 has been found in hemophilia A mice and a half-life extension of factor 4 has been found in albumin-deficient, transgenic neonatal Fc-receptor mice (see Examples). For AbD2CD2_SC, an in vivo half-life extended by a factor of 2.2 has been found in said mice. An increase in half-life can be analyzed on the level of FVIII antigen or on the level of activity, e.g., chromogenic activity, or both. Preferably, it is analyzed on the level of chromogenic activity.

The invention may thus employ FVIII-ABD proteins, wherein the in vivo half-life of the Factor VIII-ABD protein is prolonged (i.e. increased) by a factor of at least 1.2, preferably, by a factor of at least 1.5, optionally, at least 2 or at least 2.5 in comparison to a recombinant Factor VIII protein of SEQ ID NO: 28 (ReFacto AF®). While the increase in in vivo half-life may be analyzed in model systems, e.g., mice, rats or dogs, such as in hemophilia A mice or albumin-deficient Tg32 mice having a knock-out of murine albumin and expressing human FcRn a-chain instead of the murine one (B6.Cg-Tg(FCGRT)32Dcr Alb^{em12Mvw} Fcgrt^{tm1Dcr}/MvwJ), the observed increase in in vivo half-life may be underestimated, because human albumin has a longer half-life than e.g. murine albumin, and it is expected that an increase seen in a murine model will be still more pronounced in humans.

The protein may further be glycosylated and/or sulfated. Preferably, post-translational modifications such as glycosylation and/or sulfation of the protein occur in a human cell. A particularly suitable profile of post-translational modifications can be achieved using human cell lines for production, e.g. CAP cells, in particular CAP-T cells or CAP-Go cells (WO 2001/36615; WO 2007/056994; WO 2010/094280; WO 2016/110302). CAP cells, available from Cevec Pharmaceuticals GmbH (Cologne, Germany), originate from human amniocytes as they were isolated trans-abdominally during routine amniocentesis. Obtained amniocytes were transformed with adenoviral functions (E1A, E1B, and pIX functions) and subsequently adapted to growth in suspension in serum-free medium.

During post-translational modification, the FVIII protein of the invention may be sulfated, e.g., on one, two, three, four, five or six tyrosines in the acidic regions a1, a2 and a3.

De-Immunized Factor VIII Proteins

Optionally, the recombinant Factor VIII protein used in the pharmaceutical composition of the invention is a de-immunized protein, i.e., the protein has a reduced immunogenicity as compared to wt FVIII in hemophilia patients. Immunogenicity can be determined as described in WO 2019/197524 A1.

De-immunization may already be achieved by the binding of albumin to the FVIII-ABDs of the invention, as the albumin may shield the FVIII from binding of the inhibitory antibodies that are present in some patients. Additionally, FVIII-ABD-bound albumin might reduce the risk of inhibitory antibody development by preventing FVIII-ABD processing normally followed by immunogenic FVIII-derived peptides presentation on human HLA molecules of antigen-presenting cells due to recycling of FVIII-ABD:albumin complexes via the neonatal Fc receptor.

In a de-immunized FVIII-ABD protein of the invention, for example, certain mutations, preferably, substitutions, have been introduced to avoid the presence of epitopes which can be presented on human HLA molecules, preferably, common human HLA molecules. Preferred mutations are disclosed in WO 2019/197524 A1 which is fully incorporated herein by reference. Any of the mutations disclosed therein, or combinations of mutations disclosed therein, may also be incorporated into the FVIII-ABD proteins of the invention.

For example, the Factor VIII-ABD protein that may be used in the invention may comprise at least three amino acid substitutions at positions selected from the group consisting of Y748, L171, S507, N79, I80, I105, S112, L160, V184, N233, L235, V257, I265, N299, Y426, Y430, L505, F555, I610, N616, I632, L706, N754, K1837, R1936, S2030, S2037, N2038, S2077, M2123, S2125, F2215, K2226, K2258, V2313, S2315, V2333 and Q2335;

wherein substitutions of N are independently selected from the group consisting of D, H, S and E; wherein substitution of I are independently selected from the group consisting of T and V; wherein substitutions of S are independently selected from the group consisting of A, N, G, T and E; wherein substitutions of L are independently selected from the group consisting of N, Q, F and S; wherein substitutions of V are independently selected from the group consisting of A and T; wherein substitutions of Y are independently selected from the group consisting of N, H and S; wherein substitutions of F are independently selected from the group consisting of H and S; wherein substitutions of K are independently selected from the group consisting of N, D, E, Q, S and T; wherein substitutions of R are independently selected from the group consisting of Q, H and S; wherein substitutions of M are selected from the group consisting of R, Q, K and T; and/or wherein substitutions of Q are selected from the group consisting of R, D, E, H and K;

wherein the positions are specified in relation to full length human Factor VIII molecule of SEQ ID NO: 1 including numbering of the signal sequence;

and wherein the recombinant Factor VIII protein retains at least 50% coagulant activity, as determined in a chromogenic assay, compared to a Factor VIII protein consisting of SEQ ID NO: 60 (FVIII-6rs). The invention also provides a fusion protein of said recombinant Factor VIII protein.

The inventors found that specific substitutions tested were particularly advantageous with regard both to a reduced immunogenicity and maintenance of functional activity in coagulation. Accordingly, the amino acid substitutions in a de-immunized recombinant Factor VIII-ABD protein of the invention are preferably selected from the group consisting of Y748S, L171Q, S507E, N79S, I80T, I105V, S112T, L160S, V184A, N233D, L235F, V257A, I265T, N299D, Y426H, Y430H, L505N, F555H, I610T, N616E, I632T, L706N, N754D, K1837E, R1936Q, S2030A, S2037G, N2038D, S2077G, M2123K, S2125G, F2215H, K2226Q, K2258Q, V2313A, S2315T, V2333A and Q2335H (again referring to sequence ID NO: 1).

In all de-immunized recombinant FVIII proteins used in the invention, the positions are specified in relation to full length human Factor VIII molecule of SEQ ID NO: 1. In the state of the art, annotation of amino acids in the FVIII molecule differs between authors. This is mainly due to the 19 amino acid signal sequence, which can be included into the amino acid count or can be omitted. This variation of plus or minus 19 amino acids is in general the only difference in numeration for full-length FVIII sequences. For B-domain deleted FVIII sequences, the deletion may also lead to a shift in numeration. For the heavy chain the numeration correlates with the numeration of the full-length FVIII. From the B-domain deletion on the numeration of the light chain is either kept the same as for the full-length FVIII molecule (e.g. Q763 in front of the deletion is followed by D1582 after the deletion) or can be continued as if no deletion has occurred (e.g. Q763 is followed by D764 despite missing amino acids). The continued numeration complicates the comparison of amino acid sequences if it is not known how many amino acids were deleted. The continued numeration is rare and most authors keep the numeration of the full-length FVIII molecule despite B-domain deletion. In accordance with this, in the present invention, the positions of substitutions in the recombinant FVIII protein are specified in relation to full length human FVIII molecule of SEQ ID NO: 1. Nevertheless, the secreted recombinant FVIII protein does not comprise the signal sequence, comprises the albumin-binding domains as specified herein, and typically is a B-domain deleted variant.

Throughout the invention, the recombinant Factor VIII-ABD protein used in the invention may have at least 90%, at least 95%, at least 96%, at least 97%, at least 98%, or at least 99% sequence identity to a mature (i.e., not including the signal sequence) FVIII-19M protein of SEQ ID NO: 63, wherein only the A1, a1, A2, a2, a3, A3, C1 and C2 domains (residues 20-759 and residues 1668-2351) are considered for determination of sequence identity. In other words, for determination of sequence identity, the B-domain (residues 760-1667 of the full length human sequence SEQ ID NO: 1, and the residues corresponding thereto in partially B-domain deleted proteins) and the signal sequence (residues 1-19), as well as the albumin-binding domain(s) and, optionally, linkers or other fusion partners, are not taken into account.

Accordingly, the % sequence identity to a mature full length human Factor VIII protein of SEQ ID NO: 1, or to a B-domain deleted variant thereof, e.g., according to SEQ ID NO: 61, to a FVIII protein of SEQ ID NO: 63 is the same, in particular, it is 98.67%, wherein only the A1, a1, A2, a2, a3, A3, C1 and C2 domains are considered for determination of sequence identity. Preferred FVIII proteins used in the invention have a sequence identity to SEQ ID NO: 63 of at least 98.74%, wherein only the A1, a1, A2, a2, a3, A3, C1 and C2 domains are considered for determination of sequence identity.

For example, for a mature B-domain deleted FVIII protein with only one of the recited substitutions, the % sequence identity to mature FVIII-19M protein of SEQ ID NO: 63 is determined over the A1, a1, A2, a2, a3, A3, C1 and C2 domains, i.e. 18 of 1424 amino acids are substituted, and the protein accordingly has at least 98.74% sequence identity to FVIII-19M protein of SEQ ID NO: 63. For a mature B-domain deleted FVIII protein with 3 of the recited substitutions also occurring in FVIII-19M, the % sequence identity to mature FVIII-19M protein of SEQ ID NO: 63, is determined over the A1, a1, A2, a2, a3, A3, C1 and C2 domains, i.e. 16 of 1424 amino acids are substituted, and the protein accordingly has 98.88% sequence identity to FVIII-19M protein of SEQ ID NO: 63. A mature B-domain deleted FVIII protein used in the invention with 4 of the recited substitutions also occurring in FVIII-19M has 15 of 1424 amino acids substituted, and thus has 98.95% sequence identity. A mature B-domain deleted FVIII protein incorporating all 38 recited substitutions has 19 additional substitutions compared to in FVIII-19M, and thus has 98.67% sequence identity to FVIII-19M.

If sequence identity is defined by reference to the A1, a1, A2, a2, a3, A3, C1 and C2 domains only, sequence identity is furthermore determined for the Factor VIII part (as defined, based on the A1, a1, A2, a2, a3, A3, C1 and C2 domains) of the molecule only, i.e., the albumin-binding domain(s), and any linkers (if applicable) are not taken into account, or if the protein is a fusion protein with a further fusion partner (for example, contains insertions of any size), fused or inserted parts, protein domains or regions (e.g., as further described herein) are not taken into account. Thus, for the determination of sequence identity, if present, fusion partners are ignored, and the % sequence identity to A1, a1, A2, a2, a3, A3, C1 and C2 domains is then calculated. Sequence identity can be calculated as known in the art, e.g., using the Needleman-Wunsch algorithm or, preferably, the Smith-Waterman algorithm (Smith et al., 1981. Identification of Common Molecular Subsequences, J Mol Biol. 147: 195-197).

In one embodiment, all residues of the FVIII protein, in particular, with regard to the A1, a1, A2, a2, a3, A3, C1 and C2 domains, except for the substitutions specified herein, correspond to (i.e., are identical to) residues of human Factor VIII protein of SEQ ID NO: 1. Optionally, this may also apply for the B-domain or those parts of the B-domain which are present.

In another embodiment, the FVIII protein in the pharmaceutical compositions of the invention incorporates further mutations, e.g., mutations known in the art to reduce immunogenicity either with regard to further T cell epitopes and/or B cell epitopes, and/or mutations known in the art to improve serum half-life of the protein and/or mutations facilitating purification of the protein, e.g., leading to a single chain protein. Mutations may also be introduced due to partial deletion of the B-domain and engineering of a single chain protein.

In another example, the de-immunized Factor VIII-ABD protein comprises at least 19 amino acid substitutions at positions N79, S112, L160, L171, V184, N233, 1265, N299, Y426, S507, F555, N616, L706, Y748, K1837, N2038, S2077, S2315 and V2333, wherein preferably the 19 substitutions are N79S, S112T, L160S, L171Q, V184A, N233D, 1265T, N299D, Y426H, S507E, F555H, N616E, L706N, Y748S, K1837E, N2038D, 52077G, 52315T and V2333A. Optionally, the protein has 100% sequence identity to aa 20-1533 of SEQ ID NO: 63 (FVIII-19M), i.e., the mature protein does not comprise the 19 aa N-terminal signal sequence wherein only the A1, a1, A2, a2, a3, A3, C1 and C2 domains are considered for determination of sequence identity.

Fusion Partners

In addition to the albumin-binding domain(s) contained in the FVIII protein used in the invention, the protein may be a fusion protein with another fusion partner, e.g., a fusion protein of a recombinant Factor VIII protein having at least 90%, at least 95%, at least 96%, at least 97%, at least 98% or at least 99% sequence identity to a FVIII-19M as specified in SEQ ID NO: 63, wherein only the A1, a1, A2, a2, a3, A3, C1 and C2 domains are considered for calculation of sequence identity.

For example, the fusion partner may extend the in vivo serum half-life of the FVIII protein of the invention. The fusion partner may be selected from the group comprising an Fc region, albumin, PAS polypeptides, HAP polypeptides, the C-terminal peptide of the beta subunit of chorionic gonadotropin, and combinations thereof. The FVIII protein may alternatively or additionally be covalently linked to non-protein fusion partners such as albumin-binding small molecules, and/or PEG (polyethylenglycol) and/or HES (hydroxyethyl starch). PAS polypeptides or PAS sequences are polypeptides comprising an amino acid sequence comprising mainly alanine and serine residues or comprising mainly alanine, proline and serine residues, the PAS sequences forming a random coil conformation under physiological conditions, as defined in WO 2015/023894. HAP polypeptides or sequences are homo-amino acid polymer (HAP), comprising e.g., repetitive sequences of glycine or glycine and serine, as defined in WO 2015/023894. Potential fusions, fusion partners and combinations thereof are described in more detail e.g., in WO 2015/023894.

Optionally, for therapeutic applications, the recombinant FVIII protein is at least fused to an Fc region. Fusion proteins of FVIII to Fc regions have been described in the state of the art to reduce immunogenicity (Krishnamoorthy et al., Recombinant factor VIII Fc (rFVIIIFc) fusion protein reduces immunogenicity and induces tolerance in hemophilia A mice, Cell. Immunol. 2016, http://dx.doi.org/10.1016/j.cellimm.2015.12.2008; Carcao et al., Recombinant factor VIII Fc fusion protein for immune tolerance induction in patients with severe haemophilia A with inhibitors—A retrospective analysis. Haemophilia 2018:1-8).

Fusion partners may e.g., be linked to the N-terminus or the C-terminus of the FVIII protein of the invention, but they may also be inserted within the FVIII sequence, as long as the FVIII protein remains functional as defined herein. As described above, for determination of sequence identity, insertions of, e.g., one, two, three, four, five, six, seven, eight, nine or ten fusion partners, as defined herein, are not considered to reduce sequence identity when the sequence identity is defined by reference to the A1, a1, A2, a2, a3, A3, C1 and C2 domains.

Optionally, said heterologous fusion partner may be inserted directly N-terminal or directly C-terminal to one of the albumin binding domains, e.g., C-terminal to the heavy chain, and/or C-terminal to the C2-domain, or C-terminal to the albumin binding domain(s) C-terminal to the heavy chain or C-terminal to the albumin binding domain(s) C-terminal to the heavy chain. These locations have been found by the inventors to be advantageous for fusion, while maintaining good biological activity of the FVIII protein. Optionally, the fusion protein further comprises at least one linker. In one embodiment, the FVIII protein employed in the invention does not comprise further fusion partners that are not albumin binding domains or FVIII sequences as defined herein, wherein the FVIII protein optionally comprises linkers as defined herein.

Pharmaceutical Compositions

The invention provides a pharmaceutical composition comprising the recombinant Factor VIII protein as described herein. The pharmaceutical composition preferably is for use in subcutaneous administration to treat hemophilia A. This means that the invention also provides a method for treating hemophilia A by subcutaneous administration of the pharmaceutical composition comprising the recombinant Factor VIII protein as described herein, as well as a method for preparing a pharmaceutical composition comprising the recombinant Factor VIII protein as described herein, wherein the composition is for subcutaneous administration.

Accordingly, preferably, the composition is suitable for human administration. Such pharmaceutical compositions may comprise suitable (i.e., pharmaceutically acceptable) excipients or carriers, e.g., a buffer, comprising, e.g., calcium chloride and/or sodium citrate and/or sodium phosphate and/or glycine, a stabilizing agent such as arginine and/or histidine and/or polysorbate and/or sucrose, osmolarity modifying agents, such as salt or sugar, a bulking agent, such as hydroxyethyl starch or trehalose, a preservative, such as M-cresol and/or benzyl alcohol, another (e.g., recombinant) protein or combinations thereof. Preferably, the FVIII composition does not comprise a preservative. In the context of the invention, if not explicitly stated otherwise, “a” is understood to mean one or more.

For example, a suitable buffer may comprise NaCl, CaCl₂, L-Histidine, Sucrose and Polysorbate 20. A suitable buffer for formulation of the protein of the invention may e.g. contain 205 mM NaCl, 5.3 mM CaCl₂, 6.7 mM L-Histidine, 1.3% Sucrose and 0.013% Polysorbate 20 in distilled water and have a pH of 7.0 (FVIII formulation buffer or formulation buffer). Said buffer is used in the experiments described herein if not otherwise stated. Formulations of FVIII may be sterile, e.g., sterile filtered, in particular for in vivo use.

The inventors provide FVIII proteins and compositions thereof that can advantageously be subcutaneously administered with a high bioavailability, as described herein, and that are still suitable for human administration.

Thus, they may comprise excipients suitable for human administration and in dosages suitable for human administration. For example, high amounts of polysorbate (e.g., more than 0.3 mg/kg bodyweight) are not desired.

Pharmaceutical compositions comprising FVIII can be dried, e.g., lyophilized.

In certain embodiments, the pharmaceutical composition of the invention comprising FVIII-ABD protein further comprises albumin. Preferably, the albumin used has at least 50%, preferably, at least 80% or at least 90% of the binding capacity of human albumin to the albumin binding domain(s) incorporated in the FVIII proteins.

Preferably, in particular for administration to a human patient, human albumin or serum albumin is used, optionally, recombinantly produced or purified from human blood plasma or blood serum (plasma-derived albumin). While human plasma-derived albumin has the advantage of being naturally derived and commercially available, use of recombinant albumin may be preferred by some authorities.

Albumin may, e.g., be present in the pharmaceutical composition in a concentration of 0.1-15% w/v, wherein the volume relates to the final composition for administration, such as 0.5-10% (w/v) albumin. For example, in the animal model, particularly good results have been found with 3-10% (w/v) albumin, wherein the bioavailability after subcutaneous administration was increased compared to administration with no albumin or lower amounts of albumin.

However, not all kinds of albumin bind to the albumin binding domains preferably used in the FVIII proteins as well as human albumin. In particular, murine albumin is known to bind with lower affinity to ABD2 compared to human albumin. This may decrease the effect seen in the murine model. In comparison, in a human patient, there is human albumin with good binding efficiency to the ABDs that are preferably used, which increases albumin binding.

Albumin is not only present in the blood or serum, but there also is a significant concentration in the interstitium, e.g., about 242 g albumin were shown to be in present the extravascular space of a human (while approx. 118 g are in the intravascular space [118 g/2.5 L serum=about 47 g/L]) (Merlot et al., 2014. Front Physiol. 5:299). Albumin will thus also be present in the environment into which the pharmaceutical composition of the invention is subcutaneously administered. Albumin may consequently bind to the albumin binding domain(s) of the FVIII proteins either before administration, or after administration to a human subject.

Thus, lower albumin concentrations than 3-10% (w/v) may also be used, e.g., 1-5% (w/v) or about 2% (w/v). Albumin may also be absent from the pharmaceutical composition of the invention, or present in a low concentration such as 0.1-0.5% (w/v).

In another embodiment, the pharmaceutical composition of the invention comprises the FVIII protein as defined herein and further comprises a hyaluronidase. A hyaluronidase is an enzyme capable of degrading, preferably specifically degrading, hyaluronan (formerly also known as hyaluronic acid) for subcutaneous administration. The hyaluronidase may have at least 90% sequence identity to a wildtype human hyaluronidase, e.g., PH-20 or a soluble fragment thereof, such as vorhyaluronidase alfa (SEQ ID NO: 127). The hyaluronidase may be a human hyaluronidase, e.g., a wildtype human hyaluronidase, or, preferably, a soluble fragment thereof. Hyaluronidases and doses of hyaluronidase that may be employed are known in the art. Excellent results have been obtained with vorhyaluronidase alfa (SEQ ID NO: 127), a recombinant human protein corresponding to the amino acid sequence of human hyaluronidase PH-20 at positions 36-482. Vorhyaluronidase alfa is contained in Hylenex®, Halozyme, US. Further information on vorhyaluronidase alfa is provided, e.g., in the database genome.jp as entry D06604, https://www.genome.jp/dbget-bin/www_bget?dr:D06604). If deemed appropriate by the skilled person hyaluronidases may be modified, e.g., be pegylated.

Optionally, in the pharmaceutical composition of the invention, the dose of the hyaluronidase is 10-300 U per injection, preferably, for a grown-up person, 50-300 U per injection, e.g., about 150 U per injection. The inventors have shown that a combination with hyaluronidase may significantly improve bioavailability upon subcutaneous administration.

WO 2004/078140 A2 (Halozyme Therapeutics Inc.) describes soluble hyaluronidase glycoprotein and its use to facilitate administration of other molecules. However, said document does not provide any evidence that the bioavailability of FVIII protein, let alone of the FVIII proteins comprising at least one albumin binding domain as defined herein could be improved upon subcutaneous administration in combination with hyaluronidase. The document thus does not render it obvious that clinically relevant plasma levels may be achieved using a dose as described herein for treatment of a human patient.

The inventors found that particularly good results could be obtained when the pharmaceutical composition of the invention comprised both hyaluronidase and albumin, in particular, results were better with higher concentrations of albumin. In one embodiment, thus, the pharmaceutical composition of the invention comprises the FVIII protein as defined herein, and a hyaluronidase and human albumin.

WO2009/111066 A1 and WO2009/111083 A2 mention albumin binding of hyaluronidase. WO2004/078140 A2, WO2006/091871 A1 describes that albumin optimizes enzymatic activity of hyaluronidase. WO2009/134380 A2 describes albumin to be a stabilizer of hyaluronidase. Thus, the pharmaceutical composition of the invention, if comprising hyaluronidase, preferably comprises at least amounts of albumin sufficient to stabilize the enzyme. Commercial hyaluronidase compositions often comprise albumin in such concentrations, as described in detail in the examples below. As mentioned above, albumin may be advantageous, but it is not required in the pharmaceutical compositions of the invention for treatment of a human patient, as it may be present in the environment into which the composition is subcutaneously administered.

The invention also provides a kit comprising a hyaluronidase, e.g., a human hyaluronidase, and a pharmaceutical composition for use of the invention, comprising a recombinant Factor VIII protein comprising a heavy chain portion and a light chain portion of Factor VIII and at least one albumin binding domain(s), wherein the albumin binding domain(s) is/are C-terminal to the heavy chain portion and/or C-terminal to the light chain portion. If the protein is a single chain protein, and if there are albumin binding domain(s) C-terminal to the heavy chain portion and not C-terminal to the light chain portion, these albumin binding domain(s) is/are between the heavy chain portion and the light chain portion. The pharmaceutical composition preferably is the pharmaceutical composition for use of the invention, as described herein.

The invention also provides a kit comprising albumin, e.g., human serum albumin, and a pharmaceutical composition for use of the invention, comprising a recombinant Factor VIII protein comprising a heavy chain portion and a light chain portion of Factor VIII and at least one albumin binding domain(s), wherein the albumin binding domain(s) is/are C-terminal to the heavy chain portion and/or C-terminal to the light chain portion. If the protein is a single chain protein, and if there are albumin binding domain(s) C-terminal to the heavy chain portion and not C-terminal to the light chain portion, these albumin binding domain(s) is/are between the heavy chain portion and the light chain portion. The pharmaceutical composition preferably is the pharmaceutical composition for use of the invention, as described herein.

The invention also provides a FVIII-ABD of the invention for use in subcutaneous administration to treat hemophilia A, wherein the FVIII-ABD is to be co-administered with human serum albumin and/or hyaluronidase. Co-administration requires that both agents are subcutaneously administered, preferably, at essentially the same site. The sites of administration should be, in particular, close enough to enable a combined effect of the two agents, e.g., through binding of the albumin to the albumin-binding domain(s). Co-administration may be essentially simultaneously, e.g., within 5 minutes, or sequential administration, wherein the administration of the FVIII-ABD may be first or second, e.g., with an interval of 5 min to 1 h or 10-15 min.

The FVIII-ABD may also be mixed with human serum albumin and/or hyaluronidase before administration and then injected together.

In one embodiment, the invention also provides a single chamber syringe comprising a solution comprising FVIII-ABD of the invention, for use in subcutaneous administration as described herein, and/or albumin, e.g. human serum albumin, and/or hyaluronidase. Optionally, the single chamber syringe comprises a lyophilizate thereof. If the FVIII-ABD and/or albumin and/or hyaluronidase is lyophilized, it is dissolved by drawing the syringe with a solution, e.g. a buffer or water, which may also be supplied as part of a kit.

The invention also provides a double chamber syringe, wherein one chamber comprises FVIII-ABD of the invention, for use in subcutaneous administration as described herein. The FVIII-ABD may, e.g., be lyophilized. The second chamber may comprise albumin and/or hyaluronidase, preferably, a solution thereof. Before injection, the agents in the two chambers of the syringe are mixed. If the FVIII-ABD is lyophilized, it is dissolved in the solution.

The invention also provides a double chamber syringe, wherein one chamber comprises FVIII-ABD of the invention, for use in subcutaneous administration as described herein, and/or albumin and/or hyaluronidase. The FVIII-ABD and/or albumin and/or hyaluronidase may, e.g., be lyophilized. The second chamber may comprise a solution, e.g. a buffer or water. Before injection, the agents in the two chambers of the syringe are mixed. If the FVIII-ABD and/or albumin and/or hyaluronidase is/are lyophilized, it/they is/are dissolved in the solution.

Stability may be still enhanced if one chamber of a syringe of the invention comprises the FVIII-ABD of the invention and albumin, e.g., in lyophilized form. The syringe may preferably comprise a second chamber with a buffer or water for injection, wherein the lyophilized agents from the first chamber are dissolved before injection. Optionally, the second chamber further comprises a hyaluronidase, wherein the hyaluronidase composition may also comprise albumin. A hyaluronidase may alternatively be in a third chamber of the syringe.

The invention also provides a multiple chamber syringe, wherein one chamber comprises FVIII-ABD of the invention, for use in subcutaneous administration as described herein, and/or albumin and/or hyaluronidase. The FVIII-ABD and/or albumin and/or hyaluronidase may, e.g., be lyophilized. A second chamber may comprise albumin and/or hyaluronidase dissolved in solution or, optionally, as a lyophilizate. A third chamber may comprise a solution, e.g. a buffer or water. Before injection, the agents in the multiple chambers of the syringe are mixed. If the FVIII-ABD and/or albumin and/or hyaluronidase are lyophilized, they are dissolved in the solution.

The invention also provides a pharmaceutical composition comprising the FVIII protein for use of the invention in combination with an immunosuppressive agent (e.g., methotrexate, methylprednisolone, prednisolone, dexamethasone, cyclophosphamide, rituximab, and/or cyclosporin), and/or it may be for administration at substantially the same time (e.g. within five minutes to within 12 hours) with such an agent. The invention thus also provides a kit comprising, in addition to a FVIII protein, optionally, combined with albumin, an immunosuppressive agent, e.g., an immunosuppressive agent selected from the group comprising methotrexate, methylprednisolone, prednisolone, dexamethason, cyclophosphamide, rituximab, and/or cyclosporin.

Further, due to the increase in half-life in vivo obtained with preferred FVIII-ABD proteins, and in addition, due to a sustained release from the skin reservoir, the pharmaceutical compositions of the invention may be administered at longer intervals than previous FVIII compositions. For example, they may be for use in administration every 4-21 days, preferably, every 5 to 14 days, or optionally, every 7 to 10 days.

Use of a de-immunized FVIII-ABD protein, as described herein, is particularly advantageous in settings wherein a reduced immunogenicity is desired, e.g., for use in treating a patient with hemophilia A not previously treated with any recombinant or plasmatic Factor VIII protein. According to the invention, the incidence and/or severity of generation of antibodies including inhibitory antibodies in the patient is thus reduced compared to treatment with conventional FVIII, or preferably, the generation of antibodies including inhibitory antibodies is prevented. The pharmaceutical composition for use of the invention may also be used for treatment of a patient previously already treated with a recombinant and/or plasmatic Factor VIII protein. In a patient who has an antibody including an inhibitory antibody response to a recombinant and/or plasmatic Factor VIII protein, the pharmaceutical compositions may, e.g., be used for immune tolerance induction (ITI) treatment, as it is desired to use a FVIII protein having a low immunogenicity or even tolerogenic characteristics (Carcao et al., Recombinant factor VIII Fc fusion protein for immune tolerance induction in patients with severe haemophilia A with inhibitors—A retrospective analysis. Haemophilia 2018:1-8). The compositions for use of the invention may thus also be used for rescue ITI. The pharmaceutical compositions may also be advantageously used in a patient who has had an antibody response including an inhibitory antibody response to a recombinant and/or plasmatic Factor VIII protein, e.g., who has been treated by ITI. The pharmaceutical compositions may also be advantageously used in a patient who has had an antibody response including an inhibitory antibody response to a recombinant and/or plasmatic Factor VIII protein, who has not been treated by ITI.

The invention also provides a vial comprising the pharmaceutical composition for use of the invention, e.g., a syringe. The syringe may be a pre-filled syringe, e.g., a ready-to-use syringe, such as the dual-chamber syringe described above.

The invention also provides a method of treatment, comprising administering a dose of 1-1000 U/kg bodyweight of the FVIII-ABD protein or the pharmaceutical composition of the invention to a patient in need thereof, e.g., a patient with hemophilia A, as described herein. Also provided is a method of preparing a pharmaceutical composition of the invention, wherein the pharmaceutical composition is for subcutaneous administration to a patient with hemophilia A.

All publications cited herein are fully incorporated herewith. The invention is further illustrated by the following embodiments, figures and examples, which are not to be understood as limiting the scope of the invention.

### EMBODIMENTS OF THE INVENTION

The invention further comprises the following embodiments. In particular, in embodiment 1, the invention provides a Factor VIII protein comprising at least one albumin binding domain, and wherein the bioavailability of the Factor VIII protein after subcutaneous administration is at least 25% as measured in minipigs, preferably, for use in treatment of a subject having hemophilia A.

In embodiment 2, the invention provides a Factor VIII protein comprising at least one albumin binding domain, wherein the Factor VIII protein optionally is a single chain protein, for use in treatment of a subject having hemophilia A, wherein a dose of 1-1000 U/kg bodyweight is administered to the subject subcutaneously.

In embodiment 3, the invention provides a Factor VIII protein comprising at least one albumin binding domain, wherein the Factor VIII protein optionally is a single chain protein, and wherein the bioavailability of the Factor VIII protein after subcutaneous administration is at least 25% as measured in minipigs, for use in treatment of a subject having hemophilia A, wherein a dose of 1-1000 U/kg bodyweight is administered to the subject subcutaneously.

In embodiment 4, the bioavailability of the Factor VIII protein of any of embodiments 1-3, after subcutaneous administration as measured in minipigs, is at least 30%, preferably, at least 35%. In embodiment 5, the bioavailability of the Factor VIII protein of any of embodiments 1-4, after subcutaneous administration as measured in minipigs, is at least 40%, e.g., at least 50%. In embodiment 6, the bioavailability of the Factor VIII protein of any of embodiments 1-5, after subcutaneous administration as measured in minipigs, is 30-80%, e.g., 30-60%.

In embodiment 7, the bioavailability of the Factor VIII protein of any of embodiments 1-6 after subcutaneous administration in a mouse is at least 10%, preferably, at least 15%. In embodiment 8, the bioavailability of the Factor VIII protein of any of embodiments 1-7 after subcutaneous administration in a mouse is 10-60%, e.g., 10-30%. In embodiment 9, the bioavailability of the Factor VIII protein of embodiments 1-8 after subcutaneous administration in a mouse is 15-20%.

In embodiment 10, the bioavailability of the Factor VIII protein of any of embodiments 1-9 after subcutaneous administration in a human subject is at least 15%, preferably, at least 20%. In embodiment 11, the bioavailability of the Factor VIII protein of any embodiments 1-10 after subcutaneous administration in a human subject is 30-80%, e.g., 30-60%. In embodiment 12, the bioavailability of the Factor VIII protein of embodiments 1-11 after subcutaneous administration in a human subject is at least 40%.

In embodiment 13, the Factor VIII protein of any of embodiments 1-12, is a single chain protein. In embodiment 14, the Factor VIII protein of any of embodiments 1-12 is a double chain protein.

In embodiment 15, the Factor VIII protein of any of embodiments 1-14 is at least partly B domain deleted.

In embodiment 16, the Factor VIII protein of any of embodiments 1-15 comprises at least two albumin-binding domains.

In embodiment 17, in the Factor VIII protein of any of embodiments 1-16, the albumin binding domain(s) is/are C-terminal to the heavy chain portion and/or C-terminal to the light chain portion, wherein, if the protein is a single chain protein, the albumin binding domain(s) is/are between the heavy chain portion and the light chain portion and/or C-terminal to the light chain portion. In other words, in single chain proteins of the invention, albumin binding domain(s) defined herein as C-terminal to the heavy chain portion, if any such albumin binding domain(s) is/are present, is/are N-terminal to the light chain portion.

In embodiment 18, in the Factor VIII protein of any of embodiments 1-17, at least one albumin binding domain is C-terminal to the heavy chain portion and at least one albumin binding domain is C-terminal to the light chain portion, wherein, preferably, two albumin binding domains are C-terminal to the heavy chain portion and two albumin binding domains are C-terminal to the light chain portion. Thus, if the protein is a single chain protein, at least one, preferably, two albumin binding domain(s) is/are between the heavy chain portion and the light chain portion and at least one, preferably, two albumin binding domain(s) is/are C-terminal to the light chain portion.

In embodiment 19, in the Factor VIII protein of any of embodiments 1-18, one albumin binding domain is C-terminal to the heavy chain portion and two albumin binding domains are C-terminal to the light chain portion. In embodiment 20, in the Factor VIII protein of any of embodiments 1-18, one albumin binding domain is C-terminal to the heavy chain portion and three albumin binding domains are C-terminal to the light chain portion. In embodiment 21, in the Factor VIII protein of any of embodiments 1-18, one albumin binding domain is C-terminal to the heavy chain portion and four albumin binding domains are C-terminal to the light chain portion.

In embodiment 22, in the Factor VIII protein of any of embodiments 1-18, two albumin binding domains are C-terminal to the heavy chain portion and one albumin binding domain is C-terminal to the light chain portion. In embodiment 23, in the Factor VIII protein of any of embodiments 1-18, three albumin binding domains are C-terminal to the heavy chain portion and one albumin binding domain is C-terminal to the light chain portion. In embodiment 24, in the Factor VIII protein of any of embodiments 1-18, four albumin binding domains are C-terminal to the heavy chain portion and one albumin binding domain is C-terminal to the light chain portion.

In embodiment 25, in the Factor VIII protein of any of embodiments 1-18, at least two albumin binding domains are C-terminal to the heavy chain portion and at least two albumin binding domains are C-terminal to the light chain portion, preferably, two albumin binding domains are C-terminal to the heavy chain portion and two albumin binding domains are C-terminal to the light chain portion. In embodiment 26, in the Factor VIII protein of any of embodiments 1-18, two albumin binding domains are C-terminal to the heavy chain portion and three albumin binding domains are C-terminal to the light chain portion. In embodiment 27, in the Factor VIII protein of any of embodiments 1-18, two albumin binding domains are C-terminal to the heavy chain portion and four albumin binding domains are C-terminal to the light chain portion. In embodiment 28, in the Factor VIII protein of any of embodiments 1-18, three albumin binding domains are C-terminal to the heavy chain portion and two albumin binding domains are C-terminal to the light chain portion. In embodiment 29, in the Factor VIII protein of any of embodiments 1-18, four albumin binding domains are C-terminal to the heavy chain portion and two albumin binding domains are C-terminal to the light chain portion.

In embodiment 30, in the Factor VIII protein of any of embodiments 1-15, there are two albumin binding domains that are C-terminal to the heavy chain portion, preferably, there are four albumin binding domains that are C-terminal to the heavy chain portion.

In embodiment 31, in the Factor VIII protein of any of embodiments 1-15, there are two albumin binding domains C-terminal to the light chain portion, preferably, there are four albumin binding domains that are C-terminal to the light chain portion.

In embodiment 32, in the Factor VIII protein of any of embodiments 1-31, albumin-binding domains are separated from the heavy chain portion and/or the light chain portion and/or other albumin-binding domains by a linker, wherein, preferably, albumin-binding domains are separated from the heavy chain portion and the light chain portion and other albumin-binding domains by a linker.

In embodiment 33, in the Factor VIII protein of embodiment 32, the linker comprises a thrombin-cleavable linker section that optionally has the sequence of SEQ ID NO: 39.

In embodiment 34, in the Factor VIII protein of any of embodiments 32 or 33, the linker comprises a glycine-serine linker section that optionally has the sequence of SEQ ID NO: 40 or SEQ ID NO: 41.

In embodiment 35, in the Factor VIII protein of any of embodiments 32-34, said linker is a combination of different linker sections, e.g. the linker comprises a thrombin-cleavable linker section flanked on each side by a glycine-serine linker section, wherein said linker optionally has the sequence of SEQ ID NO: 42 or SEQ ID NO: 43.

In embodiment 36, in the Factor VIII protein of any of embodiments 1-35, the albumin binding domain comprises a sequence according to SEQ ID NO: 44. In embodiment 37, in the Factor VIII protein of any of embodiments 1-36, the albumin binding domain comprises a sequence according to SEQ ID NO: 46. In embodiment 38, in the Factor VIII protein of any of embodiments 1-35, the albumin binding domain comprises a sequence according to any of SEQ ID NO: 4-40 of U.S. Pat. No. 10,364,419.

In embodiment 39, in the Factor VIII protein of any of embodiments 1-38, the heavy chain portion comprises the domains A1 and A2, and optionally comprises the domains A1-a1-A2-a2 or A1-a1-A2-a2-B, wherein B may be partly deleted.

In embodiment 40, in the Factor VIII protein of any of embodiments 1-39, the light chain portion comprises the domains A3 and C1 and C2, and optionally comprises the domains a3-A3-C1-C2, wherein a3 may be partly deleted.

In embodiment 41, the Factor VIII protein of any of embodiments 1-40 comprises, in a single chain, a heavy chain portion comprising an A1 and an A2 domain and a light chain portion comprising an A3, C1 and C2 domain of Factor VIII, wherein

a) in said recombinant Factor VIII protein, 894 amino acids corresponding to consecutive amino acids between F761 and P1659 of wild type Factor VIII as defined in SEQ ID NO: 1 are deleted, leading to a first deletion;

b) said recombinant Factor VIII protein comprises, spanning the site of the first deletion, a processing sequence comprising SEQ ID NO: 2 or a sequence having at most one amino acid substitution in SEQ ID NO: 2, wherein said processing sequence comprises a first thrombin cleavage site;

c) in said recombinant Factor VIII protein, at least the amino acids corresponding to amino acids R1664 to R1667 of wild type Factor VIII are deleted, leading to a second deletion; and

d) said recombinant Factor VIII protein comprises, C-terminal to the second deletion and N-terminal of the A3 domain, a second thrombin cleavage site.

In embodiment 42, the Factor VIII protein of any of embodiments 1-41 comprises a heavy chain portion having at least 90% sequence identity to aa20-aa768 of SEQ ID NO: 16 and a light chain portion having at least 90% sequence identity to aa769-aa1445 of SEQ ID NO: 16, wherein said sequence identities preferably are at least 95%, at least 98% or 100%. Preferably, said protein is a single chain protein.

In embodiment 43, the Factor VIII protein of any of embodiments 1-42 may be a single chain protein and comprises a heavy chain portion having at least 90% sequence identity to aa20-aa1667 of SEQ ID NO: 1 and a light chain portion having at least 90% sequence identity to aa1668-aa2351 of SEQ ID NO: 1, wherein said sequence identities optionally are at least 95%, at least 98% or 100%. In embodiment 44, the Factor VIII protein of any of embodiments 1-43 comprises one albumin binding domain between the heavy chain portion and the light chain portion and one albumin binding domain C-terminal to the light chain portion,

wherein the sequence has at least 70% sequence identity to SEQ ID NO: 47.

In embodiment 45, the Factor VIII protein of any of embodiments 13 and 15-44 is a single chain protein comprising at least two albumin binding domains between the heavy chain portion and the light chain portion and at least two albumin binding domain C-terminal to the light chain portion, wherein the protein has at least 80% sequence identity to any of SEQ ID NO: 48, 49 or 51. In embodiment 46, the Factor VIII protein of embodiment 45 has at least 80% sequence identity to SEQ ID NO: 48.

In embodiment 47, the Factor VIII protein of embodiment 46 comprises SEQ ID NO: 48. In embodiment 48, the Factor VIII protein of embodiment 46 comprises SEQ ID NO: 51.

In embodiment 49, the Factor VIII protein of any of embodiments 1-46 has a “b mutation”, i.e., a mutation of the amino acid corresponding to Y1699 to F at position 1699 and a mutation of the amino acid corresponding to Y1683 to F at position 1683 in wt Factor VIII protein of SEQ ID NO: 1.

In embodiment 50, the in vivo half-life of the Factor VIII protein of any of embodiments 1-49 in a human subject is prolonged by a factor of at least 1.2, preferably, by a factor of at least 1.5, optionally, at least 2 or at least 2.5 in comparison to a recombinant Factor VIII protein of SEQ ID NO: 28.

In embodiment 51, the Factor VIII protein of any of embodiments 1-50 is a recombinant protein, and, optionally, it is a fusion protein with at least one fusion partner selected from the group consisting of an Fc region, albumin, PAS polypeptides, HAP polypeptides, the C-terminal peptide of the beta subunit of chorionic gonadotropin, polyethylenglycol, and hydroxyethyl starch.

In embodiment 52, the Factor VIII protein of any of embodiments 1-51 is a de-immunized protein.

In embodiment 53, the Factor VIII protein of any of embodiments 1-46 and 49-52 comprises at least three amino acid substitutions at positions selected from the group consisting of Y748, L171, S507, N79, I80, I105, S112, L160, V184, N233, L235, V257, I265, N299, Y426, Y430, L505, F555, I610, N616, L706, N754, K1837, R1936, S2030, S2037, N2038, S2077, M2123, F2215, K2226, K2258, V2313, S2315, V2333 and Q2335;

wherein substitutions of N are independently selected from the group consisting of D, H, S and E; wherein substitution of I are independently selected from the group consisting of T and V; wherein substitutions of S are independently selected from the group consisting of A, N, G, T and E; wherein substitutions of L are independently selected from the group consisting of N, Q, F and S; wherein substitutions of V are independently selected from the group consisting of A and T; wherein substitutions of Y are independently selected from the group consisting of N, H and S; wherein substitutions of F are independently selected from the group consisting of H and S; wherein substitutions of K are independently selected from the group consisting of N, D, E, Q, S and T; wherein substitutions of R are independently selected from the group consisting of Q, H and S; wherein substitutions of M are selected from the group consisting of R, Q, K and T; and/or wherein substitutions of Q are selected from the group consisting of R, D, E, H and K;

wherein the positions are specified in relation to a full length human Factor VIII molecule of SEQ ID NO: 1;

and wherein the Factor VIII protein retains at least 50% coagulant activity, as determined in a chromogenic assay, compared to a Factor VIII protein consisting of SEQ ID NO: 60.

In embodiment 54, the Factor VIII protein of embodiment 53 is a fusion protein. In embodiment 55, the Factor VIII protein of any of embodiments 53-54 comprises at least three amino acid substitutions at positions selected from the group consisting of Y748, L171, S507, N79, I80, I105, S112, L160, V184, N233, L235, V257, I265, N299, Y426, Y430, L505, F555, I610, N616, L706, N754, K1837, R1936, S2030, S2037, N2038, S2077, M2123, F2215, K2226, K2258, V2313, S2315, V2333 and Q2335.

In embodiment 56, the Factor VIII protein of any of embodiments 53-55 comprises at least one amino acid substitution at a position selected from the group consisting of Y748, L171, S507, N79, I80, I105, S112, L160, V184, N233, L235, V257, I265, N299, Y426, Y430, L505, F555, I610, N616, I632, L706, N754, K1837, R1936, S2030, S2037, N2038, S2077, M2123, S2125, F2215, K2226, K2258, V2313, S2315, V2333 and Q2335;

wherein substitutions of N are independently selected from the group consisting of D, H, S and E; wherein substitution of I are independently selected from the group consisting of T and V; wherein substitutions of S are independently selected from the group consisting of A, N, G, T and E; wherein substitutions of L are independently selected from the group consisting of N, Q, F and S; wherein substitutions of V are independently selected from the group consisting of A and T; wherein substitutions of Y are independently selected from the group consisting of N, H and S; wherein substitutions of F are independently selected from the group consisting of H and S; wherein substitutions of K are independently selected from the group consisting of N, D, E, Q, S and T; wherein substitutions of R are independently selected from the group consisting of Q, H and S; wherein substitutions of M are selected from the group consisting of R, Q, K and T; and/or wherein substitutions of Q are selected from the group consisting of R, D, E, H and K;

wherein, if the mutation is at position S507, it is S507E, and if the mutation is at position N616, it is N616E, and if the mutation is at position F2215, it is F2215H;

wherein the positions are specified in relation to full length human Factor VIII molecule of SEQ ID NO: 1,

and wherein the recombinant Factor VIII protein retains at least 50% coagulant activity, as determined in a chromogenic assay, compared to a Factor VIII protein consisting of SEQ ID NO: 60. The protein may be a fusion protein.

In embodiment 57, the Factor VIII protein of any of embodiments 53-56 may e.g. comprise amino acid substitutions selected from the group consisting of Y748S, L171Q, S507E, N79S, 180T, I105V, S112T, L160S, V184A, N233D, L235F, V257A, 1265T, N299D, Y426H, Y430H, L505N, F555H, I610T, N616E, I632T, L706N, N754D, K1837E, R1936Q, S2030A, S2037G, N2038D, S2077G, M2123K, S2125G, F2215H, K2226Q, K2258Q, V2313A, S2315T, V2333A and Q2335H.

In embodiment 58, the Factor VIII protein of any of embodiments 53-57 may e.g. comprise 3-25 of said substitutions and the substitutions may be located within different immunogenic clusters.

In embodiment 59, the Factor VIII protein of any of embodiments 53-58 may e.g. comprise at least three amino acid substitutions at positions selected from the group consisting of Y748, L171, S507, N79, S112, L160, V184, N233, I265, N299, Y426, F555, N616, I632, L706, K1837, R1936, N2038, S2077, S2125, F2215, K2226, K2258, S2315, and V2333;

wherein the at least three amino acid substitutions are preferably selected from the group consisting of Y748S, L171Q, S507E, N79S, S112T, L160S, V184A, N233D, I265T, N299D, Y426H, F555H, N616E, 1632T, L706N, K1837E, R1936Q, N2038D, S2077G, S2125G, F2215H, K2226Q, K2258Q, S2315T and V2333A.

In embodiment 60, the Factor VIII protein of any of embodiments 53-59 may e.g. comprise amino acid substitutions at least at positions
a. N79S, S112T, N233D, and I265T; and/or
b. N79S, S112T, L160S, L171Q, V184A, N233D, and I265T; and/or
c. N299D, Y426H, and S507E; and/or
d. F555H, N616E, L706N, Y748S; and/or
e. F555H, N616E, I632T, L706N, and Y748S; and/or
f. S2077G, S2315T, and V2333A; and/or
g. N2038D, S2077G, S2315T, and V2333A; and/or
h. S2077G, K2258Q, S2315T, and V2333A; and/or
i. N2038D, S2077G, K2258Q, S2315T, and V2333A; and/or
j. N2038D, S2077G, S2125G, K2258Q, S2315T, and V2333A; and/or
k. L171Q, S507E, Y748S and V2333A; and/or
l. L171Q, N299D, N616E and V2333A; and/or
m. S112T, S507E, Y748S, K1837E and N2038D; and/or
n. S112T, Y426H, N754D, K1837E and N2038D

preferably, combining at least the substitutions specified under b and c, optionally further including substitutions selected from those specified under d ore and/or f, g, h, l or j and/or K1837E.

In embodiment 61, the Factor VIII protein of any of embodiments 53-60 may e.g. comprise at least amino acid substitutions at positions N79, S112, L160, L171, V184, N233, I265, N299, Y426, S507, F555, N616, L706, and Y748, wherein preferably the substitutions are N79S, S112T, L160S, L171Q, V184A, N233D, I265T, N299D, Y426H, S507E, F555H, N616E, L706N, and Y748S. In embodiment 62, the Factor VIII protein of embodiment 61, further includes K1837E. Optionally, the protein comprises the amino acid sequence according to aa 20-1533 of SEQ ID NO: 119.

In embodiment 63, the Factor VIII protein of any of embodiments 53-62 may e.g. comprise at least amino acid substitutions at positions N79, S112, L160, L171, V184, N233, I265, N299, Y426, S507, F555, N616, L706, Y748, N2038, S2077, S2315 and V2333, wherein preferably the 18 substitutions are N79S, S112T, L160S, L171Q, V184A, N233D, 1265T, N299D, Y426H, S507E, F555H, N616E, L706N, Y748S, N2038D, S2077G, S2315T and V2333A. In embodiment 64, the Factor VIII protein of any of embodiments 53-63 comprises an amino acid sequence having at least 90%, preferably, 95% sequence identity to aa 20-1533 of SEQ ID NO: 114.

In embodiment 65, the Factor VIII protein of any of embodiments 53-64 may e.g. comprise at least the amino acid substitution at position K1837, wherein preferably said substitution is K1837E. In embodiment 66, the Factor VIII protein of any of embodiments 53-65 comprises the amino acid sequence according to aa 20-1533 of SEQ ID NO: 114.

In embodiment 67, the Factor VIII protein of any of embodiments 53-66 may have a reduced immunogenicity compared to a Factor VIII protein consisting of SEQ ID NO: 60 and preferably also compared to a Factor VIII protein consisting of SEQ ID NO: 61.

In embodiment 68, in the Factor VIII protein of any of embodiments 53-67, said immunogenicity is determined by an immunogenicity score or an assay comprising co-cultivating dendritic cells incubated with said protein and regulatory T-cell-depleted CD4⁺ T cells of a donor and testing activation of said T cells, preferably, by said assay.

In embodiment 69, the Factor VIII protein of any of embodiments 1-68 may e.g. have at least 90% sequence identity to a Factor VIII protein of SEQ ID NO: 63, wherein only the A1, a1, A2, a2, a3, A3, C1 and C2 domains are considered for determination of sequence identity. It may also be a fusion protein of said recombinant Factor VIII protein. In embodiment 70, the Factor VIII protein of embodiment 69 has SEQ ID NO: 114.

In embodiment 71, the invention provides a pharmaceutical composition comprising the Factor VIII protein of any of embodiments 1-70. In embodiment 72, the pharmaceutical composition of embodiment 71 is for use in treatment of hemophilia A, wherein the composition is preferably administered subcutaneously, e.g., in a dose of 5-1000 U/kg bodyweight.

In embodiment 73, the pharmaceutical composition of embodiment 71 or 72 further comprises human albumin, wherein, preferably, the concentration of human albumin is 0.1-15% (w/v). In embodiment 74, in the pharmaceutical composition of embodiment 73, the concentration of human albumin is 0.5-10% (w/v), optionally, 3-10% (w/v). The concentration of human albumin may also be about 1% (w/v).

In embodiment 75, the pharmaceutical composition of any of embodiments 71-74 further comprises a hyaluronidase, preferably, a human hyaluronidase such as vorhyaluronidase alfa. In embodiment 76, in the pharmaceutical composition of embodiment 75, the dose of the hyaluronidase is 50-300 U per injection.

In embodiment 77, the pharmaceutical of any of embodiments 71-76 comprises human albumin and a hyaluronidase.

In embodiment 78, the pharmaceutical composition of any of embodiments 71-77 is for human administration and is pharmaceutically acceptable. It may further comprise a pharmaceutically acceptable carrier, such as water or a buffer, optionally, at a physiologic pH, preferably, FVIII formulation buffer, and/or pharmaceutically acceptable excipients.

In embodiment 79, the invention provides a pharmaceutical composition of any of embodiments 71-78 or a kit comprising said composition, said composition or kit further comprising an immunosuppressive agent, e.g., an immunosuppressive agent selected from the group comprising methotrexate, methylprednisolone, prednisolone, dexamethason, cyclophosphamide, rituximab, and/or cyclosporin.

The invention further provides, as embodiment 80, a FVIII protein or pharmaceutical composition of any of embodiments 1-79 for use in treatment of hereditary hemophilia A. The invention further provides, as embodiment 81, a FVIII protein or pharmaceutical composition for use of any of embodiments 1-79 for use in treatment of acquired hemophilia A.

In embodiment 82, the invention provides a FVIII protein or pharmaceutical composition of any of embodiments 1-81, for use in treatment of hemophilia A, wherein the treatment is immune tolerance induction (ITI). In embodiment 83, the FVIII protein or pharmaceutical composition for use of any of embodiments 1-81 is for use in treating a patient with hemophilia A selected from the group comprising a patient not previously treated with any Factor VIII protein, a patient previously treated with a Factor VIII protein, a patient who has an antibody response including an inhibitory antibody response to a Factor VIII protein, and a patient who has had an antibody response including an inhibitory antibody response to a Factor VIII protein who has been treated by ITI, or who has not been treated by ITI.

In embodiment 84, the FVIII protein or pharmaceutical composition for use of any of embodiments 1-83 is for administration every 5 to 14 days, preferably, every 7 to 10 days.

In embodiment 85, the FVIII protein or pharmaceutical composition for use of any of embodiments 1-84 is to be administered in a dose of 10-700 U/kg bodyweight. In embodiment 86, the FVIII protein or pharmaceutical composition for use of any of embodiments 1-84 is to be administered in a dose of 50-500 U/kg bodyweight.

In embodiment 87, the invention provides a vial, e.g., a prefilled or ready-to use syringe, comprising the FVIII protein or pharmaceutical composition for use of any of embodiments 1-886.

In embodiment 88, the invention provides a method of treatment, comprising administering a dose of 1-1000 U/kg bodyweight of the FVIII protein or pharmaceutical composition of any of embodiments 1-87 to a patient in need thereof, e.g., a patient with hemophilia A, which may be selected from the patient groups defined herein.

In embodiment 89, the invention provides a kit comprising a hyaluronidase, e.g., a human hyaluronidase, and a pharmaceutical composition comprising a Factor VIII protein at least one albumin binding domain, wherein, optionally, the FVIII protein comprises a heavy chain portion and a light chain portion of Factor VIII, and the albumin binding domain(s) is/are C-terminal to the heavy chain portion and/or C-terminal to the light chain portion, wherein, if the protein is a single chain protein, the albumin binding domain(s) is/are between the heavy chain portion and the light chain portion and/or C-terminal to the light chain portion, wherein the FVIII protein or pharmaceutical composition preferably is the FVIII protein or pharmaceutical composition for use of any of embodiments 1-88.
SEQ ID NO: 1 wt human FVIII
SEQ ID NO: 2 processing sequence in preferred single chain constructs
SEQ ID NO: 4 processing sequence in VO
SEQ ID NO: 5 variant processing sequence, X can be varied for de-immunization
SEQ ID NO: 6 variant processing sequence, X can be varied for de-immunization
SEQ ID NO: 7 variant processing sequence, X can be varied for de-immunization
SEQ ID NO: 8 variant processing sequence, X can be varied for de-immunization
SEQ ID NO: 9 merging sequence, e.g., in VO
SEQ ID NO: 16 single chain FVIII V0 (AC_SC)
SEQ ID NO: 28 6rs-REF
SEQ ID NO: 39 Thrombin-cleavable linker
SEQ ID NO: 40 glycine-serine linker G1, Gin position 14 is present or absent
SEQ ID NO: 41 glycine-serine linker G2
SEQ ID NO: 42 Thrombin-cleavable linker flanked on each side by a glycine-serine linker, strictly repetitive
SEQ ID NO: 43 Thrombin-cleavable linker flanked on each side by a glycine-serine linker, non-repetitive
SEQ ID NO: 44 ABD consensus sequence, see above
SEQ ID NO: 45 ABD1
SEQ ID NO: 46 ABD2
SEQ ID NO: 47 ADLCLD_SC aa
SEQ ID NO: 48 AD2CD2_SC aa
SEQ ID NO: 49 AD2CD2woL_SC aa
SEQ ID NO: 50 AD2CD2woLG_SC aa
SEQ ID NO: 51 AbD2CD2_SC aa
SEQ ID NO: 52 ADLCLD_SC na
SEQ ID NO: 53 AD2CD2_SC na
SEQ ID NO: 54 AD2CD2woL_SC na
SEQ ID NO: 55 AD2CD2woLG_SC na
SEQ ID NO: 56 AbD2CD2_SC na
SEQ ID NO: 57 optimized DNA sequence encoding SEQ ID NO: 46
SEQ ID NO: 58 exemplary DNA encoding Glycine-serine linker G1 of SEQ ID NO: 40
SEQ ID NO: 59 exemplary DNA encoding Glycine-serine linker G2 of SEQ ID NO: 41
SEQ ID NO: 60 FVIII-6rs
SEQ ID NO: 61 ReFacto AF
SEQ ID NO: 62 B-domain deleted scFVIII
SEQ ID NO: 63 FVIII-19M
SEQ ID NO: 64 FVIII-18M
SEQ ID NO: 65 FVIII-15M
SEQ ID NO: 66 FVIII-A1-7M
SEQ ID NO: 67 FVIII-A2-4M
SEQ ID NO: 68 FVIII-BA3-1M
SEQ ID NO: 69 FVIII-A3C2-4M
SEQ ID NO: 70 FVIII-GOF1
SEQ ID NO: 71 FVIII-GOF2
SEQ ID NO: 72 FVIII-LS1
SEQ ID NO: 73
SEQ ID NO: 74-108+112 immunogenic clusters
SEQ ID NO: 109 FVIII-A1A2-3M
SEQ ID NO: 110 provides a nucleic acid sequence encoding FVIII-19M.
SEQ ID NO: 111 provides a nucleic acid sequence encoding FVIII-6rs.
SEQ ID NO: 113 Single Chain VO-19M (AC-19M_SC) aa
SEQ ID NO: 114 AD2CD2-19M_SC aa
SEQ ID NO: 115 ALDLCLD-19M_SC aa
SEQ ID NO: 116 ADLCLD-19M_SC-V1 aa
SEQ ID NO: 117 ADLCLD-19M_SC-V2 aa
SEQ ID NO: 118 AD2CD-19M_SC aa
SEQ ID NO: 119 AD2CD2-15M_SC aa
SEQ ID NO: 120 Single Chain VO-19M (AC-19M_SC) na
SEQ ID NO: 121 AD2CD2-19M_SC na
SEQ ID NO: 122 ALDLCLD-19M_SC na
SEQ ID NO: 123 ADLCLD-19M_SC-V1 na
SEQ ID NO: 124 ADLCLD-19M_SC-V2 na
SEQ ID NO: 125 AD2CD-19M_SC na
SEQ ID NO: 126 AD2CD2-15M_SC na
SEQ ID NO: 127 human vorhyaluronidase alfa

### FIGURE LEGENDS

FIG. 1 shows the human serum albumin (HSA) binding of ADLCLD_SC, a FVIII protein of the invention comprising two albumin-binding domains in comparison to FVIII 6rs-Ref, a protein having the ReFacto AF sequence. Both FVIII proteins were tested in the presence (dark bars) and absence (white bars) of HSA via the albumin binding capacity assay as described.

FIG. 2 shows the von-Willebrand factor (vWF) binding capacity of different FVIII-albumin-binding-domain fusion proteins in relation of ReFacto AF. All FVIII molecules were tested for their vWF binding in either the presence (dark bars) or absence (white bars) of human albumin. The more albumin-binding domains were incorporated into FVIII, the lower was the binding to vWF in general. The pre-incubation with human albumin dramatically decreased the binding to vWF.

FIG. 3 Comparison of unpurified FVIII-ABD fusion variants and FVIII controls for their in vitro functionality. Cell culture supernatants of CAP-T cells expressing the double chain FVIII molecule 6rs-REF, the single chain FVIII molecule AC_SC, and the FVIII-ABD fusion molecules AD2CD2_SC, AD2CD2woLG_SC, AD2CD2wL_SC, ACD4woLG_SC, and ACL(GD)₄_SC were analyzed for chromogenic FVIII activity (A), FVIII clotting activity induced by Actin FSL (B) and FVIII antigen levels indicating total FVIII protein amount (C). Specific chromogenic activity was calculated as chromogenic FVIII activity to FVIII antigen ratio displayed in % (D). Specific clotting activity was calculated as FVIII clotting activity to FVIII antigen ratio displayed in % (E). n=2.

FIG. 4 Western blot analysis of unpurified FVIII-ABD fusion variants and FVIII control proteins for assessing structural properties. Cell culture supernatants of CAP-T cells expressing the double chain FVIII molecule 6rs-REF, the single chain FVIII molecule AC_SC, and the FVIII-ABD fusion molecules AD2CD2_SC, AD2CD2woLG_SC, AD2CD2wL_SC, and ACD4woLG_SC were separated by non-reduced sodium dodecyl sulfate polyacrylamide gel electrophoresis and subsequent blotting onto a PVDF membrane was performed. A purified Sheep anti-Human Factor VIII primary antibody and CF680-conjugated donkey anti-sheep IgG (H&L) antibody was used for detection. For size determination, Precision Plus All Blue was applied as marker.

FIG. 5 demonstrates the in vivo pharmacokinetics of AD2CD2_SC compared to ReFacto AF after a single intravenous injection of 200 U/kg bodyweight FVIII (with 1% human albumin) into mice having a knock-out for murine albumin and expressing the a-chain of human instead of murine neonatal Fc-receptor. Determined FVIII antigen values were normalized and are shown in percent over time.

FIG. 6 demonstrates the in vivo pharmacokinetics of AD2CD2_SC compared to ReFacto AF after a single intravenous injection of 30 U FVIII: Antigen/kg bodyweight formulated with either 1 or 10% human albumin into Göttingen minipigs. Plasma samples were withdrawn and FVIII antigen levels were measured by ELISA. Mean FVIII antigen levels are shown in U/ml over time in hours. n=3 minipigs per group.

FIG. 7 shows the total bleeding time (first column of each group, left Y-axis) and the total blood loss (second column of each group, right Y-axis) after tail transection of hemophilia A mice which were administered 20 h earlier with either Vehicle Control, ReFacto AF, Eloctate, AD2CD2_SC or ADLCLD_SC. Non-hemophilia C57BL/6NCrl mice were treated with 0.9% NaCl and used as control.

FIG. 8: Comparison of an unpurified FVIII-ABD fusion variant with (AD2CD2-19M_SC) or without (AD2CD2_SC) 19 de-immunizing amino acid substitutions with a FVIII control in terms of protein expression and in vitro functionality. Cell culture supernatants of CAP-T cells expressing the double chain FVIII molecule 6rs-REF (ReFacto sequence), the FVIII-ABD fusion molecule AD2CD2_SC, and the de-immunized FVIII-ABD fusion molecule AD2CD2-19M_SC were analyzed for chromogenic FVIII activity (A), and FVIII antigen levels indicating total FVIII protein amount (B). Specific chromogenic activity was calculated as chromogenic FVIII activity to FVIII antigen ratio displayed in % (C). n=2.

FIG. 9 demonstrates the in vivo pharmacokinetics of AD2CD2-19M_SC compared to ReFacto AF after a single intravenous injection of 200 U FVIII/kg bodyweight into hemophilia A mice. FVIII antigen values and chromogenic FVIII activity were determined. FVIII antigen values are shown over time. The protein of the invention clearly has a longer half-life in vivo.

FIG. 10 demonstrates the in vivo pharmacokinetics of AD2CD2_SC and AD2CD2-19M_SC compared to ReFacto AF after a single intravenous injection of 30 U FVIII:Ag/kg bodyweight formulated with 10% human albumin into Göttingen minipigs. Plasma samples were withdrawn and FVIII antigen levels were measured. Mean FVIII antigen levels are shown in U/mL over time in hours. n=3 minipigs per group. The proteins of the invention clearly have a longer half-life in vivo.

FIG. 11 shows the total bleeding time after tail vein transection of hemophilia A mice which were administered 30 min earlier with either Vehicle Control (group 6) or different doses (groups 1 to 5) of AD2CD2-19M_SC (200 (group 1), 70 (group 2), 20 (group 3), 7 (group 4) or 2 (group 5) U FVIII/kg bodyweight) intravenously. In addition, non-hemophilia C57BL/6NCrl mice were treated with Vehicle Control (group 7). N=10 mice per group.

FIG. 12 shows the inhibitory potential of five anti-FVIII antibodies (ESH-8, GMA-8009, GMA-8015, GMA-8026, CL20035AP) against standard human plasma (SHP), ReFacto AF, AD2CD2_SC, and AD2CD2-19M_SC.

FIG. 13 Subcutaneous administration of the recombinant FVIII molecule AD2CD2_SC in comparison to ReFacto AF® in a PK study using hemophilia A mice, example 1.1. Squares with continuous line: FVIII-ABD plus Hylenex (Halozyme Therapeutics, Inc., San Diego, US) (Group 2); triangles with point-point-streak line: FVIII-ABD plus 1% albumin (Group 3); circles with dashed line: ReFacto AF® plus Hylenex (Group 1).

FIG. 14 Subcutaneous administration of the recombinant FVIII molecule AD2CD2_SC in comparison to ReFacto AF® in a PK study using hemophilia A mice, example, 1.2. Mean FVIII concentration is shown. Group 1 (Triangles: ReFacto AF®+Hylenex®; circles: AD2CD2_SC+Hylenex®). A: Chromogenic activity, B: Antigen

FIG. 15 Mean FVIII plasma concentrations (including the applied correction factor as described in the method section) based on FVIII:Ag measurements after intravenous injection of 30 U FVIII-Antigen/kg bodyweight into minipigs. Light triangles: AD2CD2-19M_SC+1% Albumin. Dark triangles: AD2CD2-19M_SC+10% Albumin. Light circles: AD2CD2_SC+1% Albumin. Dark circles: AD2CD2_SC+10% Albumin. Light squares: ReFacto AF®+1% Albumin. Dark squares: ReFacto AF®+10% Albumin.

FIG. 16 Mean FVIII plasma concentrations (including the applied correction factor as described in the method section) based on FVIII:Ag measurements after subcutaneous injection of 300 or 150 U FVIII-Antigen/kg bodyweight into minipigs. Circles: 300 U/kg AD2CD2-19M_SC+3% Albumin. Triangles: 150 U/kg AD2CD2-19M_SC+1% Albumin+Hylenex®. Squares: 300 U/kg AD2CD2-19M_SC+1% Albumin+Hylenex®. Rhombus: 300 U/kg ReFActo AF®+1% Albumin+Hylenex®.

### EXAMPLES

1. Evaluation of Factor VIII Proteins Comprising Albumin-Binding Domains for Subcutaneous Treatment of Hemophilia A.

Material and Methods

Preparation of Constructs

Experiments were performed to find and develop a suitable backbone for integration of the albumin-binding domains. The experiments were done on the basis of a B-domain deleted version of FVIII and single chain variants of FVIII. The basic double chain construct was a codon-optimized sequence of ReFacto AF® (Pfizer), wherein for simplifying cloning, 6 restriction sites were added through silent mutations, but some of these restriction sites were again excluded due to codon-optimization. The basic double chain sequence is 6rs-REF (SEQ ID NO: 28). The basic single chain construct used was VO (SEQ ID NO: 16, EP19173440).

Firstly, the ABD protein sequence (Affibody AB, Solna, Sweden) was taken as a basis for design of the DNA sequence. If not mentioned otherwise, the ABD2 sequence was used. Moreover, codon optimized linkers were developed, which are partly cleavable by thrombin. If not otherwise stated, the glycine-serine linker was G1 and the thrombin-cleavable linker was L. Table 1 below demonstrates structures of fusion proteins with albumin-binding domains (ABD) for single chain molecules.

For the constructs encoding the FVIII of the invention and comparative constructs also analysed in this context, either the complete FVIII sequence or DNA regions carrying approx. 700-1200 bp from the FVIII a2 domain to the A3 domain were synthesized. The synthesized DNA was codon-optimized for the total target gene. The a2 to A3 DNA fragments were 5′ terminally flanked by an EcoRV restrictions site, and 3′ terminally flanked by an EcoRI restriction site, and these restriction sites were also present in the basic FVIII sequence used. For C-terminal fusion to the light chain, DNA fragments carrying approx. 1500-2100 bp were synthesized also in a codon-optimized form. Such DNA fragments were 5′ terminally flanked by an EcoRI restrictions site within the A3 domain, and 3′ terminally flanked by a NotI restriction site. Restriction of the DNA inserts and the FVIII backbone plasmid allowed for targeted ligation and generation of FVIII single chain plasmids. Completely synthesized FVIII DNA was 5′ terminally flanked by a HindIII restrictions site, and 3′ terminally flanked by a NotI restriction site.

By transformation of E. coli K12 with said plasmids, expansion of transformed bacteria under ampicillin selection and plasmid preparation, large amounts of the plasmids could be prepared. Genetic engineering work was carried out by Thermo Fisher Scientific after design with VectorNTI Software (Thermo Fisher Scientific, Massachusetts, USA).

Cultivation of CAP-T Cells

For analyzing the candidates for new recombinant FVIII molecules, the constructs, integrated in expression vectors, were transiently and stably expressed in human cell lines. The preferred cell lines are Hek293 and CAP cells, both of which originate from human amniocytes. Because of higher yields of active FVIII molecules CAP cells, in particular, CAP-T cells were chosen as the preferred expression system for transient transfection and CAP-Go cells for stable expression.

Transient transfection was performed with nucleofection programs. The supernatants were screened for FVIII activity and antigen. Purification of the recombinant proteins from CAP cells was done, including FVIII affinity chromatography.

In detail, CAP-T cells (Cevec Pharmaceuticals, Köln, Germany) were cultured in PEM medium supplemented with 4 mM GlutaMAX (Thermo Fisher Scientific, 35050038) and 5 μg/mL blasticidin (Thermo Fisher Scientific, R21001; complete PEM medium). In order to thaw the cells, the required amount of frozen vials were transferred to a 37° C. water bath. After thawing, each vial was transferred to 10 ml of chilled, complete PEM medium. The cell suspension was centrifuged at 150×g for 5 minutes. During this washing step the dimethyl sulfoxide (DMSO) used for cryopreservation was removed. The pellet was resuspended in 15 mL warm, complete PEM medium and transferred to a 125 mL shaker flask. The cells were incubated at 37° C. in a humidified incubator with an atmosphere containing 5% CO₂. The flasks were set on a shaking platform, rotating at 185 rpm with an orbit of 50 mm.

Subculturing of the cells was performed every 3 to 4 days. The fresh culture was set to 0.5×10⁶ cells/ml by transferring the required amount of cultured cell suspension to a new flask and adding complete PEM medium. In the case that the transferred cell suspension would exceed 20% of the total volume, the suspension was centrifuged at 150×g for 5 minutes and the pellet was resuspended in fresh complete PEM medium. The volume of cell suspension per shaking flask was 20% of the total flask volume.

A minimum of three subcultures were performed after thawing before transfection experiments were performed.

Protein Expression in CAP-T Cells by Transient Transfection

The CAP-T cells were transfected using the 4D-Nucleofector™ (Lonza, Basel, Switzerland). For each transfection 10×10⁶ CAP-T cells were centrifuged at 150×g for 5 minutes in 15 mL conical tubes. The cells were resuspended in 95 μL supplemented SE Buffer, taking into account the volume of the pellet and the volume of the plasmid solution. Afterwards, 5 μg of the respective plasmid were added to the cell suspension followed by gentle mixing. The solution was transferred to 100 μL Nucleocuvettes. The used transfection program was ED-100. After the transfection, the cells from one Nucleocuvette were transferred to 125 ml shaker flasks, containing 12.5 mL complete PEM medium. The cells were cultivated for 4 days as described above. At day 4 the cells were harvested by centrifugation at 150×g for 5 minutes. Larger protein amounts could be produced by combining 12.5 mL approaches as described above.

Supernatants were screened for FVIII activity and antigen directly after harvest.

The recombinant Factor VIII protein was further analyzed. FVIII activity was measured by chromogenic activity assay and clotting activity FSL assay. The antigen was estimated by FVIII antigen ELISA. As a further assay for biological activity, the cleavage of the recombinant proteins by thrombin was analyzed. Moreover, chain distribution and appearance was tested by Western Blots. Further, vWF-binding and albumin binding were tested.

Protein Expression in CAP go Cells by Stable Cell Pools

In order to produce large material amounts to conduct minipig studies, stable CAP-Go pools expressing either AD2CD2_SC or AD2CD2-19M_SC were generated at Cevec Pharmaceuticals GmbH (Cologne, Germany). Therefore, the FVIII coding sequences were cloned into CEVEC's pStbl-bsd-MCS(−) plasmid using SgrD1 and Not1 restriction sites. The fragment was subsequently separated by agarose gel electrophoresis, purified by gel filtration and cloned into the pStbl-bsd-MCS(−), which was previously cut with SgrD1 and Not1 and treated with Calf-Intestine-Phosphatase (CIP). Inserts and vector were ligated using T4-DNA ligase and transformed into chemically competent E. coli cells (XL2-Blue). The plasmid DNA was purified using the Maxi Kit from Machery-Nagel. The whole cloning processes as well as the plasmid purifications were performed in a TSE-free production process.

Prior to nucleofection, circular plasmids were linearized with Scal. Therefore, 20-40 μg plasmid DNA were incubated 5-8 h with 50-200 U of the respective enzyme at 37° C. Subsequently, the DNA was purified by phenol-chloroform-isoamyl alcohol extraction and phenol was washed away with chloroform-isoamyl alcohol. To purify the DNA by ethanol precipitation, the DNA solution was supplemented with 1/10 volume of 3M NaOAc, pH=5.2 and 2 volumes of ethanol and incubated overnight at −20° C. The DNA precipitate was pelleted by centrifugation (30 min, 13 000 rpm, 4° C.), washed with 70% ethanol, centrifuged again, air-dried, and resuspended in TE buffer. The quality of the linearized DNA was assured by a DNA agarose gel analysis.

For nucleofection, CAP-Go cells were counted by Cedex XS (Roche Applied Science, Innovatis) and viable cell density and viability were determined. For each nucleofection reaction 1×10⁷ cells were harvested by centrifugation (150×g for 5 min). The cells were resuspended in 100 μL complete nucleofector solution V (Lonza) and mixed with 5 μg linearized plasmid of the respective construct. The DNA/cell suspension was transferred into a cuvette and the nucleofection was performed using the X₀₀₁ program on the Nucleofector II (Lonza). After the pulse, cells were recovered by adding 500 μL prewarmed complete PEM medium (=supplemented with 4 mM L-alanyl-L-glutamine) to the cuvette and gently transferred into 11.5 mL complete PEM medium in a 125 mL shaking flask. The cuvette was washed once with 500 μL fresh medium to recover residual cells.

72 h post-nucleofection the cell number and cell viability of the transfected cells were determined. The cells were harvested by centrifugation and resuspended in 20 mL complete PEM medium containing 5 μg/mL basticidin as selection marker. The cells were cultured at 37° C., 5% CO₂ at 185 rpm with 5 cm amplitude in a Kühner shaking incubator. As soon as cells recovered from selection and could be expanded, cells from the stable pools were cryopreserved.

For batch production, the culture was inoculated at a viable cell density of 1×10⁶ cells/mL in either 800 mL complete PEM medium in a 2 L shake flask or, for larger productions runs, 4×2500 mL complete PEM medium each in a 5 L shake flask. The cells were incubated at 185 rpm (5 cm orbit), 37° C., 5% CO2 in a Kühner shaking incubator for 4 days. The cell supernatants containing FVIII were harvested by centrifugation and purified by affinity chromatography as described elsewhere in this document.

Protein Purification

FVIII-6rs and FVIII-19M was produced in CAP-T cells in up to 800 mL scales. Purification occurred directly from the cell culture supernatant by FPLC. The first step was either a tangential flow filtration or an ion exchange chromatography, using the strong anion exchange columns HiTrap Capto Q (GE Healthcare Europe GmbH, Freiburg). In this step the sample was concentrated, host cell proteins were lost and the buffer was exchanged. The fractions containing the eluted protein were determined according to the chromatogram. The second step was an affinity chromatography, using a column packed with the commercially available VIIISelect resin (GE Healthcare Europe GmbH, Freiburg). The fractions containing the eluted FVIII were determined according to the chromatogram. The last step was a buffer exchange to FVIII Formulation Buffer by size exclusion chromatography, using the HiTrap Desalting columns (GE Healthcare Europe GmbH, Freiburg). The fractions containing FVIII were determined according to a high UV peak and a stable conductivity peak in the chromatogram. After purification, the FVIII products were concentrated via spin columns (Merck Millipore, Darmstadt) with a molecular weight cut-off of 10 kDa. All columns were run under the conditions specified by the manufacturer.

FVIII Activity—Chromogenic Activity Assay

The activity of FVIII was determined by a chromogenic assay. In this two-step assay, FIXa and FVIIIa activate FX in the first step. In the second step, the activated FX hydrolyses a chromogenic substrate, resulting in a color change, which can be measured at 405 nm. Due to the fact that calcium and phospholipids are present in optimal amounts and an excess of FIXa and FX is available, the activation rate of FX is only dependent on the amount of active FVIII in the sample.

The reagents for this chromogenic FVIII activity assay were taken from the Coatest® SP FVIII Kit. The kit contained phospholipids, calcium chloride (CaCl₂), trace amounts of thrombin, the substrate S-2765, a mixture of FIXa and FX and the thrombin inhibitor I-2581. The inhibitor was added in order to prevent hydrolysis of the substrate by thrombin, which was built during the reaction. All dilutions were performed in distilled water or Tris-BSA (TBSA) Buffer, containing 25 mM Tris, 150 mM sodium chloride (NaCl) and 1% Bovine serum albumin (BSA), set to pH 7.4. Each sample was diluted at least 1:2 with FVIII-depleted plasma. Further dilutions were performed using the TBSA Buffer.

The assay was performed using the BCS XP (Siemens Healthcare, Erlangen, Germany), a fully automated hemostasis analyzer. All reagents including water, TBSA Buffer and the samples were inserted into the analyzer. For each sample the analyzer mixed 34 μL calcium chloride, 20 μL TBSA Buffer, 10 μL sample, 40 μL water, 11 μL phospholipids and 56 μL FIXa-FX-mixture. This mixture was incubated for 300 seconds. Afterwards, 50 μL of S-2765+I-2581 were added to the reaction. Upon addition of the substrate, the absorption at 405 nm was measured for 200 seconds.

In order to calculate the amount of active FVIII, the software of the analyzer evaluated the slope of the measured kinetic between 30 seconds and 190 seconds after starting the reaction. This result was correlated to a calibration curve, generated with a biological reference preparation (BRP) of FVIII. The activity of the BRP is indicated in IU/mL. However, IU/mL can be assumed equivalent to U/mL. The results were indicated as “% of normal”. These results were converted to U/mL, as 100% of normal FVIII activity are equivalent to 1 U FVIII activity per mL.

Clotting Activity FSL

In addition to the two-stage chromogenic assay (see above), a one-stage clotting assay was also performed in order to determine the amount of active FVIII. During this assay, FVIII-depleted plasma, CaCl₂, the activator Actin FSL and the FVIII-containing sample are mixed in one step. The activator leads to the generation of FXIa, which activates FIX. FVIIIa, FIXa and FX built the tenase complex and FX becomes activated. Further activation of prothrombin and fibrinogen finally leads to the formation of a fibrin clot. The time needed to form the clot, the activated partial thromboplastin time (aPTT), is measured. The aPTT varies, depending on the amount of FVIII.

The clotting assay was performed using the BCS XP. TBSA Buffer, FVIII-depleted plasma, Actin FSL, CaCl₂) and the sample were inserted into the analyzer. The sample was diluted at least 1:2 with FVIII-depleted plasma. Further dilutions were performed using the TBSA Buffer. For each sample the analyzer mixed 45 μL TBSA Buffer, 5 μL sample, 50 μL FVIII-depleted plasma and 50 μL Actin FSL. The reaction was started by the addition of 50 μL CaCl₂. The analyzer measured the time needed for clot formation.

In order to calculate the amount of active FVIII, the software of the analyzer evaluated a baseline extinction at 405 nm at the beginning of the reaction. All of the following extinction values, within a time of 200 seconds, were analysed regarding their difference to the baseline extinction. The first time point exceeding a defined threshold was determined as the clotting time. This result was correlated to a calibration curve, generated with a BRP of FVIII.

Thrombin Generation Assay (TGA)

In the Thrombin Generation Assay (TGA), the amount of generated thrombin is measured. The clotting cascade takes place, started via the extrinsic pathway by tissue factor. The thrombin finally generated cleaves a fluorogenic substrate which can be measured at 460 nm. The assay was performed with FVIII diluted in FVIII-deficient plasma. FVIII concentrations up to 0.25 U/ml were analyzed. TGA reagent C low and TGA substrate, both commercially available by Technoclone (Vienna), were added to each sample well referring to the manufacturer's protocol. TGA reagent low consist of low concentrations of phospholipid micelles containing recombinant human tissue factor, in order to initiate the clotting cascade. The substrate is the fluorogenic substrate finally cleaved by the generated thrombin. The reaction was performed at 37° C. in a plate reader and the development of the fluorogenic substrate was measured for two hours. In addition to the samples, a calibration curve was measured using the TGA Cal Set, also available by Technoclone (Vienna). The amount of generated thrombin was calculated based on the calibration curve. Additionally, the area under the curve and the time to maximum thrombin generation was calculated based on the first deviation of the generated curve.

FVIII Antigen ELISA

The amount of human FVIII antigen was determined using the Asserachrom® VIII:Ag ELISA (Diagnostica Stago, Asnières sur Seine Cedex, France). In this sandwich ELISA, the applied FVIII is bound by mouse monoclonal anti-human FVIII F(ab′)₂ fragments, which are coated to the plate by the manufacturer. The detection of the bound FVIII occurs via mouse monoclonal anti-human FVIII antibodies, which are coupled to a peroxidase. In the case that FVIII is present, the peroxidase-coupled antibody binds to FVIII and can be detected by the addition of a tetramethylbenzidine (TMB) solution. TMB turns from a clear to a blue-green solution upon reaction with peroxidase. After a short time, this reaction is stopped by the addition of sulfuric acid (H₂SO₄), which turns the solution yellow. The amount of bound FVIII correlates with the intensity of the yellow color, which can be measured at 450 nm. The final amounts of FVIII are calculated using a calibration curve generated by the measurement of at least five serial dilutions of a calibrator with a known antigen concentration.

The supplied calibrator and control were reconstituted with 500 μL of distilled water, 30 minutes before starting the ELISA. After this incubation time, the calibrator was diluted 1:10 in the supplied phosphate buffer. This represented the starting concentration. The calibrator was further serially diluted 1:2 up to a dilution of 1:64. As the concentration of the calibrator contained approximately 1 U/mL FVIII, depending on the batch, the starting concentration was equivalent to 0.1 U/mL FVIII whereas the last dilution contained approximately 0.0016 U/mL FVIII. The control was diluted 1:10 and 1:20 with the phosphate buffer. All samples were diluted with the phosphate buffer, depending on their previously determined activity (see above) with the aim to be in the middle of the calibration curve. After the dilution of FVIII samples, control and calibrator, 200 μL of each solution were applied per well in duplicates. In addition to that, two wells were filled with 200 μL of phosphate buffer as a blank control. The plate was incubated for 2 hours at room temperature covered with a film. During this time, the peroxidase-coupled anti-human FVIII antibodies were reconstituted with 8 ml phosphate buffer and incubated 30 minutes at room temperature. After the antigen immobilization, the wells were washed five times with the supplied washing solution, which was previously diluted 1:20 with distilled water. Immediately after the washing, 200 μL of the peroxidase-coupled anti-human FVIII antibodies were added to each well and incubated for 2 hours at room temperature covered by a film. Afterwards, the plate was washed five times as before. In order to reveal the amount of bound FVIII, 200 μL of TMB solution were added to each well and incubated for exactly 5 minutes at room temperature. This reaction was stopped by the addition of 50 μL 1 M H₂SO₄ to each well. After an incubation time of 15 minutes at room temperature, the absorbance of each well was measured at 450 nm using the POLARstar Omega plate reader (BMG LABTECH, Ortenberg, Germany).

The results of the ELISA were calculated using the MARS software (BMG Labtech). In a first step, all wells were blank corrected and the mean of the duplicates was calculated. Afterwards, a 4-parameter fit was applied, in order to calculate the concentrations from the calibration curve. According to this calibration curve the amount of FVIII antigen in each well was determined. In the last step, the values were corrected by the dilution factor, resulting in the FVIII antigen amount of each sample.

Adapted FVIII Antigen ELISA for Measuring Göttingen Minipig Samples

The supplied calibrator and control of the Asserachrom® VIII:Ag ELISA (Diagnostica Stago, Asnières sur Seine Cedex, France, Cat. No. 00280) were reconstituted with 500 μL of distilled water, 30 minutes before starting the ELISA. After this incubation time, the calibrator was diluted 1:5 (i.e. 1+4) in Göttingen minipig plasma resulting in the calibrator stock solution. Further, this calibrator stock solution was 6-times serially diluted 1:2 with minipig plasma. The calibrator stock solution as well as each serial dilution step was 1:2 diluted within the supplied phosphate buffer resulting in final calibrator concentrations of 96, 48, 24, 12, 6, 3, and 1.5 mU/mL. All samples and assay controls were diluted with minipig plasma, except for a last dilution step, which was performed 1:2 in the phosphate buffer. All dilutions aimed for the middle of the calibration curve. After the dilution of FVIII samples, controls, and calibrator, 100 μL of each solution were applied per well in duplicates (volume reduced by 50% in comparison to the manual). In addition, two wells were filled with 100 μL of phosphate buffer as a blank control. The plate was incubated for 2 hours at room temperature covered with a film. During this time, the peroxidase-coupled anti-human FVIII antibodies were reconstituted with 8 mL phosphate buffer and incubated 30 minutes at room temperature. After the antigen immobilization, the wells were washed five times with the supplied washing solution, which was previously diluted 1:20 with distilled water. Immediately after the washing, 200 μL of the peroxidase-coupled anti-human FVIII antibodies were added to each well and incubated for 2 hours at room temperature covered by a film. Afterwards, the plate was washed five times as before. In order to reveal the amount of bound FVIII, 200 μL of TMB solution were added to each well and incubated for exactly 5 minutes at room temperature. This reaction was stopped by the addition of 50 μL 1 M H₂SO₄ to each well. After an incubation time of 15 minutes at room temperature, the absorbance of each well was measured at 450 nm using the POLARstar Omega plate reader (BMG LABTECH, Ortenberg, Germany).

The results of the ELISA were calculated using the MARS software (BMG Labtech). In a first step, all wells were blank corrected and the mean of the duplicates was calculated. Afterwards, a 4-parameter fit was applied, in order to calculate the concentrations from the calibration curve. According to this calibration curve the amount of human FVIII antigen in each well was determined and the values were corrected by the dilution factor, resulting in the FVIII antigen amount of each sample. Since AD2CD2_SC and AD2CD2-19M_SC detection was reduced in the presence of albumin, a correction factor was determined by spiking the application solution into minipigs plasma for at least two or three different concentrations in the range of what was expected after i.v. administration in the plasma of model animals, e.g. 0.5 to 20 U/mL, herein 9.23 and 4.62 U/mL. A pre-correction factor for each test item group and each spiking concentration was calculated (=100/recovery in %). Correction factors for each test item group were calculated as mean of pre-correction factors of all spiking concentrations. The resulting correction factors were applied to calculate specific concentrations used for further pharmacokinetic evaluation.

Albumin Binding Capacity Assay

20% human serum albumin (HSA) was diluted 1:4000 in PBS. 96-well ELISA plates were filled with 100 μL/well with diluted HSA solution and coated during a 2 h incubation at 37° C. and 400 rpm on a thermoshaker. ELISA plates were washed 3-times with 300 μL/well washing buffer. Standard control and FVIII samples either with or without Albumin pre-incubation were diluted with Tris/NaCl pH 7.4 to a concentration of 0.5 U/mL chromogenic activity and 100 μL/well were added as 7-step 1:2 serial dilution. Incubation was performed for 1 h at 37° C. covered on a thermoshaker. In the meantime, FIXa and FX were resolved together in 10 mL aqua dest., substrate (S-2765 and I-2581) was solved in 12 mL aqua dest. After FVIII incubation, plates were washed again 3 times with 300 μL/well washing buffer. Phospholipides and the FIXa/FX solution were mixed 1:5 and subsequently 50 μL/well of this solution were added and incubated for 5 min at 37° C. Without any washing step 25 μL CaCl₂ was added to each well, followed by 5 min incubation at 37° C. Finally, 50 μL/well substrate were added and detection of activated FX-mediated substrate turnover was performed at 405 nm for 25 cycles followed by end point measurement using an ELISA reader.

vWF Binding Capacity Assay

1 U/mL of each FVIII molecule was either pre-incubated or not with 40 mg/mL albumin for 30 min at RT to promote ABD-albumin binding and an assay for determining the vWF binding capacity was performed as follows:

Plasma purified vWF (Biotest AG) was diluted with 0.9% NaCl solution to a concentration of 0.1 U/mL. Coating onto 96-well ELISA plates was done by transferring 100 μL of this solution to each well followed by an 2 h incubation at 37° C. and 400 rpm. The wells were washed 3 times with 300 μL of washing buffer (8 mM sodium phosphate, 2 mM potassium phosphate, 0.14M NaCl, 10 mM KCl, 0.05% Tween-20, pH 7.4). FVIII standard (commercial rFVIII without vWF) and samples were pre-diluted with dilution buffer (25 mM Tris, 150 mM NaCl, pH 7.4) to a concentration of 0.25 U/mL according to chromogenic activity and transferred as a 7-step, serial 1:2 dilution into each plate well (100 μL/well). Incubation was carried out for 1 h at 37° C. and 400 rpm. In the meantime, FIXa and FX were resolved together in 10 mL aqua dest., substrate (S-2765 and 1-2581) was solved in 12 mL aqua dest. After FVIII incubation, plates were washed again 3 times with 300 μL/well washing buffer. Phospholipides and the FIXa/FX solution were mixed 1:5 and subsequently 50 μL/well of this solution were added and incubated for 5 min at 37° C. Without any washing step 25 μL CaCl₂ was added to each well, followed by 5 min incubation at 37° C. Finally, 50 μL/well substrate were added and detection of activated FX-mediated substrate turnover was performed at 405 nm for 25 cycles followed by end point measurement using an ELISA reader.

Western Blot

Reducing Sodium Dodecyl Sulfate Polyacrylamide Gel Electrophoresis (SDS-PAGE)

Cell supernatants, cell lysates or purified material of FVIII variants were appropriately diluted with 1×NuPAGE LDS Sample Buffer (4×, Thermo Fisher Scientific, NP0007) and further diluted 1:2 with reducing sample buffer. Reducing sample buffer was produced by combining 2.5 parts of NuPAGE LDS Sample Buffer with 1 part of NuPAGE Sample Reducing Agent (10×, Thermo Fisher Scientific, NP0004). 20 μL of each sample were mixed with 20 μL of reducing sample buffer in a 1.5 mL vial and heated for 10 min at 70° C. using a thermoshaker (Eppendorf). A NuPAGE 4-12% Bis-Tris Protein Gel (Thermo Fisher Scientific) was inserted into the XCell SureLock Mini-Cell Electrophoresis System (Thermo Fisher Scientific) and inner and outer chambers were filled with 1×NuPAGE MOPS SDS Running buffer (Thermo Fisher Scientific, NP0001). 500 μL of NuPAGE Antioxidant (Thermo Fisher Scientific) was added to the inner chamber. 10 μL of the each prepared sample and 4 μL of Precision Plus Protein All Blue Standard (Bio-Rad, 161-0373) diluted 1/10 in 1×LDS Sample Buffer were loaded onto the gel. The sample separation was achieved by running the gel at a constant voltage of 200 V for 50-60 min.

Non-Reducing SDS-PAGE

20 μL of cell supernatants, cell lysates or purified material of FVIII variants were, either pre-diluted or not, appropriately diluted with 10 μL of NuPAGE LDS Sample Buffer (4×, Thermo Fisher Scientific, NP0007) and 10 μL aqua dest. Samples were heated for 10 min at 70° C. using a thermoshaker (Eppendorf). A NuPAGE™ 3-8% Tris-Acetate Protein Gel (Thermo Fisher Scientific) was inserted into the XCell SureLock Mini-Cell Electrophoresis System (Thermo Fisher Scientific) and inner and outer chambers were filled with 1×NuPAGE™ Tris-Acetate SDS Running Buffer (Thermo Fisher Scientific, LA0041). 10 μL of the each prepared sample and 4 μL of Precision Plus Protein All Blue Standard (Bio-Rad, 161-0373) diluted 1/10 in 1×LDS Sample Buffer were loaded onto the gel. The sample separation was achieved by running the gel at a constant voltage of 150 V for 55-70 min.

Western Blotting and Detection

To investigate the separated proteins by immunofluorescence detection, they were transferred onto an Odyssey nitrocellulose membrane (Li-Cor) or an Amersham Hybond Low Fluorescence 0.2 μm polyvinylidene fluoride (PVDF) membrane (GE Healthcare Life Sciences) by using the XCell II blot module (Thermo Fisher Scientific) for semi-wet protein transfer. The PVDF membrane was activated in methanol and then applied to the SDS gel, whereas the nitrocellulose membrane was directly applied to the SDS gel. The system was filled with NuPAGE transfer buffer (20×, Thermo Fisher Scientfic) according to the manufacturer's instructions. Protein blotting was performed for 1 h at 30 V. After protein transfer, the membrane was blocked over night at 4° C. in Odyssey blocking buffer (Li-Cor). Afterwards the membrane was incubated for 1 h at room temperature simultaneously with either a rabbit anti-coagulation factor VIII monoclonal antibody (Sino Biological, 13909-R226, 1:1000) and a mouse anti-human factor VIII monoclonal antibody (Merck, MAB038, 1:2500) or with 0.0004 μg/μL sheep anti-human factor VIII:C polyclonal antibody (Cedarlane, CL20035AP, 1.5000), each diluted in Odyssey Blocking buffer containing 0.05% Tween 20. After incubation, the membrane was washed 4-times for 5 min in 0.1% PBST. For detection of FVIII heavy and light chain the membrane was incubated with 0.067 μg/mL IRDye 800CW donkey anti-mouse (Li-Cor, 926-32212, 1:15000) and 0.067 μg/mL IRDye 680RD donkey anti-rabbit (Li-Cor, 926-68073, 1:15000) diluted in Odyssey blocking buffer containing 0.05% Tween 20 for 1 h at room temperature. Alternatively, the CF680 donkey anti-sheep IgG (H&L) antibody (Biotium, 20062-1) was used in a 1:5000 dilution in Odyssey blocking buffer for binding the respective primary antibody. Finally, the membrane was washed 4 times for 5 min in 0.1% PBST, 2 times for 5 min in PBS and rinsed in water. The membrane was visualized using the Licor Odyssey Imager.

Pharmacodynamic Studies

Coagulation factors were administered by a single intravenous tail vein injection into female haemophilia A mice with doses of up to 200 U/kg body weight or respective amounts of a control solution. After either 0.5, 4 or 20 h post dosing, a tail vein transection bleeding assay was performed as follows: The animals were anaesthetized with 5% isoflurane in 30% O2 and 70% N2O, and immediately placed in prone position on a heating pad at +37° C. Tail vein transection was performed as described by Johansen et al., 2016. Haemophilia 22(4):625-631.

Bleeding was monitored for 60 min and bleeding time was determined using a stop clock. Primary bleeding time was noted until first bleeding cessation. After the primary bleeding, the tail was put into a new centrifuge tube filled with pre-warmed saline. If the mouse was not bleeding at 15, 30 and 45 min post injury, the tail was lifted out of the saline and the wound was challenged by gently wiping it twice with a saline wetted gauze swab in the distal direction. Immediately after the challenge, the tail was re-submerged into the saline. The cumulative bleeding time of all following bleeds constitute the secondary bleeding time. The total bleeding time is defined as the sum of the primary and all secondary bleeding times.

Following the determination of bleeding time, the tubes were centrifuged at 4140 g at room temperature for 3 minutes. Apart from 1 mL, the supernatant was removed. The cell pellet was resuspended and hemoglobin content was determined by using a method similar to that described by Elm et al. (2012).

### Results and Discussion

Generation and prescreening of several different FVIII-ABD fusion proteins covering FVIII double chain and single chain constructs was very promising in initial experiments and similar in both single chain and double chain backbones.

The formation of single chain FVIII molecule was increased compared to double chain forms when ABD was fused in between heavy chain and light chain (data not shown).

The FVIII proteins further developed and produced shown herein are listed in Table 1.

TABLE 1
Structure of exemplary variants of FVIII with ABD fusion.
A = FVIII A1 + a1 + A2 + a2 + truncated B domain,
C = FVIII a3 (optionally truncated) + A3 + C1 + C2 domains,
L = Thrombin cleavable linker,
G = flexible glycine-serine linker 1 (G1), D = ABD2,

| Construct name | Structure |
|---|---|
| AD2CD2_SC | ALDGLGDLCLDGLGD_SC |
| ADLCLD_SC | ADLCLD_SC |
| AbD2CD2_SC | ALDGLGDLCLDGLGD_SC + |
| | Y1699F + Y1683F |
| AD2CD_SC | ALDGLGDLCLD_SC |
| ACLD_SC | ACLD_SC |
| ADLC_SC | ADLC_SC |
| AD2C_SC | AD2C_SC |
| ACLDGLGD_SC | ACLDGLGD_SC |
| AD2CD2woLG_SC | ADDCDD_SC |
| AD2CD2woL_SC | AGDGDGCGDGD_SC |
| ACD4woLG_SC | ACDDDD_SC |
| ACL(GD)4_SC | ACLGDGDGDGD_SC |

Six single chain FVIII-ABD fusion molecules were generated in silico and respective DNA constructs were tested for their expression in either HEK293 or CAP-T cells (cf. Table 2). As all of those FVIII-ABD variants were expressed, secreted and functional, based on results of the chromogenic FVIII activity measurement, all molecules were produced in midi-scale CAP-T cell culture and successfully purified in larger amounts as needed for further characterizations and PK (pharmacokinetic) analysis.

TABLE 2
FVIII-ABD fusion proteins analyzed in supernatants of
transfected HEK293 or CAP-T cells.

| | | | | Specific | |
|---|---|---|---|---|---|
| | Chromo- | | | chromo- | Expres- |
| | genic | Clotting | | genic | sion |
| | activity | activity | FVIII:Ag | activity | Cell |
| Construct | [U/mL] | [U/mL] | [U/mL] | [%] | line |
| ADLC_SC | 0.98 | 0.9 | 0.47 | 209 | HEK293 |
| ACLD_SC | 1.26 | 0.79 | 0.72 | 175 | HEK293 |
| ADLCLD_SC | 1 | 0.45 | n/a | n/a | HEK293 |
| AD2CD2_SC | 1.45 | 0.35 | 0.71 | 204 | CAP-T |
| AD2CD_SC | 1.3 | 0.5 | 0.48 | 271 | CAP-T |
| AD2C_SC | 2.0 | 1.0 | n/a | n/a | CAP-T |
| ADLC_SC | 0.9 | n/a | n/a | n/a | CAP-T |
| ADLCLD_SC | 1.9 | n/a | n/a | n/a | CAP-T |

(n/a: not available)

All six purified FVIII-ABD fusion variants were extensively characterized by several methods including determination of FVIII antigen and chromogenic activity, Actin FSL clotting, heavy and light chain detection by western blotting (WB), thrombin-cleavage analysis and binding to vWF and albumin. Table 3 gives an overview of produced FVIII-ABD variants in terms of chromogenic and clotting activity as well as antigen levels in the final solutions. Measurement of these values indicated that FVIII-ABD fusion proteins are still capable of their biological function: Bridging factor IXa and factor X leading thereby to the activation of the latter one. Comparison of the specific chromogenic activity (chromogenic activity/antigen*100) demonstrates that ADLC_SC and ReFacto AF® are similar (109% vs 104%). However, the specific chromogenic activities of all other FVIII-ABDs are much better, ranging from 130% to 206%.

Interestingly, the results indicate that increasing numbers of ABD motifs within one FVIII molecule decrease the clotting activity and also the capability of vWF binding. The decrease in clotting activity may be caused by the setup of the assay, which is strictly time-dependent. This may not mirror in vivo clotting activity.

TABLE 3
Measurements of chromogenic FVIII activity,
FSL clotting activity, and antigen levels. The
indicated specific activity was calculated by the
ratio of chromogenic activity and antigen.

| | chromogenic | FSL | | Specific |
|---|---|---|---|---|
| FVIII-ABD | activity | clotting | Antigen | activity |
| variant | [U/ml] | [U/ml] | [U/ml] | [%] |
| ADLC_SC | 147 | 101 | 135 | 109 |
| ACLD_SC | 150 | 100 | 101 | 149 |
| ADLCLD_SC | 128 | 80 | 62 | 206 |
| AD2CD_SC | 120 | 61 | 92 | 130 |
| AD2CD2_SC | 164 | 42 | 96 | 171 |
| AD2C_SC | 112 | 72 | 78 | 144 |
| ReFacto AF ® | 253 | 242 | 243 | 104 |
| (Control) | | | | |

Western blot was used to observe the heavy and light chain patterns of the different FVIII-ABD fusion molecules in comparison to ReFacto AF® (data not shown). The analysis demonstrated that, in contrast to ReFacto AF®, the FVIII-ABD variants were mostly expressed as single chain molecules.

Activation of FVIII-ABD variants was investigated by direct incubation with thrombin at 37° C. for 8 min and subsequent provision for reducing SDS-PAGE followed by western blotting. Band patterns of thrombin-activated or untreated FVIII-ABD molecules show that all FVIII-ABD molecules were activated by thrombin in a comparable manner as ReFacto AF® (data not shown).

Albumin binding of the ADLCLD_SC variant was tested by an assay in comparison to FVIII 6rs-Ref, demonstrating the capability of albumin binding (FIG. 1). An excess of unbound soluble albumin inhibited the binding to plate-bound albumin.

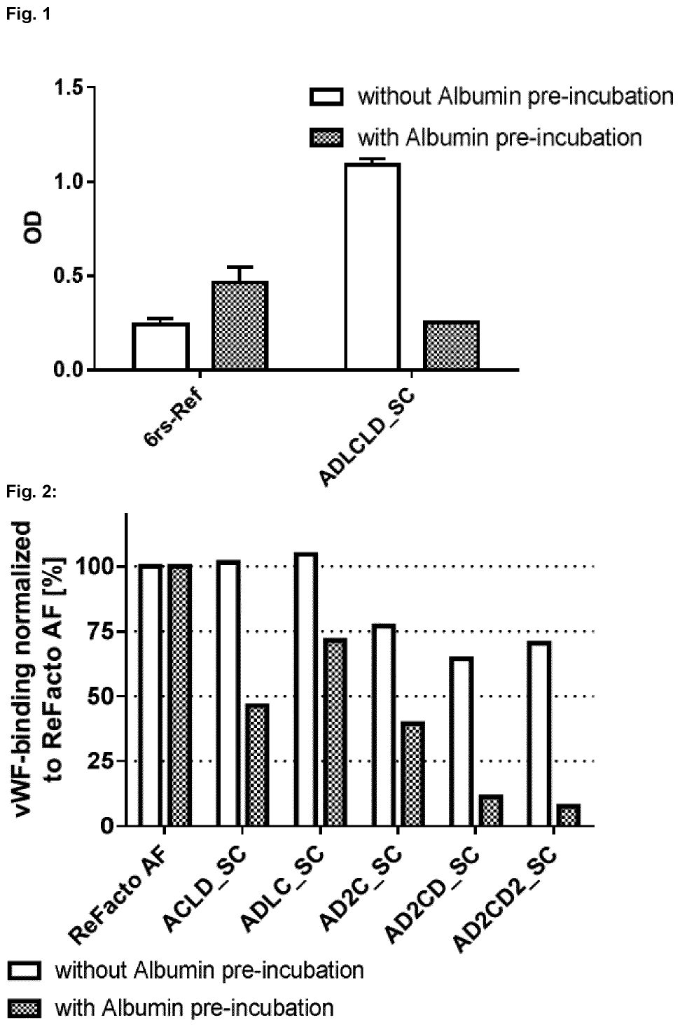

The influence of ABD and linker modifications on the binding between FVIII and vWF were investigated in two settings: 1. directly, without the presence of albumin; 2. after a 30 min pre-incubation with physiological concentrations of albumin promoting the ABD-albumin binding. As demonstrated in FIG. 2, the vWF binding of FVIII-ABD fusion proteins directly decreases by an increasing number of ABD motifs. However, only one ABD motif per FVIII does not have an influence on the vWF binding in the absence of albumin, independent from its position within the molecule. When FVIII-ABDs were pre-incubated with albumin, a decreased vWF binding was observed for all FVIII-ABD variants. This reduction of vWF binding was higher the more ABD domains were incorporated into FVIII.

To investigate the impact of linkers on the production and functionality of FVIII-ABD variants, one preferred variant, AD2CD2_SC, was also produced (I) without any linkers between FVIII and ABD-Domains (AD2CD2woLG_SC) and (II) with G1 linkers but without thrombin-cleavable L linkers (AD2CD2woL_SC). These variants were compared to the double chain FVIII 6rs-Ref (ReFacto amino acid sequence), single chain FVIII backbone AC_SC and two FVIII-ABD variants having four C-terminal ABD domains either without any linkers (ACD4woLG_SC) or with one thrombin-cleavable linker followed by four ABD domains separated by G1 linkers (ACL(GD)4_SC).

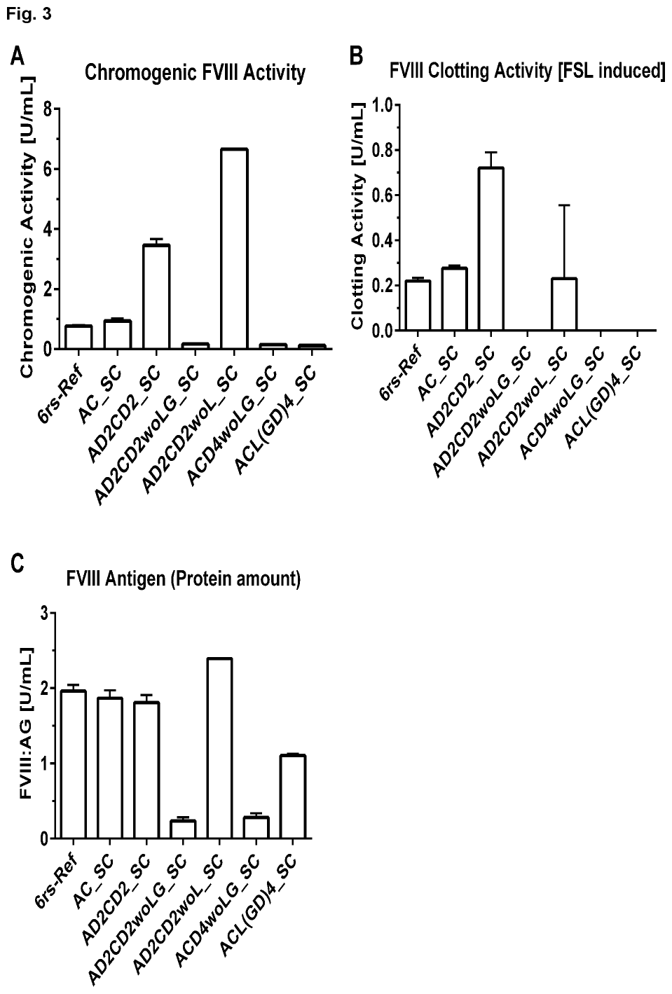

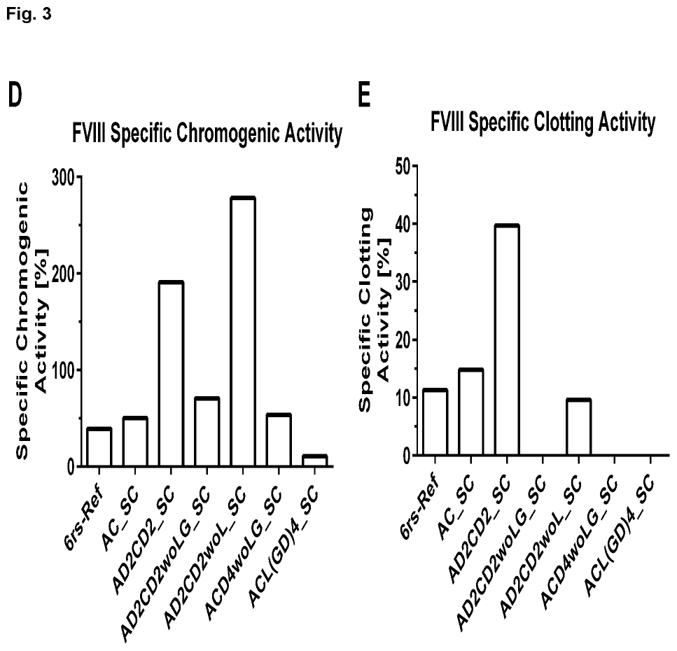

Respective plasmids encoding the different FVIII variants were nucleofected into CAP-T cells and 4-day cell culture supernatants were tested for chromogenic FVIII activity, FVIII clotting activity and FVIII antigen levels according to the above-described methods. As shown in FIG. 3, AD2CD2woLG_SC, ACD4woLG_SC, and ACL(GD)4_SC were expressed in only low amounts and chromogenic activity was strongly decreased. AD2CD2woLG_SC was not expressed in high amounts, but had some specific chromogenic activity. No FVIII clotting activity could be detected for any of these variants. In comparison to all other controls, AD2CD2_SC and AD2CD2woL_SC demonstrated good FVIII antigen levels and great FVIII chromogenic and clotting activities, resulting in superior specific chromogenic activity values of approx. 200% or higher. AD2CD2_SC demonstrate an especially high specific clotting activity.

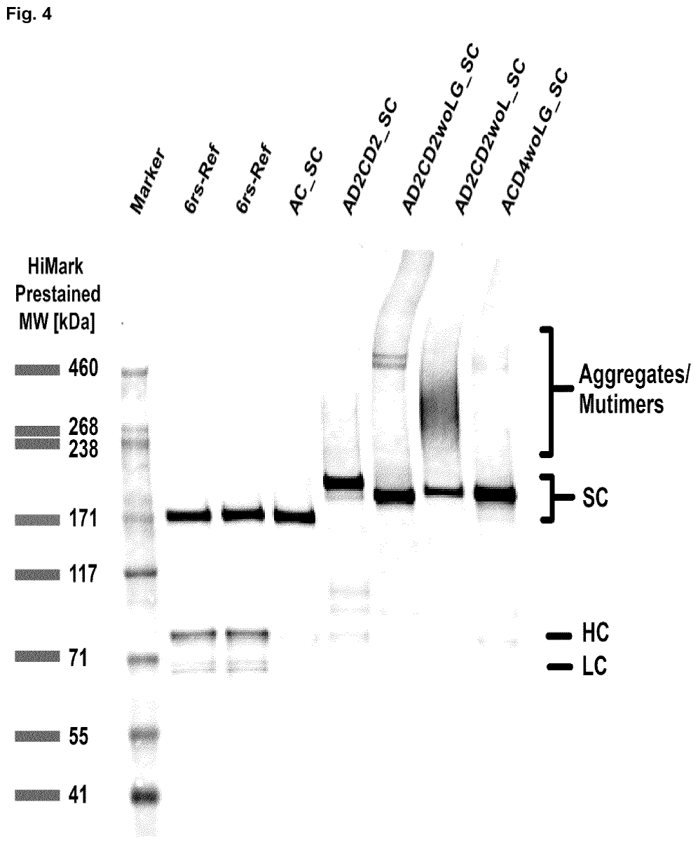

A western blot analysis based on a non-reduced SDS-PAGE separation of these variants is demonstrated in FIG. 4. Except for FVIII 6rs-Ref, all other variants are mainly present as single chain FVIII molecules. However, AD2CD2woLG_SC and AD2CD2woL_SC tend to form multimers or aggregates, which are not observed for the AD2CD2_SC variant.

3. Pharmacokinetic Experiments

Purification of FVIII ABD variants was performed for in vivo experiments, based on supernatants of transfected CAP-T cells, by strong anion exchange chromatography and affinity chromatography.

To investigate the half-life prolongation effect of ABD motifs introduced into the FVIII molecules, two pharmacokinetic (PK) studies were performed in hemophilia A mice. 12 mice per test item were used, 2 or 3 for each time point. All FVIII-ABD molecules were administered in a single dose of 200 U/kg body weight (6 ml/kg) into the tail vein by a single intravenous tail vein injection into female haemophilia A mice (B6, 12954-F8<tm1Kaz>/J). Plasma samples taken 0.5, 4, 8, 12, and 20 h (and 24 h) post injection were analyzed regarding FVIII chromogenic activity and antigen levels in citrate plasma which was subsequently extracted by centrifugation. Plasma samples were stored at −80° C. and analyzed for FVIII antigen and chromogenic activity. ReFacto AF® was tested as control beside the FVIII-ABD variants.

Results are shown in Table 4.

TABLE 4
Calculated t_{1/2} Of FVIII-ABD variants.

| | | |
|---|---|---|
| FVIII-ABD variant | Antigen: t_{1/2} | Chromogenic activity: t_{1/2} |

Study 1

| | | |
|---|---|---|
| ACLD_SC | 4.8 | 4.3 |
| AD2CD_SC | 4.3 | 5.0 |
| AD2CD2_SC | 14.5 | 9.4 |
| ReFacto ® (control) | 5.7 | 4.1 |

Study 2

| | | |
|---|---|---|
| ADLC_SC | 7.8 | 6.8 |
| ADLCLD_SC | 10.4 | 10.5 |
| AD2C_SC* | 13.0 | 6.1 |
| ReFacto ® (control) | 7.0 | 6.8 |

*AD2C_SC data were highly variable.
t_{1/2} is specified in hours

Thus, by intravenous pharmacokinetic studies in hemophilia A mice, preferred FVIII proteins of the invention were identified which show a half-life prolonged up to 2.5× (e.g., ADLCLD_SC—about 1.5×; AD2CD2_SC—about 2.5×). Pharmacokinetics of AbD2CD2_SC were tested in a separate study and were similar to AD2CD2_SC.

It is noted that the hemophilia A mouse model may even underestimate half-life extension due to the discrepancy of murine and human albumin (murine albumin only has a half-life of about two days). Nevertheless, the observed relative extended half-life of the FVIII proteins of the invention already allows a potential reduction of intravenous FVIII injection in hemophilia patients from 2-3 days to a once weekly dosing.

Moreover, a pharmacokinetic proof of concept study in albumin-deficient Tg32 mice having a knock-out of murine albumin and expressing human FcRn a-chain instead of the murine one (B6.Cg-Tg(FCGRT)32Dcr Alb^{em12Mvw} FCgrt^{tm1Dcr}/MvwJ, JAX Stock 025201) was performed. This mouse model (Alb⁻/mFcRn⁻/hFcRn⁺), compared to hemophilia A mice, reveals a situation closer to humans as injected human albumin has a half-life of approx. 20 days which is similar to the half-life in humans. Intravenous FVIII injection (AD2CD2_SC; ReFacto AF®) was done with 200 U/kg (based on chromogenic activity) plus 1% human albumin.

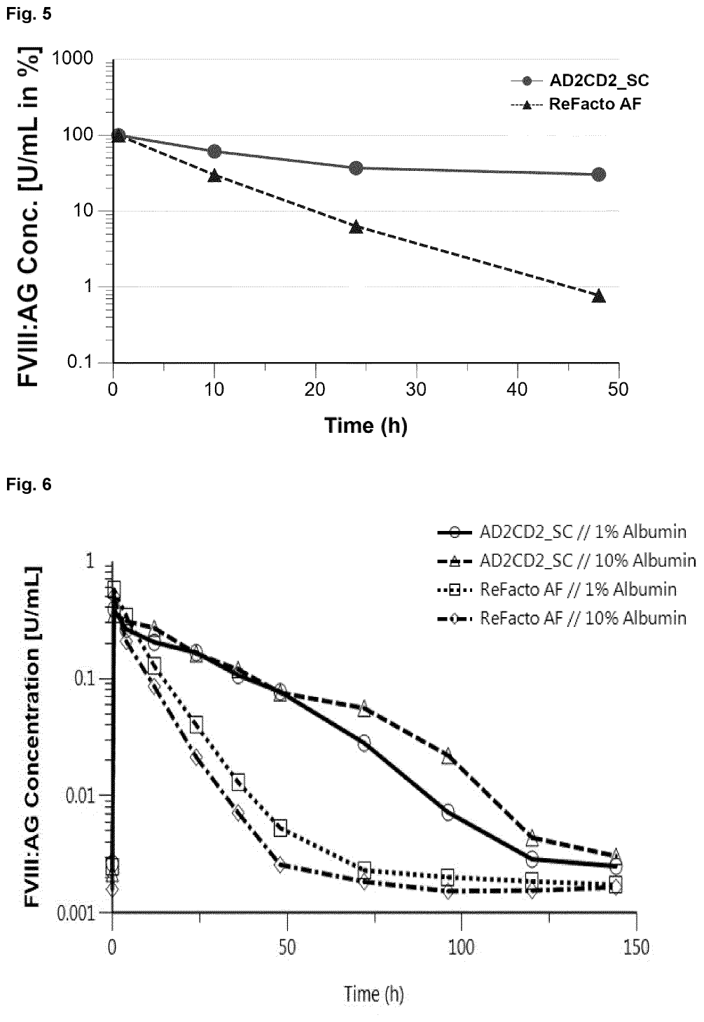

The results, shown in FIG. 5 demonstrated a half-life extension of AD2CD2_SC in comparison to ReFacto AF® of about 4×, allowing a potential reduction in patients of i.v. FVIII injection from 2-3 days to a 8-12 days dosing.

In addition, an intravenous pharmacokinetic study was performed in Göttingen Minipigs. Three animals per group were injected with 30 U FVIII antigen/kg body weight with either (I) ReFacto AF®+1% human serum Albumin (HSA), (II) ReFacto AF®+10% HSA, (Ill) AD2CD2_SC+1% HSA or (IV) AD2CD2_SC+10% HSA via the ear vein. Blood samples were taken predose, 4, 12, 36, 48, and 120 h post administration and citrate plasma was isolated immediately by centrifugation. Bioanalytical sample measurement was performed by FVIII antigen ELISA, which is specific for human FVIII and does not detect any porcine FVIII. Evaluation by non-compartment analysis (FIG. 6) obtained half-lives for (I) ReFacto AF®+1% HSA of 7.1 h, (II) ReFacto AF®+10% HSA of 6.4 h, (Ill) AD2CD2_SC+1% HSA of 18.6 h, and (IV) AD2CD2_SC+10% HSA of 20.7 h. Thus, an half-life extension of approx. 3-fold was observed for AD2CD2_SC compared to ReFacto AF® in this model.

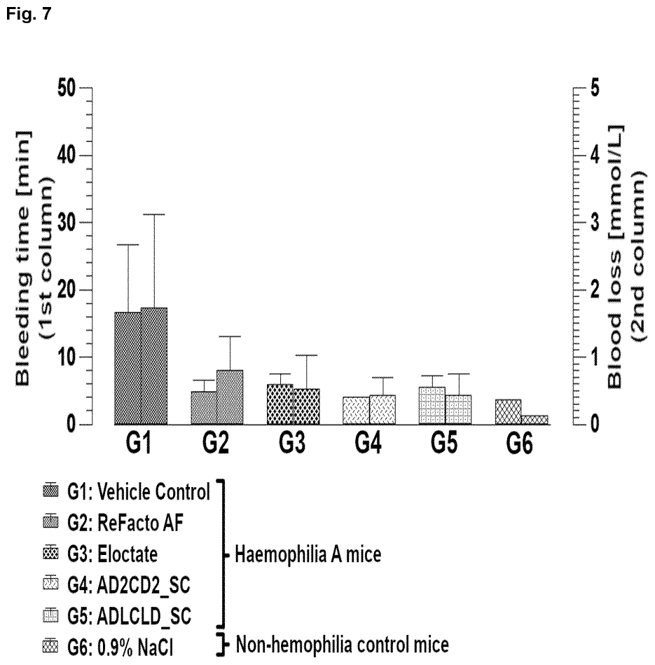

Moreover, pharmacodynamics studies have been performed. Hemophilia A mice (Jackson No. B6; 129S4-F8<tm1Kaz>/J) and control mice (Jackson No. C57BL/6NCrl) were intravenously injected with 200 U/kg (based on chromogenic FVIII activity) of each FVIII variant (ReFacto®, Eloctate®, AD2CD2_SC, ADLCLD_SC) or control solutions (Vehicle Control, 0.9% NaCl) and weight loss through bleeding, bleeding time and Hb amount by OD550 have been analyzed. Additional plasma sampling (0.5 h p.a. by retro-orbital withdraw, after experiment) have been done for analysis of FVIII activity. As shown in FIG. 7, all FVIII proteins of the invention diminish total bleeding time and blood loss similar to that of control mice indicating in vivo functionality of AD2CD2_SC and ADLCLD_SC.

4. De-Immmunized FVIII-ABD Proteins

The 19 de-immunizing amino acid substitutions of FVIII-19M were incorporated into the FVIII-ABD fusion molecules on the DNA level. The DNA sequences was generated in silico using VectorNTI (Thermo Fisher Scientific, Massachusetts, USA), and afterwards the full FVIII sequence was synthesized and cloned into the target vector. By transformation of E. coli K12 with said plasmids, expansion of transformed bacteria under ampicillin selection and plasmid preparation, large amounts of the plasmids could be prepared. Genetic engineering work was carried out by Thermo Fisher Scientific.

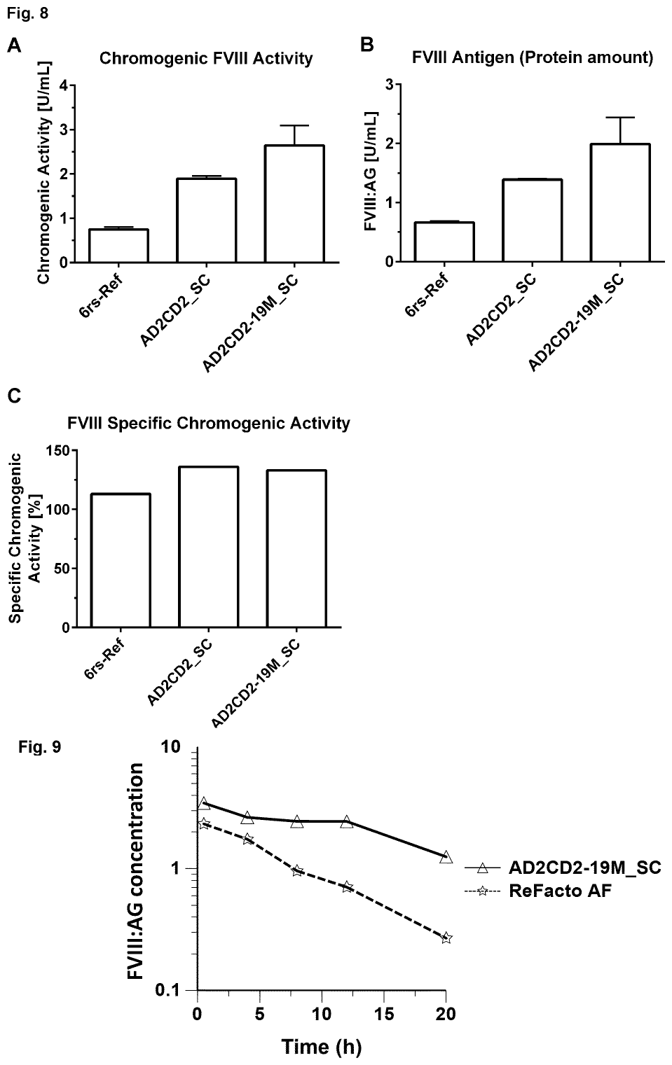

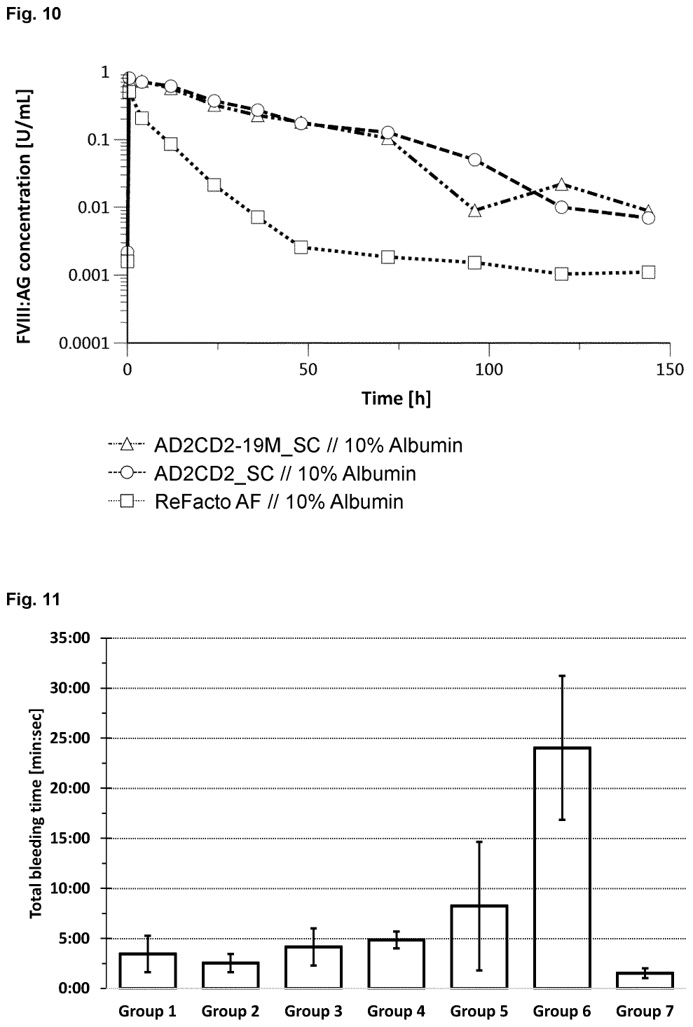

Cultivation of CAP-T cells and expression of the FVIII encoding plasmids by transient transfection was done as described elsewhere in this document. In order to verify expression levels and functionality of de-immunized, FVIII proteins fused with albumin-binding domains, plasmids encoding the de-immunized FVIII-ABD variant AD2CD2-19M_SC, and the fusion molecule AD2CD2_SC, both including 4 albumin-binding domains, and the FVIII control 6rs-Ref (ReFacto sequence) were nucleofected into CAP-T cells. 4-day cell culture supernatants were tested for chromogenic FVIII activity and FVIII antigen levels according to the above-described methods. As shown in FIG. 8, both FVIII chromogenic activity and FVIII antigen levels were at least 3-times higher for AD2CD2-19M_SC compared to 6rs-Ref. Interestingly, AD2CD2-19M_SC resulted in a better chromogenic activity and FVIII antigen levels compared to AD2CD2_SC (chromogenic activity: 2.64 vs 1.90 U/mL and FVIII antigen: 2.00 vs 1.40 U/mL, respectively). The specific chromogenic activity was 113% for 6rs-Ref, while AD2CD2_SC and AD2CD2-19M_SC resulted in 136% and 133%, respectively.

First in vivo pharmacokinetic experiments were performed in hemophilia A mice (B6, 129S4-F8<tmlKaz>/J) with affinity chromatography-purified FVIII material to investigate the half-life prolongation effect of AD2CD2-19M_SC. 12 mice per test item were used, 3 for each time point. AD2CD2-19M_SC and ReFacto AF (control) were administered in a single dose of 200 U/kg body weight (7.14 ml/kg) into the tail vein by a single intravenous tail vein injection into female mice. Blood samples were taken 0.5, 4, 8, 12, and 20 h post injection and citrate plasma was extracted by centrifugation. Plasma samples were stored at −80° C. and analyzed for FVIII antigen and chromogenic activity as described. For pharmacokinetic evaluation, a non-compartmental analysis was performed using Phoenix WinNonlin (Certara USA Inc., USA). Mean values of the FVIII antigen levels over time are shown in FIG. 9. For AD2CD2-19M_SC terminal half-lives of 12.45 h and 11.58 h were detected for chromogenic activity and FVIII antigen, respectively and based on the mean of individual animals. In comparison, Refacto AF® resulted in terminal half-lives of 6.48 h for chromogenic activity and 6.08 h for FVIII antigen. Thus, a half-life prolongation of approx. 2-times was verified in this model. An additional evaluation using the median instead of mean resulted in half-life extensions of approx. 3-times.

Furthermore, a pharmacokinetic study testing the AD2CD2-19M_SC molecule was performed in Göttingen Minipigs (Ellegaard, Dalmos, DK). Three animals per group were injected with 30 U FVIII antigen/kg bodyweight with either (I) ReFacto AF®+1% human serum Albumin (HSA), (II) ReFacto AF®+10% HSA, (Ill) AD2CD2_SC+1% HSA, (IV) AD2CD2_SC+10% HSA, (V) AD2CD2-19M_SC+1% HSA, (VI) AD2CD2-19M_SC+10% HSA via the ear vein. Blood samples were taken predose, 4, 12, 36, 48, and 120 h post administration and citrate plasma was isolated immediately by centrifugation. Bioanalytical sample measurement was performed by an adapted FVIII antigen ELISA as described above. Evaluation by non-compartment analysis (FIG. 24, showing only groups II, IV, and VI for clearity) obtained half-lives for (I) ReFacto AF®+1% HSA of 7.1 h, (II) ReFacto AF®+10% HSA of 6.4 h, (Ill) AD2CD2_SC+1% HSA of 18.6 h, (IV) AD2CD2_SC+10% HSA of 20.7 h, (V) AD2CD2-19M_SC+1% HSA of 19.2 h, and (VI) AD2CD2-19M_SC+10% HSA of 21.0 h. Thus, besides the half-life extension of approx. 3-fold for AD2CD2_SC over ReFacto AF® in this model, a similar or even higher half-life extension was observed for

AD2CD2-19M_SC was additionally tested for its in vivo functionality using a tail vein transection assay as described for pharmacodynamics studies. Hemophilia A mice (Jackson No. B6; 129S4-F8<tm1Kaz>/J) were intravenously injected with different doses of AD2CD2-19M_SC, covering 200 U/kg (group 1), 70 U/kg (group 2), 20 U/kg (group 3), 7 U/kg (group 4), and 2 U/kg (group 5) (all based on chromogenic FVIII activity) or formulation buffer (group 6) (n=10 mice per group). Non-hemophilia C57BL/6NCrl mice were used as control (group 7). The tail vein transection assay was performed 30 min post test item administration. Weight loss through bleeding, bleeding time and Hb amount by OD550 were analyzed as readouts. Additional plasma sampling (0.25 h p.a. by retro-orbital withdraw and after the experiment) have been done for analysis of FVIII activity. As shown in FIG. 11, AD2CD2-19M_SC reduced the total bleeding time to that of control mice in a dose concentration-dependent manner, clearly indicating its in vivo functionality.

In order to evaluate if de-immunized FVIII-ABD fusion proteins maintain a certain FVIII activity in the presence of inhibitory anti-FVIII antibodies originally raised against WT or B-domain truncated FVIII (bypassing activity), a modified Nijmegen-Bethesda assay was performed. The Bethesda assay is widely used to quantitate the concentration of a factor VIII inhibitor (inhibitory antibody). 1 Bethesda Unit (BU) is defined as the amount of an inhibitor that will neutralize 50% of 1 unit of FVIII activity in normal plasma after 120 minutes incubation at 37° C. Therefore, five different anti-FVIII antibodies (ESH-8, GMA-8009, GMA-8015, GMA-8026 and CL20035AP), all having inhibitory probabilities to human FVIII activity, were spiked 1:100 into imidazole buffer (Siemens Healthcare Diagnostics, Germany, #OQAA33), which served as stock solutions. Recombinant FVIII variants ReFacto AF®, AD2CD2_SC, and AD2CD2-19M_SC were spiked to a final concentration of 1 U/mL into FVIII-depleted plasma (Siemens Healthcare Diagnostics, Germany, #OTXW17). Standard human serum (Siemens Healthcare Diagnostics, Germany, #ORKL17) was reconstituted in imidazole buffer resulting in a FVIII activity of 1 U/mL serving as further control. Anti-FVIII antibody stocks were diluted 1:2 up to 1:1024 (1:2 serial dilutions) in FVIII-depleted plasma containing the FVIII products. Additionally, each FVIII product diluted 1:2 with FVIII-depleted plasma was determined as baseline FVIII activity (should result in approx. 0.5 U/mL). A FVIII-inhibitor plasma standard (Technoclone, Austria, #5159008, 16.0 BU/ml) diluted 1:2 to 1:128 (1:2 serial dilution series) with FVIII-depleted plasma was used as positive control. All samples were incubated for 2 h at 37° C. and the activity was determined by chromogenic FVIII activity measurements. The remaining FVIII activity within each samples was calculated by the following formula:

Chromogenic FVIII activity sample [U/mL]/chromogenic FVIII activity baseline [U/mL]*100

Subsequently, Bethesda units were calculated in remaining activity ranges of 25-75% using the following formula:

2−Log(remaining FVIII activity)/0.30103*dilution factor

Afterwards, the Bethesda units of each sample were divided by the Bethesda units of the positive control of each run for a more stringent comparison.

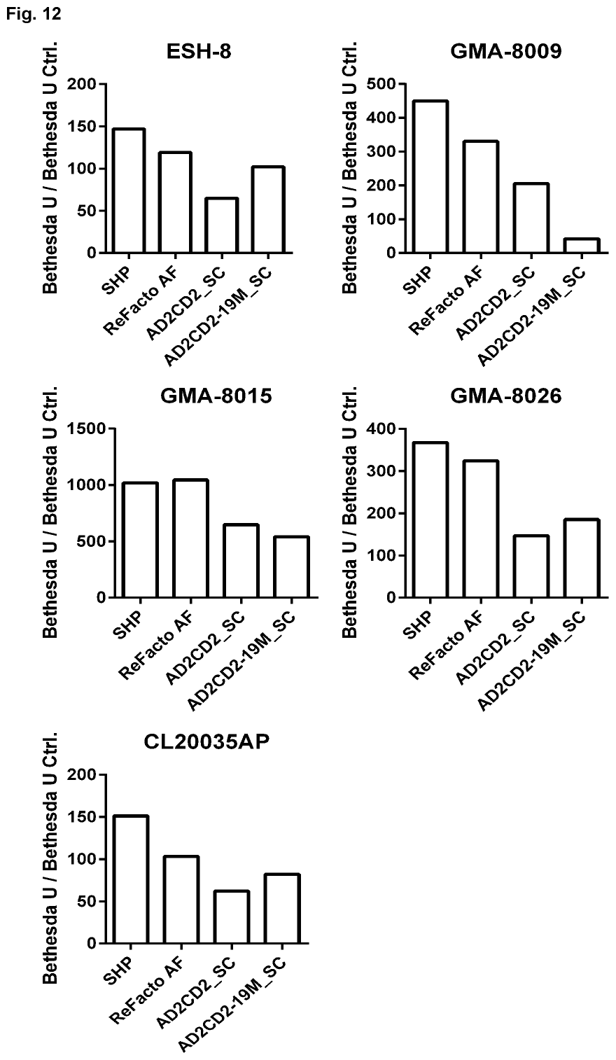

FVIII bypassing activity results of AD2CD2_SC and AD2CD2-19M_SC in comparison to a standard human plasma (SHP) and ReFacto AF® against the five inhibitory anti-FVIII antibodies ESH-8, GMA-8009, GMA-8015, GMA-8026, and CL20035AP are shown in FIG. 12. In general, highest FVIII inactivation was observed for SHP followed by ReFacto AF®. In contrast, the FVIII activity of AD2CD2_SC and AD2CD2-19M_SC was affected to a much lower extend by all anti-FVIII inhibitors. Interestingly, the anti-FVIII antibody GMA-8009 had a high inhibitor potential against SHP and ReFacto AF®, a medium inhibitory potential to AD2CD2_SC and only a marginally inhibitory potential against AD2CD2-19M_SC indicating the elimination of a B cell epitope by one of the introduced de-immunizing mutations.

5. Subcutaneous Administration

For a proof of concept study for subcutaneous administration, FVIII proteins incorporating at least one albumin binding domain were tested in hemophilia A-mice and in minipig in comparison to ReFacto® AF (Pfizer) which is one of the most common B-domain deleted FVIII products.

Because the albumin binding domains as integrated into the FVIII fusion protein, bind human albumin with a higher affinity compared to murine and porcine albumin (binding affinities are about 1:10 or 1:100), the FVIII protein incorporating at least one albumin binding domain was administered in the presence of human albumin. Co-administered albumin might also have additional effects of stabilization in terms of shielding the FVIII-ABD fusion polypeptide from cellular and enzymatic degradation, and increase the bioavailability by albumin-mediated transport pathways. Further, the compound hyaluronidase, which is known to increase availability especially in subcutaneous administration, was tested in combination with the FVIII with at least one albumin binding domain. The formulation of hyaluronidase as available already contains addition of human albumin (0.1%).

For all tests shown herein, the FVIII single chain construct AD2CD2_SC (38_ALDGLGDLCLDGLGD_SC, also designated AD2CD2, SEQ ID NO: 48) or, for the minipigs, single chain AD2CD2-19M_SC (SEQ ID NO: 114) was used, wherein two albumin binding domains sequences (D) are inserted between A2 and A3 domain. Two further are located at the C-terminus.

5.1 Pharmacokinetic Studies of Recombinant FVIII Molecule AD2CD2 SC by Subcutaneous Administration in Hemophilia a Mice

5.1.1 Subcutaneous administration of the recombinant FVIII molecule AD2CD2 in Comparison to ReFacto AF® in a PK Study Using Hemophilia a Mice

The aim of this study was to investigate the feasibility of subcutaneous (s.c.) administration of AD2CD2_SC in the presence of either 1% human albumin or Hylenex® (recombinant human hyaluronidase, vorhyaluronidase alfa). It was compared to the commercially available rFVIII product ReFacto AF® co-administered with Hylenex®. Coagulation factors were administered by a single s.c. bolus injection into the back region of female haemophilia A mice. Blood sampling was performed 1, 4, and 20 h post treatment and citrate plasma was subsequently extracted by centrifugation. Plasma samples were further analyzed for chromogenic FVIII activity.

Hemophilia A mice (B6; 129S-F8^{tm1Kaz}/J),
15 female animals (five per group)

| | |
|---|---|
| Test items | a) ReFacto AF ® |
| | b) AD2CD2_SC |
| | c) Hylenex ® |
| | d) HSA (Albiomin ®, Biotest AG, Dreieich) |
| | 20% (200 g/L) |
| Vehicle | FVIII Formulation Buffer |
| Route of administration | Subcutaneous bolus injection into the back |
| | region |
| Administration volume | 6.67 mL/kg b.w. |
| Injection speed | Dose/Approx. 15 sec |

Frequency of administration Single dosing on test day 1
Group 1: 400 U ReFacto®/kg b.w.+400 U Hylenex/kg b.w.
Group 2: 400 U AD2CD2_SC/kg b.w.+400 U Hylenex/kg b.w.
Group 3: 400 U AD2CD2_SC/kg b.w.+1% albumin (FVIII formulation buffer)

Hylenex® (Halozyme Therapeutics, Inc): human Hyaluronidase, 150 U/ml, 8.5 mg/ml sodium chloride, 1.4 mg/ml dibasic sodium phosphate, 1 mg/ml human albumin, 1.5 mg/ml L-methionine, 0.2 mg/ml polysorbate 80

Thus, the concentration of HSA in application solutions of group 1 and group 2 was 0.4 mg/ml, while the HSA concentration of group 3 was 10 mg/ml.

Tests showed that albumin and Hylenex® itself have no influence on chromogenic FVIII activity (incubation 60 min at room temperature).

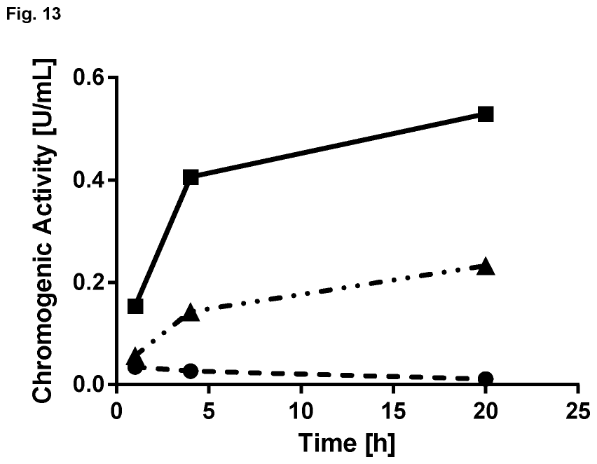

Results are shown in FIG. 13. During this study ReFacto AF® co-administered with Hylenex® did not reach relevant FVIII plasma levels after subcutaneous injection. Compared to ReFacto AF®, administration of AD2CD2_SC plus 1% albumin showed a good activity of about 0.2 U/ml after 20 h. Co-administration of AD2CD2_SC and Hylenex® showed even more promising results with an activity of about 0.5 U/ml after 20 h.

5.1.2 Subcutaneous Administration and Bioavailability of the Recombinant FVIII Molecule AD2CD2_SC in Comparison to ReFacto AF® in a PK Study Using Hemophilia A mice

The aim of this study was to investigate the bioavailability and pharmacokinetics of AD2CD2_SC in the presence of Hylenex® (recombinant human hyaluronidase) after subcutaneous (s.c.) administration. It was compared to the commercially available recombinant FVIII product ReFacto AF® co-administered with Hylenex®. This study extended the previously performed s.c. PK study, while lowering the administered dose to 200 U/kg b.w. comparable to previous i.v. injections. Coagulation factors were administered by a single s.c. bolus injection into the back region of female hemophilia A mice. Blood sampling was performed 4, 12, 24, 36, 48 and 60 h post treatment (using satellite mice) and citrate plasma was subsequently extracted by centrifugation. Plasma samples were further analyzed for chromogenic FVIII activity and FVIII antigen concentrations.

Hemophilia A mice (B6; 129S-F8^{tm1Kaz}/J), 20 female animals, 10 mice per group

| | |
|---|---|
| Test items | a) ReFacto AF ® |
| | b) AD2CD2_SC |
| | c) Hylenex ® |
| | d) HAS (Albiomin ®, Biotest AG, Dreieich) 20% |

The test items were diluted with FVIII formulation buffer to a final concentration of 33.33 U/mL. The administration volume was 6 mL/kg b.w.

Group 1: 200 U ReFacto AF®/kg b.w.+200 U Hylenex®/kg b.w.

Group 2: 200 U AD2CD2/kg b.w.+200 U Hylenex®/kg b.w.

Thus, the concentration of HSA in application solutions of each group was 0.333 mg/ml.

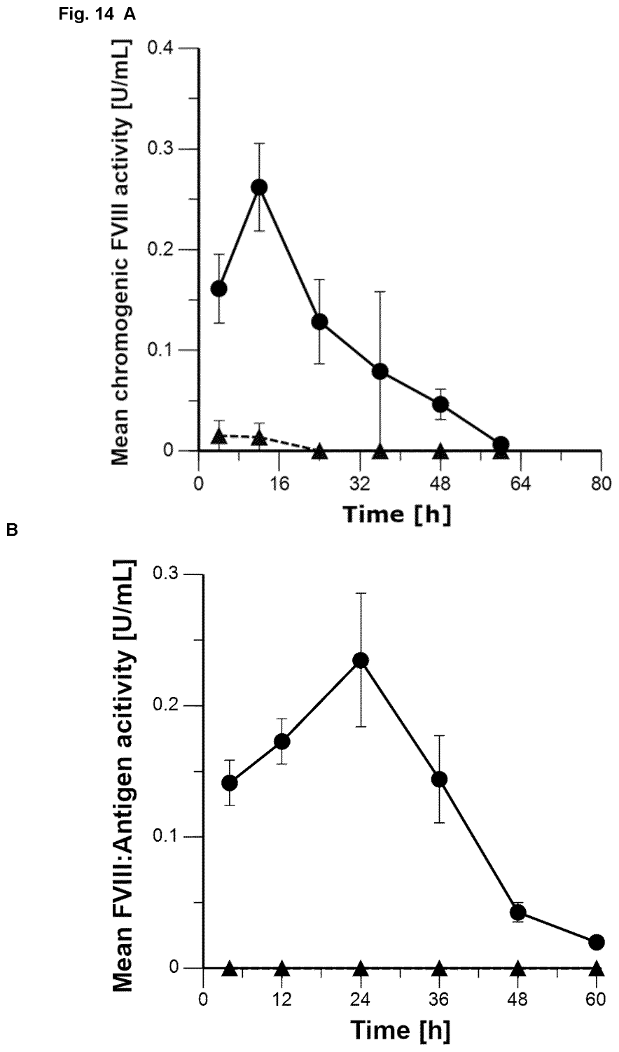

The mean results are shown in FIG. 14 by FVIII activity and FVIII antigen. Table A below shows the activity, Table B the antigen.

TABLE A

| | | Half-life | Tₘₐₓ | Cₘₐₓ | AUCₗₐₛₜ |
|---|---|---|---|---|---|
| | Test item | [h] | [h] | [U/mL] | [U/mL*h] |
| Mean | ReFacto AF ® + | — | 4.00 | 0.02 | 0.14 |
| | Hylenex ® | | | | |
| | AD2CD2_SC + | 8.73 | 12.00 | 0.26 | 6.67 |
| | Hylenex ® | | | | |
| Median | ReFacto AF ® + | — | — | 0.00 | 0.00 |
| | Hylenex ® | | | | |
| | AD2CD2_SC + | 22.76 | 12.00 | 0.26 | 6.13 |
| | Hylenex ® | | | | |

TABLE B

| | | Half-life | Tₘₐₓ | Cₘₐₓ | AUCₗₐₛₜ |
|---|---|---|---|---|---|
| | Test item | [h] | [h] | [U/mL] | [U/mL*h] |
| Mean | ReFacto AF ® + | — | — | 0.00 | 0.00 |
| | Hylenex ® | | | | |
| | AD2CD2_SC + | 8.38 | 24.00 | 0.24 | 7.75 |
| | Hylenex ® | | | | |
| Median | ReFacto AF ® + | — | — | 0.00 | 0.00 |
| | Hylenex ® | | | | |
| | AD2CD2_SC + | 10.45 | 24.00 | 0.19 | 7.07 |
| | Hylenex ® | | | | |

Analysis of bioavailability is based on mean and median activities for AD2CD2_SC plus Hylenex® in subcutaneous and intravenous administration from prior studies. The bioavailability is calculated according to the following formula:

$$F [ \% ] = \frac{{AUC}_{0 - inf , sc}}{{AUC}_{0 - inf , iv}} * 100$$

Where

AUC_{0-inf} is the AUC from dosing time extrapolated to infinity, based on the last observed concentration (Cₗₐₛₜ), i.e., the elimination rate constant λ_{z} is used to estimate the AUC_{t-inf} (Cₗₐₛₜ/λ_{z}) from the last observed concentration until the time point of concentration zero is reached, which is added to the AUC₀₋ₜ, calculated for the period from predose, which is at maximum 2 h before injection, over the maximum observed blood concentration possible until the lower limit of quantification (LLOQ), but at least until a concentration of 0.01 U/mL is reached:

$${AUC}_{0 - inf} = {AUC}_{0 - t} + \frac{{C}_{last}}{{λ}_{z}}$$

Table C compares the bioavailability of AD2CD2_SC upon s.c. administration with or without Hylenex®.

TABLE C

| | | Half-life | | Cₘₐₓ | AUC_{0-inf} | |
|---|---|---|---|---|---|---|
| | Test item | [h] | Tₘₐₓ [h] | [U/mL] | [U/mL*h] | F [%] |
| Mean | s.c. AD2CD2_SC + | 8.73 | 12.00 | 0.26 | 6.79 | 15.3 |
| | Hylenex ® | | | | | |
| | i.v. AD2CD2_SC | 12.00 | 0.50 | 3.06 | 44.47 | |
| Median | s.c. AD2CD2_SC + | 22.76 | 12.00 | 0.26 | 7.87 | 18.6 |
| | Hylenex ® | | | | | |
| | i.v. AD2CD2_SC | 10.33 | 0.50 | 3.28 | 42.39 | |

In this study AD2CD2_SC plus Hylenex® and ReFacto AF® plus Hylenex® (each 200 U/kg b.w.) are compared after subcutaneous administration in hemophilia A mice for up to 60 hours. FVIII activity (chromogenic activity) and human FVIII antigen (ELISA) have been analysed. ReFacto AF® co-administered with Hylenex® did not demonstrate relevant FVIII plasma levels after s.c. injections. In comparison, AD2CD2_SC co-administered with Hylenex® resulted in considerable FVIII plasma levels even up to 60 hours. The combined (s.c. depot+plasma) half-life of AD2CD2_SC was 8.73 h (mean) and 22.76 h (median) based on the chromogenic activity, whereas ReFacto® was not really detectable at all after subcutaneous administration. It is noted that, due to the relatively low numbers of animals, outliers have a higher influence on the mean than on the median. The bioavailability of AD2CD2_SC in the mice was approx. 18%. This was calculated by comparing the area under the curve (AUC) with the AUC of identical i.v. doses from previous studies.

5.2 Pharmacokinetic Study of Recombinant FVIII Molecule AD2CD2 SC in Minipigs by a Comparative Study with Intravenous and Subcutaneous Administration

To further investigate the FVIII proteins comprising at least one albumin binding domain after subcutaneous administration, the minipig, in particular, the Göttingen minipig (Ellegaard, Dalmos, DK) was chosen as a relevant model based on the similarity of porcine and human dermal tissue. In this study, a FVIII-ABD fusion molecule having 19 deimmunizing amino acid substitutions within the FVIII regions incorporated to prevent human FVIII inhibitor development or potentially enable a certain bypassing activity in case of present inhibitory anti-FVIII antibodies was also tested. This molecule is designated as AD2CD2-19M_SC.

5.2.1 Comparative Study with Intravenous Administration in Minipigs

To compare the observed effect of the construct AD2CD2_SC after subcutaneous administration, firstly the construct was investigated for its pharmacokinetic properties and to determine a baseline level after intravenous administration in the minipig model. Therefore, 18 male naiive Göttingen minipigs (3 per group) were intravenously treated with 30 U/kg FVIII:Ag of either ReFacto AF® or AD2CD2_SC or AD2CD2-19M_SC co-formulated with either 1 or 10% human albumin (Albiomin® 20%, Biotest). Blood samples (˜1.8 mL) were taken at the timepoints: predose, 0.5, 4, 12, 24, 36, 48, 72, 96, 120 and 144 h post dose. Sodium citrate was used as anti-coagulant. Plasma was directly isolated by centrifugation.

Plasma samples were measured by FVIII antigen ELISA. As albumin-binding to AD2CD2_SC and AD2CD2-19M_SC had an influence on antibody-binding, a correction factor was determined (see below in method section).

In order to evaluate if measurements of chromogenic FVIII activity, clotting FVIII activity and FVIII antigen ELISA are applicable with the Göttingen minipig plasma matrix, preceding tests were performed by spiking ReFacto AF® and AD2CD2_SC in minipig plasma and determining the recovery. As the minipigs are not hemophilia minipigs, they have endogenous FVIII activity. Thus, for chromogenic FVIII activity and clotting FVIII activity, high background levels of approx. 4 to 7.5 U/ml were found. Thus, low concentrations of recombinant FVIII, e.g. at the terminal phase of a pharmacokinetic study, disappear in this high background. In comparison, the FVIII antigen ELISA was found to be specific for human FVIII and it does not detect porcine FVIII. However, interactions of ReFacto AF® and especially AD2CD2_SC and AD2CD2-19M_SC with porcine von-Willbrand Factor (vWF) and porcine and human albumin demonstrate influences on the measurements. Therefore, the FVIII Antigen ELISA was adapted as described in the method section.

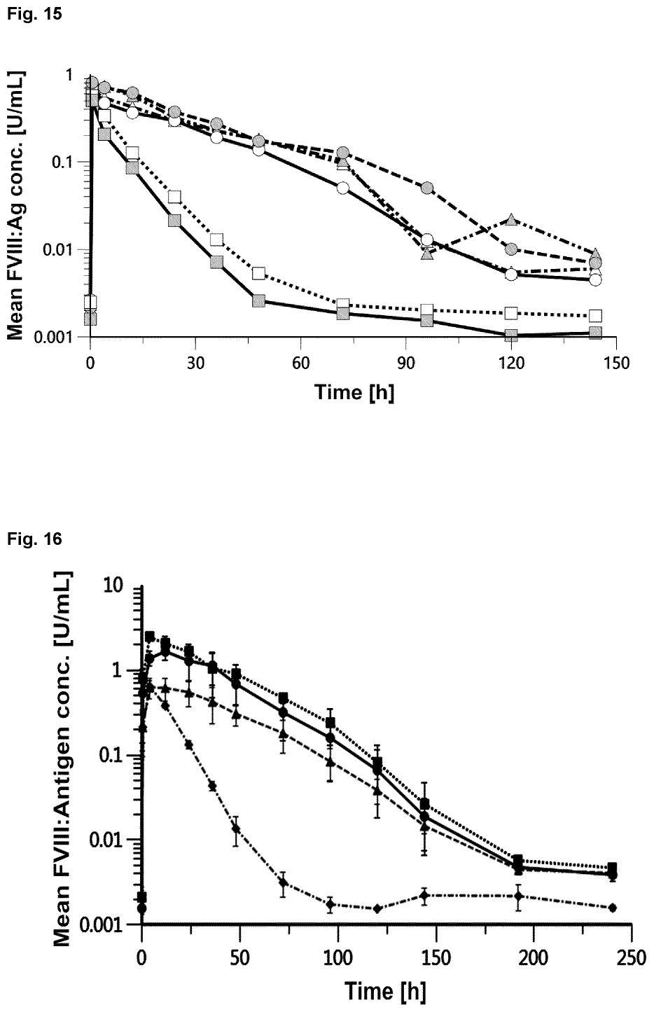

Mean FVIII plasma concentrations based on FVIII:Ag measurements after intravenous injection of 30 U FVIII:Ag/kg bodyweight are shown in FIG. 15 and Table D below.

TABLE D

| | Mean | | Median | |
|---|---|---|---|---|
| | | HL- | | HL- |
| | t_{1/2} | extension | t_{1/2} | extension |
| Component | [h] | [x-times] | [h] | [x-times] |
| ReFacto AF ® + 1% HSA | 7.07 | 1 | 6.28 | 1 |
| ReFacto AF ® + 10% HSA | 6.44 | 1 | 5.96 | 1 |
| AD2CD2_SC + 1% HSA | 18.63 | 2.64 | 18.62 | 2.96 |
| AD2CD2_SC + 10% HSA | 20.68 | 3.21 | 20.82 | 3.49 |
| AD2CD2-19M_SC + 1% | 19.15 | 2.71 | 18.70 | 2.98 |
| HSA | | | | |
| AD2CD2-19M_SC + 10% | 21.00 | 3.26 | 20.52 | 3.44 |
| HSA | | | | |

In a comparative study 18 minipigs were intravenously treated with 30 U/kg (based on FVIII:Ag) of either ReFacto AF®, AD2CD2_SC or AD2CD2-19M_SC in combination with human serum albumin (HSA) at 1% and 10%. Plasma samples were taken at several time points, analyzed by FVIII:Ag ELISA and raw values were evaluated by performing a non-compartmental analysis (NCA) using Phoenix WinNonlin. ReFacto AF® resulted in a terminal half-life of approx. 7 h, while half-lives for AD2CD2_SC and AD2CD2-19M_SC resulted in approx. 19 h in the presence of 1% HSA or in approx. 21 h in the presence of 10% HSA (mean values). Thus, incorporation of four albumin binding domains led to a half-life (HL) extension in intravenous administration of up to 3.3-times in this model compared to ReFacto AF®.

5.2.2 Pharmacokinetic Study with Subcutaneous Administration in Minipigs

The aim of this study was to investigate the bioavailability and pharmacokinetics of AD2CD2-19M_SC in the presence of Hylenex® (recombinant human hyaluronidase) plus 1% human albumin or in the presence of 3% albumin after subcutaneous (s.c.) administration in minipigs (twelve male Göttingen minipigs).

AD2CD2-19M_SC was compared to the commercially available recombinant FVIII product ReFacto AF® co-administered with Hylenex® plus 1% human albumin. The administered dose for AD2CD2-19M_SC respectively ReFacto AF® was 300 U FVIII:Ag/kg b.w., one group obtained a dose for AD2CD2-19M_SC of 150 U/kg b.w. The dose for co-administered Hylenex® was 16.13 U/kg b.w. Coagulation factors were administered by a single s.c. injection of the minipigs behind the ear. 1 h prior to FVIII administration, all animals were dosed via intravenous (i.v.) injection (saphenous vein) with 1.25 mL Albiomin® 20% (a 200 mg/mL HSA solution, Biotest AG, Dreieich)/kg bodyweight. Blood samples were collected from the vena cava into commercial vacuum blood collection tubes containing sodium citrate at the time points: pre-dose, 0.5, 4, 12, 24, 36, 48, 72, 96, 120, 144, 192 and 240 h post dose. Plasma samples were further analyzed for human FVIII antigen (ELISA) adapted to minipig matrix as described above.

The dose formulation preparation and the dosing regime was is specified in Table E.

TABLE E
Dose Formulation Preparation
Each formulation was prepared immediately prior to dose administration based on the method
supplied by the sponsor. The details of each preparation are shown in the table below:

| | | | | | Volume | | | |
|---|---|---|---|---|---|---|---|---|
| | | | Volume of | Volume of | of | Volume of | Total Volume | |
| | | | Test Item | Albumin | Hylenex | Formulation | of | Formulation |
| | | | Added | Added | Added | Buffer | Formulation | Concentration |
| Group | Test Item | Vehicle | (mL) | (mL) | (mL) | Added (mL) | (mL) | (U/mL) |
| 1 | ReFacto ® | 1% HSA// | 8.4 | 0.6 | 3 | N/A | 12 | 700 |
| | | Hylenex | | | | | | |
| 2 | AD2CD2- | 3% HSA | 8.4 | 1.8 | N/A | 1.8 | 12 | 700 |
| | 19M_SC | | | | | | | |
| 3 | AD2CD2- | 1% HSA// | 8.4 | 0.6 | 3 | N/A | 12 | 700 |
| | 19M_SC | Hylenex | | | | | | |
| 4 | AD2CD2- | 1% HSA// | 4.2 | 0.6 | 3 | 4.2 | 12 | 350 |
| | 19M_SC | Hylenex | | | | | | |

Dosing Regimen
Animals were dosed via intravenous (IV) injection with 1.25 mL/kg Albiomin ® 20% (a 200 mg/ml HSA solution)
ca 1 h before dosing test items. Animals were dosed subcutaneously (SC) according to the following design:

| | | | | Dose | |
|---|---|---|---|---|---|
| | | | Dose Level | Concentration | Dose Volume |
| Group | Test Item/Dose Vehicle | Route | (U/kg b.w.) | (U/mL) | (mL/kg b.w.) |
| 1 | ReFacto ®//1% HSA//Hylenex ® | SC | 300 | 700 | 0.43 |
| 2 | AD2CD2-19M_SC//3% HSA | | | | |
| 3 | AD2CD2-19M_SC//1% HSA//Hylenex ® | | | | |
| 4 | AD2CD2-19M_SC//1% HSA//Hylenex ® | | 150 | 350 | |

Mean FVIII plasma concentrations based on FVIII:Ag measurements after subcutaneous injection of 300 U/kg or 150 U/kg FVIII are shown in FIG. 16. The bioavailability of each group containing AD2CD2-19M_SC was calculated based on data observed from the previous intravenous treatment of AD2CD2-19M_SC containing 1% Albiomin®. Intravenous and subcutaneous ReFacto groups were used to calculate its subcutaneous bioavailability. Each bioavailability was calculated as described in 5.1.2. Results are demonstrated in Table F.

TABLE F

| | FVIII | Half- | Cₘₐₓ | AUCₗₐₛₜ | | Bioavail- |
|---|---|---|---|---|---|---|
| Construct | dose | life (h) | (U/ml) | (U/ml*h) | HLE* | ability (%) |
| ReFacto AF ®//1% | 300 U/kg | 8.55 | 0.65 | 10.70 | 1 | 20 |
| HSA//Hylenex ® | | | | | | |
| AD2CD2-19M_SC// | 300 U/kg | 19.54 | 1.67 | 81.17 | 2.3 | 38 |
| 3% HSA | | | | | | |
| AD2CD2-19M_SC// | 300 U/kg | 20.33 | 2.47 | 105.39 | 2.4 | 50 |
| 1% HSA//Hylenex ® | | | | | | |
| AD2CD2-19M_SC// | 150 U/kg | 23.47 | 0.63 | 35.61 | 2.75 | 34 |
| 1% HSA//Hylenex ® | | | | | | |

*HLE = Half-life extension

These results show that in minipigs, the pharmaceutical compositions of the invention have an even better bioavailability after s.c. administration than in mice, with a relative increase of at least 70% with a reduced dose of FVIII-ABD compared to higher dose of ReFacto®, while with the same dose of FVIII-ABD, there was an relative increase in bioavailability of at least 150%. Even without Hylenex®, but with an increased amount of human serum albumin, the bioavailability was 90% increased compared to ReFacto®.

The study was performed in 12 Göttingen minipigs. 300 U/kg or 150 U/kg of either ReFacto AF® or AD2CD2-19M_SC were injected subcutaneously in the presence of human serum albumin (HSA) and in the presence or absence of Hylene®. Bioavailability of up to 50% were observed for AD2CD2-19M_SC at doses of 300 U/kg in the presence of 1% HSA and Hylenex in comparison to the intravenous treatment of the comparative study. In comparison, ReFacto AF® resulted in 20% bioavailability. In the absence of Hylenex® and in the presence of 3% HSA, bioavailability of AD2CD2-19M_SC was only slightly lower with 38%. Regarding the time-vs-concentration areas under the curve until the last measured concentration (AUCₗₐₛₜ), ReFacto AF® resulted in approx. 11 U/ml*h, while for AD2CD2-19M_SC AUCₗₐₛₜ values of 81 (with 3% HSA) and 105 (with 1% HSA and Hylene®) were observed. The results of all studies demonstrate a clear benefit of FVIII-ABD (with or without 19M) in plasma half-life, but especially in the opportunity to dose FVIII subcutaneously.

1. A Factor VIII (FVIII) protein comprising at least one albumin binding domain, wherein the bioavailability of the Factor VIII protein after subcutaneous administration is at least 25% as measured in minipigs,
for use in treatment of a subject having hemophilia A, wherein a dose of 1-1000 U/kg bodyweight is administered to the subject subcutaneously.

2. The Factor VIII protein for use of claim 1, wherein the bioavailability of the Factor VIII protein after subcutaneous administration is at least 30%, optionally, 30-60% as measured in minipigs.

3. The Factor VIII protein for use of claim 1, wherein the Factor VIII protein comprises at least two albumin-binding domains.

4. The Factor VIII protein for use of claim 1, wherein the FVIII protein is a single chain protein, wherein the FVIII protein preferably is at least partly B domain deleted.

5. The Factor VIII protein for use of claim 1, wherein the FVIII protein comprises a heavy chain portion and a light chain portion of Factor VIII, and wherein the albumin binding domain(s) is/are C-terminal to the heavy chain portion and/or C-terminal to the light chain portion,
wherein, if the protein is a single chain protein, the albumin binding domain(s) is/are between the heavy chain portion and the light chain portion and/or C-terminal to the light chain portion.

6. The Factor VIII protein for use of claim 5, wherein at least one albumin binding domain is C-terminal to the heavy chain portion and, if the protein is a single chain protein, between the heavy chain portion and the light chain portion, and at least one albumin binding domain is C-terminal to the light chain portion,
wherein, preferably, two albumin binding domains are C-terminal to the heavy chain portion and, if the protein is a single chain protein, between the heavy chain portion and the light chain portion, and two albumin binding domains are C-terminal to the light chain portion.

7. The Factor VIII protein for use of claim 1, wherein albumin binding domains are separated from the heavy chain portion and/or the light chain portion and/or other albumin-binding domains by a linker selected from the group comprising
a) a linker comprising a thrombin-cleavable linker that optionally has the sequence of SEQ ID NO: 39, and b) a linker comprising a glycine-serine linker that optionally has the sequence of SEQ ID NO: 40 or SEQ ID NO: 41, and c) a linker comprising a thrombin-cleavable linker flanked on each side by a glycine-serine linker that optionally has the sequence of SEQ ID NO: 42 or SEQ ID NO: 43.

8. The Factor VIII protein for use of claim 1, wherein the albumin binding domain comprises a sequence according to SEQ ID NO: 44, wherein, preferably, the sequence is SEQ ID NO: 46.

9. The Factor VIII protein for use of claim 1, wherein the FVIII protein optionally is a single chain protein, wherein said protein comprises a heavy chain portion having at least 90% sequence identity to aa20-aa768 of SEQ ID NO: 16 and a light chain portion having at least 90% sequence identity to aa769-aa1445 of SEQ ID NO: 16.

10. The Factor VIII protein for use of claim 1, wherein the FVIII protein is a single chain protein comprising at least two albumin binding domains between the heavy chain portion and the light chain portion and at least two albumin binding domain C-terminal to the light chain portion, wherein the protein has at least 80% sequence identity to any of SEQ ID NO: 48, 49 or 51,
wherein the protein preferably has at least 80% sequence identity to SEQ ID NO: 48, wherein the protein optionally comprises SEQ ID NO: 48.

11. The Factor VIII protein for use of claim 1, wherein the protein has at least 90% sequence identity to a Factor VIII protein of SEQ ID NO: 63, wherein only the A1, a1, A2, a2, a3, A3, C1 and C2 domains are considered for determination of sequence identity, wherein the protein optionally has SEQ ID NO: 114.

12. A pharmaceutical composition comprising the FVIII protein for use of claim 1, wherein the composition preferably is for human administration and optionally comprises a pharmaceutically acceptable excipient.

13. The pharmaceutical composition for use of claim 12, further comprising human albumin, wherein, preferably, the concentration of human albumin is 0.1-15% (w/v).

14. The pharmaceutical composition for use of claim 12, further comprising a hyaluronidase, preferably, a human hyaluronidase, wherein the dose of the hyaluronidase per injection optionally is 50-300 U.

15. A kit comprising a hyaluronidase, preferably, a human hyaluronidase, and a Factor VIII protein comprising at least one albumin binding domain(s),
wherein, optionally, the Factor VIII protein comprises a heavy chain portion and a light chain portion of Factor VIII and the albumin binding domain(s) is/are C-terminal to the heavy chain portion and/or C-terminal to the light chain portion, wherein, if the protein is a single chain protein, the albumin binding domain(s) is/are between the heavy chain portion and the light chain portion and/or C-terminal to the light chain portion.
